(19) United States
(12) Patent Application Publication
(10) Pub. No.: US 2005/0239853 A1
(43) Pub. Date: Oct. 27, 2005

(54) New compounds

(76) Inventors: Tjeerd Barf, Uppsala (SE); Guido Kurz, Stockholm (SE); Sofia Nordin, Uppsala (SE); Lars Tedenborg, Uppsala (SE); Jerk Vallgarda, Uppsala (SE); Meredith Williams, Uppsala (SE)

Correspondence Address: FISH & RICHARDSON PC, P.O. BOX 1022, MINNEAPOLIS, MN, 55440-1022, US

(21) Appl. No.: 11/050,178

(22) Filed: Feb. 3, 2005

Related U.S. Application Data
(60) Provisional application No. 60/555,808, filed on Mar. 24, 2004.
(60) Provisional application No. 60/650,777, filed on Jan. 31, 2005.

(30) Foreign Application Priority Data
Feb. 4, 2004 (SE) 0400227-5
May 24, 2004 (SE) 0401324-9
Oct. 15, 2004 (SE) 0402509-4

(51) Int. Cl. A61K 31/427; C07D 417/02

(52) U.S. Cl. 514/370; 548/181

(57) ABSTRACT

The present invention relates to compounds with the formula (I) 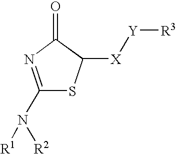
wherein R¹, R², R³, X, and Y are as defined herein, and also to pharmaceutical compositions comprising the compounds, as well as to the use of the compounds in medicine and for the preparation of a medicament which acts on the human 11-hydroxysteroid dehydrogenase type 1 enzyme.

### RELATED APPLICATIONS

This application claims priority to Swedish application number 0400227-5, filed on Feb. 4, 2004; Swedish application number 0401324-9, filed on May 24, 2004; Swedish application number 0402509-4, filed on Oct. 15, 2004; U.S. provisional application Ser. No. 60/555,808, filed on Mar. 24, 2004, and U.S. provisional application Ser. No.______, filed on Jan. 31, 2005, attorney docket number 13425-160P01, titled “New Compounds,” having the following inventors: Martin Henriksson, Evert Homan, Lars Johansson, Jerk Vallgårda and Meredith Willaims, the contents of each of which is incorporated herein by reference.

### TECHNICAL FIELD

The present invention relates to novel compounds, to pharmaceutical compositions comprising the compounds, as well as to the use of the compounds in medicine and for the preparation of a medicament which acts on the human 11-β-hydroxysteroid dehydrogenase type 1 enzyme (11βHSD1).

### BACKGROUND

1. Glucorticoids, Diabetes and Hepatic Glucose Production

It has been known for more than half a century that glucocorticoids have a central role in diabetes. For example, the removal of the pituitary gland or the adrenal gland from a diabetic animal alleviates the most severe symptoms of diabetes and lowers the concentration of glucose in the blood (Long, C. D. and Leukins, F. D. W. (1936) J. Exp. Med. 63: 465-490; Houssay, B. A. (1942) Endocrinology 30: 884-892). It is also well established that glucocorticoids enable the effect of glucagon on the liver.

The role of 11βHSD1 as an important regulator of local glucocorticoid effect and thus of hepatic glucose production is well substantiated (see, e.g., Jamieson et al. (2000) J. Endocrinol. 165: 685-692). Hepatic insulin sensitivity was improved in healthy human volunteers treated with the non-specific 11βHSD1 inhibitor carbenoxolone (Walker, B. R. et al. (1995) J. Clin. Endocrinol. Metab. 80: 3155-3159). Furthermore, the expected mechanism has been established by different experiments with mice and rats. These studies showed that the mRNA levels and activities of two key enzymes in hepatic glucose production were reduced, namely: the rate-limiting enzyme in gluconeogenesis, phosphoenolpyruvate carboxykinase (PEPCK), and glucose-6-phosphatase (G6Pase), the enzyme catalyzing the last common step of gluconeogenesis and glycogenolysis. Finally, blood glucose levels and hepatic glucose production are reduced in mice in which the 11βHSD1 gene is knocked-out. Data from this model also confirm that inhibition of 11βHSD1 will not cause hypoglycemia, as predicted since the basal levels of PEPCK and G6Pase are regulated independently of glucocorticoids (Kotelevtsev, Y. et al., (1997) Proc. Natl. Acad. Sci. USA 94: 14924-14929).

2. Possible Reduction of Obesity and Obesity Related Cardiovascular Risk Factors

Obesity is an important factor in syndrome X as well as in the majority (>80%) of type 2 diabetes, and omental fat appears to be of central importance. Abdominal obesity is closely associated with glucose intolerance, hyperinsulinemia, hypertriglyceridemia, and other factors of the so-called syndrome X (e.g., increased blood pressure, decreased levels of HDL and increased levels of VLDL) (Montague & O'Rahilly, Diabetes 49: 883-888, 2000). Inhibition of the 11βHSD1 in pre-adipocytes (stromal cells) has been shown to decrease the rate of differentiation into adipocytes. This is predicted to result in diminished expansion (possibly reduction) of the omental fat depot, i.e., reduced central obesity (Bujalska, I. J., S. Kumar, and P. M. Stewart (1997) Lancet 349: 1210-1213).

Inhibition of 11βHSD1 in mature adipocytes is expected to attenuate secretion of the plasminogen activator inhibitor 1 (PAI-1)—an independent cardiovascular risk factor (Halleux, C. M. et al. (1999) J. Clin. Endocrinol. Metab. 84: 4097-4105). Furthermore, there is a clear correlation between glucocorticoid “activity” and cardiovascular risk factor suggesting that a reduction of the glucocorticoid effects would be beneficial (Walker, B. R. et al. (1998) Hypertension 31: 891-895; Fraser, R. et al. (1999) Hypertension 33: 1364-1368).

Adrenalectomy attenuates the effect of fasting to increase both food intake and hypothalamic neuropeptide Y expression. This supports the role of glucocorticoids in promoting food intake and suggests that inhibition of 11βHSD1 in the brain might increase satiety and therefore reduce food intake (Woods, S. C. et al. (1998) Science, 280: 1378-1383).

3. Possible Beneficial Effect on the Pancreas

Inhibition of 11βHSD1 in isolated murine pancreatic β-cells improves glucose-stimulated insulin secretion (Davani, B. et al. (2000) J. Biol. Chem. 2000 Nov. 10; 275(45): 34841-4). Glucocorticoids were previously known to reduce pancreatic insulin release in vivo (Billaudel, B. and B. C. J. Sutter (1979) Horm. Metab. Res. 11: 555-560). Thus, inhibition of 11βHSD1 is predicted to yield other beneficial effects for diabetes treatment, besides the effects on liver and fat.

4. Possible Beneficial Effects on Cognition and Dementia

Stress and glucocorticoids influence cognitive function (de Quervain, D. J.-F., B. Roozendaal, and J. L. McGaugh (1998) Nature 394: 787-790). The enzyme 11βHSD1 controls the level of glucocorticoid action in the brain and thus contributes to neurotoxicity (Rajan, V., C. R. W. Edwards, and J. R. Seckl, J. (1996) Neuroscience 16: 65-70; Seckl, J. R., Front. (2000) Neuroendocrinol. 18: 49-99). Unpublished results indicate significant memory improvement in rats treated with a non-specific 11βHSD1 inhibitor (J. Seckl, personal communication). Based the above and on the known effects of glucocorticoids in the brain, it may also be suggested that inhibiting 11βHSD1 in the brain may result in reduced anxiety (Tronche, F. et al. (1999) Nature Genetics 23: 99-103). Thus, taken together, the hypothesis is that inhibition of 11βHSD1 in the human brain would prevent reactivation of cortisone into cortisol and protect against deleterious glucocorticoid-mediated effects on neuronal survival and other aspects of neuronal function, including cognitive impairment, depression, and increased appetite.

5. Possible Use of Immuno-Modulation using 11βHSD1 Inhibitors

The general perception is that glucocorticoids suppress the immune system. But in fact there is a dynamic interaction between the immune system and the HPA (hypothalamo-pituitary-adrenal) axis (Rook, G. A. W. (1999) Baillièr's Clin. Endocrinol. Metab. 13: 576-581). The balance between the cell-mediated response and humoral responses is modulated by glucocorticoids. A high glucocorticoid activity, such as at a state of stress, is associated with a humoral response. Thus, inhibition of the enzyme 11βHSD1 has been suggested as a means of shifting the response towards a cell-based reaction.

In certain disease states, including tuberculosis, lepra and psoriasis the immune reaction is normaly biased towards a humoral response when in fact the appropriate response would be cell based. Temporal inhibition of 11βHSD1, local or systemic, might be used to push the immune system into the appropriate response (Mason, D. (1991) Immunology Today 12: 57-60; Rook et al., supra).

An analogous use of 11βHSD1 inhibition, in this case temporal, would be to booster the immune response in association with immunization to ensure that a cell based response would be obtained, when desired.

6. Reduction of Intraocular Pressure

Recent data suggest that the levels of the glucocorticoid target receptors and the 11βHSD enzymes determines the susceptibility to glaucoma (Stokes, J. et al. (2000) Invest. Ophthalmol. 41: 1629-1638). Further, inhibition of 11βHSD1 was recently presented as a novel approach to lower the intraocular pressure (Walker E. A. et al, poster P3-698 at the Endocrine society meeting Jun. 12-15, 1999, San Diego). Ingestion of carbenoxolone, a non-specific inhibitor of 11βHSD1, was shown to reduce the intraocular pressure by 20% in normal subjects. In the eye, expression of 11βHSD1 is confined to basal cells of the corneal epithelium and the non-pigmented epithelialium of the cornea (the site of aqueous production), to ciliary muscle and to the sphincter and dilator muscles of the iris. In contrast, the distant isoenzyme 11βHSD2 is highly expressed in the non-pigmented ciliary epithelium and corneal endothelium. None of the enzymes is found at the trabecular meshwork, the site of drainage. Thus, 11βHSD1 is suggested to have a role in aqueous production, rather than drainage, but it is presently unknown if this is by interfering with activation of the glucocorticoid or the mineralocorticoid receptor, or both.

7. Reduced Osteoporosis

Glucocorticoids have an essential role in skeletal development and function but are detrimental in excess. Glucocorticoid-induced bone loss is derived, at least in part, via inhibition of bone formation, which includes suppression of osteoblast proliferation and collagen synthesis (Kim, C. H., Cheng, S. L. and Kim, G. S. (1999) J. Endocrinol. 162: 371-379). The negative effect on bone nodule formation could be blocked by the non-specific inhibitor carbenoxolone suggesting an important role of 11βHSD1 in the glucocorticoid effect (Bellows, C. G., Ciaccia, A. and Heersche, J. N. M. (1998) Bone 23: 119-125). Other data suggest a role of 11βHSD1 in providing sufficiently high levels of active glucocorticoid in osteoclasts, and thus in augmenting bone resorption (Cooper, M. S. et al. (2000) Bone 27: 375-381). Taken together, these different data suggest that inhibition of 11βHSD1 may have beneficial effects against osteoporosis by more than one mechanism working in parallel.

8. Reduction of Hypertension

Bile acids inhibit 11β-hydroxysteroid dehydrogenase type 2. This results in a shift in the overall body balance in favour of cortisol over cortisone, as shown by studying the ratio of the urinary metabolites (Quattropani C., Vogt B., Odermatt A., Dick B., Frey B. M., Frey F. J. (2001) J Clin Invest. November;108(9):1299-305. “Reduced activity of 11beta-hydroxysteroid dehydrogenase in patients with cholestasis”.). Reducing the activity of 11βHSD1 in the liver by a selective inhibitor is predicted to reverse this imbalance, and acutely counter the symptoms such as hypertension, while awaiting surgical treatment removing the biliary obstruction.

9. Wound Healing

Cortisol performs a broad range of metabolic functions and other functions. The multitude of glucocorticoid action is exemplified in patients with prolonged increase in plasma glucocorticoids, so called “Cushing's syndrome”. Patients with Cushing's syndrome have prolonged increase in plasma glucocorticoids and exhibit impaired glucose tolerance, type 2 diabetes, central obesity, and osteoporosis. These patients also have impaired wound healing and brittle skin (Ganong, W. F. Review of Medical Physiology. Eighteenth edition ed. Stamford, Conn.: Appleton & Lange; 1997).

Glucocorticoids have been shown to increase risk of infection and delay healing of open wounds (Anstead, G. M. Steroids, retinoids, and wound healing. Adv Wound Care 1998;11(6):277-85). Patients treated with glucocorticoids have 2-5-fold increased risk of complications when undergoing surgery (Diethelm, A. G. Surgical management of complications of steroid therapy. Ann Surg 1977;185(3):251-63).

The European patent application No. EP 0902288 discloses a method for diagnosing the status of wound healing in a patient, comprising detecting cortisol levels in said wound. The authors suggest that elevated levels of cortisol in wound fluid, relative to normal plasma levels in healthy individuals, correlates with large, non-healing wounds (Hutchinson, T. C., Swaniker, H. P. Wound diagnosis by quantitating cortisol in wound fluids. European patent application No. EP 0 902 288, published 17, Mar. 1999).

In humans, the 11βHSD catalyzes the conversion of cortisol to cortisone, and vice versa. The parallel function of 11βHSD in rodents is the interconversion of corticosterone and 11-dehydrocorticosterone (Frey F. J., Escher, G., Frey, B. M. Pharmacology of 11 beta-hydroxysteroid dehydrogenase. Steroids 1994;59(2):74-9). The ratio of total cortisone to cortisol in human plasma is 0.2 in adults. However, the concentration of free cortisol and cortisone are almost equal, since most cortisol, but very little cortisone is protein bound. Cortisone thus functions as a large precursor pool for active glucocorticoids (Hammami, M. M, Siiteri, P. K. Regulation of 11 beta-hydroxysteroid dehydrogenase activity in human skin fibroblasts: enzymatic modulation of glucocorticoid action. J Clin Endocrinol Metab 1991;73(2):326-34). Two isoenzymes of 11βHSD, 11βHSD1 and 11βHSD2, have been characterized, and differ from each other in function and tissue distribution (Albiston, A. L., Obeyesekere, V. R., Smith, R. E., Krozowski, Z. S., Cloning and tissue distribution of the human 11 beta-hydroxysteroid dehydrogenase type 2 enzyme. Mol Cell Endocrinol 1994;105(2):R11-7).

Mineralocorticoid receptors (MR) have similar affinity for cortisol and aldosterone, the active mineralocorticoid and circulating levels of glucocorticoids are substantially higher than levels of mineralocorticoids. Thus, for selectivity of mineralocorticoids in mineralocorticoid-target tissues, an additional mechanism must operate. This paradox led to the finding of the physiological role of 11βHSD2 in kidneys and other mineralocorticoid tissues. 11βHSD2 catalyses the inactivation of cortisol to cortisone, and thereby protects MR from circulating glucocorticoids and confers specificity of aldosterone for MR (Funder, J. W., Pearce, P. T., Smith, R., Smith, A. I. Mineralocorticoid action: target tissue specificity is enzyme, not receptor, mediated. Science 1988;242(4878):583-5). 11βHSD2 is expressed in kidney, salivary glands, placenta, ileum, distal colon and epithelia of respiratory tract, where it is co-localized with MR (Hirasawa, G., Sasano, H., Takahashi, K., Fukushima, K., Suzuki, T., Hiwatashi, N., et al. Colocalization of 11 beta-hydroxysteroid dehydrogenase type II and mineralocorticoid receptor in human epithelia. J Clin Endocrinol Metab 1997;82(11):3859-63; Krozowski, Z., MaGuire, J. A., Stein-Oakley, A. N., Dowling, J., Smith, R. E., Andrews, R,K. Immunohistochemical localization of the 11 beta-hydroxysteroid dehydrogenase type II enzyme in human kidney and placenta. J Clin Endocrinol Metab 1995;80(7):2203-9).

In vitro 11β-HSD1 has the capacity to act both as oxidase and reductase, but in vivo it mainly functions as a reductase, i.e. converting cortisone to cortisol and thereby locally increase glucocorticoid action. In contrast to 11β-HSD2 which uses NAD as co-factor, 11β-HSD1 is NADP dependent (Mercer W R, Krozowski Z S. Localization of an 11 beta hydroxysteroid dehydrogenase activity to the distal nephron. Evidence for the existence of two species of dehydrogenase in the rat kidney. Endocrinology 1992; 130(1):540-3). Like GR, 11β-HSD1 is expressed in numerous tissues like liver, adipose tissue, adrenal cortex, gonads, lung, pituitary, brain, eye etc (Monder C, White P C. 11 beta-hydroxysteroid dehydrogenase. Vitam Horm 1993;47:187-271; Stewart P M, Krozowski Z S. 11 beta-Hydroxysteroid dehydrogenase. Vitam Horm 1999;57:249-324; Stokes J, Noble J, Brett L, Phillips C, Seckl J R, O'Brien C, et al. Distribution of glucocorticoid and mineralocorticoid receptors and 11beta-hydroxysteroid dehydrogenases in human and rat ocular tissues. Invest Ophthalmol Vis Sci 2000;41(7): 1629-38). The function of 11β-HSD1 is to fine-tune local glucocorticoid action. It acts to amplify glucocorticoid action in certain cells to maintain basal metabolic function during for example the diurnal nadir of glucocorticoid secretion. 11β-HSD activity has been shown in the skin of humans and rodents, in human fibroblasts and in rat skin pouch tissue (Hammami et al., supra; Cooper M S, Moore J, Filer A, Buckley C D, Hewison M, Stewart P M. 11beta-hydroxysteroid dehydrogenase in human fibroblasts: expression and regulation depends on tissue of origin. ENDO 2003 Abstracts 2003; Teelucksingh S, Mackie A D, Burt D, McIntyre M A, Brett L, Edwards CR. Potentiation of hydrocortisone activity in skin by glycyrrhetinic acid. Lancet 1990;335(8697):1060-3; Slight S H, Chilakamarri V K, Nasr S, Dhalla A K, Ramires F J, Sun Y, et al. Inhibition of tissue repair by spironolactone: role of mineralocorticoids in fibrous tissue formation. Mol Cell Biochem 1998; 189(1-2):47-54).

Wound healing consists of serial events including inflammation, fibroblast proliferation, secretion of ground substances, collagen production, angiogenesis, wound contraction and epithelialization. It can be divided in three phases; inflammatory, proliferative and remodeling phase (reviewed in Anstead et al., supra).

In surgical patients, treatment with glucocorticoids increases risk of wound infection and delay healing of open wounds. It has been shown in animal models that restraint stress slows down cutaneous wound healing and increases susceptibility to bacterial infection during wound healing. These effects were reversed by treatment with the glucocorticoid receptor antagonist RU486 (Mercado, A. M., Quan, N., Padgett, D. A., Sheridan, J. F., Marucha, P. T. Restraint stress alters the expression of interleukin-1 and keratinocyte growth factor at the wound site: an in situ hybridization study. J Neuroimmunol 2002;129(1-2):74-83; Rojas, I. G., Padgett, D. A., Sheridan, J. F., Marucha, P. T. Stress-induced susceptibility to bacterial infection during cutaneous wound healing. Brain Behav Immun 2002;16(1):74-84). Glucocorticoids produce these effects by suppressing inflammation, decrease wound strength, inhibit wound contracture and delay epithelialization (Anstead et al., supra). Glucocorticoids influence wound healing by interfering with production or action of cytokines and growth factors like IGF, TGF-β, EGF, KGF and PDGF (Beer, H. D., Fassler, R., Werner, S. Glucocorticoid-regulated gene expression during cutaneous wound repair. Vitam Horm 2000;59:217-39; Hamon, G. A., Hunt, T. K., Spencer, E. M. In vivo effects of systemic insulin-like growth factor-I alone and complexed with insulin-like growth factor binding protein-3 on corticosteroid suppressed wounds. Growth Regul 1993;3(1):53-6; Laato, M., Heino, J., Kahari, V. M., Niinikoski, J., Gerdin, B. Epidermal growth factor (EGF) prevents methylprednisolone-induced inhibition of wound healing. J Surg Res 1989;47(4):354-9; Pierce, G. F., Mustoe, T. A., Lingelbach, J., Masakowski, V. R., Gramates, P., Deuel, T. F. Transforming growth factor beta reverses the glucocorticoid-induced wound-healing deficit in rats: possible regulation in macrophages by platelet-derived growth factor. Proc Natl Acad Sci U S A 1989;86(7):2229-33). It has also been shown that glucocorticoids decrease collagen synthesis in rat and mouse skin in vivo and in rat and human fibroblasts (Oishi, Y., Fu, Z. W., Ohnuki, Y., Kato, H., Noguchi, T. Molecular basis of the alteration in skin collagen metabolism in response to in vivo dexamethasone treatment: effects on the synthesis of collagen type I and III, collagenase, and tissue inhibitors of metalloproteinases. Br J Dermatol 2002;147(5):859-68).

### SUMMARY OF THE INVENTION

The compounds according to the present invention solves the above problems and embraces a novel class of compounds which has been developed and which inhibit the human 11-β-hydroxysteroid dehydrogenase type 1 enzyme (11-β-HSD₁), and may therefore be of use in the treating disorders such as diabetes, obesity, glaucoma, osteoporosis, cognitive disorders, immune disorders, hypertension, and wound healing.

One object of the present invention is a compound of the general formula (I) 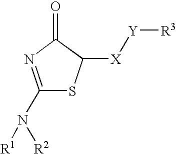
wherein
R¹ and R² are each independently selected from hydrogen; C₁₋₈-alkyl; C₃₋₁₀-cycloalkyl optionally independently substituted by one or more of C₁₋₈-alkyl; C₂₋₈-alkenyl; C₃₋₁₀-cycloalkyl-C₁₋₈-alkyl; C₃₋₁₀-cycloalkenyl; C₃₋₁₀-cycloalkenyl-C₁₋₈-alkyl; C₁₋₈-acyl; heterocyclyl optionally independently substituted by one or more of C₁₋₈-alkyl; heterocyclyl-C₁₋₈-alkyl; aryl optionally independently substituted by one or more of halogen, C₁₋₈-alkyl, halo-C₁₋₈-alkyl, C₁₋₈-alkoxy, and heterocyclyl; indanyl; aryl-C₁₋₈-alkyl optionally independently substituted by one or more of halogen and C₁₋₈-alkyl; aryl-C₃₋₁₀-cycloalkyl optionally independently substituted by one or more of halogen; heteroaryl optionally independently substituted by one or more of aryloxy; heterocyclyl-C₁₋₈-alkyl; or form together with the nitrogen atom bonded thereto heterocyclyl;
X is CH₂;
Y is CH₂, CO or a single bond;
R³ is hydrogen; C₁₋₈-alkyl; C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl substituted by one or more of halogen or hydroxy;
or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally independently substituted by one or more of halogen, C₁₋₈-alkyl and aryl; heteroaryl-C₁₋₈-alkylcarbonyl; C₃₋₁₀-cycloalkylcarbonyl; heterocyclylcarbonyl; heteroaryl; COOR⁶, wherein R⁶ is selected from C₁₋₈-alkyl and aryl; CONR⁷R⁸, wherein R⁷ and R⁸ are each independently selected from hydrogen and C₁₋₈-alkyl; or wherein R³ is OCONR⁹R¹⁰, wherein R⁹ and R¹⁰ are each independently selected from aryl optionally independently substituted by one or more of halogen, nitro, and aryloxy; or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro;
or pharmaceutically acceptable salts, solvates, hydrates, geometrical isomers, tautomers, optical isomers, N-oxides and prodrug forms thereof;
with the provisos that:
   R¹ and R² are each independently selected from hydrogen; 2-butyl; isobutyl; tert-butyl; 2-methylbutyl; 1,1,3,3-tetramethylbutyl; cyclopropyl; cyclopentyl; cycloheptyl; cyclooctyl; C₃₋₁₀-bicycloalkyl; C₃₋₁₀-tricycloalkyl; cyclopropylmethyl; cyclohexylmethyl; 2,2,3,3-tetramethylcyclopropyl; (1R,2R,3R,5S)-2,6,6-trimethylbicyclo[3.1.1]hept-3-yl; (1S,2S,3S,5R)-2,6,6-trimethylbicyclo[3.1.1]hept-3-yl; C₃₋₁₀-cycloalkyl-C₁₋₈-alkyl; C₃₋₁₀-cycloalkenyl; C₃₋₁₀-cycloalkenyl-C₁₋₈-alkyl; heterocyclyl substituted by one or more of C₁₋₈-alkyl; heterocyclyl-C₁₋₈-alkyl; 1-naphthyl; phenyl substituted by one or more of halogen, C₁₋₈-alkyl, halo-C₁₋₈-alkyl, C₁₋₈-alkoxy, and heterocyclyl; indanyl; 4-chlorobenzyl; 4-methylbenzyl; (1R)-1-phenylethyl; (1S)-1-phenylethyl; 2-phenylethyl; (2R)-2-phenylpropyl; (2S)-2-phenylpropyl; aryl-C₃₋₁₀-cycloalkyl optionally independently substituted by one or more of halogen; heteroaryl substituted by one or more of aryloxy; heterocyclyl-C₁₋₈-alkyl; or form together with the nitrogen atom bonded thereto azepan-1-yl;
   when either R¹ or R² is 1-naphthyl; phenyl substituted by one or more of halogen, C₁₋₈-alkyl, halo-C₁₋₈-alkyl, C₁₋₈-alkoxy, and heterocyclyl, then R⁴ and R⁵ are each independently selected from C₃₋₁₀-cycloalkyl optionally substituted by one or more of C₁₋₈-alkyl; cyclopropylmethyl; C₃₋₁₀-cycloalkylcarbonyl; 2-phenylethyl; 2-chloro-6-fluorobenzyl; 3-chloro-2-methylbenzyl; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally independently substituted by one or more of halogen, C₁₋₈-alkyl and aryl; heteroaryl-C₁₋₈-alkylcarbonyl; C₃₋₁₀-cycloalkylcarbonyl; heterocyclylcarbonyl; heteroaryl; or either of R⁴ and R⁵ is hydrogen and the other of R⁴ and R⁵ is selected from C₃₋₁₀-cycloalkyl optionally substituted by one or more of C₁₋₈-alkyl; cyclopropylmethyl; C₃₋₁₀-cycloalkylcarbonyl; 2-phenylethyl; 2-chloro-6-fluorobenzyl; 3-chloro-2-methylbenzyl; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally substituted by one or more of halogen, C₁₋₈-alkyl and aryl; heteroaryl-C₁₋₈-alkylcarbonyl; C₃₋₁₀-cycloalkylcarbonyl;
   when both R¹ and R² are hydrogen, then R³ is C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl substituted by one or more of halogen or hydroxy;
      or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally independently substituted by one or more of halogen, C₁₋₈-alkyl and aryl; heteroaryl-C₁₋₈-alkylcarbonyl; C₃₋₁₀-cycloalkylcarbonyl; heterocyclylcarbonyl; heteroaryl; COOR⁶, wherein R⁶ is selected from C₁₋₈-alkyl and aryl; CONR⁷R⁸, wherein R⁷ and R⁸ are each independently selected from hydrogen and C₁₋₈-alkyl; or wherein R³ is OCONR⁹R¹⁰, wherein R⁹ and R¹⁰ are each independently selected from aryl optionally independently substituted by one or more of halogen, nitro, and aryloxy;
      or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro.

In some embodiments of the present invention, the provisos above do not apply and instead:
when either R¹ or R² is optionally substituted aryl, then neither R⁴ nor R⁵ is optionally substituted aryl;
neither R¹ nor R² is methyl, ethyl, allyl, benzyl, acetyl, phenyl and do not form together with the nitrogen atom bonded thereto piperidine;
when either R¹ or R² is optionally substituted aryl, then neither R⁴ nor R⁵ is C₁₋₈-alkyl, benzyl, cyclohexylmethyl;
the following compounds are excluded:
2-(cyclohexylamino)-N-(2,4-dimethoxyphenyl)-4,5-dihydro-4-oxo-5-thiazoleacetamide;
N-cyclohexyl-2-(cyclohexylamino)-4,5-dihydro-4-oxo-5-thiazoleacetamide;
2-(cyclohexylamino)-N-(3,4-dimethylphenyl)-4,5-dihydro-4-oxo-5-thiazoleacetamide;
2-(cyclohexylamino)-N-(2,6-dimethylphenyl)-4,5-dihydro-4-oxo-5-thiazoleacetamide;
N-(4-chloro-2,5-dimethoxyphenyl)-2-(cyclohexylamino)-4,5-dihydro-4-oxo-5-thiazoleacetamide;
2-(cyclohexylamino)-N-(2,5-dichlorophenyl)-4,5-dihydro-4-oxo-5-thiazoleacetamide;
2-(cyclohexylamino)-N-(2,5-dimethylphenyl)-4,5-dihydro-4-oxo-5-thiazoleacetamide;
2-(cyclohexylamino)-4,5-dihydro-N-(4-methylphenyl)-4-oxo-5-thiazoleacetamide;
2-(cyclohexylamino)-4,5-dihydro-N-(3-methylphenyl)-4-oxo-5-thiazoleacetamide;
2-(cyclohexylamino)-4,5-dihydro-N-(2-methylphenyl)-4-oxo-5-thiazoleacetamide;
2-(cyclohexylamino)-N-(2,4-dimethylphenyl)-4,5-dihydro-4-oxo-5-thiazoleacetamide;
2-(cyclohexylamino)-4,5-dihydro-4-oxo-N-phenyl-5-thiazoleacetamide;
4-[[4,5-dihydro-2-(4-morpholinyl)-4-oxo-5-thiazolyl]acetyl]morpholine;
4,5-dihydro-2-[(4-methylphenyl)amino]-4-oxo-5-thiazolecarboxamide;
2-amino-4,5-dihydro-4-oxo-5-ethylthiazole; and
2-amino-4,5-dihydro-4-oxo-5-methylthiazole.

In some embodiments:
R¹ and R² are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkenyl; C₃₋₁₀-cycloalkenyl-C₁₋₈-alkyl; heterocyclyl optionally independently substituted by one or more of C₁₋₈-alkyl; heterocyclyl-C₁₋₈-alkyl; aryl optionally independently substituted by one or more of halogen, C₁₋₈-alkyl, halo-C₁₋₈-alkyl, C₁₋₈-alkoxy, and heterocyclyl; indanyl; aryl-C₁₋₈-alkyl optionally independently substituted by one or more of halogen and C₁₋₈-alkyl; aryl-C₃₋₁₀-cycloalkyl optionally independently substituted by one or more of halogen; heteroaryl optionally independently substituted by one or more of aryloxy; heterocyclyl-C₁₋₈-alkyl; or form together with the nitrogen atom bonded thereto heterocyclyl;
X is CH₂;
Y is CH₂, CO or a single bond;
R³ is hydrogen; C₁₋₈-alkyl; C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl optionally independently substituted by one or more of halogen or hydroxy;
or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally substituted by one or more of halogen, C₁₋₈-alkyl and aryl; heteroaryl-C₁₋₈-alkylcarbonyl; C₃₋₁₀-cycloalkylcarbonyl; heteroaryl; or wherein R³ is OCONR⁹R¹⁰, wherein R⁹ and R¹⁰ are each independently selected from aryl substituted by one or more of halogen, nitro, and aryloxy;
or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro.

In some embodiments:
R¹ and R² are selected from hydrogen; 2-butyl; isobutyl; tert-butyl; 2-methylbutyl; 1,1,3,3-tetramethylbutyl; cyclopropyl; cyclopentyl; cyclohexyl; cycloheptyl; bicyclo[2.2.1]hept-2-yl; cyclooctyl; 1-adamantyl; tricyclo[3.3.1.0˜3,7˜]non-3-yl; cyclopropylmethyl; cyclohexylmethyl; 2,2,3,3-tetramethylcyclopropyl; (1R,2R,3R,5S)-2,6,6-trimethylbicyclo[3.1.1]hept-3-yl; (1S,2S,3S,5R)-2,6,6-trimethylbicyclo[3.1.1]hept-3-yl; bicyclo[2.2.1]hept-5-en-2-yl; (1R)-1-cyclohexylethyl; (1S)-1-cyclohexylethyl; 2-(1-cyclohexenyl)ethyl; 4-(2,2,6,6-tetramethyl)piperidyl; 2-(4-morpholinyl)ethyl; 1-naphthyl; 2-fluorophenyl; 3-chloro-2-methylphenyl; mesityl; 3,5-di(trifluoromethyl)phenyl; 2,6-dimethylphenyl, 4-(4-morpholinyl)phenyl; 2-methylphenyl; 2-isopropylphenyl; 2-methoxyphenyl; 2-indanyl; 4-chlorobenzyl; 4-methylbenzyl; (1R)-1-phenylethyl; (1S)-1-phenylethyl; 2-phenylethyl; (2R)-2-phenylpropyl; (2S)-2-phenylpropyl; 1-(4-chlorophenyl)cyclobutyl; 6-phenoxy-3-pyridyl; 2-(4-morpholinyl)ethyl; or R¹ and R² form together with the nitrogen atom bonded thereto azepan-1-yl;
R³ is hydrogen; methyl; ethyl; isopropyl; cyclohexyl; bromo; 1-hexahydroazepinyl; 4-morpholinyl; N-phthalimidyl; piperidin-1-yl; 4-methylpiperidin-1-yl; 1-(1,2,3,4-tetrahydroquinolinyl); 2-(1,2,3,4-tetrahydroisoquinolinyl); 8-methyl-1-(1,2,3,4-tetrahydroquinolinyl); 1-[7-(trifluoromethyl)-1,2,3,4-tetrahydroquinolinyl; 3,4-dihydroisoquinolin-2(1H)-yl; 6,7-dimethoxy-3,4-dihydroisoquinolin-2(1H)-yl; 4-benzylpiperidin-1-yl; azepan-1-yl; azocan-1-yl; 1-oxa-4-azaspiro[4.5]dec-4-yl; 2-decahydroisoquinolinyl; 1,4-diazepan-1-ium; 1,3-dihydro-2H-isoindol-2-yl; 2,3-dihydro-1H-indol-1-yl; pyrrolidin-1-yl; 3-pyridinyl; 3-indolyl; 1,3-benzoxazol-2-yl; 1,3-benzothiazol-2-yl; 1H-benzimidazol-2-yl; 4-hydroxy-4-phenylpiperidin-1-yl; 5-(2-chlorophenyl)-1,3,4-oxadiazol-2-yl; 4-chlorophenyl; 4-hydroxyphenyl; 3,4-dihydroxyphenyl;
or wherein R³ is NR⁴R⁵, wherein R⁴ and R⁵ are each independently selected from hydrogen; methyl; ethyl; n-propyl; isopropyl; n-butyl; cyclohexyl; cycloheptyl; (1R,2R,4S)-bicyclo[2.2.1]hept-2-yl; 4-methylcyclohexyl; cyclopropylmethyl; cyclohexylmethyl; cyclohexylcarbonyl; 1-adamantylcarbonyl; phenyl; 1-naphthyl; 4-bromophenyl; 2-chlorophenyl; 3-chlorophenyl; 4-chlorophenyl; 4-fluorophenyl; 2,6-difluorophenyl; 3-chloro-2-methylphenyl; 2-methylphenyl; 3-methylphenyl; 4-methylphenyl; 4-methoxyphenyl; 4-(trifluoromethoxy)phenyl; 2-benzoylphenyl; 3-carboxyphenyl; benzyl; 2-phenylethyl; 2-chloro-6-fluorobenzyl; 3-chloro-2-methylbenzyl; 2,2-dimethylpropionamido; phenoxyacetyl; 2-chlorobenzoyl; 2-fluorobenzoyl; 4-chlorobenzoyl; 2,5-difluorobenzoyl; 2,6-difluorobenzoyl; 2-chloro-6-fluorobenzoyl; 2,4-dichlorobenzoyl; 2,4,6-trichlorobenzoyl; 2-methoxybenzoyl; 4-methoxybenzoyl; 2-bromo-5-methoxybenzoyl; 2,4-dimethoxybenzoyl; 2,6-dimethoxybenzoyl; 4-(trifluoromethyl)benzoyl; 2-fluoro-4-(trifluoromethyl)benzoyl; 2,5-di(trifluoromethyl)benzoyl; 2-fluoro-5-(trifluoromethyl)benzoyl; 4-cyanobenzoyl; 2-chloro-6-fluorophenylacetyl; 2-chlorophenylsulfonyl; 2,6-difluorophenylsulfonyl; 2-chloro-3-pyridylcarbonyl; 2-furylcarbonyl; 2-thienylcarbonyl; 5-isoxazolylcarbonyl; 5-methyl-3-phenylisoxazol-4-ylcarbonyl; 2-thienylmethylcarbonyl; cyclopropylcarbonyl; cyclohexylcarbonyl; isopentanoyl; indazol-6-yl;
OCONR⁹R¹⁰, wherein R⁹ and R¹⁰ are each independently selected from 2-chlorophenyl; 4-bromo-2,6-difluorophenyl; 4-chloro-3-nitrophenyl; 3-phenoxyphenyl;
NHCONR¹¹R¹², wherein R¹¹ and R¹² are each independently selected from hydrogen, cyclopentyl, cyclohexyl, 2-chlorophenyl, 2-fluorophenyl, 4-fluorophenyl, 2,4-difluorophenyl, 2,6-difluorophenyl, 2-chloro-5-(trifluoromethyl)phenyl, 4-fluoro-2-(trifluoromethyl)phenyl, 2-methoxyphenyl, 2,4-dimethoxyphenyl, 5-chloro-2-methoxyphenyl, 2,6-dichloropyridin-4-yl;
   OR¹³, wherein R¹³ is selected from hydrogen; phenyl; 2-chlorophenyl; 4-chloro-3-methylphenyl; 2-methoxyphenyl; 4-carbomethoxy-2-chlorophenyl; 3-(4-morpholinyl)phenyl; 4-phenoxyphenyl; 2-chlorobenzyl; 2-methylbenzyl; 2-methoxybenzyl; 3-(dimethylamino)benzyl; benzoyl; 2-chlorobenzoyl; 2,4-dichlorobenzoyl; 3,4-dichlorobenzoyl; 2,5-difluorobenzoyl; 2,6-difluorobenzoyl; 3,4-difluorobenzoyl; 2-chloro-6-fluorobenzoyl; 2,4,6-trichlorobenzoyl; 2,3,4-trifluorobenzoyl; 3-methylbenzoyl; 4-methylbenzoyl; 4-tert-butylbenzoyl; 3-methoxybenzoyl; 4-n-butoxybenzoyl; 2,4-dimethoxybenzoyl; 2,6-dimethoxybenzoyl; 2-bromo-5-methoxybenzoyl; 3-(trifluoromethyl)benzoyl; 2,5-di(trifluoromethyl)benzoyl; 3,5-di(trifluoromethyl)benzoyl; 2-fluoro-4-(trifluoromethyl)benzoyl; 2-fluoro-5-(trifluoromethyl)benzoyl; and 4-chloro-3-nitrobenzoyl.

Preferred compounds are Examples 1-10, 17-47, 50-55, 57, 58, 60-69, 71-96, 99-108, 110-121, 123-128, 131, 132, 134, 136-139, 141-146, 150-153, 155, 156, 161-163, 167-171, 173, 174, 176-184, 187-189, 191-193, 195-284, 286-288, 290-342, 344-346, 348-365, 368, 370-378, 381, and 383-388.

Another object of the present invention is a process for the preparation of a compound according to any one of claims 1 to 4, comprising at least one of the following steps:
a) reaction of an isothiocyanate with ammonia to give a thiourea,
b) reaction of an amine with ethoxycarbonylisothiocyanate to give a thiourea,
c) reaction of a thiourea with maleic anhydride to give a thiazolone carboxylic acid,
d) reaction of a thiazolone carboxylic acid with 2-chloro-1-methylpyridinium iodide in the presence of an amine to give a thiazolone amide,
e) reaction of a thiourea with 2-bromo-1-butyrolactone to give a thiazolone alcohol,
f) reaction of a thiazolone alcohol with an acid chloride or an isocyanate in the presence of a base to give a thiazolone ester,
g) reaction of a thiazolone alcohol with triphenylphosphine and then with a benzyl alcohol in the presence of dietyl azodicarboxylate to give a thiazolone ether,
h) reaction of a thiourea with an N-substituted 3-bromo-1-phenylpyrrolidin-2-one to give a thiazolone amine,
i) reaction of a thiazolone alcohol with triphenylphosphine dibromide to give a thiazolone bromide,
j) reaction of a thiazolone bromide with an N-substituted aniline to give a thiazolone amine,
k) reaction of a thiourea with 3-(4-chlorobenzoyl)acrylic acid to give a thiazolone,
l) reaction of a thiourea with 3-bromopyrrolidin-2-one to give a thiazolone amine,
m) reaction of a thiazolone amine with a benzoyl chloride or a sulfonyl chloride to give a thiazolone amide or a thiazolone sulfonamide, respectively,
n) hydrolysis of a thiazolone amide with hydrazine to give a thiazolone amine,
o) esterification of a thiazolone carboxylic acid with a phenol in the presence of a base and a coupling agent to give a thiazolone phenol ester,
p) reaction of a thiourea with a 1H-pyrrole-2,5-dione to give a thiazolone amide,
q) reaction of a thiazolone carboxylic acid with diphenylphosphoryl azide and then with a benzoyl chloride to give a thiazolone amide,
r) amidification of a thiazolone carboxylic acid with an amine in the presence of a base and a coupling agent to give a thiazolone amide,
s) reaction of an N—C₃₋₁₀-cycloalkylthiourea with a carboxylic acid to give a thiazolone,
t) reaction of an N—C₃₋₁₀-cycloalkylthiourea with a bromo substituted carboxylic ester to give a thiazolone,
u) reaction of a thiazolone carboxylic acid with 2-chlorobenzohydrazide in the presence of POCl₃ to give a thiazolone containing a triazole group,
v) reaction of a thiazolone carboxylic acid with an aromatic amine to give a thiazoline containing a benzimidazole, benzoxazole or a benzothiazole group, and
w) alkylation of a thiazolone amine.

Another object of the present invention is a compound of the general formula (I) 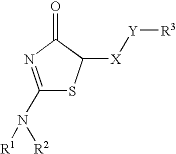
wherein
R¹ and R² are each independently selected from hydrogen; C₁₋₈-alkyl; C₃₋₁₀-cycloalkyl optionally independently substituted by one or more of C₁₋₈-alkyl; C₂₋₈-alkenyl; C₃₋₁₀-cycloalkyl-C₁₋₈-alkyl; C₃₋₁₀-cycloalkenyl; C₃₋₁₀-cycloalkenyl-C₁₋₈-alkyl; C₁₋₈-acyl; heterocyclyl optionally independently substituted by one or more of C₁₋₈-alkyl; heterocyclyl-C₁₋₈-alkyl; aryl optionally independently substituted by one or more of halogen, C₁₋₈-alkyl, halo-C₁₋₈-alkyl, C₁₋₈-alkoxy, and heterocyclyl; indanyl; aryl-C₁₋₈-alkyl optionally independently substituted by one or more of halogen and C₁₋₈-alkyl; aryl-C₃₋₁₀-cycloalkyl optionally independently substituted by one or more of halogen; heteroaryl optionally independently substituted by one or more of aryloxy; heterocyclyl-C₁₋₈-alkyl; or form together with the nitrogen atom bonded thereto heterocyclyl;
X is CH₂;
Y is CH₂, CO or a single bond;
R³ is hydrogen; C₁₋₈-alkyl; C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl substituted by one or more of halogen or hydroxy;
or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally independently substituted by one or more of halogen, C₁₋₈-alkyl and aryl; heteroaryl-C₁₋₈-alkylcarbonyl; C₃₋₁₀-cycloalkylcarbonyl; heterocyclylcarbonyl; heteroaryl; COOR⁶, wherein R⁶ is selected from C₁₋₈-alkyl and aryl; CONR⁷R⁸, wherein R⁷ and R⁸ are each independently selected from hydrogen and C₁₋₈-alkyl; or wherein R³ is OCONR⁹R¹⁰, wherein R⁹ and R¹⁰ are each independently selected from aryl optionally independently substituted by one or more of halogen, nitro, and aryloxy; or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro;
or pharmaceutically acceptable salts, solvates, hydrates, geometrical isomers, tautomers, optical isomers, N-oxides and prodrug forms thereof;
   for use in therapy.

Also featured are methods for treating a patient comprising administering a compound of formula (I) of any of claims 6 to 9.

In some embodiments:
R¹ and R² are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkenyl; C₃₋₁₀-cycloalkenyl-C₁₋₈-alkyl; heterocyclyl optionally independently substituted by one or more of C₁₋₈-alkyl; heterocyclyl-C₁₋₈-alkyl; aryl optionally independently substituted by one or more of halogen, C₁₋₈-alkyl, halo-C₁₋₈-alkyl, C₁₋₈-alkoxy, and heterocyclyl; indanyl; aryl-C₁₋₈-alkyl optionally independently substituted by one or more of halogen and C₁₋₈-alkyl; aryl-C₃₋₁₀-cycloalkyl optionally independently substituted by one or more of halogen; heteroaryl optionally independently substituted by one or more of aryloxy; heterocyclyl-C₁₋₈-alkyl; or form together with the nitrogen atom bonded thereto heterocyclyl;
X is CH₂;
Y is CH₂, CO or a single bond;
R³ is hydrogen; C₁₋₈-alkyl; C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl optionally independently substituted by one or more of halogen or hydroxy;
or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally substituted by one or more of halogen, C₁₋₈-alkyl and aryl; heteroaryl-C₁₋₈-alkylcarbonyl; C₃₋₁₀-cycloalkylcarbonyl; heteroaryl; or wherein R³ is OCONR⁹R¹⁰, wherein R⁹ and R¹⁰ are each independently selected from aryl substituted by one or more of halogen, nitro, and aryloxy;
or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro.

In some embodiments:
R¹ and R² are selected from hydrogen; 2-butyl; isobutyl; tert-butyl; 2-methylbutyl; 1,1,3,3-tetramethylbutyl; cyclopropyl; cyclopentyl; cyclohexyl; cycloheptyl; bicyclo[2.2.1]hept-2-yl; cyclooctyl; 1-adamantyl; tricyclo[3.3.1.0˜3,7˜]non-3-yl; cyclopropylmethyl; cyclohexylmethyl; 2,2,3,3-tetramethylcyclopropyl; (1R,2R,3R,5S)-2,6,6-trimethylbicyclo[3.1.1]hept-3-yl; (1S,2S,3S,5R)-2,6,6-trimethylbicyclo[3.1.1]hept-3-yl; bicyclo[2.2.1]hept-5-en-2-yl; (1R)-1-cyclohexylethyl; (1S)-1-cyclohexylethyl; 2-(1-cyclohexenyl)ethyl; 4-(2,2,6,6-tetramethyl)piperidyl; 2-(4-morpholinyl)ethyl; 1-naphthyl; 2-fluorophenyl; 3-chloro-2-methylphenyl; mesityl; 3,5-di(trifluoromethyl)phenyl; 2,6-dimethylphenyl, 4-(4-morpholinyl)phenyl; 2-methylphenyl; 2-isopropylphenyl; 2-methoxyphenyl; 2-indanyl; 4-chlorobenzyl; 4-methylbenzyl; (1R)-1-phenylethyl; (1S)-1-phenylethyl; 2-phenylethyl; (2R)-2-phenylpropyl; (2S)-2-phenylpropyl; 1-(4-chlorophenyl)cyclobutyl; 6-phenoxy-3-pyridyl; 2-(4-morpholinyl)ethyl; or R¹ and R² form together with the nitrogen atom bonded thereto azepan-1-yl;
R³ is hydrogen; methyl; ethyl; isopropyl; cyclohexyl; bromo; 1-hexahydroazepinyl; 4-morpholinyl; N-phthalimidyl; piperidin-1-yl; 4-methylpiperidin-1-yl; 1-(1,2,3,4-tetrahydroquinolinyl); 2-(1,2,3,4-tetrahydroisoquinolinyl); 8-methyl-1-(1,2,3,4-tetrahydroquinolinyl); 1-[7-(trifluoromethyl)-1,2,3,4-tetrahydroquinolinyl; 3,4-dihydroisoquinolin-2(1H)-yl; 6,7-dimethoxy-3,4-dihydroisoquinolin-2(1H)-yl; 4-benzylpiperidin-1-yl; azepan-1-yl; azocan-1-yl; 1-oxa-4-azaspiro[4.5]dec-4-yl; 2-decahydroisoquinolinyl; 1,4-diazepan-1-ium; 1,3-dihydro-2H-isoindol-2-yl; 2,3-dihydro-1H-indol-1-yl; pyrrolidin-1-yl; 3-pyridinyl; 3-indolyl; 1,3-benzoxazol-2-yl; 1,3-benzothiazol-2-yl; 1H-benzimidazol-2-yl; 4-hydroxy-4-phenylpiperidin-1-yl; 5-(2-chlorophenyl)-1,3,4-oxadiazol-2-yl; 4-chlorophenyl; 4-hydroxyphenyl; 3,4-dihydroxyphenyl;
or wherein R³ is NR⁴R⁵, wherein R⁴ and R⁵ are each independently selected from hydrogen; methyl; ethyl; n-propyl; isopropyl; n-butyl; cyclohexyl; cycloheptyl; (1R,2R,4S)-bicyclo[2.2.1]hept-2-yl; 4-methylcyclohexyl; cyclopropylmethyl; cyclohexylmethyl; cyclohexylcarbonyl; 1-adamantylcarbonyl; phenyl; 1-naphthyl; 4-bromophenyl; 2-chlorophenyl; 3-chlorophenyl; 4-chlorophenyl; 4-fluorophenyl; 2,6-difluorophenyl; 3-chloro-2-methylphenyl; 2-methylphenyl; 3-methylphenyl; 4-methylphenyl; 4-methoxyphenyl; 4-(trifluoromethoxy)phenyl; 2-benzoylphenyl; 3-carboxyphenyl; benzyl; 2-phenylethyl; 2-chloro-6-fluorobenzyl; 3-chloro-2-methylbenzyl; 2,2-dimethylpropionamido; phenoxyacetyl; 2-chlorobenzoyl; 2-fluorobenzoyl; 4-chlorobenzoyl; 2,5-difluorobenzoyl; 2,6-difluorobenzoyl; 2-chloro-6-fluorobenzoyl; 2,4-dichlorobenzoyl; 2,4,6-trichlorobenzoyl; 2-methoxybenzoyl; 4-methoxybenzoyl; 2-bromo-5-methoxybenzoyl; 2,4-dimethoxybenzoyl; 2,6-dimethoxybenzoyl; 4-(trifluoromethyl)benzoyl; 2-fluoro-4-(trifluoromethyl)benzoyl; 2,5-di(trifluoromethyl)benzoyl; 2-fluoro-5-(trifluoromethyl)benzoyl; 4-cyanobenzoyl; 2-chloro-6-fluorophenylacetyl; 2-chlorophenylsulfonyl; 2,6-difluorophenylsulfonyl; 2-chloro-3-pyridylcarbonyl; 2-furylcarbonyl; 2-thienylcarbonyl; 5-isoxazolylcarbonyl; 5-methyl-3-phenylisoxazol-4-ylcarbonyl; 2-thienylmethylcarbonyl; cyclopropylcarbonyl; cyclohexylcarbonyl; isopentanoyl; indazol-6-yl;
OCONR⁹R¹⁰, wherein R⁹ and R¹⁰ are each independently selected from 2-chlorophenyl; 4-bromo-2,6-difluorophenyl; 4-chloro-3-nitrophenyl; 3-phenoxyphenyl;
NHCONR¹¹R¹², wherein R¹¹ and R¹² are each independently selected from hydrogen, cyclopentyl, cyclohexyl, 2-chlorophenyl, 2-fluorophenyl, 4-fluorophenyl, 2,4-difluorophenyl, 2,6-difluorophenyl, 2-chloro-5-(trifluoromethyl)phenyl, 4-fluoro-2-(trifluoromethyl)phenyl, 2-methoxyphenyl, 2,4-dimethoxyphenyl, 5-chloro-2-methoxyphenyl, 2,6-dichloropyridin-4-yl;
OR¹³, wherein R¹³ is selected from hydrogen; phenyl; 2-chlorophenyl; 4-chloro-3-methylphenyl; 2-methoxyphenyl; 4-carbomethoxy-2-chlorophenyl; 3-(4-morpholinyl)phenyl; 4-phenoxyphenyl; 2-chlorobenzyl; 2-methylbenzyl; 2-methoxybenzyl; 3-(dimethylamino)benzyl; benzoyl; 2-chlorobenzoyl; 2,4-dichlorobenzoyl; 3,4-dichlorobenzoyl; 2,5-difluorobenzoyl; 2,6-difluorobenzoyl; 3,4-difluorobenzoyl; 2-chloro-6-fluorobenzoyl; 2,4,6-trichlorobenzoyl; 2,3,4-trifluorobenzoyl; 3-methylbenzoyl; 4-methylbenzoyl; 4-tert-butylbenzoyl; 3-methoxybenzoyl; 4-n-butoxybenzoyl; 2,4-dimethoxybenzoyl; 2,6-dimethoxybenzoyl; 2-bromo-5-methoxybenzoyl; 3-(trifluoromethyl)benzoyl; 2,5-di(trifluoromethyl)benzoyl; 3,5-di(trifluoromethyl)benzoyl; 2-fluoro-4-(trifluoromethyl)benzoyl; 2-fluoro-5-(trifluoromethyl)benzoyl; and 4-chloro-3-nitrobenzoyl.

Preferred compounds are Examples 1-388.

The compound (I) of any of claims 6 to 9 may advantageously be used in the prophylaxis or treatment of a 11-hydroxysteroid dehydrogenase type 1 enzyme-mediated disorder or achieving immuno-modulation.

Another object of the present invention is a pharmaceutical formulation comprising a compound of any of claims 6 to 9 as active ingredient, in combination with a pharmaceutically acceptable diluent or carrier, especially for use in the prophylaxis or treatment of a 11-β-hydroxysteroid dehydrogenase type 1 enzyme-mediated disorder or achieving immuno-modulation. The pharmaceutical formulation can include a second active ingredient. The second active ingredient can be an inhibitor of 11-β-hydroxysteroid dehydrogenase type 1 or it can have some other activity.

Another object of the present invention is a method for the prophylaxis or treatment of a 11-β-hydroxysteroid dehydrogenase type 1 enzyme-mediated disorder or achieving immuno-modulation comprising administering the compound of any of claims 6 to 9 to an individual.

Another object of the present invention is a method for inhibiting a 11-β-hydroxysteroid dehydrogenase type 1 enzyme comprising administering the compound of any of claims 6 to 9 to an individual.

Another object of the present invention is the use of a compound of any of claims 6 to 9 for the manufacture of a medicament for use in the prophylaxis or treatment of a 11-β-hydroxysteroid dehydrogenase type 1 enzyme-mediated disorder or achieving immuno-modulation.

Examples of 11-β-hydroxysteroid dehydrogenase type 1 enzyme-mediated disorders include: diabetes, syndrome X, obesity, glaucoma, hyperlipidemia, hyperglycemia, hyperinsulinemia, hypertension, osteoporosis, dementia, depression, virus diseases, and inflammatory diseases.

The compound of any of claims 6 to 9 may be used for the treatment or prophylaxis of a disorder involving delayed or impaired wound healing.

In some embodiments, the disorder involving delayed or impaired wound healing is diabetes.

In some embodiments, the disorder involving delayed or impaired wound healing is caused by treatment with glucocorticoids.

The compound of any of claims 6 to 9 may be used for the promotion of wound healing in chronic wounds, such as diabetic ulcers, venous ulcers or pressure ulcers.

In some embodiments, the immuno-modulation is selected from tuberculosis, lepra, and psoriasis.

Also within the scope of this invention is a method for making a compound of formula (I) with the proviso. The method includes taking any intermediate compound delineated herein, reacting it with one or more reagents to form a compound of formula (I) with the proviso including any processes specifically delineated herein.

Other features and advantages of the invention will be apparent from the detailed description and claims.

### DETAILED DESCRIPTION OF THE INVENTION

The compounds according to the present invention may be used in several indications which involve 11-β-hydroxysteroid dehydrogenase type 1 enzyme. Thus, the compounds according to the present invention may be used against dementia (see WO97/07789), osteoporosis (see Canalis, E. 1996, Mechanisms of glucocorticoid action in bone: implications to glucocorticoid-induced osteoporosis, Journal of Clinical Endocrinology and Metabolism, 81, 3441-3447) and may also be used disorders in the immune system (see Franchimont et al, “Inhibition of Th1 immune response by glucocorticoids: dexamethasone selectively inhibits IL-12-induced Stat 4 phosphorylation in T lymphocytes”, The journal of Immunology 2000, Feb. 15, vol 164 (4), pages 1768-74) and also in the above listed indications.

The various terms used, separately and in combinations, in the above definition of the compounds having the formula (I) will be explained.

The term “aryl” in the present description is intended to include aromatic rings (monocyclic or bicyclic) having from 6 to 10 ring carbon atoms, such as phenyl (Ph), naphthyl, and indanyl (i.e., 2,3-dihydroindenyl), which optionally may be substituted by C₁₋₆-alkyl. Examples of substituted aryl groups are benzyl, and 2-methylphenyl.

The term “heteroaryl” means in the present description a monocyclic, bi- or tricyclic aromatic ring system (only one ring need to be aromatic) having from 5 to 14, preferably 5 to 10 ring atoms such as 5, 6, 7, 8, 9 or 10 ring atoms (mono- or bicyclic), in which one or more of the ring atoms are other than carbon, such as nitrogen, sulfur, oxygen and selenium as part of the ring system. Examples of such heteroaryl rings are pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline (i e 2,3-dihydroindole), isoindoline (i e 1,3-dihydroisoindole), benzothiophene, benzofuran, isobenzofuran, benzoxazole, 2,1,3-benzoxadiazole, benzopyrazole; benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5-naphthyridine, 1,8-naphthyridine, acridine, fenazine and xanthene.

The term “heterocyclic” and “heterocyclyl” in the present description is intended to include unsaturated as well as partially and fully saturated mono-, bi- and tricyclic rings having from 4 to 14, preferably 4 to 10 ring atoms having one or more heteroatoms (e.g., oxygen, sulfur, or nitrogen) as part of the ring system and the reminder being carbon, such as, for example, the heteroaryl groups mentioned above as well as the corresponding partially saturated or fully saturated heterocyclic rings. Exemplary saturated heterocyclic rings are azetidine, pyrrolidine, piperidine, piperazine, morpholine, thiomorpholine, 1,4-oxazepane, azepane, phthalimide, indoline, isoindoline, 1,2,3,4-tetrahydroquinoline, 1,2,3,4-tetrahydroisoquinoline, hexahydroazepine, 3,4-dihydro-2(1H)isoquinoline, 2,3-dihydro-1H-indole, 1,3-dihydro-2H-isoindole, azocane, 1-oxa-4-azaspiro[4.5]dec-4-ene, decahydroisoquinoline, and 1,4-diazepane.

C₁₋₈-alkyl in the compound of formula (I) according to the present application may be a straight or branched alkyl group containing 1-8 carbon atoms. Exemplary alkyl groups include methyl, ethyl, n-propyl, isopropyl, n-butyl, sec-butyl, tert-butyl, pentyl, isopentyl, hexyl, isohexyl, n-heptyl, and n-octyl. For parts of the range “C₁₋₈-alkyl” all subgroups thereof are contemplated such as C₁₋₇-alkyl, C₁₋₆-alkyl, C₁₋₅-alkyl, C₁₋₄-alkyl, C₂₋₈-alkyl, C₂₋₇-alkyl, C₂₋₆-alkyl, C₂₋₅-alkyl, C₃₋₇-alkyl, C₄₋₆-alkyl, etc.

C₁₋₈-alkoxy in the compound of formula (I) according to the present application may be a straight or branched alkoxy group containing 1-8 carbon atoms. Exemplary alkoxy groups include methoxy, ethoxy, propoxy, isopropoxy, butoxy, sec-butoxy, tert-butoxy, pentyloxy, isopentyloxy, hexyloxy, isohexyloxy, n-heptyloxy, and n-octyloxy. For parts of the range “C₁₋₆-alkoxy” all subgroups thereof are contemplated such as C₁₋₇-alkoxy, C₁₋₆-alkoxy, C₁₋₅-alkoxy, C₁₋₄-alkoxy, C₂₋₈-alkoxy, C₂₋₇-alkoxy, C₂₋₆-alkoxy, C₂₋₅-alkoxy, C₃₋₇-alkoxy, C₄₋₆-alkoxy, etc.

C₁₋₈-acyl in the compound of formula (I) according to the present application may be a straight or branched acyl group containing 1-8 carbon atoms. Exemplary acyl groups include formyl, acetyl, propionyl, butyryl, isobutyryl, valeryl, isovaleryl, n-hexanoyl, n-heptanoyl, and n-octanoyl. For parts of the range “C₁₋₈-acyl” all subgroups thereof are contemplated such as C₁₋₇-acyl, C₁₋₆-acyl, C₁₋₅-acyl, C₁₋₄-acyl, C₂₋₈-acyl, C₂₋₇-acyl, C₂₋₄-acyl, C₂₋₅-acyl, C₃₋₇-acyl, C₄₋₆-acyl, etc.

C₂₋₈-alkenyl in the compound of formula (I) according to the present application may be a straight or branched acyl group containing 2-8 carbon atoms. Exemplary alkenyl groups include vinyl, 1-propenyl, 2-propenyl, isopropenyl, 1-butenyl, 2-butenyl, 1-pentenyl, 2-pentenyl, 1-hexenyl, 2-hexenyl, 1-heptenyl, and 1-octenyl. For parts of the range “C₂₋₈-alkenyl” all subgroups thereof are contemplated such as C₂₋₇-alkenyl, C₂₋₆-alkenyl, C₂₋₅-alkenyl, C₂₋₄-alkenyl, C₃₋₈-alkenyl, C₃₋₇-alkenyl, C₃₋₄-alkenyl, C₃₋₅-alkenyl, C₄₋₇-alkenyl, C₅₋₆-alkenyl, etc.

C₃₋₁₀-cycloalkyl is either of C₃₋₁₀-monocycloalkyl, C₃₋₁₀-bicycloalkyl, and C₃₋₁₀-tricycloalkyl.

C₃₋₁₀-monocycloalkyl in the compound of formula (I) according to the present application may be an optionally alkyl substituted monocyclic alkyl group containing totally 3-10 carbon atoms. Exemplary monocycloalkyl groups include cyclopropyl, cyclobutyl, cyclopentyl, cyclohexyl, cycloheptyl, cyclooctyl, cyclononyl, and cyclodecyl. For parts of the range “C₃₋₁₀-monocycloalkyl” all subgroups thereof are contemplated such as C₃₋₉-monocycloalkyl, C₃₋₈-monocycloalkyl, C₃₋₇-monocycloalkyl, C₃₋₆-monocycloalkyl, C₃₋₅-monocycloalkyl, C₄₋₁₀-monocycloalkyl, C₅₋₁₀-monocycloalkyl, C₆₋₁₀-monocycloalkyl, C₇₋₁₀-monocycloalkyl, C₈₋₉-monocycloalkyl, etc.

C₃₋₁₀-bicycloalkyl in the compound of formula (I) according to the present application may be an optionally alkyl substituted bicyclic alkyl group containing totally 3-10 carbon atoms. Exemplary bicycloalkyl groups include bicyclo[2.2.1]hept-2-yl, (1R,2R,3R,5S)-2,6,6-trimethylbicyclo[3.1.1]hept-3-yl, and (1S,2S,3S,5R)-2,6,6-trimethylbicyclo[3.1.1]hept-3-yl. For parts of the range “C₃₋₁₀-bicycloalkyl” all subgroups thereof are contemplated such as C₃₋₉-bicycloalkyl, C₃₋₈-bicycloalkyl, C₃₋₇-bicycloalkyl, C₃₋₆-bicycloalkyl, C₃₋₅-bicycloalkyl, C₄₋₁₀-bicycloalkyl, C₅₋₁₀-bicycloalkyl, C₆₋₀-bicycloalkyl, C₇₋₁₀-bicycloalkyl, C₈₋₉-bicycloalkyl, etc.

C₃₋₁₀-tricycloalkyl in the compound of formula (I) according to the present application may be an optionally alkyl substituted tricyclic alkyl group containing totally 3-10 carbon atoms. Exemplary tricycloalkyl groups include 1-adamantyl, noradamantyl, and tricyclo[3.3.1.0˜3,7˜]non-3-yl. For parts of the range “C₃₋₁₀-tricycloalkyl” all subgroups thereof are contemplated such as C₃₋₉-tricycloalkyl, C₃₋₈-tricycloalkyl, C₃₋₇-tricycloalkyl, C₃₋₆-tricycloalkyl, C₃₋₅-tricycloalkyl, C₄₋₁₀-tricycloalkyl, C₅₋₁₀-tricycloalkyl, C₆₋₁₀-tricycloalkyl, C₇₋₁₀-tricycloalkyl, C₈₋₉-tricycloalkyl, etc.

C₃₋₁₀-cycloalkenyl in the compound of formula (I) according to the present application may be an optionally alkyl substituted cyclic, bicyclic or tricyclic alkenyl group containing totally 3-10 carbon atoms. Exemplary cycloalkenyl groups include cyclopropenyl, cyclobutenyl, cyclopentenyl, cyclohexenyl, cycloheptenyl, cyclooctenyl, cyclononenyl, cyclodecenyl, and bicyclo[2.2.1]hept-5-en-2-yl. For parts of the range “C₃₋₁₀-cycloalkenyl” all subgroups thereof are contemplated such as C₃₋₉-cycloalkenyl, C₃₋₈-cycloalkenyl, C₃₋₇-cycloalkenyl, C₃₋₆-cycloalkenyl, C₃₋₅-cycloalkenyl, C₄₋₁₀-cycloalkenyl, C₅₋₁₀-cycloalkenyl, C₆₋₁₀-cycloalkenyl, C₇₋₁₀-cycloalkenyl, C₈₋₉-cycloalkenyl, etc.

The term “halogen” in the present description is intended to include fluorine, chlorine, bromine and iodine.

The term “sulfanyl” in the present description means a thio group.

With the expression “mono- or di-substituted” is meant in the present description that the functionalities in question may be substituted with independently C₁₋₈-acyl, C₂₋₈-alkenyl, C₁₋₈-(cyclo)alkyl, aryl, pyridylmethyl, or heterocyclic rings e.g. azetidine, pyrrolidine, piperidine, piperazine, morpholine and thiomorpholine, which heterocyclic rings optionally may be substituted with C₁₋₈-alkyl. With the expression “optionally mono- or disubstituted” is meant in the present description that the functionalities in question may also be substituted with independently hydrogen.

When two of the above-mentioned terms are used together, it is intended that the latter group is substituted by the former. For example, C₃₋₁₀-cycloalkyl-C₁₋₈-alkyl means a C₁₋₈-alkyl group that is substituted by a C₃₋₁₀-cycloalkyl group. Likewise, a halo-C₁₋₈-alkyl means a C₁₋₈-alkyl group that is substituted by a halogen atom.

As used herein:
DCM means dichloromethane,
DEAD means diethyl azocarboxylate,
DMF means dimethylformamide,
EDCI means 1-(3-dimethylaminopropyl)-3-ethylcarbodiimide hydrochloride,
Ether means diethyl ether,
EtOAc means ethylacetate,
HOBt means 1-hydroxybenzotriazole,
HPLC means high performance liquid chromatography,
LC means liquid chromatography,
MeCN means acetonitrile,
MS means mass spectroscopy,
DPPA means diphenylphosphoryl azide,
RT means room temperature,
SM means starting material,
TEA means triethylamine, and
THF means tetrahydrofuran.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic administration to a subject for the treatment of disease, 11-β-HSD1 inhibition, 11-β-HSD1-mediated disease).

The term “prodrug forms” in the present description means a pharmacologically acceptable derivative, such as an ester or an amide, which derivative is biotransformed in the body to form the active drug (see Goodman and Gilman's, The Pharmacological basis of Therapeutics,8^{th} ed., McGraw-Hill, Int. Ed. 1992, “Biotransformation of Drugs, p. 13-15).

“Pharmaceutically acceptable” means in the present description being useful in preparing a pharmaceutical composition that is generally safe, non-toxic and neither biologically nor otherwise undesirable and includes being useful for veterinary use as well as human pharmaceutical use.

“Pharmaceutically acceptable salts” mean in the present description salts which are pharmaceutically acceptable, as defined above, and which possess the desired pharmacological activity. Such salts include acid addition salts formed with organic and inorganic acids, such as hydrogen chloride, hydrogen bromide, hydrogen iodide, sulfuric acid, phosphoric acid, acetic acid, glycolic acid, maleic acid, malonic acid, oxalic acid, methanesulfonic acid, trifluoroacetic acid, fumaric acid, succinic acid, tartaric acid, citric acid, benzoic acid, ascorbic acid and the like. Base addition salts may be formed with organic and inorganic bases, such as sodium, ammonia, potassium, calcium, ethanolamine, diethanolamine, N-methylglucamine, choline and the like. Included in the invention are pharmaceutically acceptable salts or compounds of any of the formulae herein.

Pharmaceutical compositions according to the present invention contain a pharmaceutically acceptable carrier together with at least one of the compounds comprising the formula (I) as described herein above, dissolved or dispersed therein as an active, antimicrobial, ingredient. In a preferred embodiment, the therapeutic composition is not immunogenic when administered to a human patient for therapeutic purposes, unless that purpose is to induce an immune response.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

The active ingredient may be mixed with excipients, which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition may contain minor amounts of auxiliary substances such as wetting or emulsifying agents, pH buffering agents and the like which enhance the effectiveness of the active ingredient. Adjuvants may also be present in the composition.

Pharmaceutically acceptable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes.

Liquid compositions can also contain liquid phases in addition to and to the exclusion of water. Exemplary of such additional liquid phases are glycerine, vegetable oils such as cottonseed oil, organic esters such as ethyl oleate, and water-oil emulsions.

The pharmaceutical composition according to one of the preferred embodiments of the present invention comprising compounds comprising the formula (I), may include pharmaceutically acceptable salts of that component therein as set out above. Pharmaceutically acceptable salts include the acid addition salts (formed with the free amino groups of the polypeptide) that are formed with inorganic acids such as, for example, hydrochloric or phosphoric acids, or such organic acids as acetic acid, tartaric acid, mandelic acid and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

The preparations according to the preferred embodiments may be administered orally, topically, intraperitoneally, intraarticularly, intracranially, intradermally, intramuscularly, intraocularly, intrathecally, intravenously, subcutaneously. Other routes are known to those of ordinary skill in the art.

The orally administrable compositions according to the present invention may be in the form of tablets, capsules, powders, granules, lozenges, liquid or gel preparations, such as oral, topical or sterile parenteral solutions or suspensions. Tablets and capsules for oral administration may be in unit dose presentation form and may contain conventional excipients such as binding agents, for example syrup, acacia, gelatin, sorbitol, traganath or polyvinyl-pyrrolidone; fillers e.g. lactose, sugar, maize-starch, calcium phosphate, calcium hydrogen phosphate, sodium starch glycolate, sorbitol or glycine; tabletting lubricant e.g. magnesium stearate, talc, polyethylene glycol or silicon dioxide (optionally colloidal); disintegrants e.g. potato starch, or acceptable wetting agents such as sodium lauryl sulfate. The tablets may be coated according to methods well known in normal pharmaceutical practice. Oral liquid preparations may be in the form of e.g. aqueous or oily suspensions, solutions, emulsions, syrups or elixirs or may be presented as a dry product for reconstitution with water or other suitable vehicle before use. Such liquid preparations may contain conventional additives such as suspending agents, e.g. sorbitol, syrup, methyl cellulose (optionally microcrystalline), glucose syrup, gelatin hydrogenated edible fats; emulsifying agents e.g. lecithin, sorbitan monooleate or acacia, non-aqueous vehicles (which may include edible oils), e.g. almond oil, fractionated coconut oil, oily esters such as glycerine, propylene glycol, or ethyl alcohol; preservatives e.g. methyl or propyl p-hydroxybenzoate or sorbic acid, and if desired conventional flavouring or colouring agents.

“An effective amount” refers to an amount of a compound which confers a therapeutic effect on the treated subject. The therapeutic effect may be objective (i.e., measurable by some test or marker) or subjective (i.e., subject gives an indication of or feels an effect). A pharmaceutical composition according to the present invention, may comprise typically an amount of at least 0.1 weight percent of compound comprising the formula (I) per weight of total therapeutic composition. A weight percent is a ratio by weight of total composition. Thus, for example, 0.1 weight percent is 0.1 grams of compound comprising the formula (I) per 100 grams of total composition. A suitable daily oral dose for a mammal, preferably a human being, may vary widely depending on the condition of the patient. However a dose of compound comprising the formula (I) of about 0.1 to 300 mg/kg body weight may be appropriate.

The compositions according to the present invention may also be used veterinarily and thus they may comprise a veterinarily acceptable excipient or carrier. The compounds and compositions may be thus administered to animals, e.g., cats, dogs, or horses, in treatment methods.

The compounds of the present invention in labelled form, e.g. isotopically labelled, may be used as a diagnostic agent.

This invention relates to methods of making compounds of any of the formulae herein comprising reacting any one or more of the compounds of the formulae delineated herein, including any processes delineated herein. The compounds of formula (I) above may be prepared by, or in analogy with, conventional methods, and especially according to or in analogy with the following methods. Further, the pharmacology in-vitro was studied using the following reagents and methods.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

All publications mentioned herein are hereby incorporated by reference. By the expression “comprising” means “including but not limited to.” Thus, other non-mentioned substances, additives or carriers may be present.

The invention will now be described in reference to the following Examples. These Examples are not to be regarded as limiting the scope of the present invention, but shall only serve in an illustrative manner.

### EXAMPLES

### Experimental Methods

Scintillation Proximity Assay

[1,2(n)-³H]-cortisone was purchased from Amersham Pharmacia Biotech. Anti-cortisol monoclonal mouse antibody, clone 6D6.7 was obtained from Immunotech and Scintillation proximity assay (SPA) beads coated with monoclonal antimouse antibodies were from Amersham Pharmacia Biotech. NADPH, tetrasodium salt was from Calbiochem and glucose-6-phosphate (G-6-P) was supplied by Sigma. The human 11-β-hydroxysteroid dehydrogenase type-1 enzyme (11-β-HSD₁) was expressed in Pichia pastoris. 18-β-glycyrrhetinic acid (GA) was obtained from Sigma. The serial dilutions of the compounds were performed on a Tecan Genesis RSP 150. Compounds to be tested were dissolved in DMSO (1 mM) and diluted in 50 mM Tris-HCl, pH 7.2 containing 1 mM EDTA.

The multiplication of plates was done on a WallacQuadra. The amount of the product [³H]-cortisol, bound to the beads was determined in a Packard, Top Count microplate liquid scintillation counter.

The 11-β-HSD₁ enzyme assay was carried out in 96 well microtiter plates (Packard, Optiplate) in a total well volume of 220 μL and contained 30 mM Tris-HCl, pH 7.2 with 1 mM EDTA, a substrate mixture tritiated Cortisone/NADPH (175 nM/181 μM), G-6-P (1 mM) and inhibitors in serial dilutions (9 to 0.15 μM). Reactions were initiated by the addition of human 11-β-HSD₁, either as Pichia pastoris cell homogenate or microsomes prepared from Pichia pastoris (the final amount of enzyme used was varied between 0.057 to 0.11 mg/mL). Following mixing, the plates were shaken for 30 to 45 minutes at room temperature. The reactions were terminated with 10 μL 1 mM GA stop solution. Monoclonal mouse antibody was then added (10 μL of 4 μM) followed by 100 μL of SPA beads (suspended according to the manufacturers instructions). Appropriate controls were set up by omitting the 11-β-HSD1 to obtain the non-specific binding (NSB) value.

The plates were covered with plastic film and incubated on a shaker for 30 minutes, at room temperature, before counting. The amount of [³H]-cortisol, bound to the beads was determined in a microplate liquid scintillation counter. The calculation of the Kᵢ values for the inhibitors was performed by use of Activity Base. The Kᵢ value is calculated from IC₅₀ and the Kₘ value is calculated using the Cheng Prushoff equation (with reversible inhibition that follows the Michaelis-Menten equation): Kᵢ=IC₅₀(1+[S]/Kₘ) [Cheng, Y. C.; Prushoff, W. H. Biochem. Pharmacol. 1973, 22, 3099-3108]. The IC₅₀ is measured experimentally in an assay wherein the decrease of the turnover of cortisone to cortisol is dependent on the inhibition potential of each substance. The Ki values of the compounds of the present invention for the 11-β-HSD1 enzyme lie typically between about 10 nM and about 10 μM. Illustrative Ki values for some Examples according to the present invention are given below.

| Example | Ki (nM) |
|---|---|
| 109 | 709 |
| 125 | 384 |
| 261 | 559 |
| 335 | 535 |

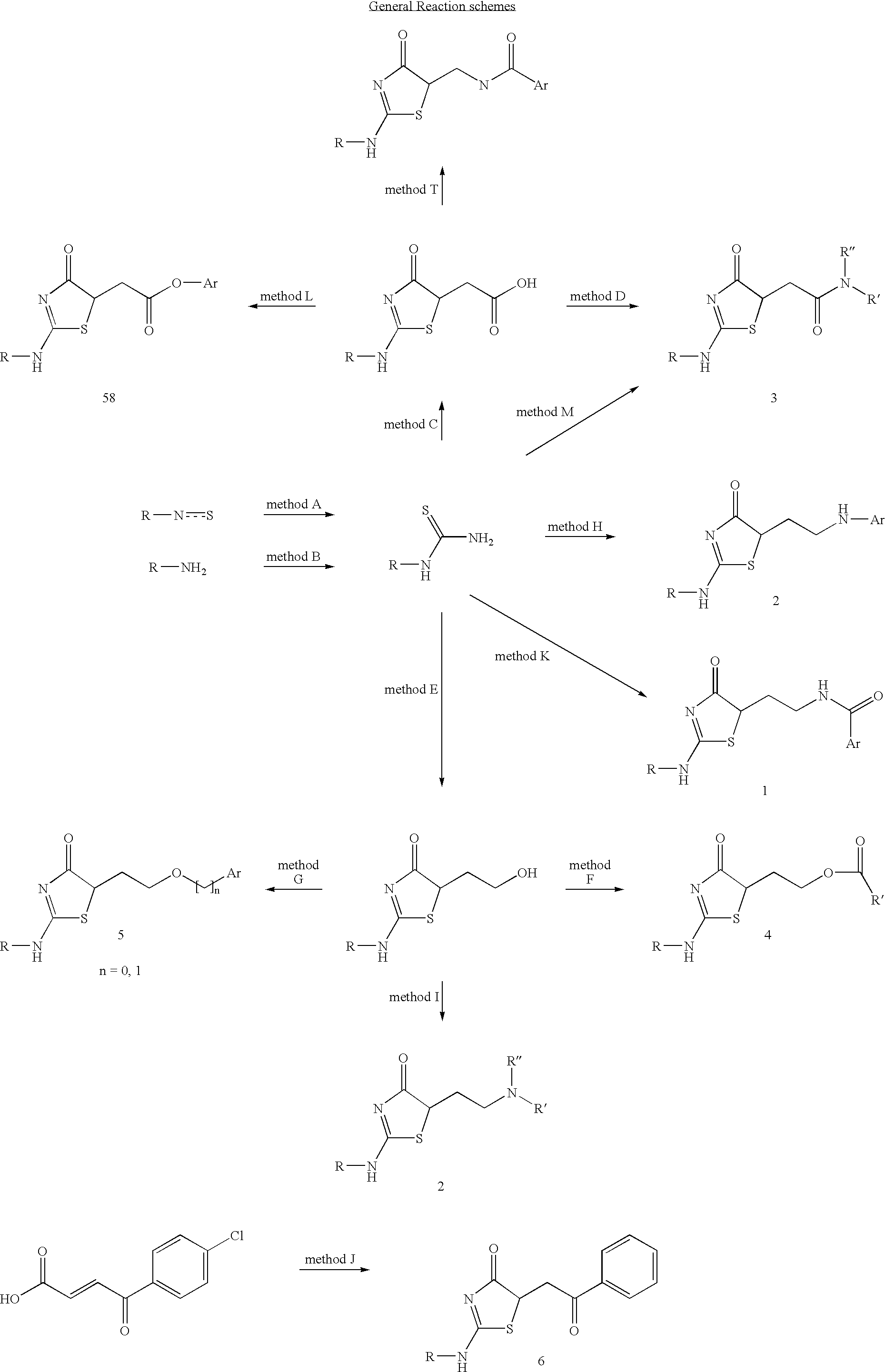

All commercial starting materials are used without any purification.

If the appropriate a-bromo carboxylic acid or ester not is commercially availiable, the substances has been prepared in accordance to this method:

The 2-amino-carboxylic acid (1.0 eq.) was suspended in 2.0 M H₂SO₄ (4 eq.), KBr (8 eq.) was added and the mixture was cooled in an ice-bath. NaNO₂ (1.3 eq.) dissolved in water was added slowly. The reaction mixture was stirred for 4 h at ice-bath, before allowed to reach room temperature. The reaction mixture was extracted with EtOAc. The organic phase was dried over MgSO₄ before concentrated in vacuum. This gave the crude product which was used in the next step without further purification (J. Org. Chem. 2002, 67 (11), 3595-3600; Xinhua Qian; Bin Zheng; Brian Burke; Manohar T. Saindane and David R. Kronenthal).

### Compound Preparation

General Comments:

¹H nuclear magnetic resonance (NMR) and ¹³C NMR were recorded on a Bruker PMR 500 spectrometer at 500.1 MHz and 125.1 MHz, respectively or on a JEOL eclipse 270 spectrometer at 270.0 MHz and 67.5 MHz, respectively. All spectra were recorded using residual solvent or tetramethylsilane (TMS) as internal standard. IR spectra were recorded on a Perkin-Elmer Spectrum 1000 FT-IR spectrometer. Electrospray mass spectrometry (MS) was obtained using an Agilent MSD mass spectrometer. Accurate mass measurements were performed on a Micromass LCT dual probe. Elemental analyses were performed on a Vario El instrument or sent to Mikro Kemi in Uppsala.

Analytical HPLC were performed on Agilent 1100 system equipped with System A: ACE 3 (C8, 50×3.0 mm) or System B: YMC ODS-AQ, (33×3.0 mm) using the eluent system: water/0.1% TFA and CH₃CN, 1 mL/min, with a gradient time of 3 min.

Preparative HPLC was performed on a Gilson system equipped with System A: ACE 5 C8 column (50×20 mm) gradient time 5 min, system B: YMC ODS-AQ (150×30 mm) gradient time 8.5 min or system C: YMC ODS-AQ (50×20 mm) gradient time 5 min using the eluent system: water/0.1% TFA and CH₃CN. Preparative flash chromatography was performed on Merck silica gel 60 (230-400 mesh). The compounds were automatically named using ACD6.0.

General Methods

Method A or B was used depending if the isothiacyanate or of the corresponing amine was used. The amines or isothiacyanate was purchased from either Maybridge plc or from Sigma-Aldrich co.

Method A

1.0 eq. of the appropriate isothiocyanate was stirred in 2 M ammonia in ethanol (5 eq.) for 18 h at RT. Evaporation in vacuo afforded the crude product, which crystallized upon addition of DCM. The crystals were collected on a filter and air-dried to afford the thiourea.

Method B

1.0 eq. of the amine and ethoxycarbonylisothiocyanate (1.0 eq) were mixed in a test tube. A violently exothermic reaction resulted in a white paste. This was taken up in 5M KOH solution and stirred at 70° C. for 2 hours at which point LC analysis indicated full hydrolysis of the intermediate. The mixture was cooled, diluted with water and extracted 3 times with chloroform. Subsequent preparative LC yielded the desired thiourea as a colourless oil.

Method C

1.0 eq. of the appropriate thiourea and maleic anhydride (1.0 eq.) were heated to reflux in acetone for 5 h, yielding a white emulsion. Evaporation in vacuo afforded a white solid. The product was triturated with DCM, collected on a filter and air-dried giving the product as a white powder.

Method D

The carboxylic acid 1.0 eq. and 2-chloro-1-methylpyridinium iodide (1.2 eq.) were mixed in DCM for 10 minutes before the amine (1.0 eq.) was added followed by Et₃N (1.5 eq.). The reaction mixture was stirred at RT for 16 h, full conversion of the SM. The reaction mixture was poured on a Hydromatrix column (pretreated with 1 M HCl) and the crude product was eluted with DCM. The obtained crude product was purified by reverse phase.

Method E

1.0 eq. of the thiourea and 2-bromo-1-butyrolactone (1.0 eq.) were heated to reflux in acetone for 3 h. Evaporation in vacuo gave a colorless oil which was taken up in saturated NaHCO₃ and extracted with DCM. The combined organic layers were dried over Na₂SO₄, filtered and evaporated in vacuo leaving a white solid.

Method F

The alcohol (1.0 eq.) and the appropriate acid chloride or isocyanate (1.0 eq.) were dissolved in DCM and triethylamine (3.0 eq.). The reaction mixture was stirred over night at RT. The solvent was removed under reduced pressure and the product was purified using preparative HPLC.

Method G

1.0 eq. of the alcohol and triphenylphosphine (1.2 eq.) were dissolved in THF. The reaction mixture was stirred at RT for 10 min. before the appropriate benzyl alcohol (1.2 eq.) and DEAD (1.2 eq.) were added. The reaction mixture was stirred at RT over night. The solvent was removed under reduced pressure and the crude was dissolved in DCM and washed with brine. The organic layer was dried (MgSO₄) and the solvent was removed under reduced pressure. Purification using preparative afforded the product.

Method H

The appropriate N-substituted 3-bromo-1-phenylpyrrolidin-2-one (1.0 eq.) and thiourea (1.0 eq.) in acetone was heated to reflux for 3 h. NaHCO₃ (sat. solution) was added and extracted with DCM. The organic phase was dried (Na₂SO₄) and concentrated in vacuum to give the product as a solid.

Method I

1.0 eq. of the alcohol and triphenylphosphine dibromide (2.5 eq.) was dissolved in DCM and stirred at RT for 16 h. The reaction mixture was washed with water and dried (MgSO₄) the solvent was evaporated and the obtained solid crude product was purified by flash chromatography using MeCN as eluent.

1.0 eq. of the obtained bromide and 10 eq. of the appropriate N-substituted aniline were dissolved in DMSO and stirred at 60° C. for 16 h. The reaction mixture was mixed with water and the aqueous phase was extracted twice with ether. The combined organic phases were dried (MgSO₄) and the solvent was evaporated. The obtained crude product was purified by preparative HPLC.

Method J

1.0 eq. of the thiourea and 3-(4-chlorobenzoyl)acrylic acid (1.0 eq.) in water were heated to reflux for 18 h. The precipitate was collected on a filter after cooling and recrystallized from ethanol, yielding the product as white crystals.

Method K

1.0 eq. of 3-bromopyrrolidin-2-one (J. Med. Chem. 1987, 30, 1995-1998. H. Ikuta, H. Shirota, S. Kobayashi, Y. Yamagashi, K. Yamada, I. Yamatsu, K. Katayama) and 1.0 eq. of the appropriate thiourea was dissolved in acetone and heated to reflux for 8 h. The rection mixture cooled to RT and NaHCO₃ (sat. solution) was added and the aqueous phase was extracted with DCM. The organic phase was separated and concentrated in vacuum to give the crude product. The obtained crude product was dissolved in pyridine and a few drops of DMF was added followed by the appropriate benzoyl chloride (3.0 eq.) and the reaction mixture was shaken at RT. (2.0 eq.) of the benzoyl chloride was added after lh and the reaction mixture was shaken at RT over night. 10% HCl was added and extracted with DCM. The organic phase was concentrated in vaccum. Purification was performed using preparative HPLC.

Method L

1.0 eq. of the carboxylic acid, HOBt (1.0 eq.) and EDCI (1.0 eq.) were suspended in DCM. Triethylamine (2 eq) was added and the resulting suspension was stirred for 30 min at ambient temperature. Then 3.0 eq. of the phenol of choice was added, and stirring continued for 3 h. The reaction mixture was eluted over a column containing hydromatrix (5×1 cm) treated with 2M HCl and thoroughly washed with DCM. Evaporation in vacuo afforded the crude product.

Method M

1.0 eq. of the appropriate 1-phenyl-1H-pyrrole-2,5-dione in absolute ethanol was treated with the thiourea (1.05 eq.) and stirred for 18 h at 50° C. The clear solution was reduced to dryness on a rotavapor and the resulting white foam was recrystallised from acetonitrile.

Method T

The carboxylic acid (1.0 eq. 14 mmol) was dissolved in dry acetonitrile, Et₃N (1.0 eq,) and DPPA (1.0 eq.) were added. The reaction mixture was stirred at 50° C. for 2 hours. The reaction mixture was cooled to rt, and 1M HCl (6 ml) was added. The reaction mixture was heated to reflux for 5 hours. The acetonitrile was evaporated and the remaining aqueous solution was saturated with solid NaCO₃, before the aqueous phase was extracted with DCM. The organic phase was evaporated and the obtained crude product was dissolved in DCM and excess of the benzoyl chloride was added. The reaction mixture was stirred at RT for 2 hours. Water was added and the phases were separated. The organic phase was evaporated and the crude product was purified by preparative HPLC.

### EXAMPLES

Compounds of Type 1

### Example 1 (BVT.59212)

### N-{2-[2-(bicyclo[2.2.1]hept-5-en-2-ylamino)-4-oxo-4,5-dihydro-1,3-thiazol-5-yl]ethyl}-2-chloro-6-fluorobenzamide

Prepared according to method K

17.9 mg, 44% yield of orange oil.

¹H NMR (270 MHz, CHLOROFORM-D) δ ppm 1.64-1.86 (m, 4H) 2.07-2.25 (m, 1H) 2.4-2.622 (m, 1H) 3.04 (s, 2H) 3.37 (dd, J=6.93, 3.22 Hz, 1H) 3.43-3.61 (m, 1H) 3.89-4.05 (m, 1H) 4.38-4.49 (m, 1H) 6.02-6.10 (m, 1H) 6.28 (dd, J=5.44, 2.97 Hz, 1H) 6.57 (s, 1H) 7.04 (t, J=8.54 Hz, 1H) 7.18-7.26 (m, 1H) 7.27-7.38 (m, 1H). MS (ESI+) for C₁₉H₁₉ClFN₃O₂S m/z 408 (M+H)⁺.

### Example 2 (BVT.59213)

### N-{2-[2-(bicyclo[2.2.1]hept-5-en-2-ylamino)-4-oxo-4,5-dihydro-1,3-thiazol-5-yl]ethyl}-2,6-dimethoxybenzamide

Prepared according to method K

28.6 mg, 70% of orange oil.

¹H NMR (270 MHz, CHLOROFORM-D) δ ppm 1.54-1.89 (m, 3H) 2.02-2.21 (m, 1H) 2.44-2.60 (m, 1H) 3.03 (d, J=6.93 Hz, 2H) 3.33-3.45 (m, 2H) 3.78-3.82 (m, 6H) 3.91 (s, 1H) 4.01-4.20 (m, 1H) 4.54-4.63 (m, 1H) 6.03-6.10 (m, 1H) 6.25-6.31 (m, 1H) 6.52-6.60 (m, 3H) 7.28-7.37 (m, 1H). MS (ESI+) for C₂₁H₂₅N₃O₄S m/z 416 (M+H)⁺.

### Example 3 (BVT.59436)

### N-{2-[2-(bicyclo[2.2.1]hept-5-en-2-ylamino)-4-oxo-4,5-dihydro-1,3-thiazol-5-yl]ethyl}-2,4-dimethoxybenzamide

Prepared according to method K

0.0055 g, 7% yield.

¹H NMR (270 MHz, CHLOROFORM-D) δ ppm 1.65-1.80 (m, 4H) 2.00 (s, 2H) 2.12-2.26 (m, 1H) 2.45-2.55 (m, 1H) 2.98-3.03 (m, 2H) 3.35-3.37 (m, 1H) 3.49-3.66 (m, 1H) 3.85 (s, 3H) 3.96 (s, 3H) 4.27-4.33 (m, 1H) 6.04-6.08 (m, 1H) 6.26-6.29 (m, 1H) 6.48-6.49 (m, 1H) 6.55-6.60 (m, 1H) 8.00-8.11 (m, 2H). MS (ESI+) for C₂₁H₂₅N₃O₄S m/z 416 (M+H)⁺.

### Example 4 (BVT.59387)

### N-{2-[2-(bicyclo[2.2.1]hept-5-en-2-ylamino)-4-oxo-4,5-dihydro-1,3-thiazol-5-yl]ethyl}-2,6-difluorobenzamide

Prepared according to method K

0.0099 g, 13% yield.

¹H NMR (270 MHz, CHLOROFORM-D) δ ppm 1.67-1.78 (m, 4H) 2.12-2.26 (m, 1H) 2.46-2.55 (m, 1H) 3.01-3.05 (m, 2H) 3.35-3.39 (m, 1H) 3.51-3.64 (m, 1H) 3.86-3.96 (m, 1H) 4.31-4.38 (m, 1H) 6.05-6.08 (m, 1H) 6.27-6.30 (m, 1H) 6.45 (s, 1H) 6.96 (t, J=8.66 Hz, 2H) 7.37-7.45 (m, 1H). MS (ESI+) for C₁₉H₁₉F₂N₃O₂S m/z 392 (M+H)⁺.

### Example 5 (BVT.56664)

### 2-{2-[2-(bicyclo[2.2.1]hept-5-en-2-ylamino)-4-oxo-4,5-dihydro-1,3-thiazol-5-yl]ethyl}-1H-isoindole-1,3(2H)-dione

Prepared according to method K

Crude 2-bromo-4-(1,3-dioxo-1,3-dihydro-2H-isoindol-2-yl)butanoic acid (0.204 g, 0.654 mmol) and N-bicyclo[2.2.1]hept-5-en-2-ylthiourea (0.112 g, 0.666 mmol) were dissolved in aceton (15 ml) and heated to reflux for 8 h. The reaction mixture was allowed to cool to room temperature. NaHCO₃ (sat. solution) was added and extracted with DCM. The organic phase was concentrated in vacuum to give the crude product (0.269 g) of which 10 mg was purified using preparative LC-MS (System C, 20-80% MeCN). This afforded 6.73 mg of pure product.

Only small part of the crude was purified, the rest used in next step.

6.73 mg of pure product.

¹H NMR (270 MHz, CHLOROFORM-D) δ ppm 1.65-1.84 (m, 4H) 2.16-2.37 (m, 1H) 2.57-2.73 (m, 1H) 2.99-3.11 (m, 2H) 3.37 (t, J=4.58 Hz, 1H) 3.73-3.88 (m, 1H) 3.96-4.12 (m, 1H) 4.20 (dd, J=10.27, 3.59 Hz, 1H) 6.03-6.10 (m, 1H) 6.29 (dd, J=5.69, 2.97 Hz, 1H) 7.73-7.81 (m, 2H) 7.81-7.91 (m, 2H). MS (ESI+) for C₂₀H₁₉N₃O₃S m/z 382 (M+H)⁺.

### Example 6 (BVT.59209)

### N-{2-[2-(bicyclo[2.2.1]hept-5-en-2-ylamino)-4-oxo-4,5-dihydro-1,3-thiazol-5-yl]ethyl}-2,5-difluorobenzamide

Prepared according to method K

5-(2-aminoethyl)-2-(bicyclo[2.2.1]hept-5-en-2-ylamino)-1,3-thiazol-4(5H)-one (0.025 g, 0.101 mmol) was dissolved in a few drops of DMF and Pyridine (2 ml). 2,5-difluorobenzoyl chloride (0.053 g, 0.302 mmol) was added and the reaction mixture was shaken in room temperature. Additional 2,5-difluorobenzoyl chloride (0.036 g, 0.202 mmol) was added after 1 h and the reaction mixture was shaken in room temperature over night. 10% HCl was added and extraction with DCM performed. The organic phase was concentrated in vaccum. Purification using preparative LC-MS (System C, 30-80% MeCN) gave 0.022 g (56%) of product as yellow oil.

¹H NMR (270 MHz, METHANOL-D4) δ ppm 1.43-1.79 (m, 4H) 2.07-2.22 (m, 1H) 2.37-2.52 (m, 1H) 2.86-3.02 (m, 2H) 3.48-3.69 (m, 2H) 3.78 (dd, J=7.79, 2.85 Hz, 1H) 4.35-4.46 (m, 1H) 6.05-6.12 (m, 1H) 6.20-6.30 (m, 1H) 7.18-7.35 (m, 2H) 7.42-7.51 (m, 1H). HPLC 98%, R_{T}=1.91 min (System A, 10-97% MeCN over 3 min). 99%, R_{T}=1.65 min (System B, 10-97% MeCN over 3 min). MS (ESI+) for C₁₉H₁₉F₂N₃O₂S m/z 392 (M+H)⁺.

### Example 7 (BVT.59210)

### N-{2-[2-(bicyclo[2.2.1]hept-5-en-2-ylamino)-4-oxo-4,5-dihydro-1,3-thiazol-5-yl]ethyl}-2-chlorobenzamide

Prepared according to method K

14.1 mg, 36% yield, orange oil.

¹H NMR (270 MHz, CHLOROFORM-D) δ ppm 1.61-1.85 (m, 4H) 2.10-2.28 (m, 1H) 2.43-2.60 (m, 1H) 2.99-3.08 (m, 2H) 3.38 (dd, J=6.93, 3.46 Hz, 1H) 3.49-3.65 (m, 1H) 3.86-4.01 (m, 1H) 4.35-4.46 (m, 1H) 6.06 (dd, J=5.44, 2.97 Hz, 1H) 6.29 (dd, J=5.69, 2.97 Hz, 1H) 6.75 (t, J=5.07 Hz, 1H) 7.29-7.44 (m, 4H) 7.57-7.63 (m, 1H). MS (ESI+) for C₁₉H₂₀ClN₃O₂S m/z 390 (M+H)⁺.

### Example 8 (BVT.59211)

### N-{2-[2-(bicyclo[2.2.1]hept-5-en-2-ylamino)-4-oxo-4,5-dihydro-1,3-thiazol-5-yl]ethyl}-2-bromo-5-methoxybenzamide

Prepared according to method K

23.7 mg, 52% yield, yellow oil.

¹H NMR (270 MHz, CHLOROFORM-D) δ ppm 1.67 (s, 2H) 1.71-1.85 (m, 2H) 2.07-2.25 (m, 1H) 2.45-2.62 (m, 1H) 3.03 (d, J=8.41 Hz, 2H) 3.38 (dd, J=7.18, 3.46 Hz, 1H) 3.43-3.60 (m, 1H) 3.79 (s, 3H) 3.84-4.00 (m, 1H) 4.42-4.52 (m, 1H) 6.06 (dd, J=5.20, 3.22 Hz, 1H) 6.28 (dd, J=5.57, 2.85 Hz, 1H) 6.61 (s, 1H) 6.84 (dd, J=8.78, 2.60 Hz, 1H) 7.03 (d, J=2.97 Hz, 1H) 7.45 (d, J=8.91 Hz, 1H). MS (ESI+) for C₂₀H₂₂BrN₃O₃S m/z 466 (M+H)⁺.

### Example 9 (BVT.59330)

### N-{2-[2-(bicyclo[2.2.1]hept-5-en-2-ylamino)-4-oxo-4,5-dihydro-1,3-thiazol-5-yl]ethyl}-2-fluoro-4-(trifluoromethyl)benzamide

Prepared according to method K

0.0100 g, 11% yield.

¹H NMR (270 MHz, METHANOL-D4) δ ppm 1.34-71.70 (m, 4H) 1.95-2.10 (m, 1H) 2.32-2.42 (m, 1H) 2.79-2.80 (m, 2H) 3.46-3.55 (m, 2H) 3.70-3.74 (m, 1H) 4.23-4.30 (m, 1H) 5.95-6.00 (m, 1H) 6.10-6.13 (m, 1H) 7.47-7.52 (m, 2H) 7.76-7.82 (m, 1H). MS (ESI+) for C₂₀H₁₉F₄N₃O₂S m/z 442 (M+H)⁺.

### Example 10 (BVT.59331)

### N-{2-[2-(bicyclo[2.2.1]hept-5-en-2-ylamino)-4-oxo-4,5-dihydro-1,3-thiazol-5-yl]ethyl}-2,4-dichlorobenzamide

Prepared according to method K

0.0106 g, 12% yield.

¹H NMR (270 MHz, METHANOL-D4) δ ppm 1.36-1.71 (m, 4H) 1.96-2.05 (m, 1H) 2.21-2.39 (m, 1H) 2.80-2.83 (m, 2H) 3.31-3.55 (m, 2H) 3.68-3.72 (m, 1H) 4.27-4.32 (m, 1H) 5.96-6.02 (m, 1H) 6.10-6.14 (m, 1H) 7.29-7.44 (m, 3H). MS (ESI+) for C₁₉H₁₉Cl₂N₃O₂S m/z 424 (M+H)⁺.

### Example 11 (BVT067002)

### 2-chloro-N-{2-[2-(cyclohexylamino)-4-oxo-4,5-dihydro-1,3-thiazol-5-yl]ethyl}benzamide

Method K

5-(2-aminoethyl)-2-(cyclohexylamino)-1,3-thiazol-4(5H)-one (36 mg, 0.15 mmol) was suspended in 5% NaOH (aq.) (5 mL) and 2-chloro-benzoyl chloride (38 μL, 0.3 mmol) was added. The reaction mixture was stirred overnight. EtOAc (5 mL) was added and the reaction mixture was stirred for 10 min. The organic layer was collected and the solvent was removed under reduced pressure. Purification using preparative HPLC (20-70% MeCN over 10 min followed by 100% MeCN for 5 min) afforded the product in 19% yield, 11 mg.

¹H NMR (270 MHz, METHANOL-D4) δ ppm 1.15-1.48 (m, 5H) 1.57-1.89 (m, 3H) 1.92-2.21 (m, 3H) 2.37-2.54 (m, 1H) 3.39-3.52 (m, 1H) 3.51-3.68 (m, 1H) 3.73-3.88 (m, 1H) 4.43 (dd, J=9.77, 4.08 Hz, 1H) 7.30-7.48 (m, 4H). HPLC 93% R_{T}=1.88 (System A. 10-97% MeCN over 3 min), 96% R_{T}=1.70 (System B. 10-97% MeCN over 3 min). MS m/z: (M+H) 381.

### Example 12 (BVT067003)

### N-{2-[2-(cyclohexylamino)-4-oxo-4,5-dihydro-1,3-thiazol-5-yl]ethyl}-2,6-difluorobenzamide

Prepared according to method K

18 mg, 31% yield.

¹H NMR (270 MHz, METHANOL-D4) δ ppm 1.17-1.55 (m, 5H) 1.57-1.88 (m, 3H) 1.90-2.20 (m, 3H) 2.38-2.53 (m, 1H) 3.38-3.55 (m, 1H) 3.57-3.76 (m, 1H) 3.77-3.91 (m, 1H) 4.39 (dd, J=10.02, 4.08 Hz, 1H) 6.98-7.10 (m, 1H) 7.40-7.54 (m, 1H). MS (ESI+) for C₁₈H₂₁F₂N₃O₂S m/z 382 (M+H)⁺.

### Example 13 (BVT067004)

### N-{2-[2-(cyclohexylamino)-4-oxo-4,5-dihydro-1,3-thiazol-5-yl]ethyl}-2,6-dimethoxybenzamide

Prepared according to method K

5 mg, 8% yield.

¹H NMR (270 MHz, METHANOL-D4) δ ppm 1.12-1.48 (m, 5H) 1.58-2.07 (m, 6H) 2.35-2.53 (m, 1H) 3.35-3.44 (m, 1H) 3.60-3.78 (m, 1H) 3.72-3.84 (m, 1H) 3.79 (s, 6H) 4.50 (dd, J=10.89, 3.71 Hz, 1H) 6.64-6.70 (m, 2H) 7.28-7.34 (m, 1H). MS (ESI+) for C₂₀H₂₇N₃O₄S m/z 406 (M+H)⁺.

### Example 14 (BVT067005)

### 2-bromo-N-{2-[2-(cyclohexylamino)-4-oxo-4,5-dihydro-1,3-thiazol-5-yl]ethyl}-5-methoxybenzamide

Prepared according to method K

12 mg, 17% yield.

¹H NMR (270 MHz, METHANOL-D4) δ ppm 1.15-1.46 (m, 5H) 1.58-1.88 (m, 3H) 1.91-2.14 (m, 3H) 2.38-2.53 (m, 1H) 3.42-3.66 (m, 2H) 3.81 (s, 3H) 3.78-3.91 (m, 1H) 4.41-4.49 (m, 1H) 6.90-6.94 (m, 1H) 7.00-7.03 (m, 1H) 7.47-7.53 (m, 1H). MS (ESI+) for C₁₉H₂₄BrN₃O₃S m/z 456 (M+H)⁺.

### Example 15 (BVT067006)

### 2-chloro-N-{2-[2-(cyclohexylamino)-4-oxo-4,5-dihydro-1,3-thiazol-5-yl]ethyl}-6-fluorobenzamide

Prepared according to method K

21 mg, 35% yield.

¹H NMR (270 MHz, METHANOL-D4) δ ppm 1.17-1.47 (m, 5H) 1.68-1.71 (m, 1H) 1.71-1.88 (m, 1H) 1.92-2.07 (m, 3H) 2.39-2.53 (m, 1H) 3.33-3.47 (m, 1H) 3.55-3.72 (m, 1H) 3.78-3.90 (m, 1H) 4.39 (dd, J=10.27, 4.08 Hz, 1H) 7.14-7.20 (m, 1H) 7.31 (d, J=8.16 Hz, 1H) 7.40-7.49 (m, 1H). MS (ESI+) for C₁₈H₂₁ClFN₃O₂S m/z 399 (M+H)⁺.

### Example 16 (BVT067007)

### 2,4-dichloro-N-{2-[2-(cyclohexylamino)-4-oxo-4,5-dihydro-1,3-thiazol-5-yl]ethyl}benzamide

Prepared according to method K

19 mg, 31% yield.

¹H NMR (270 MHz, METHANOL-D4) δ ppm 1.15-1.44 (m, 5H) 1.57-1.66 (m, 1H) 1.67-1.87 (m, 2H) 1.93-2.08 (m, 3H) 2.39-2.52 (m, 1H) 3.40-3.49 (m, 1H) 3.50-3.64 (m, 1H) 3.83-3.94 (m, 1H) 4.30 (dd, J=9.77, 4.08 Hz, 1H) 7.36 (dd, J=8.16, 1.98 Hz, 1H) 7.45 (d, J=8.64 Hz 1H) 7.49 (d, J=1.73 Hz, 1H). MS (ESI+) for C₁₈H₂₁Cl₂N₃O₂S m/z 415 (M+H)⁺

### Example 17 (BVT067008)

### 2-chloro-N-{2-[4-oxo-2-(tricyclo[3.3.1.0˜3,7˜]non-3-ylamino)-4,5-dihydro-1,3-thiazol-5-yl]ethyl}benzamide

Prepared according to method K

11 mg, 18% yield.

¹H NMR (270 MHz, METHANOL-D4) δ ppm 1.50-1.69 (m, 4H) 1.94-2.57 (m, 11H) 3.42-3.63 (m, 2H) 4.30 (dd, J=9.65, 3.96 Hz, 1H) 7.31-7.49 (m, 4H). MS (ESI+) for C₂₁H₂₄ClN₃O₂S m/z 419 (M+H)⁺.

### Example 18 (BVT067009)

### 2,6-difluoro-N-{2-[4-oxo-2-(tricyclo[3.3.1.0˜3,7˜]non-3-ylamino)-4,5-dihydro-1,3-thiazol-5-yl]ethyl}benzamide

Prepared according to method K

9 mg, 14% yield.

¹H NMR (270 MHz, METHANOL-D4) δ ppm 1.52-1.73 (m, 4H) 1.86-2.24 (m, 7H) 2.20-2.34 (m, 2H) 2.35-2.57 (m, 2H) 3.28-3.50 (m, 1H) 3.52-3.69 (m, 1H) 4.25 (dd, J=10.02, 3.84 Hz, 1H) 6.97-7.09 (m, 2H) 7.40-7.52 (m, 1H). MS (ESI+) for C₂₁H₂₃F₂N₃O₂S m/z 420 (M+H)⁺.

### Example 19 (BVT067013)

### 2-chloro-6-fluoro-N-{2-[4-oxo-2-(tricyclo[3.3.1.0˜3,7˜]non-3-ylamino)-4,5-dihydro-1,3-thiazol-5-yl]ethyl}benzamide

Prepared according to method K

11 mg, 17% yield.

¹H NMR (270 MHz, METHANOL-D4) δ ppm 1.52-1.73 (m, 4H) 1.85-2.57 (m, 11H) 3.34-3.46 (m, 1H) 3.52-3.72 (m, 1H) 4.30 (dd, J=10.14, 3.96 Hz, 1H) 7.11-7.20 (m, 1H) 7.29 (d, J=7.92 Hz, 1H) 7.4137-7.46 (m, 1H). MS (ESI+) for C₂₁H₂₃ClFN₃O₂S m/z 437 (M+H)⁺.

### Example 20 (BVT067018)

### 2,4-dichloro-N-{2-[4-oxo-2-(tricyclo[3.3.1.0˜3,7˜]non-3-ylamino)-4,5-dihydro-1,3-thiazol-5-yl]ethyl}benzamide

Prepared according to method K

9 mg, 13% yield.

¹H NMR (270 MHz, METHANOL-D4) δ ppm 1.48-1.68 (m, 4H) 1.96-2.16 (m, 7H) 2.21-2.30 (m, 2H) 2.34-2.55 (m, 2H) 3.42-3.52 (m, 1H) 3.50-3.66 (m, 1H) 4.24 (dd, J=9.53, 4.08 Hz, 1H) 7.33 (dd, J=8.16, 1.98 Hz, 1H) 7.45 (d, J=1.98 Hz, 1H). MS (ESI+) for C₂₁H₂₃Cl₂N₃O₂S m/z 453 (M+H)⁺.

### Example 21 (BVT067022)

### 2-chloro-N-(2-{2-[(cyclohexylmethyl)amino]-4-oxo-4,5-dihydro-1,3-thiazol-5-yl}ethyl)benzamide

Prepared according to method K

9 mg, 15% yield.

¹H NMR (270 MHz, METHANOL-D4) δ ppm 0.89-1.10 (m, 2H) 1.10-1.37 (m, 4H) 1.55-1.82 (m, 6H) 1.94-2.09 (m, 1H) 2.40-2.53 (m, 1H) 3.18 (d, J=6.68 Hz, 1H) 3.42-3.67 (m, 2H) 4.34-3.42 (m, 1H) 7.35-50 (m, 4H). MS (ESI+) for C₁₉H₂₄ClN₃O₂S m/z 395 (M+H)⁺.

### Example 22 (BVT067025)

### 2-bromo-N-(2-{2-[(cyclohexylmethyl)amino]-4-oxo-4,5-dihydro-1,3-thiazol-5-yl}ethyl)-5-methoxybenzamide

Prepared according to method K

2 mg, 3% yield.

¹H NMR (270 MHz, METHANOL-D4) δ ppm 0.89-1.38 (m, 6H) 1.55-1.83 (m, 6H) 1.97-2.12 (m, 1H) 2.40-2.54 (m, 1H) 3.20 (d, J=6.68 Hz, 1H) 3.40-3.67 (m, 2H) 3.81 (s, 3H) 4.44 (dd, J=9.65, 4.21 Hz, 1H) 6.88-6.95 (m, 1H) 7.01 (d, J=2.97 Hz, 1H) 7.46-7.52 (m, 1H). MS (ESI+) for C₂₀H₂₆BrN₃O₃S m/z 470 (M+H)⁺.

### Example 23 (BVT067027)

### 2,4-dichloro-N-(2-{2-[(cyclohexylmethyl)amino]-4-oxo-4,5-dihydro-1,3-thiazol-5-yl}ethyl)benzamide

Prepared according to method K

8 mg, 12% yield.

¹H NMR (270 MHz, METHANOL-D4) δ ppm 0.86-1.35 (m, J=69.77 Hz, 6H) 1.53-1.84 (m, 6H) 1.96-2.08 (m, 1H) 2.35-2.52 (m, 1H) 3.14 (d, J=6.68 Hz, 1H) 3.38-3.65 (m, 2H) 4.26-4.37 (m, 1H) 7.32-7.48 (m, 3H). MS (ESI+) for C₁₉H₂₃Cl₂N₃O₂S m/z 429 (M+H)⁺.

### Example 24 (BVT067030)

### 2-chloro-N-{2-[2-(cycloheptylamino)-4-oxo-4,5-dihydro-1,3-thiazol-5-yl]ethyl}benzamide

Prepared according to method K

2 mg, 3% yield.

¹H NMR (270 MHz, METHANOL-D4) δ ppm 1.40-1.84 (m, 10H) 1.92-2.11 (m, 3H) 2.37-2.51 (m, 1H) 3.34-3.65 (m, 2H) 3.98-4.13 (m, 1H) 4.32-4.43 (m, 1H) 7.31-7.52 (m, 4H). MS (ESI+) for C₁₉H₂₄ClN₃O₂S m/z 395 (M+H)⁺.

### Example 25 (BVT067031)

### N-{2-[2-(cycloheptylamino)-4-oxo-4,5-dihydro-1,3-thiazol-5-yl]ethyl}-2,6-difluorobenzamide

Prepared according to method K

13 mg, 22% yield.

¹H NMR (270 MHz, METHANOL-D4) δ ppm 1.45-1.81 (m, 10H) 1.94-2.16 (m, 3H) 2.38-2.52 (m, 1H) 3.37-3.53 (m, 1H) 3.52-3.73 (m, 1H) 3.99-4.12 (m, 1H) 4.35 (dd, J=10.02, 4.08 Hz, 1H) 6.98-7.09 (m, 2H) 7.41-7.54 (m, 1H). MS (ESI+) for C₁₉H₂₃F₂N₃O₂S m/z 396 (M+H)⁺.

### Example 26 (BVT067032)

### N-{2-[2-(cycloheptylamino)-4-oxo-4,5-dihydro-1,3-thiazol-5-yl]ethyl}-2,6-dimethoxybenzamide

Prepared according to method K

3 mg, 5% yield.

¹H NMR (270 MHz, METHANOL-D4) δ ppm 1.42-2.15 (m, 13H) 2.38-2.57 (m, 1H) 3.52-3.83 (m, 2H) 3.81 (s, 6H) 3.98-4.14 (m, 1H) 4.47-4.54 (m, 1H) 6.64-6.72 (m, 2H) 7.27-7.38 (m, 1H). MS (ESI+) for C₂₁H₂₉N₃O₄S m/z 420 (M+H)⁺.

### Example 27 (BVT067033)

### 2-bromo-N-{2-[2-(cycloheptylamino)-4-oxo-4,5-dihydro-1,3-thiazol-5-yl]ethyl}-5-methoxybenzamide

Prepared according to method K

15 mg, 21% yield.

¹H NMR (270 MHz, METHANOL-D4) δ ppm 1.44-1.83 (m, 10H) 1.90-2.14 (m, 3H) 2.38-2.52 (m, 1H) 3.40-3.66 (m, 2H) 3.81 (s, 3H) 4.01-4.13 (m, 1H) 4.41 (dd, J=9.53, 4.08 Hz, 1H) 6.90-6.95 (m, 1H) 6.99-7.02 (m, 1H) 7.47-7.53 (m, 1H). MS (ESI+) for C₂₀H₂₆BrN₃O₃S m/z 470 (M+H)⁺.

### Example 28 (BVT062688)

### 2-chloro-N-{2-[2-(cycloheptylamino)-4-oxo-4,5-dihydro-1,3-thiazol-5-yl]ethyl}-6-fluorobenzamide

Prepared according to method K

21 mg, 34% yield.

¹H NMR (270 MHz, METHANOL-D4) δ ppm 1.44-1.77 (m, 10H) 1.86-2.06 (m, 3H) 2.37-2.51 (m, 1H) 3.34-3.47 (m, 1H) 3.49-3.71 (m, 1H) 4.01-4.13 (m, 1H) 4.33 (dd, J=10.14, 3.96 Hz, 1H) 7.10-7.18 (m, 1H) 7.29 (d, J=8.16 Hz, 1H) 7.37-7.47 (m, 1H). MS (ESI+) for C₁₉H₂₃ClFN₃O₂S m/z 412 (M+H)⁺.

### Example 29 (BVT067359)

### 2,4-dichloro-N-{2-[2-(cycloheptylamino)-4-oxo-4,5-dihydro-1,3-thiazol-5-yl]ethyl}benzamide

Prepared according to method K

14 mg, 20% yield.

¹H NMR (270 MHz, METHANOL-D4) δ ppm 1.44-1.84 (m, 10H) 1.93-2.17 (m, 3H) 2.37-54 (m, 1H) 3.42-3.67 (m, 2H) 3.97-4.12 (m, 1H) 4.38 (dd, J=9.53, 4.08 Hz, 1H) 7.37-7.56 (m, 3H). MS (ESI+) for C₁₉H₂₃Cl₂N₃O₂S m/z 428 (M+H)⁺.

### Example 30 (BVT67360)

### N-{2-[2-(cycloheptylamino)-4-oxo-4,5-dihydro-1,3-thiazol-5-yl]ethyl}-2,5-difluorobenzamide

Prepared according to method K

11 mg, 16% yield.

¹H NMR (270 MHz, METHANOL-D4) δ ppm 1.44-1.82 (m, 10H) 1.93-2.22 (m, 3H) 2.38-2.53 (m, 1H) 3.46-3.69 (m, 2H) 3.98-4.11 (m, 1H) 4.35 (dd, J=9.28, 4.08 Hz, 1H) 7.17-7.35 (m, 2H) 7.42-7.50 (m, 1H). MS (ESI+) for C₁₉H₂₃F₂N₃O₂S m/z 396 (M+H)⁺.

### Example 31 (BVT067361)

### N-{2-[2-(cycloheptylamino)-4-oxo-4,5-dihydro-1,3-thiazol-5-yl]ethyl}-2,5-bis(trifluoromethyl)benzamide

Prepared according to method K

14 mg, 18% yield.

¹H NMR (270 MHz, METHANOL-D4) δ ppm 1.43-1.82 (m, 10H) 1.93-2.16 (m, 3H) 2.34-2.50 (m, 1H) 3.42-3.66 (m, 2H) 4.00-4.15 (m, 1H) 4.35 (dd, J=9.15, 4.21 Hz, 1H) 7.90-8.04 (m, 3H). MS (ESI+) for C₂₁H₂₃F₆N₃O₂S m/z 496 (M+H)⁺.

### Example 32 (BVT067362)

### N-{2-[2-(cycloheptylamino)-4-oxo-4,5-dihydro-1,3-thiazol-5-yl]ethyl}-2-fluoro-5-(trifluoromethyl)benzamide

Prepared according to method K

10 mg, 13% yield.

¹H NMR (270 MHz, METHANOL-D4) δ ppm 1.41-1.84 (m, 10H) 1.90-2.18 (m, 3H) 2.37-2.53 (m, 1H) 3.47-3.69 (m, 2H) 3.99-4.10 (m, 1H) 4.34 (dd, J=9.28, 4.08 Hz, 1H) 7.42 (t, J=9.40 Hz, 1H) 7.81-7.91 (m, 1H) 8.05 (dd, J=6.31, 2.35 Hz, 1H). MS (ESI+) for C₂₀H₂₃F₄N₃O₂S m/z 446 (M+H)⁺.

### Example 33 (BVT067363)

### 2-chloro-N-{2-[2-(cycloheptylamino)-4-oxo-4,5-dihydro-1,3-thiazol-5-yl]ethyl}nicotinamide

Prepared according to method K

0.2 mg, 0.3% yield.

¹H NMR (270 MHz, METHANOL-D4) δ ppm 1.48-1.81 (m, 10H) 1.92-2.13 (m, 3H) 2.40-2.52 (m, 1H) 4.01-4.12 (m, 1H) 4.38 (dd, J=9.40, 4.21 Hz, 1H) 7.45 (dd, J=7.67, 4.95 Hz, 1H) 7.94 (dd, J=7.55, 1.86 Hz, 1H) 8.44 (dd, J=4.95, 1.98 Hz, 1H). MS (ESI+) FOR C₁₈H₂₃ClN₄O M/Z m/z: (M+H) 395.

### Example 34 (BVT067365)

### N-{2-[2-(cycloheptylamino)-4-oxo-4,5-dihydro-1,3-thiazol-5-yl]ethyl}-2-furamide

Prepared according to method K

7 mg, 12% yield.

¹H NMR (270 MHz, METHANOL-D4) δ ppm 1.43-1.80 (m, 10H) 1.92-2.19 (m, 3H) 2.37-2.53 (m, 1H) 3.44-3.67 (m, 2H) 3.94-4.06 (m, 1H) 4.34 (dd, J=9.40, 3.96 Hz, 1H) 6.54-6.61 (m, 1H) 7.07-7.13 (m, 1H) 7.64-7.68 (m, 1H). MS (ESI+) for C₁₈H₂₃ClN₄O₂S m/z 350 (M+H)⁺.

### Example 35 (BVT067366)

### N-{2-[2-(cycloheptylamino)-4-oxo-4,5-dihydro-1,3-thiazol-5-yl]ethyl}thiophene-2-carboxamide

Prepared according to method K

11 mg, 19% yield.

¹H NMR (270 MHz, METHANOL-D4) δ ppm 1.42-1.79 (m, 10H) 1.92-2.21 (m, 3H) 2.38-2.49 (m, 1H) 3.46-3.67 (m, 2H) 3.94-4.05 (m, 1H) 4.35 (dd, J=9.03, 4.08 Hz, 1H) 7.07-7.14 (m, 1H) 7.61-7.70 (m, 2H). MS (ESI+) for C₁₇H₂₃N₃O₂S₂ m/z 366 (M+H)⁺.

### Example 36 (BVT067367)

### N-{2-[2-(cycloheptylamino)-4-oxo-4,5-dihydro-1,3-thiazol-5-yl]ethyl}-2-(2-thienyl)acetamide

Prepared according to method K

12 mg, 20% yield.

¹H NMR (270 MHz, METHANOL-D4) δ ppm 1.43-1.81 (m, 10H) 1.84-2.09 (m, 3H) 2.26-2.43 (m, 1H) 3.23-3.50 (m, 2H) 3.71 (d, J=3.51 Hz, 2H) 3.99-4.10 (m, 1H) 4.24 (dd, J=9.65, 4.21 Hz, 1H) 6.88-6.96 (m, 2H) 7.23-7.28 (m, 1H). MS (ESI+) for C₁₈H₂₅N₃O₂S₂ m/z 381 (M+H)⁺.

### Example 37 (BVT067368)

### N-{2-[2-(cycloheptylamino)-4-oxo-4,5-dihydro-1,3-thiazol-5-yl]ethyl}cyclopropanecarboxamide

Prepared according to method K

2 mg, 4% yield.

¹H NMR (270 MHz, METHANOL-D4) δ ppm 0.69-0.89 (m, 4H) 1.44-1.81 (m, 11H) 1.87-2.09 (m, 3H) 2.28-2.42 (m, 1H) 3.23-3.49 (m, 2H) 3.98-4.09 (m, 1H) 4.27 (dd, J=9.90, 3.96 Hz, 1H). MS (ESI+) for C₁₆H₂₅N₃O₂S m/z 381 (M+H)⁺.

### Example 38 (BVT067369)

### N-{2-[2-(cycloheptylamino)-4-oxo-4,5-dihydro-1,3-thiazol-5-yl]ethyl}-3-methylbutanamide

Prepared according to method K

3 mg, 5% yield.

¹H NMR (270 MHz, METHANOL-D4) δ ppm 0.88-1.01 (m, 6H) 1.44-1.81 (m, 10H) 1.87-2.12 (m, 6H) 2.26-2.43 (m, 1H) 3.20-3.48 (m, 2H) 3.96-4.08 (m, 1H) 4.28 (dd, J=9.77, 4.08 Hz, 1H). MS (ESI+) for C₁₇H₂₉N₃O₂S m/z 340 (M+H)⁺.

### Example 39 (BVT.63235)

### N-{2-[2-(bicyclo[2.2.1]hept-5-en-2-ylamino)-4-oxo-4,5-dihydro-1,3-thiazol-5-yl]ethyl}cyclohexanecarboxamide

Prepared according to method K

0.0079 g, yield 37%).

¹H NMR (270 MHz, CHLOROFORM-D) δ ppm 1.12-1.49 (m, 5H) 1.62-1.88 (m, 9H) 1.94-2.16 (m, 2H) 2.30-2.47 (m, 1H) 3.01 (d, J=11.13 Hz, 2H) 3.22-3.49 (m, 1H) 3.58-3.75 (m, 1H) 4.18 (dd, J=10.27, 4.08 Hz, 1H) 5.95 (s, 1H) 6.05 (dd, J=5.44, 3.22 Hz, 1H) 6.27 (dd, J=5.69, 3.22 Hz, 1H). MS (ESI+) for C₁₉H₂₇N₃O₂S m/z 362 (M+H)⁺.

### Example 40 (BVT.63237)

### N-{2-[2-(bicyclo[2.2.1]hept-5-en-2-ylamino)-4-oxo-4,5-dihydro-1,3-thiazol-5-yl]ethyl}isoxazole-5-carboxamide

Prepared according to method K

0.0105 g, yield 13%.

¹H NMR (270 MHz, METHANOL-D4) δ ppm 1.39-1.79 (m, 4H) 2.01-2.20 (m, 1H) 2.38-2.53 (m, 1H) 2.84-2.99 (m, 2H) 3.47-3.66 (m, 2H) 3.80 (dd, J=7.67, 2.72 Hz, 1H) 4.29-4.39 (m, 1H) 6.07 (dd, J=5.57, 3.34 Hz, 1H) 6.21 (dd, J=5.57, 2.85 Hz, 1H) 6.94 (s, 1H) 8.10 (t, J=7.05 Hz, 1H) 8.51 (s, 1H) 8.87 (d, J=5.44 Hz, 1H). MS (ESI+) for C₁₆H₁₈N₄O₃S m/z 347 (M+H)⁺.

### Example 41 (BVT.66767)

### N-{2-[2-(bicyclo[2.2.1]hept-5-en-2-ylamino)-4-oxo-4,5-dihydro-1,3-thiazol-5-yl]ethyl}-2,4,6-trichlorobenzamide

Prepared according to method K

0.0043 g, yield 9%.

¹H NMR (270 MHz, METHANOL-D4) δ ppm 1.43-1.63 (m, 3H) 1.68-1.81 (m, 1H) 1.89-2.08 (m, 1H) 2.39-2.56 (m, 1H) 2.87-2.98 (m, 2H) 3.36-3.52 (m, 1H) 3.54-3.71 (m, 1H) 3.83 (dd, J=7.92, 2.97 Hz, 1H) 4.37-4.47 (m, 1H) 6.05-6.13 (m, 1H) 6.19-6.25 (m, 1H) 7.55 (s, 2H). MS (ESI+) for C₁₉H₁₈Cl₃N₃O₂S m/z 460 (M+H)⁺.

### Example 42 (BVT066958)

### N-[(2-azepan-1-yl-4-oxo-4,5-dihydro-1,3-thiazol-5-yl)methyl]-2-fluorobenzamide

Method T

The carboxylic acid (1.0 eq. 14 mmol) was dissolved in dry acetonitrile, Et₃N (1.0 eq,) and DPPA (1.0 eq.) were added. The reaction mixture was stirred at 50° C. for 2 hours. The reaction mixture was cooled to rt, and 1M HCl (6 ml) was added. The reaction mixture was heated to reflux for 5 hours. The acetonitrile was evaporated in vacuo and the remaining aqueous solution was saturated with solid NaCO₃, and the aqueous phase was extracted with DCM. The organic phase was evaporated in vacuo and the received intermediate amine was dissolved in DCM (3 ml) and 2-fluorobenzoyl chloride (50 μl) was added. The reaction mixture was stirred for 2 hours and the product was purified by prep-HPLC (15-60% MeCN/H₂O) to yield 3.2 mg (7%).

¹H NMR (400 MHz, CDCl₃) δ ppm 1.55 (s, 4H) 1.74-1.88 (m, 4H) 3.53 (t, J=6.0 Hz, 2H) 3.78-3.93 (m, 2H) 3.96-4.07 (m, 1H) 4.08-4.21 (m, 1H) 4.44 (t, J=5.4 Hz, 1H) 5.45 (s, 1H) 7.11 (dd, J=11.6, 8.4 Hz, 1H) 7.38-7,52 (m, 2H) 8.01 (t, J=7.7 Hz, 1H). MS (ES+) for C₁₇H₂₀FN₃O₂S m/z 350 (M+H)⁺. HPLC 95% R_{T}=2.65 min (System A. 10-97% MeCN over 3 min), 95% R_{T}=1.15 min (System B. 2-95% MeCN over 2 min).

Compounds of Type 2

### Example 43 (BVT.51528)

### 2-(bicyclo[2.2.1]hept-5-en-2-ylamino)-5-{2-[methyl(phenyl)amino]ethyl}-1,3-thiazol-4(5H)-one

Method I

2-(bicyclo[2.2.1]hept-5-en-2-ylamino)-5-(2-bromoethyl)-1,3-thiazol-4(5H)-one (0.03 g, 0.1 mmol) and N-methyl aniline (0.11 g, 1 mmol) was dissolved in DMSO (2 mL) and stirred at 60° C. for 16 h. The reaction mixture was mixed with water and the aqueous phase was extracted with ether twice. The combined organic phases were dried (MgSO₄) and the solvent was evaporated. The obtained crude product was purified by preparative reverse phase (10-90) to give 8.38 mg of the desired product. Yield 25%, 89% pure.

¹H NMR (270 MHz, METHANOL-D) δ ppm 1.58-1.42 (m, 3H) 1.72-1.67 (m, 1H) 2.29-2.18 (m, 2H) 2.95-2.85 (m, 2H) 3.11 (s, 3H) 3.38-3.35 (m, 1H) 3.76-3.62 (m, 2H) 4.44-4.37 (m, 1H) 6.10-6.04 (m, 1H) 6.29-6.20 (m, 1H) 7.26-7.14 (m, 3H) 7.45-7.40 (m, 2H). MS (ESI+) for C₁₉H₂₃N₃OS m/z 342 (M+H)⁺.

### Example 44 (BVT.51579)

### 2-(bicyclo[2.2.1]hept-5-en-2-ylamino)-5-[2-(2,3-dihydro-1H-indol-1-yl)ethyl]-1,3-thiazol-4(5H)-one

Prepared according to method I

0.0225 g, yield 80%.

¹H NMR (270 MHz, METHANOL-D4) δ ppm 1.71-1.42 (m, 4H) 2.65-2.37 (m, 2H) 3.10-2.83 (m, 5H) 3.60-3.38 (m, 4H) 4.45-4.51 (m, 1H) 6.03-5.94 (m, 1H) 6.21-6.16 (m, 1H) 6.74-6.69 (m, 2H) 7.05-6.97 (m, 2H). MS (ESI+) for C₂₀H₂₃N₃OS m/z 354 (M+H)⁺.

### Example 45 (BVT051580)

### 2-(bicyclo[2.2.1]hept-5-en-2-ylamino)-5-[2-(3,4-dihydroquinolin-1(2H)-yl)ethyl]-1,3-thiazol-4(5H)-one

Prepared according to method I

0.013 g, yield 30%.

¹H NMR (270 MHz, METHANOL-D4) δ ppm 1.71-1.47 (m, 4H) 2.01-1.93 (m, 2H) 2.41-2.23 (m, 2H) 2.80-2.65 (m, 2H) 2.96-2.87 (m, 2H) 3.19-3.13 (m, 1H) 3.41-3.28 (m, 3H) 3.63-3.52 (m, 2H) 6.09-6.02 (m, 1H) 6.23-6.21 (m, 1H) 6.70-6.61 (m, 1H) 6.83-6.80 (m, 1H) 7.04-6.93 (m, 2H). MS (ESI+) for C₂₁H₂₅N₃OS m/z 368 (M+H)⁺.

### Example 46 (BVT.51583)

### 2-(bicyclo[2.2.1]hept-5-en-2-ylamino)-5-[2-(1,3-dihydro-2H-isoindol-2-yl)ethyl]-1,3-thiazol-4(5H)-one

Prepared according to method I

0.029 g, yield 70%.

¹H NMR (270 MHz, METHANOL-D4) δ ppm 1.61-1.45 (m, 4H) 1.77-1.70 (m, 2H) 2.58-2.35 (m, 2H) 3.00-2.89 (m, 2H) 3.57-3.39 (m, 2H) 3.78-3.69 (m, 2H) 3.86-3.83 (m, 1H) 4.50-4.45 (m, 1H) 6.10-6.07 (m, 1H) 6.24-6.21 (m, 1H) 7.45-7.30 (m, 4H). MS (ESI+) for C₂₀H₂₃N₃OS m/z 354 (M+H)⁺.

### Example 47 (BVT.51590)

### 2-(bicyclo[2.2.1]hept-5-en-2-ylamino)-5-(2-piperidin-1-ylethyl)-1,3-thiazol-4(5H)-one

Prepared according to method I

0.0085 g, yield 23%.

¹H NMR (270 MHz, METHANOL-D4) δ ppm 1.61-1.43 (m, 4H) 1.98-1.67 (m, 6H) 2.48-2.26 (m, 2H) 2.97-2.88 (m, 4H) 3.15-3.10 (m, 1H) 3.39-3.37 (m, 1H) 3.60-3.50 (m, 2H) 3.86-3.83 (m, 1H) 4.49-4.43 (m, 1H) 6.09-6.07 (m, 1H) 6.23-6.21 (m, 1H). MS (ESI+) for C₁₇H₂₅N₃OS m/z 320 (M+H)⁺.

### Example 48 (BVT.61777B)

### 5-(2-Anilinoethyl)-2-[(2-methylphenyl)amino]-1,3-thiazol-4(5H)-one hydrobromide

Prepared according to method H

363 mg as a white solid, 99% yield.

¹H NMR (400 MHz, DMSO-D6) δ ppm 2.13-2.26 (m, 1H) 2.26 (s, 3H) 2.76 (m, 1H) 3.97 (m, 2H) 5.10 (t, J=8.6 Hz, 1H) 7.22 (t, J=7.4 Hz, 1H) 7.31-7.45 (m, 6H) 7.66 (d, J=7.8 Hz, 1H) 9.08 (s br, 1H) 9.89 (s br, 1H). MS (ESI+) for C₁₈H₁₉N₃OSHBr m/z 326 (M+H)⁺.

### Example 49 (BVT.61778B)

### 5-(2-Anilinoethyl)-2-[(2-methoxyphenyl)amino]-1,3-thiazol-4(5H)-one hydrobromide

Prepared according to method H

345 mg as a white solid, 99% yield.

¹H NMR (400 MHz, DMSO-D6) δ ppm 2.20 (m, 1H) 2.76 (m, 1H) 3.85 (s, 3H) 3.97 (m, 2H) 5.05 (t, J=8.8 Hz, 1H) 7.07 (dt, J=8.2 Hz, J=1.2 Hz, 1H) 7.21-7.25 (m, 2H) 7.33 (dd, J=7.8 Hz, J=1.6 Hz, 1H) 7.40-7.47 (m, 3H) 7.66 (m, 2H) 9.16 (s br, 1H) 9.87 (s br, 1H) 11.42 (s br, 1H). MS (ESI+) for C₁₈H₁₉N₃O₂SHBr m/z 342 (M+H)⁺.

### Example 50 (BVT.61779B)

### 5-(2-Anilinoethyl)-2-{[(1R,2R,3R,5S)-2,6,6-trimethylbicyclo[3.1.1]hept-3-yl]amino}-1,3-thiazol-4(5H)-one hydrobromide

Method H;

N-[(1R,2R,3R,5S)-2,6,6-trimethylbicyclo[3.1.1]hept-3-yl]thiourea (120 mg, 0.565 mmol) and 3-bromo-1-phenylpyrrolidin-2-one (136 mg, 0.566 mmol) in acetone (3 mL) were heated at 60° C. for 6 h. The solvent was removed to yield the give the product as a white solid (250 mg, 98% yield).

¹H NMR (400 MHz, DMSO-D6) δ ppm 1.01 (s, 3H) 1.04-1.10 (m, 4H) 1.19 (s, 3H) 1.64 (m, 1H) 1.78 (m, 1H) 1.93 (m, 1H) 2.06-2.17 (m, 2H) 2.29-2.37 (m, 1H) 2.61 (m, 1H) 2.66-2.80 (m, 1H) 3.90-4.00 (m, 2H) 4.01-4.11 (m, 1H) 4.99 (m, 1H) 7.19-7.24 (m, 1H) 7.38-7.45 (m, 2H) 7.63-7.68 (m, 2H) 9.43 (s br, 1H) 9.59 (s br, 1H) 10.07 (t, J=8.9 Hz, 1H). MS (ESI+) for C₂₁H₂₉N₃OS m/z 372 (M+H)⁺.

### Example 51 (BVT.61780B)

### 5-(2-Anilinoethyl)-2-{[(1S,2S,3S,5R)-2,6,6-trimethylbicyclo[3.1.1]hept-3-yl]amino}-1,3-thiazol-4(5H)-one hydrobromide

Prepared according to method H

255 mg as a white solid, 100% yield.

¹H NMR (400 MHz, DMSO-D6) δ ppm 1.01 (s, 3H) 1.04-1.10 (m, 4H) 1.19 (s, 3H) 1.64 (m, 1H) 1.78 (m, 1H) 1.93 (m, 1H) 2.06-2.18 (m, 2H) 2.29-2.38 (m, 1H) 2.61 (m, 1H) 2.66-2.80 (m, 1H) 3.90-4.00 (m, 2H) 4.01-4.11 (m, 1H) 4.98 (q, J=8.5 Hz, 1H) 7.17-7.25 (m, 1H) 7.38-7.45 (m, 2H) 7.63-7.68 (m, 2H) 9.42 (s br, 1H) 9.59 (s br, 1H) 10.07 (t, J=9.0 Hz, 1H). MS (ESI+) for C₂₁H₂₉N₃OSHBr m/z 372 (M+H)⁺.

### Example 52 (BVT.61791B)

### 5-(2-Anilinoethyl)-2-(tricyclo[3.3.1.0˜3,7˜]non-3-ylamino)-1,3-thiazol-4(5H)-one

Prepared according to method H

110 mg as a white solid, 71% yield.

¹H NMR (400 MHz, DMSO-D6) δ ppm 1.44-1.59 (m, 4H) 1.71 (m, 2H) 1.87 (m, 2H) 2.10 (m, 1H) 2.32 (m, 2H) 2.51 (m, 1H) 2.63 (m, 1H) 2.69 (m, 1H) 3.92-4.02 (m, 2H) 4.99 (t, J=8.8 Hz, 1H) 7.23 (m, 1H) 7.43 (m, 2H) 7.64 (m, 2H) 8.32 (s br, 1H) 9.80 (s br, 1H) 10.43 (s, 1H). MS (ESI+) for C₂₀H₂₅N₃OSHBr m/z 356 (M#H)⁺.

### Example 53 (BVT.59495)

### 5-(2-anilinoethyl)-2-(bicyclo[2.2.1]hept-2-ylamino)-1,3-thiazol-4(5H)-one

Prepared according to method H

0.736 g, yield 99% of white crystals.

¹H NMR (270 MHz, DMSO-D6) δ ppm 0.99-1.19 (m, 3H) 1.30-1.54 (m, 4H) 1.66 (dd, J=11.51, 9.03 Hz, 1H) 1.73-1.90 (m, 1H) 2.13-2.38 (m, 3H) 3.01-3.15 (m, 2H) 3.73 (d, J=4.21 Hz, 1H) 4.17-4.32 (m, 1H) 6.52 (t, J=7.67 Hz, 3H) 7.05 (t, J=7.79 Hz, 2H) 9.10 (d, J=6.68 Hz, 1H). MS (ESI+) for C₁₈H₂₃N₃OS m/z 330 (M+H)⁺.

### Example 54 (BVT.59587)

### 5-(2-anilinoethyl)-2-[(2-cyclohex-1-en-1-ylethyl)amino]-1,3-thiazol-4(5H)-one

Prepared according to method H

28.9 mg, 16% yield.

1H NMR (400 MHz, METHANOL-D4) δ ppm 1.56 (m, 2H) 1.65 (m, 2H) 1.99 (m, J=4.15 Hz, 4H) 2.16 (m, 1H) 2.31 (t, J=6.84 Hz, 2H) 2.79 (m, 1H) 3.48 (m, 2H) 4.04 (m, 2H) 4.71 (m, 1H) 5.54 (s, 1H) 7.26 (t, J=7.45 Hz, 1H) 7.42 (t, J=7.93 Hz, 2H) 7.63 (d, J=8.30 Hz, 2H). MS (ES+) for C₁₉H₂₅N₃OS m/z 344 (M+H)⁺.

### Example 55 (BVT.61703B)

### 5-(2-anilinoethyl)-2-[(1,1,3,3-tetramethylbutyl)amino]-1,3-thiazol-4(5H)-one hydrobromide

Prepared according to method H

31.6 mg, yield 28%.

1H NMR (400 MHz, DMSO-D6) δ ppm 0.98 (s, 9H) 1.43 (s, 3H) 1.45 (s, 3H) 1.69 (d, J=15.38 Hz, 1H) 1.86 (m, 1H) 2.12 (m, 1H) 2.69 (m, 1H) 3.97 (m, 2H) 4.91 (t, J=8.91 Hz, 1H) 7.24 (t, J=7.32 Hz, 1H) 7.44 (t, J=7.81 Hz, 2H) 7.64 (d, J=7.81 Hz, 2H) 8.62 (s, 1H) 9.86 (s, 1H). MS (ES+) for C₁₉H₂₉N₃OSHBr m/z 348 (M+H)⁺.

### Example 56 (BVT.61802)

### 5-(2-anilinoethyl)-2-(2,3-dihydro-1H-inden-2-ylamino)-1,3-thiazol-4(5H)-one

Prepared according to method H

115 mg, 63% yield.

¹H NMR (400 MHz, DMSO-D6) δ ppm 2.03 (m, 1H) 2.66 (m, 1H) 2.94 (dd, J=16.11, 5.37 Hz, 2H) 3.35 (m, 2H) 3.90 (m, 2H) 4.50 (m, 1H) 4.88 (t, J=8.67 Hz, 1H) 7.18 (m, 5H) 7.38 (t, J=7.93 Hz, 2H) 7.59 (d, J=7.81 Hz, 2H) 9.39 (s, 1H) 9.68 (s, 1H) 10.28 (d, J=6.84 Hz, 1H). MS (ESI+) for C₂₀H₂₁N₃OS m/z 352 (M+H)⁺.

### Example 57 (BVT.61804B)

### 5-(2-anilinoethyl)-2-[(cyclohexylmethyl)amino]-1,3-thiazol-4(5H)-one

Prepared according to method H

85 mg, 44% yield.

¹H NMR (400 MHz, DMSO-D6) δ ppm 0.85-1.77 (m, 11H) 2.12 (m, 1H) 2.71 (m, 1H) 3.19 (d, J=7.08 Hz, 2H) 3.95 (m, 2H) 4.90 (t, J=8.79 Hz, 1H) 7.23 (t, J=7.45 Hz, 1H) 7.43 (m, 2H) 7.65 (d, J=8.79 Hz, 2H). MS (ESI+) for C₁₉H₂₅N₃OS m/z 332 (M+H)⁺.

### Example 58 (BVT.61805C)

### 5-(2-anilinoethyl)-2-[(2,2,6,6-tetramethylpiperidin-4-yl)amino]-1,3-thiazol-4(5H)-one

Prepared according to method H

30 mg, 17% yield.

¹H NMR (400 MHz, DMSO-D6) δ ppm 1.4-2.0 (m, 17H) 2.12 (m, 1H) 2.71 (m, 1H) 3.96 (m, 2H) 5.07 (t, J=8.67 Hz, 1H) 7.22 (t, J=7.32 Hz, 1H) 7.43 (t, J=7.81 Hz, 2H) 7.64 (d, J=8.06 Hz, 2H). MS (ESI+) for C₂₀H₃₀N₄OS m/z 375 (M+H)⁺.

### Example 59 (BVT.61983B)

### 5-(2-anilinoethyl)-2-(cyclohexylamino)-1,3-thiazol-4(5H)-one hydrobromide

Prepared according to method H

120 mg, 30% yield.

¹H NMR (400 MHz, DMSO-D6) δ ppm 1.0-2.0 (m, 10H) 2.11 (m, 1H) 2.70 (m, 1H) 3.66 (s, 1H) 3.96 (m, 2H) 4.92 (t, J=8.67 Hz, 1H) 7.23 (t, J=7.32 Hz, 1H) 7.43 (t, J=7.81 Hz, 2H) 7.64 (d, J=8.30 Hz, 2H) 9.31 (s, 1H) 9.58 (s, 1H) 9.92 (d, J=7.81 Hz, 1H). MS (ESI+) for C₁₇H₂₃N₃OS m/z 318 (M+H)⁺.

### Example 60 (BVT.61995C)

### 5-(2-anilinoethyl)-2-{[(1R)-1-phenylethyl]amino}-1,3-thiazol-4(5H)-one hydrochloride

Prepared according to method H

0.057 g 43% yield.

¹H NMR (400 MHz, DMSO-D6) δ ppm 1.52 (d, J=6.10 Hz, 3H) 2.11 (m, 1H) 2.69 (m, 1H) 3.94 (m, J=7.32, 3.66 Hz, 2H) 5.10 (m, 1H) 5.23 (m, 1H) 7.22 (t, J=7.32 Hz, 1H) 7.31 (m, 1H) 7.39 (m, 2H) 7.44 (t, J=8.06 Hz, 4H) 7.64 (d, J=7.08 Hz, 2H). MS (ES+) for C₁₉H₂₁N₃OSHCl m/z 340 (M+H)⁺.

### Example 61 (BVT.61996C)

### 5-(2-anilinoethyl)-2-{[(1S)-1-phenylethyl]amino}-1,3-thiazol-4(5H)-one hydrochloride

Prepared according to method H

0.0512 g, 36% yield.

¹H NMR (400 MHz, DMSO-D6) δ ppm 1.52 (d, J=6.35 Hz, 3H) 2.10 (m, 1H) 2.70 (m, 1H) 3.94 (m, 2H) 5.07 (m, 1H) 5.21 (m, 1H) 7.22 (t, J=7.20 Hz, 1H) 7.32 (m, 1H) 7.41 (m, 6H) 7.64 (d, J=7.57 Hz, 2H). MS (ES+) for C₁₉H₂₁N₃OSHCl m/z 340 (M+H)⁺.

### Example 62 (BVT.61997C)

### 5-(2-anilinoethyl)-2-{[(2R)-2-phenylpropyl]amino}-1,3-thiazol-4(5H)-one hydrochloride

Prepared according to method H

0.0465 g, 22% yield.

¹H NMR (400 MHz, DMSO-D6) δ ppm 1.25 (dd, J=6.84, 1.46 Hz, 3H) 2.04 (m, 1H) 2.61 (m, 1H) 3.09 (m, 1H) 3.56 (m, 2H) 3.91 (m, 2H) 4.84 (td, J=8.55, 2.44 Hz, 1H) 7.23 (m, 2H) 7.32 (m, 4H) 7.42 (t, J=7.93 Hz, 2H) 7.63 (d, J=8.30 Hz, 2H). MS (ES+) for C₂₀H₂₃N₃OSHCl m/z 354 (M+H)⁺.

### Example 63 (BVT.61824B)

### 5-(2-anilinoethyl)-2-(cycloheptylamino)-1,3-thiazol-4(5H)-one

Prepared according to method H

0.0144 g, 34% yield of white crystals.

¹H NMR (270 MHz, DMSO-D6) δ ppm 1.31-1.69 (m, 12H) 1.81-2.04 (m, 3H) 3.09-3.46 (m, 1H) 3.95 (s, 2H) 7.11 (d, J=7.18 Hz, 3H) 7.32 (d, J=7.42 Hz, 2H) 9.62 (s, 1H). MS (ESI+) for C₁₈H₂₅N₃OS m/z 332 (M+H)⁺.

### Example 64 (BVT.59446)

### 5-(2-anilinoethyl)-2-[(4-methylbenzyl)amino]-1,3-thiazol-4(5H)-one

Prepared according to method H

17.52 mg, yield 6.2%.

¹H NMR (400 MHz, METHANOL-D4) δ ppm 2.15 (m, 1H) 2.33 (m, 3H) 2.78 (m, 1H) 4.03 (m, 2H) 4.54 (m, 2H) 4.76 (m, 1H) 7.25 (m, 5H) 7.41 (m, 2H) 7.60 (m, 2H). MS (ESI+) for C₁₉H₂₁N₃OS m/z 340 (M+H)⁺.

### Example 65 (BVT.62617B)

### 5-(2-anilinoethyl)-2-(cyclooctylamino)-1,3-thiazol-4(5H)-one hydrobromide

Prepared according to method H

0.0795 g, yield 85%, as white crystals.

¹H NMR (270 MHz, DMSO-D6) δ ppm 1.40-1.89 (m, 15H) 1.99-2.22 (m, 2H) 2.69 (d, J=7.42 Hz, 1H) 3.77-4.01 (m, 2H) 4.88 (t, J=8.78 Hz, 1H) 7.23 (t, J=7.30 Hz, 1H) 7.43 (t, J=7.92 Hz, 2H) 7.64 (d, J=7.92 Hz, 2H) 9.95 (d, J=8.16 Hz, 1H). MS (ESI+) for C₁₉H₂₇N₃OS m/z 346 (M+H)⁺.

### Example 66 (BVT062639B)

### 5-(2-anilinoethyl)-2-{[(1R)-1-cyclohexylethyl]amino}-1,3-thiazol-4(5H)-one

Prepared according to method H

32.4 mg, yield 14% as a white solid.

¹H NMR (400 MHz, DMSO-d₆) δ ppm 0.96 (m, 2H) 1.14 (d, J=6.6 Hz, 3H) 1.14 (m, 3H) 1.44 (m, 1H) 1.61 (d, J=10.5 Hz, 1H) 1.71 (d, J=10.5 Hz, 4H) 2.12 (m, 1H) 2.70 (m, 1H) 3.51 (m, 1H) 3.68 (d, J=5.6 Hz, 1H) 3.95 (m, 2H) 4.89 (td, J=8.7, 4.4 Hz, 1H) 7.23 (t, J=7.5 Hz, 1H) 7.43 (t, J=7.9 Hz, 2H) 7.64 (d, J=8.1 Hz, 2H) 9.86 (d, J=8.8 Hz, 1H). MS (ES+) for C₁₉H₂₇N₃OS m/z 346 (M+H)⁺.

### Example 67 (BVT062640B)

### 5-(2-anilinoethyl)-2-{[(1S)-1-cyclohexylethyl]amino}-1,3-thiazol-4(5H)-one

Prepared according to method H

87.0 mg, yield 33% as a white solid.

¹H NMR (400 MHz, DMSO-d₆) δ ppm 0.98 (m, 2H) 1.14 (d, J=6.4 Hz, 3H) 1.15 (m, 3H) 1.45 (m, 1H) 1.61 (d, J=9.8 Hz, 1H) 1.71 (d, J=10.0 Hz, 4H) 2.12 (m, 1H) 2.70 (m, 1H) 3.35 (m, 1H) 3.73 (s, 1H) 3.94 (m, 2H) 4.94 (m, 1H) 7.22 (t, J=7.3 Hz, 1H) 7.43 (t, J=7.9 Hz, 2H) 7.64 (d, J=7.8 Hz, 2H) 9.87 (d, J=8.6 Hz, 1H). MS (ES+) for C₁₉H₂₇N₃OS m/z 346 (M+H)⁺.

### Example 68 (BVT063212B)

### 5-(2-anilinoethyl)-2-azepan-1-yl-1,3-thiazol-4(5H)-one

Prepared according to method A followed by method H.

83.4 mg, yield 5% over two steps.

¹H NMR (400 MHz, DMSO-d₆) δ ppm 1.54 (m, 4H) 1.74 (m, 4H) 2.18 (m, 1H) 2.73 (m, 1H) 3.40 (s, 1H) 3.68 (m, 2H) 3.79 (m, 2H) 3.97 (m, 2H) 4.97 (m, 1H) 7.23 (t, J=7.5 Hz, 1H) 7.43 (t, J=7.9 Hz, 2H) 7.65 (d, J=8.1 Hz, 2H). MS (ES+) for C₁₇H₂₃N₃OS m/z 318 (M+H)⁺.

### Example 69 (BVT063213B)

### 5-(2-anilinoethyl)-2-{[(2S)-2-phenylpropyl]amino}-1,3-thiazol-4(5H)-one

Prepared according to method A followed by method H.

61.0 mg, yield 83%.

¹H NMR (400 MHz, DMSO-d₆) δ ppm 1.25 (dd, J=6.8, 2.0 Hz, 3H) 2.06 (m, 1H) 2.60 (m, 1H) 3.07 (m, 1H) 3.53 (m, 2H) 3.92 (m, 2H) 4.73 (t, J=8.8 Hz, 1H) 7.23 (m, 2H) 7.31 (m, 4H) 7.43 (t, J=7.8 Hz, 2H) 7.63 (d, J=8.3 Hz, 2H). MS (ES+) for C₂₀H₂₃N₃OS m/z 354 (M+H)⁺.

### Example 70 (BVT.63334B)

### 2-Anilino-5-(2-anilinoethyl)-1,3-thiazol-4(5H)-one hydrobromide

Prepared according to method H.

84 mg, yield 55% as a white solid.

1H NMR (400 MHz, METHANOL-D4) δ ppm 2.23 (m, 1H) 2.85 (m, 1H) 4.08 (m, 2H) 4.93 (t, J=8.55 Hz, 1H) 7.27 (t, J=7.45 Hz, 1H) 7.44 (m, 5H) 7.55 (m, 2H) 7.65 (d, J=7.81 Hz, 2H). MS (ES) for C₁₇H₁₇N₃OS m/z 312 (M+H)⁺.

### Example 71 (BVT.66791T)

### 2-[(cyclohexylmethyl)amino]-5-{2-[(4-fluorophenyl)amino]ethyl}-1,3-thiazol-4(5H)-one trifluoroacetate

Prepared according to method H.

9.8 mg, yield 3%.

1H NMR (400 MHz, CHLOROFORM-D) δ ppm 1.07 (m, 5H) 1.64 (m, 6H) 2.12 (m, 1H) 2.81 (m, 1H) 3.20 (m, 2H) 3.96 (m, 2H) 4.45 (m, 1H) 7.09 (m, 2H) 7.50 (m, 2H) MS (ES) for C₁₈H₂₄FN₃OS m/z 350 (M+H)⁺.

Compounds of Type 3

### Example 72 (BVT.51282)

### 2-(bicyclo[2.2.1]hept-5-en-2-ylamino)-5-[2-(3,4-dihydroquinolin-1(2H)-yl)-2-oxoethyl]-1,3-thiazol-4(5H)-one

Method D

[2-(Bicyclo[2.2.1]hept-5-en-2-ylamino)-4-oxo-4,5-dihydro-1,3-thiazol-5-yl]acetic acid (0.10 g, 0.375 mmol) and 2-chloro-1-methylpyridinium iodide (0.115 g, 0.451 mmol) was mixed in DCM (3 mL) for 10 minutes before 1,2,3,4-tetrahydroquinoline (0.05 g, 0.375 mmol) was added followed by Et₃N (0.057 g, 0.563 mmol). The reaction mixture was stirred for 16 h, full conversion of the SM. The reaction mixture was poured on a Hydromatrix column (pretreated with water) and the crude product was eluted with DCM. The obtained crude product was purified by preparative reverse phase (30-60) as gradient. This gave 0.090 g of the title compound, 98% pure, yield 63%.

¹H NMR (270 MHz, DMSO-D6) δ ppm 1.57-1.40 (m, 4H) 1.95-1.78 (m, 2H) 2.73-2.68 (m, 2H) 2.97-2.80 (m, 3H) 3.61-3.41 (m, 2H) 3.83-3.68 (m, 2H) 4.34-4.26 (m, 1H) 6.12-6.06 (m, 1H) 6.25-6.20 (m, 1H) 7.24-7.10 (m, 4H) 9.30 (br.d, 7.43 Hz, 1H, N—H). MS (ESI+) for C₂₁H₂₃N₃O₂S m/z 382 (M+H)⁺.

### Example 73 (BVT.47556)

### 2-[2-(Bicyclo[2.2.1]hept-5-en-2-ylamino)-4-oxo-4,5-dihydro-1,3-thiazol-5-yl]-N-(3-chloro-2-methylphenyl)acetamide

Prepared according to method D

83 mg, 28% yield as a white solid. Mp 201-202° C. ¹H NMR (400 MHz, DMSO-D6) δ ppm 1.41-1.62 (m, 4H) 2.21 (s, 3H) 2.67-2.86 (m, 3H) 3.20-3.27 (m, 1H, obscured by HDO peak) 3.75-3.76 (m, 1H) 4.34-4.40 (m, 1H) 6.08 (dd, J1=5.52, J2=3.01 Hz, 1H) 6.21 (dd, J1=5.52, J2=2.76 Hz, 1H) 7.18 (t, J=7.91 Hz, 1H) 7.27-7.33 (m, 2H) 9.37 (br s, NH) 9.76 (br s, NH). MS (EI⁺) for C₁₉H₂₀ClN₃O₂S m/z 390.0 (M+H)⁺.

### Example 74 (BVT.49940)

### N-Benzyl-2-[2-(bicyclo[2.2.1]hept-5-en-2-ylamino)-4-oxo-4,5-dihydro-1,3-thiazol-5-yl]acetamide

Prepared according to method D

30 mg of white crystals: Mp 206-207° C. ¹H NMR (400 MHz, DMSO-D6) δ ppm 1.41-1.59 (m, 4H) 2.45-2.53 (m, 1H) 2.79-2.86 (m, 2H) 3.04-3.11 (m, 1H) 3.73-3.77 (m, 1H) 4.22-4.34 (m, 3H) 6.07-6.10 (m, 1H) 6.19-6.24 (m, 1H) 7.21-7.26 (m, 3H) 7.29-7.33 (m, 2H) 8.53-8.57 (m, NH) 9.26 (d, J=7.03 Hz, NH). MS (EI+) for C₁₉H₂₁N₃O₂S m/z 377.2 (M+H)⁺.

### Example 75 (BVT.51283)

### N-benzyl-2-[2-(bicyclo[2.2.1]hept-5-en-2-ylamino)-4-oxo-4,5-dihydro-1,3-thiazol-5-yl]-N-phenylacetamide

Prepared according to method D

0.091 g, yield 57%.

¹H NMR (270 MHz, DMSO-D6) δ ppm 1.64-1.42 (m, 4H) 2.46-2.25 (m, 1H) 3.00-2.75 (m, 3H) 3.80-3.65 (m, 1H) 4.34-4.26 (m, 1H) 4.90-4.85 (m, 2H) 6.08-6.04 (m, 1H) 6.26-6.20 (m, 1H) 7.41-7.18 (m, 10H) 9.26 (d, J=6.93 Hz, 1H; N—H). MS (ESI+) for C₂₅H₂₅N₃O₂S m/z 432 (M+H)⁺.

### Example 76 (BVT.51284)

### 2-[2-(bicyclo[2.2.1]hept-5-en-2-ylamino)-4-oxo-4,5-dihydro-1,3-thiazol-5-yl]-N-(4-methoxyphenyl)-N-methylacetamide

Prepared according to method D

0.095 g, yield 66%.

¹H NMR (270 MHz, DMSO-D6) δ ppm 1.56-1.40 (m, 4H) 2.30-2.23 (m, 1H) 2.92-2.74 (m, 4H) 3.13 (s, 3H) 3.78 (s, 3H) 4.26-4.17 (m, 1H) 6.08-6.04 (m, 1H) 6.26-6.18 (m, 1H) 7.01-6.98 (m, 2H) 7.32-7.26 (m, 2H) 9.32 (br.d, J=6.83 Hz, 1 H., N—H). MS (ESI+) for C₂₀H₂₃N₃O₃S m/z 386 (M+H)⁺.

### Example 77 (BVT.51285)

### 2-[2-(bicyclo[2.2.1]hept-5-en-2-ylamino)-4-oxo-4,5-dihydro-1,3-thiazol-5-yl]-N-methyl-N-phenylacetamide

Prepared according to method D

0.11 g, yield 83%.

¹H NMR (270 MHz, DMSO-D6) δ ppm 1.58-1.39 (m, 4H) 2.44-2.25 (m, 1H) 2.97-2.75 (m, 4H) 3.17 (s, 3H) 4.27.4.17 (m, 1H) 6.08-6.06 (m, 1H) 6.26-6.19 (m, 1H) 7.49-7.30 (m, 5H) 9.34 (br.d, J=7.17 Hz, 1H, N—H). MS (ESI+) for C₁₉H₂₁N₃O₂S m/z 356 (M+H)⁺.

### Example 78 (BVT.51286)

### 2-(bicyclo[2.2.1]hept-5-en-2-ylamino)-5-[2-(1,3-dihydro-2H-isoindol-2-yl)-2-oxoethyl]-1,3-thiazol-4(5H)-one

Prepared according to method D

0.08 g, yield 6%.

¹H NMR (270 MHz, DMSO-D6) δ ppm 1.62-1.43 (m, 4H) 2.88-2.75 (m, 3H) 3.31-3.29 (m, 2H) 4.35-4.28 (m, 1H) 4.65 (s, 2H) 4.84 (s, 2H) 6.12-6.10 (m, 1H) 6.24-6.22 (m, 1H) 7.37-7.27 (m, 4H) 9.35 (br.d, J=9.63 Hz, 1H, N—H). MS (ESI+) for C₂₀H₂₁N₃O₂S m/z 368 (M+H)⁺.

### Example 79 (BVT.51296)

### 2-(bicyclo[2.2.1]hept-5-en-2-ylamino)-5-[2-(2,3-dihydro-1H-indol-1-yl)-2-oxoethyl]-1,3-thiazol-4(5H)-one

Prepared according to method D

0.007 g, yield 5%.

¹H NMR (270 MHz, DMSO-D6) δ ppm 1.63-1.42 (m, 4H) 2.92-2.82 (m, 3H) 3.17-3.11 (m, 3H) 3.80-3.70 (m, 1H) 4.12-4.03 (m, 2H) 4.39-4.30 (m, 1H) 6.12-6.10 (m, 1H) 6.24-6.21 (m, 1H) 7.03-6.98 (m, 1H) 7.19-7.16 (m, 1H) 7.26-7.23 (m, 1H) 8.03 (d, J=7.92 Hz, 1H) 9.34 (br.d, J=7.17 Hz, 1HH, N—H). MS (ESI+) for C₂₀H₂₁N₃O₂S m/z 368 (M+H)⁺.

### Example 80 (BVT056660)

### 2-[2-(bicyclo[2.2.1]hept-5-en-2-ylamino)-4-oxo-4,5-dihydro-1,3-thiazol-5-yl]-N-ethyl-N-(3-methylphenyl)acetamide

Prepared according to method D

34.15 mg, yield 45%.

¹H NMR (270 MHz, DMSO-D6) δ ppm 0.97-1.02 (m, 3H) 1.40-1.59 (m, 4H) 2.20-2.32 (m, 5H) 2.75-2.87 (m, 3H) 3.20-3.30 (m, 1H) 3.60-3.72 (m, 2H) 4.17-4.23 (m, 1H, 6.06-6.10 (m, 1H) 6.19-6.26 (m, 1H) 7.10-7.24 (m, 3H) 7.33-7.43 (m, 1H) 9.26-9.29 (m, 1H, N—H). MS (ESI+) for C₂₁H₂₅N₃O₂S m/z 384 (M+H)⁺.

### Example 81 (BVT056661)

### 2-(bicyclo[2.2.1]hept-5-en-2-ylamino)-5-[2-(3,4-dihydroisoquinolin-2(1H)-yl)-2-oxoethyl]-1,3-thiazol-4(5H)-one

Prepared according to method D

9.08 mg, yield 12%.

¹H NMR (270 MHz, DMSO-D6) δ ppm 1.39-1.64 (m, 4H) 2.75-2.93 (m, 5H) 3.30-3.40 (m, 1H) 3.59-3.78 (m, 4H) 4.23-4.33 (m, 1H) 4.58-4.69 (m, 1H) 6.05-6.17 (m, 1H) 6.19-6.27 (m, 1H) 4.06 (br.s, 4H) 9.30-9.32 (m, 1H, N—H). MS (ESI+) for C₂₁H₂₃N₃O₂S m/z 382 (M+H)⁺.

### Example 82 (BVT056662)

### 2-[2-(bicyclo[2.2.1]hept-5-en-2-ylamino)-4-oxo-4,5-dihydro-1,3-thiazol-5-yl]-N-methyl-N-[4-(trifluoromethoxy)phenyl]acetamide

Prepared according to method D

16.26 mg, yield 19%.

¹H NMR (270 MHz, DMSO-D6) δ ppm 1.37-1.62 (m, 4H) 2.75-2.96 (m, 4H) 3.13-3.23 (m, 4H) 3.66-3.79 (m, 1H) 4.17-4.28 (m, 1H) 6.03-6.12 (m, 1H) 6.17-6.26 (m, 1H) 7.37-7.59 (m, 4H) 9.27-9.30 (m, 1H, N—H). MS (ESI+) for C₂₀H₂₀F₃N₃O₃S m/z 440 (M+H)⁺.

### Example 83 (BVT056663)

### 2-[2-(bicyclo[2.2.1]hept-5-en-2-ylamino)-4-oxo-4,5-dihydro-1,3-thiazol-5-yl]-N-ethyl-N-[4-(trifluoromethoxy)phenyl]acetamide

Prepared according to method D

10.19 mg, yield 11%.

¹H NMR (270 MHz, DMSO-D6) δ ppm 0.97-1.08 (m, 3H) 1.34-1.64 (m, 4H) 2.21-2.45 (m, 2H) 2.77-2.94 (m, 3H) 3.62-3.77 (m, 3H) 4.18-4.28 (m, 1H) 6.06-6.13 (m, 1H) 6.19-6.26 (m, 1H) 7.45-7.55 (m, 4H) 9.26-9.29 (m, 1H, N—H). MS (ESI+) for C₂₁H₂₂F₃N₃O₃S m/z 454 (M+H)⁺.

### Example 84 (BVT056666)

### 2-(bicyclo[2.2.1]hept-5-en-2-ylamino)-5-{2-oxo-2-[7-(trifluoromethyl)-3,4-dihydroquinolin-1(2H)-yl]ethyl}-1,3-thiazol-4(5H)-one

Prepared according to method D

11.5 mg, yield 13%.

¹H NMR (270 MHz, DMSO-D6) δ ppm 0.97-1.05 (m, 3H) 1.37-1.61 (m, 5H) 2.21-2.42 (m, 1H) 2.75-2.92 (m, 3H) 3.40-3.50 (m, 1H) 3.59-3.77 (m, 3H) 4.16-4.26 (m, 1H) 6.05-6.15 (m, 1H) 6.19-6.26 (m, 1H) 7.43-7.5 (m, 3H) 9.26-9.29 (m, 1H, N—H). MS (ESI+) for C₂₂H₂₂F₃N₃O₂S m/z 450 (M+H)⁺.

### Example 85 (BVT056668)

### 2-[2-(bicyclo[2.2.1]hept-5-en-2-ylamino)-4-oxo-4,5-dihydro-1,3-thiazol-5-yl]-N-methyl-N-(2-methylphenyl)acetamide

Prepared according to method D

10.99 mg, yield 15%.

¹H NMR (270 MHz, DMSO-D6) δ ppm 1.38-1.62 (m, 4H) 1.95-2.37 (m, 4H) 2.52 (s, 3H) 2.76-2.91 (m, 2H) 3.08 (s, 3H) 4.16-4.31 (m, 1H) 6.04-6.12 (m, 1H) 6.19-6.28 (m, 1H) 7.23-7.42 (m, 4H) 9.36-9.38 (m, 1H, N—H). MS (ESI+) for C₂₀H₂₃N₃O₂S m/z 370 (M+H)⁺.

### Example 86 (BVT056669)

### 2-[2-(bicyclo[2.2.1]hept-5-en-2-ylamino)-4-oxo-4,5-dihydro-1,3-thiazol-5-yl]-N-ethyl-N-phenylacetamide

Prepared according to method D

23.29 mg, yield 32%.

¹H NMR (270 MHz, DMSO-D6) δ ppm 0.98-1.03 (m, 3H) 1.37-1.59 (m, 4H) 2.19-2.34 (m, 1H) 2.72-2.89 (m, 3H) 3.60-3.70 (m, 3H) 4.17-4.27 (m, 1H) 6.04-6.12 (m, 1H) 6.19-6.26 (m, 1H) 7.32-7.52 (m, 5H) 9.34-9.37 (m, 1H, N—H). MS (ESI+) for C₂₀H₂₃N₃O₂S m/z 370 (M+H)⁺.

### Example 87 (BVT056670)

### 2-[2-(bicyclo[2.2.1]hept-5-en-2-ylamino)-4-oxo-4,5-dihydro-1,3-thiazol-5-yl]-N-methyl-N-(4-methylphenyl)acetamide

Prepared according to method D

21.33 mg, yield 29%.

¹H NMR (270 MHz, DMSO-D6) δ ppm 1.37-1.62 (m, 4H) 2.22-2.40 (m, 2H) 2.33 (s, 3H) 2.75-2.93 (m, 3H) 3.14 (s, 3H) 3.20-3.35 (m, 1H) 4.17-4.27 (m, 1H) 6.06-6.13 8m, 1H) 6.19-6.27 (m, 1H) 7.16-7.31 (m, 4H) 9.32-9.35 (m, 1H, N—H). MS (ESI+) for C₂₀H₂₃N₃O₂S m/z 370 (M+H)⁺.

### Example 88 (BVT056671)

### 2-[2-(bicyclo[2.2.1]hept-5-en-2-ylamino)-4-oxo-4,5-dihydro-1,3-thiazol-5-yl]-N-(4-bromophenyl)-N-methylacetamide

Prepared according to method D

17.54 mg, yield 20%.

¹H NMR (270 MHz, DMSO-D6) δ ppm 1.38-1.59 (m, 4H) 2.22-2.57 (m, 3H) 2.74-2.97 (m, 4H) 3.16 (s, 3H) 4.15-4.27 (m, 1H) 6.06-6.12 (m, 1H) 6.19-6.26 (m, 1H) 7.29-7.41 (m, 1H) 7.60-7.73 (m, 1H) 9.32-9.34 (m, 1H, N—H). MS (ESI+) for C₁₉H₂₀BrN₃O₂S m/z 434 (M+H)⁺.

### Example 89 (BVT056672)

### 2-[2-(bicyclo[2.2.1]hept-5-en-2-ylamino)-4-oxo-4,5-dihydro-1,3-thiazol-5-yl]-N-(4-chlorophenyl)-N-methylacetamide

Prepared according to method D

15.8 mg, yield 21%.

¹H NMR (270 MHz, DMSO-D6) δ ppm 1.38-1.68 (m, 4H) 2.27-2.54 (m, 2H) 2.76-2.99 (m, 3H) 3.16 (s, 3H) 4.14-4.30 (m, 1H) 6.06-6.13 (m, 1H) 6.19-6.24 (m, 1H) 7.36-7.58 (m, 4H) 9.33-9.35 (m, 1H, N—H). MS (ESI+) for C₁₉H₂₀ClN₃O₂S m/z 390 (M+H)⁺.

### Example 90 (BVT056673)

### 2-[2-(bicyclo[2.2.1]hept-5-en-2-ylamino)-4-oxo-4,5-dihydro-1,3-thiazol-5-yl]-N-(4-fluorophenyl)-N-methylacetamide

Prepared according to method D

19.62 mg, yield 25%.

¹H NMR (270 MHz, DMSO-D6) δ ppm 1.38-1.63 (m, 4H) 2.66-2.40 (m, 1H) 2.74-2.97 (m, 3H) 3.15 (s, 3H) 3.62-3.73 (m, 1H) 4.16-4.28 (m, 1H) 6.03-6.11 (m, 1H) 6.19-6.26 (m, 1H) 7.22-7.33 (m, 2H) 7.39-7.51 (m, 2H) 9.33-9.36 (m, 1H, N—H). MS (ESI+) for C₁₉H₂₀FN₃O₂S m/z 374 (M+H)⁺.

### Example 91 (BVT056674)

### 2-[2-(bicyclo[2.2.1]hept-5-en-2-ylamino)-4-oxo-4,5-dihydro-1,3-thiazol-5-yl]-N-(3-chlorophenyl)-N-methylacetamide

Prepared according to method D

19.62 mg, yield 25%.

¹H NMR (270 MHz, DMSO-D6) δ ppm 1.40-1.62 (m, 4H) 2.38-2.55 (m, 1H) 2.76-2.97 (m, 3H) 3.18 (s, 3H) 3.70-3.60 (m, 1H) 4.20-4.30 (m, 1H) 6.05-6.13 (m, 1H) 6.19-6.26 (m, 1H) 7.32-7.63 (m, 4H) 9.34-9.37 (m, 1H, N—H). MS (ESI+) for C₁₉H₂₀ClN₃O₂S m/z 390 (M+H)⁺.

### Example 92 (BVT056675)

### 2-[2-(bicyclo[2.2.1]hept-5-en-2-ylamino)-4-oxo-4,5-dihydro-1,3-thiazol-5-yl]-N-ethyl-N-(2-methylphenyl)acetamide

Prepared according to method D

35.45 mg, yield 47%.

¹H NMR (270 MHz, DMSO-D6) δ ppm 0.99-1.07 (m, 3H) 1.38-1.59 (m, 4H) 1.92-2.34 (m, 4H) 2.76-2.87 (m, 4H) 3.08-3.24 (m, 1H) 3.66-3.71 (m, 1H) 4.22-4.31 (m, 1H) 6.06-6.12 (m, 1H) 6.20-6.46 (m, 1H) 7.15-7.46 (m, 4H) 9.35-9.37 (m, 1H, N—H). MS (ESI+) for C₂₁H₂₅N₃O₂S m/z 384 (M+H)⁺.

### Example 93 (BVT.59056)

### 2-(bicyclo[2.2.1]hept-5-en-2-ylamino)-5-[2-(8-methyl-3,4-dihydroquinolin-1(2H)-yl)-2-oxoethyl]-1,3-thiazol-4(5H)-one

Prepared according to method D

0.00709 g, yield 36%.

¹H NMR (270 MHz, CHLOROFORM-D) δ ppm 1.56-1.78 (m, 4H) 2.18-3.05 (m, 6H) 2.30 (s, 3H) 3.29-3.40 (m, 1H) 4.28-4.54 (m, 1H) 4.72-4.82 (m, 1H) 6.02-6.09 (m, 1H) 6.20-6.28 (m, 1H) 6.97-7.19 (m, 3H). MS (ESI+) for C₂₂H₂₅N₃O₂S m/z 396 (M+H)⁺.

### Example 94 (BVT.59097)

### 2-(bicyclo[2.2.1]hept-5-en-2-ylamino)-5-(2-oxo-2-piperidin-1-ylethyl)-1,3-thiazol-4(5H)-one

Prepared according to method D

31.16 mg, yield 25%.

¹H NMR (270 MHz, CHLOROFORM-D) δ ppm 1.77-1.43 (m, 10H) 2.81-2.70 (m 1H) 3.03-2.97 (m 2H) 3.65-3.33 (m, 6H) 4.43-4.39 (m 1H) 6.05-6.00 (m, 1H) 6.27-6.24 (m 1H). MS (ESI+) for C₁₇H₂₃N₃O₂S m/z 334 (M+H)⁺.

### Example 95 (BVT.59098)

### 2-[2-(bicyclo[2.2.1]hept-5-en-2-ylamino)-4-oxo-4,5-dihydro-1,3-thiazol-5-yl]-N-isopropyl-N-phenylacetamide

Prepared according to method D

1.9 mg, yield 1.3%.

¹H NMR (270 MHz, CHLOROFORM-D) δ ppm 1.31-1.24 (m, 6H) 2.05-1.61 (m, 5H) 3.05-2.98 (m, 3H) 3.41-3.33 (m, 1H) 3.66-3.54 (m, 1H) 4.55-4.46 (m, 1H) 6.09-6.02 (m, 1H) 6.28-6.24 (m, 1H) 7.00-6.94 (m, 2H) 7.26-7.14 (m, 2H) 7.73-7.68 (m, 1H). MS (ESI+) for C₂₁H₂₅N₃O₂S m/z 384 (M+H)⁺.

### Example 96 (BVT.59099)

### 2-[2-(bicyclo[2.2.1]hept-5-en-2-ylamino)-4-oxo-4,5-dihydro-1,3-thiazol-5-yl]-N-(2,6-difluorophenyl)acetamide

Prepared according to method D

1 mg, yield 0.7%.

¹H NMR (270 MHz, CHLOROFORM-D) δ ppm 1.13-1.04 (m, 3H) 1.88-1.65 (m, 2H) 3.04-2.95 (m, 3H) 3.41-3.32 (m, 1H) 4.37-4.30 (m, 1H) 5.01-4.88 (m, 1H) 6.07-6.04 (m, 1H) 6.31-6.23 (m, 1H) 7.26-7.04 (m, 3H) 7.49-7.40 (m, 2H). MS (ESI+) for C₁₈H₁₇F₂N₃O₂S m/z 378 (M+H)⁺.

### Example 97 (BVT.39225)

### N-(2-Chlorophenyl)-2-[2-(cyclohexylamino)-4-oxo-4,5-dihydro-1,3-thiazol-5-yl]acetamide

Method M

A solution of 1-(2-chlorophenyl)-1H-pyrrole-2,5-dione (100 mg, 0.48 mmol) in absolute ethanol (3 mL) was treated with 1-cyclohexyl-2-thiourea (80 mg, 0.50 mmol) and stirred for 18 h at 50° C. The clear solution was reduced to dryness on a rotavapor and the resulting white foam was recrystallised from acetonitrile giving 148 mg (84%) of white crystals: Mp 183° C. ¹H NMR (400 MHz, DMSO-D6) δ ppm 1.11-1.33 (m, 5H) 1.54-1.57 (m, 1H) 1.68-1.71 (m, 2H) 1.84-1.87 (m, 2H) 2.73 (dd, J1=16.31, J2=11.29 Hz, 1H) 3.27-3.29 (m, 1H, obscured by HDO peak) 3.75-3.81 (m, 1H) 4.35 (dd, J1=11.29, J2=3.51 Hz, 1H) 7.17-7.21 (m, 1H) 7.30-7.34 (m, 1H) 7.49 (d, J=8.03 Hz, 1H) 7.65 (d, J=8.53 Hz, 1H) 9.15 (d, J=7.53 Hz, NH) 9.75 (s, NH). MS (ESI+) for C₁₇H₂₀ClN₃O₂S m/z 366 (M+H)⁺.

### Example 98 (BVT.39226)

### N-(2-Chlorophenyl)-2-(4-oxo-2-piperidin-1-yl-4,5-dihydro-1,3-thiazol-5-yl)acetamide

Prepared according to method M

156 mg, 93% yield of white crystals.

Mp 177° C. ¹H NMR (400 MHz, DMSO-D6) δ ppm 1.53-1.65 (m, 6H) 2.76 (dd, J1=16.82, J2=11.29 Hz, 1H) 3.30-3.34 (m, 1H, obscured by HDO peak) 3.44-3.47 (m, 2H) 3.73-3.86 (m, 2H) 4.43 (dd, J1=11.17, J2=3.39 Hz, 1H) 7.19 (t, J=7.15 Hz, 1H) 7.30-7.34 (m, 1H) 7.49 (dd, J1=8.03, J2=1.25 Hz, 1H) 7.67 (d, J=7.78 Hz, 1H) 9.77 (s, NH). MS (EI⁺) for C₁₆H₁₈ClN₃O₂S m/z 352.2 (M+H)⁺.

### Example 99 (BVT.47436)

### 2-[2-(Bicyclo[2.2.1]hept-5-en-2-ylamino)-4-oxo-4,5-dihydro-1,3-thiazol-5-yl]-N-phenylacetamide

Prepared according to method M

306 mg, 90% yield, as white crystals.

Mp 216-217° C. ¹H NMR (400 MHz, DMSO-D6) δ ppm 1.41-1.60 (m, 4H) 2.64-2.73 (m, 1H) 2.80-2.86 (m, 2H) 3.22-3.28 (m, 1H) 3.72-3.79 (m, 1H) 4.34-4.40 (m, 1H) 6.09 (dd, J1=5.65, J2=3.14 Hz, 1H) 6.21 (dd, J1=5.52, J2=2.76 Hz, 1H) 7.03 (t, J=7.40 Hz, 1H) 7.29 (t, J=7.91 Hz, 2H) 7.54-7.57 (m, 2H) 9.29 (d, J=5.27 Hz, NH) 10.10 (br s, NH); MS (EI⁺) for C₁₈H₁₉N₃O₂S m/z 342.0 (M+H)⁺.

### Example 100 (BVT.47437)

### 2-[2-(Bicyclo[2.2.1]hept-5-en-2-ylamino)-4-oxo-4,5-dihydro-1,3-thiazol-5-yl]-N-1H-indazol-6-ylacetamide

Prepared according to method M

110 mg, 29% yiled as off-white crystals.

Mp 222-223° C. ¹H NMR (400 MHz, DMSO-D6) δ ppm 1.42-1.60 (m, 4H) 2.65-2.74 (m, 1H) 2.81-2.86 (m, 2H) 3.25-3.30 (m, 1H) 3.75-3.78 (m, 1H) 4.34-4.42 (m, 1H) 6.09 (dd, J1=5.65, J2=3.14 Hz, 1H) 6.21 (dd, J1=5.52, J2=2.76 Hz, 1H) 7.36-7.40 (m, 1H) 7.46-7.48 (m, 1H) 8.00 (s, 1H) 8.09 (d, J=1.00 Hz, 1H) 9.29 (dd, J1=6.90, J2=2.13 Hz, NH) 10.11 (br s, NH) 12.97 (br s, NH). MS (EI⁺) for C₁₉H₁₉N₅O₂S m/z 382.2 (M+H)⁺.

### Example 101 (BVT.47438)

### 2-[2-(Bicyclo[2.2.1]hept-5-en-2-ylamino)-4-oxo-4,5-dihydro-1,3-thiazol-5-yl]-N-(4-fluorophenyl)acetamide

Prepared according to method M

297 mg yield 83%, as of white crystals.

Mp 215° C. ¹H NMR (400 MHz, DMSO-D6) δ ppm 1.41-1.60 (m, 4H) 2.64-2.72 (m, 1H) 2.80-2.86 (m, 2H) 3.21-3.27 (m, 1H) 3.72-3.78 (m, 1H) 4.34-4.40 (m, 1H) 6.08 (dd, J1=5.52, J2=3.01 Hz, 1H) 6.20 (dd, J1=5.65, J2=2.89 Hz, 1H) 7.13 (t, J=8.41 Hz, 2H) 7.54-7.59 (m, 2H) 9.28 (m, NH) 10.17 (br s, NH). MS (EI⁺) for C₁₈H₁₈FN₃O₂S m/z 360.2 (M+H)⁺.

### Example 102 (BVT.47439)

### N-(2-benzoylphenyl)-2-[2-(Bicyclo[2.2.1]hept-5-en-2-ylamino)-4-oxo-4,5-dihydro-1,3-thiazol-5-yl]acetamide

Prepared according to method M

256 mg, 57% yield, as a white powder.

Mp 191-192° C. ¹H NMR (400 MHz, DMSO-D6) δ ppm 1.38-1.59 (m, 4H) 2.27-2.37 (m, 1H) 2.77-2.92 (m, 3H) 3.70-3.74 (m, 1H) 4.02-4.09 (m, 1H) 6.07 (dd, J1=5.65, J2=3.14 Hz, 1H) 6.20 (dd, J1=5.65, J2=2.89 Hz, 1H) 7.27-7.32 (m, 1H) 7.39-7.42 (m, 1H) 7.45-7.50 (m, 3H) 7.55-7.65 (m, 4H) 9.26 (d, J=7.03 Hz, NH) 10.18 (br s, NH). MS (EI⁺) for C₂₅H₂₃N₃O₃S m/z 446.2 (M+H)⁺.

### Example 103 (BVT.47489)

### 3-({[2-(Bicyclo[2.2.1]hept-5-en-2-ylamino)-4-oxo-4,5-dihydro-1,3-thiazol-5-yl]acetyl}amino)benzoic acid

Prepared according to method M

290 mg, 75% yield.

Mp 242° C. ¹H NMR (400 MHz, DMSO-D6) δ ppm 1.41-1.59 (m, 4H) 2.66-2.75 (m, 1H) 2.80-2.86 (m, 2H) 3.23-3.29 (m, 1H, obscured by HDO) 3.74-3.77 (m, 1H) 4.33-4.40 (m, 1H) 6.08 (dd, J1=5.40, J2=3.14 Hz, 1H) 6.20 (dd, J1=5.52, J2=2.76 Hz, 1H) 7.41 (t, J=7.78 Hz, 1H) 7.61 (d, J=7.53 Hz, 1H) 7.74 (d, J=7.28 Hz, 1H) 8.21 (s, 1H) 9.29 (dd, J1=6.90, J2=1.88 Hz, NH) 10.29 (s, NH) 12.90 (br s, OH); MS (EI⁺) for C₁₉H₁₉N₃O₄S m/z 386.0 (M+H)⁺.

### Example 104 (BVT.47502)

### 2-[2-(Bicyclo[2.2.1]hept-5-en-2-ylamino)-4-oxo-4,5-dihydro-1,3-thiazol-5-yl]-N-ethylacetamide

Prepared according to method M

92 mg, 31% yield, as white crystals.

Mp 187-188° C.; ¹H NMR (400 MHz, DMSO-D6) δ ppm 0.97-1.02 (m, 3H) 1.39-1.60 (m, 4H) 2.33-2.41 (m, 1H) 2.79-2.86 (m, 2H) 2.96 (dd, J1=15.81, J2=3.26 Hz, 1H) 3.02-3.09 (m, 2H) 3.71-3.77 (m, 1H) 4.20-4.27 (m, 1H) 6.08 (dd, J1=5.40, J2=3.39 Hz, 1H) 6.20 (dd, J1=5.77, J2=2.76 Hz, 1H) 7.98-8.02 (m, NH) 9.23 (d, J=5.02 Hz, NH). MS (EI⁺) m/z 294.2 (M+H)⁺.

### Example 105 (BVT.47491)

### 2-[2-(Bicyclo[2.2.1]hept-5-en-2-ylamino)-4-oxo-4,5-dihydro-1,3-thiazol-5-yl]-N-methylacetamide

Prepared according to method M

170 mg, 61% yield, as a white powder.

Mp 205° C. ¹H NMR (400 MHz, DMSO-D6) δ ppm 1.41-1.57 (m, 4H) 2.34-2.42 (m, 1H) 2.56-2.59 (m, 3H) 2.79-2.86 (m, 2H) 2.95-3.00 (m, 1H) 3.72-3.75 (m, 1H) 4.22-4.27 (m, 1H) 6.08 (dd, J1=5.40, J2=3.14 Hz, 1H) 6.20 (dd, J1=5.90, J2=2.89 Hz, 1H) 7.94-7.98 (m, NH) 9.24 (d, J=6.53 Hz, NH). MS (EI⁺) for C₁₃H₁₇N₃O₂S m/z 280.2 (M+H)⁺.

### Example 106 (BVT.50132)

### 2-(Bicyclo[2.2.1]hept-5-en-2-ylamino)-5-(2-morpholin-4-yl-2-oxoethyl)-1,3-thiazol-4(5H)-one

Prepared according to method M

41 mg, 16% yield. Crystallisation from ethanol gave 30 mg of white crystals.

¹H NMR (400 MHz, DMSO-D6) δ ppm. 1.38-1.59 (m, 4H) 2.60-2.86 (m, 3H) 3.20-3.25 (m, 1H) 3.39-3.43 (m, 4H) 3.51-3.56 (m, 4H) 3.72-3.75 (m, 1H) 4.19-4.25 (m, 1H) 6.07-6.09 (m, 1H) 6.19-6.23 (m, 1H) 9.23 (d, J=7.03 Hz, NH). MS (EI⁺) for C₁₆H₂₁N₃O₃S m/z 336.2 (M+H)⁺.

### Example 107 (BVT.50369)

### 2-[2-(Bicyclo[2.2.1]hept-5-en-2-ylamino)-4-oxo-4,5-dihydro-1,3-thiazol-5-yl]-N-(2-chlorophenyl)-N-methylacetamide

Prepared according to method M

45 mg, 20% yield.

Mp 149-150° C. ¹H NMR (400 MHz, DMSO-D6) δ ppm 1.38-1.60 (m, 4H) 2.67-2.86 (m, 3H) 2.62-2.68 (m, 0.5H) 2.75-2.92 (m, 2.5H) 3.09 (s, 3H) 3.20-3.27 (m, 1H) 3.66-3.72 (m, 1H) 4.18-4.28 (m, 1H) 6.03-6.10 (m, 1H) 6.17-6.23 (m, 1H) 7.45-7.70 (m, 4H) 9.26 (d, J=7.03 Hz, 0.8NH) 9.89 (br s, 0.2NH). MS (EI⁺) for C₁₉H₂₀ClN₃O₂S m/z 390.0 (M+H)⁺.

### Example 108 (BVT.56860)

### 2-(Bicyclo[2.2.1]hept-2-ylamino)-5-[2-(3,4-dihydroquinolin-1(2H)-yl)-2-oxoethyl]-1,3-thiazol-4(5H)-one

Prepared according to method D

0.0360 g, yield 25%.

¹H NMR (270 MHz, CHLOROFORM-D) δ ppm 1.19 (m, 3H) 1.56 (m, 3H) 1.77 (m, 2H) 2.38 (m, 2H) 2.83 (m, 3H) 3.32 (t, J=5.44 Hz, 1H) 3.70 (m, 3H) 4.41 (d, J=11.63 Hz, 1H) 4.57 (s, 1H) 4.72 (s, 1H) 7.13 (m, 4H). MS (EI+) for C₂₁H₂₅N₃O₂S m/z 384 (M+H)⁺.

### Example 109 (BVT.59085)

### 2-{2-[(3-chloro-2-methylphenyl)amino]-4-oxo-4,5-dihydro-1,3-thiazol-5-yl}-N-methyl-N-phenylacetamide

Prepared according to method D

82.3 mg, 32% yield

¹H NMR (400 MHz, METHANOL-D4) δ ppm 2.17 (s, 3H) 2.57 (dd, J=17.09, 9.77 Hz, 1H) 2.92 (dd, J=17.21, 3.05 Hz, 1H) 3.16 (s, 3H) 4.33 (dd, J=9.52, 3.17 Hz, 1H) 6.83 (d, J=7.57 Hz, 1H) 7.09 (t, J=7.81 Hz, 1H) 7.16 (m, 1H) 7.22 (d, J=7.57 Hz, 2H) 7.35 (m, 1H) 7.42 (t, J=7.20 Hz, 2H). MS (ES+) for C₁₉H₁₈ClN₃O₂S m/z 388 (M+H)⁺.

### Example 110 (BVT.59088)

### 2-[(3-chloro-2-methylphenyl)amino]-5-[2-(3,4-dihydroquinolin-1(2H)-yl)-2-oxoethyl]-1,3-thiazol-4(5H)-one

Prepared according to method D

50 mg, 18% yield.

¹H NMR (400 MHz, METHANOL-D4) δ ppm 1.94 (m, 2H) 2.20 (s, 3H) 2.69 (s, 2H) 3.09 (m, 1H) 3.43 (s, 1H) 3.67 (m, 1H) 3.77 (s, 1H) 4.45 (d, J=6.35 Hz, 1H) 6.86 (d, J=7.81 Hz, 1H) 7.15 (m, 6H). MS (ES+) for C₂₁H₂₀ClN₃O₂S m/z 414 (M+H)⁺.

### Example 111 (BVT.59107)

### 2-[2-(1-Adamantylamino)-4-oxo-4,5-dihydro-1,3-thiazol-5-yl]-N-methyl-N-phenylacetamide

Prepared according to method D

0.012 g, yield 9%.

¹H NMR (270 MHz, CHLOROFORM-D) δ ppm 1.68 (m, J=12.12 Hz, 6H) 2.10 (m, 10H) 2.53 (dd, J=17.69, 12.25 Hz, 1H) 3.16 (dd, J=17.57, 3.22 Hz, 1H) 3.30 (s, 3H) 4.33 (dd, J=12.12, 3.22 Hz, 1H) 7.15 (m, 2H) 7.43 (m, 3H). MS (EI+) for C₂₂H₂₇N₃O₂S m/z 398 (M+H)⁺.

### Example 112 (BVT.59117)

### 2-[2-(1-adamantylamino)-4-oxo-4,5-dihydro-1,3-thiazol-5-yl]-N-(2,6-difluorophenyl)acetamide

Prepared according to method D

0.0200 g, yield 15%.

¹H NMR (270 MHz, CHLOROFORM-D) δ ppm 1.70 (s, 5H) 2.16 (m, 10H) 3.31 (m, J=17.07 Hz, 1H) 3.62 (m, 1H) 4.48 (m, 1H) 6.95 (m, J=7.92, 7.92 Hz, 2H) 7.24 (m, J=7.92 Hz, 1H). MS (EI+) for C₂₁H₂₃F₂N₃O₂S m/z 420 (M+H)⁺.

### Example 113 (BVT.59124C)

### 2-[2-(tert-butylamino)-4-oxo-4,5-dihydro-1,3-thiazol-5-yl]-N-methyl-N-phenylacetamide

Prepared according to method D

150 mg, 53% yield.

¹H NMR (400 MHz, DMSO-D6) δ ppm 1.32 (s, 9H) 2.26 (m, 1H) 2.89 (m, 1H) 3.16 (s, 3H) 4.11 (m, 1H) 7.41 (m, 5H) 8.96 (s, 1H). MS (ESI+) for C₁₆H₂₁N₃O₂S m/z 320 (M+H)⁺.

### Example 114 (BVT.59134)

### 2-[2-(cyclopropylamino)-4-oxo-4,5-dihydro-1,3-thiazol-5-yl]-N-methyl-N-phenylacetamide

Prepared according to method D

58 mg, 39% yield.

¹H NMR (270 MHz, CHLOROFORM-D) δ ppm 0.64-1.00 (m, 4H) 2.51 (dd, J=17.57, 11.88 Hz, 1H) 2.66-2.73 (m, 1H) 3.12 (dd, J=17.57, 3.46 Hz, 1H) 3.29 (s, 3H) 4.32 (dd, J=11.88, 3.22 Hz, 1H) 7.14-7.20 (m, 2H) 7.33-7.48 (m, 3H). MS (ESI+) for C₁₅H₁₇N₃O₂S m/z 304 (M+H)⁺.

### Example 115 (BVT.59135)

### 2-(cyclopentylamino)-5-[2-(3,4-dihydroisoquinolin-2(1H)-yl)-2-oxoethyl]-1,3-thiazol-4(5H)-one

Prepared according to method D

72 mg, 46% yield.

¹H NMR (270 MHz, CHLOROFORM-D) δ ppm 1.45-1.75 (m, 2H) 1.70-2.00 (m, 4H) 1.94-2.17 (m, 2H) 2.64-2.81 (m, 1H) 2.78-2.93 (m, 2H) 3.53-3.72 (m, 2H) 3.70-3.94 (m, 2H) 4.34-4.43 (m, 1H) 4.57 (br.s, 1H) 4.71 (s, 1H) 7.07-7.27 (m, 4H). MS (ESI+) for C₁₉H₂₃N₃O₂S m/z 358 (M+H)⁺.

### Example 116 (BVT.59136)

### 2-[2-(cyclopentylamino)-4-oxo-4,5-dihydro-1,3-thiazol-5-yl]-N-methyl-N-phenylacetamide

Prepared according to method D

80 mg 56% yield.

¹H NMR (270 MHz, CHLOROFORM-D) δ ppm 1.42-1.64 (m, 2H) 1.65-1.90 (m, 4H) 1.88-2.10 (m, 2H) 2.40 (dd, J=11.88, 17.55 Hz, 1H) 3.12 (dd, J=3.24, 17.55 Hz, 1H) 3.26 (s, 3H) 3.67-3.78 (m, 1H) 4.28 (dd, J=12.00, 3.09 Hz, 1H) 7.12-7.18 (m, 2H) 7.30-7.44 (m, 3H). MS (ESI+) for C₁₇H₂₁N₃O₂S m/z 332 (M+H)⁺.

### Example 117 (BVT.59137)

### 5-[2-(3,4-dihydroisoquinolin-2(1H)-yl)-2-oxoethyl]-2-(isobutylamino)-1,3-thiazol-4(5H)-one

Prepared according to method D

54 mg 34% yield.

¹H NMR (270 MHz, CHLOROFORM-D) δ ppm 0.84-1.04 (m, 6H) 1.98-2.18 (m, 1H) 2.62-2.94 (m, 3H) 3.14 (d, J=6.93 Hz, 2H) 3.50-3.68 (m, 2H) 3.63-3.94 (m, 1H) 4.32-4.44 (m, 1H) 4.57 (br. s, 1H) 4.65-4.73 (m, 1H) 7.05-7.27 (m, 4H). MS (ESI+) for C₁₈H₂₃N₃O₂S m/z 346 (M+H)⁺.

### Example 118 (BVT.59138)

### 2-[2-(isobutylamino)-4-oxo-4,5-dihydro-1,3-thiazol-5-yl]-N-methyl-N-phenylacetamide

Prepared according to method D

63 mg 43% yield.

¹H NMR (270 MHz, CHLOROFORM-D) δ ppm 0.98 (d, J=6.68 Hz, 5H) 1.94-2.12 (m, 1H) 2.54 (dd, J=17.57, 11.88 Hz, 1H) 3.09 (d, J=3.22 Hz, 1H) 3.17 (d, J=7.18 Hz, 2H) 3.27 (s, 3H) 4.35 (dd, J=11.88, 3.46Hz, 1H) 7.12-7.19 (m, 2H) 7.33-7.48 (m, 3H). MS (ESI+) for C₁₆H₂₁N₃O₂S m/z 320 (M+H)⁺.

### Example 119 (BVT.59139)

### 2-(1-adamantylamino)-5-[2-(3,4-dihydroquinolin-1(2H)-yl)-2-oxoethyl]-1,3-thiazol-4(5H)-one

Prepared according to method D

0.0130 g, yield 10%.

¹H NMR (270 MHz, CHLOROFORM-D) δ ppm 1.68 (m, 5H) 2.11 (m, 9H) 2.80 (m, 3H) 3.65 (m, 2H) 3.87 (m, J=5.69 Hz, 1H) 4.42 (d, J=12.12 Hz, 1H) 4.58 (m, 1H) 4.72 (d, J=6.19 Hz, 1H) 7.11 (m, 4H). MS (EI+) for C₂₄H₂₉N₃O₂S m/z 424 (M+H)⁺.

### Example 120 (BVT.59140)

### 2-(cyclopropylamino)-5-[2-(3,4-dihydroisoquinolin-2(1H)-yl)-2-oxoethyl]-1,3-thiazol-4(5H)-one

Prepared according to method D

53 mg, 33% yield.

¹H NMR (270 MHz, CHLOROFORM-D) δ ppm 0.93 (d, J=7.83 Hz, 4H) 2.66-2.94 (m, 4H) 3.50-368 (m, 2H) 3.70-3.96 (m, 1H) 4.38-4.45 (m, 1H) 4.57-4.62 (m, 1H) 4.74 (s, 1H) 7.07-7.26 (m, 4H). MS (EI+) for C₁₇H₁₉N₃O₂S m/z 330 (M+H)⁺.

### Example 121 (BVT.59147T)

### 5-[2-(3,4-dihydroquinolin-1(2H)-yl)-2-oxoethyl]-2-(mesitylamino)-1,3-thiazol-4(5H)-one

Prepared according to method D

55 mg, 40% yield.

¹H NMR (400 MHz, DMSO-D6) δ ppm 1.87 (m, 2H) 2.08 (s, 6H) 2.23 (s, 3H) 2.68 (m, 2H) 2.96 (dd, J=16.85, 9.64 Hz, 1H) 3.35 (d, J=16.48 Hz, 1H) 3.59 (m, 1H) 3.74 (m, 1H) 4.35 (d, J=8.42 Hz, 1H) 6.89 (s, 2H) 7.10 (td, J=7.26, 0.85 Hz, 1H) 7.16 (m, 2H) 7.45 (d, J=6.10 Hz, 1H). MS (ESI+) for C₂₃H₂₅N₃O₂S m/z 408 (M+H)⁺.

### Example 122 (BVT.59215T)

### 2-(2-{[3,5-Bis(trifluoromethyl)phenyl]amino}-4-oxo-4,5-dihydro-1,3-thiazol-5-yl)-N-methyl-N-phenylacetamide trifluoroacetate

Prepared according to method D

16.8 mg, 11% yield.

¹H NMR (400 MHz, CHLOROFORM-D) δ ppm 2.60 (dd, J=17.3 Hz, J=10.6 Hz, 1H) 3.04 (dd, J=17.3 Hz, J=3.2 Hz, 1H) 3.25 (s, 3H) 4.42 (dd, J=10.6 Hz, J=3.2 Hz, 1H) 7.17 (m, 2H) 7.38 (m, 1H) 7.43 (m, 1H) 7.49 (s, 2H) 7.65 (s 1H). MS (ESI+) for C₂₀H₁₅F₆N₃O₂SC₂HF₃O₂ m/z 476 (M+H)⁺.

### Example 123 (BVT.59260)

### N-(2-chlorophenyl)-2-{2-[(2-methylbutyl)amino]-4-oxo-4,5-dihydro-1,3-thiazol-5-yl}acetamide

Prepared according to method D

12.3 mg, 4% yield.

¹H NMR (400 MHz, METHANOL-D4) δ ppm 0.94 (m, 6H) 1.21 (m, 1H) 1.46 (m, 1H) 1.71 (m, 1H) 2.93 (dd, J=16.60, 10.50 Hz, 1H) 3.13 (m, 1H) 3.44 (m, 2H) 4.57 (dd, J=10.50, 3.42 Hz, 1H) 7.17 (t, J=7.57 Hz, 1H) 7.29 (t, J=7.69 Hz, 1H) 7.43 (d, J=8.06 Hz, 1H) 7.73 (d, J=6.84 Hz, 1H). MS (ESI+) for C₁₆H₂₀ClN₃O₂S m/z 354 (M+H)⁺.

### Example 124 (BVT.59261)

### N-methyl-2-{2-[(2-methylbutyl)amino]-4-oxo-4,5-dihydro-1,3-thiazol-5-yl}-N-phenylacetamide

Prepared according to method D

29.5 mg, 11% yield.

¹H NMR (400 MHz, CHLOROFORM-D) δ ppm 0.91 (t, J=7.32 Hz, 3H) 0.96 (dd, J=6.71, 1.34 Hz, 3H) 1.22 (m, 1H) 1.43 (m, 1H) 1.83 (m, 1H) 2.50 (dd, J=17.58, 11.96 Hz, 1H) 3.12 (m, 2H) 3.25 (t, J=4.88 Hz, 1H) 3.28 (s, 3H) 4.34 (dd, J=11.96, 3.17 Hz, 1H) 7.16 (d, J=7.32 Hz, 2H) 7.40 (m, 3H). MS (ESI+) for C₁₇H₂₃N₃O₂S m/z 334 (M+H)⁺.

### Example 125 (BVT.59262)

### 5-[2-(3,4-dihydroquinolin-1(2H)-yl)-2-oxoethyl]-2-[(2-methylbutyl)amino]-1,3-thiazol-4(5H)-one

Prepared according to method D

19.3 mg, yield 6%.

¹H NMR (400 MHz, METHANOL-D4) δ ppm 0.92 (m, 6H) 1.18 (m, 1H) 1.44 (m, 1H) 1.68 (m, 1H) 1.97 (m, 2H) 2.73 (m, J=5.62 Hz, 2H) 2.99 (m, J=14.04, 10.38 Hz, 1H) 3.12 (m, 1H) 3.40 (m, 1H) 3.51 (dd, J=11.60, 3.78 Hz, 1H) 3.71 (m, 1H) 3.79 (d, J=5.62 Hz, 1H) 4.50 (dd, J=10.50, 3.17 Hz, 1H) 7.19 (s, 4H). MS (ESI+) for C₁₉H₂₅N₃O₂S m/z 360 (M+H)⁺.

### Example 126 (BVT.59263)

### N-methyl-2-{4-oxo-2-[(2-phenylethyl)amino]-4,5-dihydro-1,3-thiazol-5-yl}-N-phenylacetamide

Prepared according to method D

6.1 mg, 4% yield.

¹H NMR (400 MHz, CHLOROFORM-D) δ ppm 2.43 (dd, J=17.46, 11.84 Hz, 1H) 3.02 (t, J=7.45 Hz, 2H) 3.08 (dd, J=17.46, 3.05 Hz, 1H) 3.57 (t, J=7.45 Hz, 2H) 4.30 (dd, J=11.72, 2.93 Hz, 1H) 7.18 (m, 5H) 7.30 (t, J=7.20 Hz, 2H) 7.41 (m, 3H). MS (ESI+) for C₂₀H₂₁N₃O₂S m/z 368 (M+H)⁺.

### Example 127 (BVT.59264)

### 5-[2-(3,4-dihydroquinolin-1(2H)-yl)-2-oxoethyl]-2-[(2-phenylethyl)amino]-1,3-thiazol-4(5H)-one

Prepared according to method D

2.7 mg, 2% yield.

¹H NMR (400 MHz, METHANOL-D4) δ ppm 1.97 (m, 2H) 2.75 (m, J=5.62 Hz, 2H) 2.91 (t, J=7.08 Hz, 2H) 2.97 (m, 2H) 3.60 (m, 1H) 3.72 (t, J=6.47 Hz, 2H) 3.77 (m, 1H) 4.50 (m, 1H) 7.23 (m, 9H). MS (ESI+) for C₂₂H₂₃N₃O₂S m/z 394 (M+H)⁺.

### Example 128 (BVT.59300T)

### 2-(2-{[3,5-Bis(trifluoromethyl)phenyl]amino}-4-oxo-4,5-dihydro-1,3-thiazol-5-yl)-N-(2-chloro-6-fluorobenzyl)acetamide trifluoroacetate

Prepared according to method D

21.5 mg, 19% yield.

¹H NMR (400 MHz, DMSO-D6) δ ppm 2.70 (m, 1H) 2.97 (m, 1H) 4.42 (m, 1H) 4.48 (m, 2H) 7.14-7.42 (m, 3H) 7.55 (s, 2H) 7.81 (s, 1H) 8.38 (s, 1H). MS (ESI+) for C₂₀H₁₃ClF₇N₃O₂SC₂HF₃O₂ m/z 528 (M+H)⁺.

### Example 129 (BVT.59344)

### 2-{2-[(2,6-dimethylphenyl)amino]-4-oxo-4,5-dihydro-1,3-thiazol-5-yl}-N-phenylacetamide

Prepared according to method D

252.7 mg, 64% yield as a white solid.

¹H NMR (400 MHz, DMSO-D6) δ ppm 2.05 (s, 3H) 2.09 (s, 3H) 2.86 (dd, J=16.36, 9.28 Hz, 1H) 3.16 (dd, J=16.36, 3.42 Hz, 1H) 4.45 (dd, J=8.91, 3.30 Hz, 1H) 6.94 (m, 1H) 7.02 (m, 3H) 7.26 (t, J=7.81 Hz, 2H) 7.50 (d, J=7.81 Hz, 2H) 10.05 (s, 1H) 11.62 (s, 1H). MS (ESI+) for C₁₉H₁₉N₃O₂S m/z 354 (M+H)⁺.

### Example 130 (BVT.59345)

### N-methyl-2-{2-[(4-morpholin-4-ylphenyl)amino]-4-oxo-4,5-dihydro-1,3-thiazol-5-yl}-N-phenylacetamide

Prepared according to method D

29.9 mg, 23% yield.

¹H NMR (400 MHz, DMSO-D6) δ ppm 3.10 (m, 4H) 3.15 (s, 3H) 3.17 (s, 2H) 3.71 (m, 4H) 4.25 (d, J=10.74 Hz, 1H) 6.94 (m, 3H) 7.36 (d, J=7.32 Hz, 3H) 7.45 (d, J=6.84 Hz, 2H) 7.56 (d, J=8.55 Hz, 1H). MS (ESI+) for C₂₂H₂₄N₄O₃S m/z 425 (M+H)⁺.

### Example 131 (BVT.59346)

### 5-[2-(3,4-dihydroquinolin-1(2H)-yl)-2-oxoethyl]-2-[(4-morpholin-4-ylphenyl)amino]-1,3-thiazol-4(5H)-one

Prepared according to method D

17.5 mg, 13% yield.

¹H NMR (400 MHz, METHANOL-D4) δ ppm 1.97 (m, 2H) 2.75 (m, 2H) 3.08 (d, J=25.39 Hz, 1H) 3.34 (s, 3H) 3.52 (m, 1H) 3.75 (m, 3H) 3.92 (d, J=2.20 Hz, 4H) 4.51 (m, 1H) 7.23 (m, 7H) 7.62 (d, J=8.06 Hz, 1H). MS (ESI+) for C₂₄H₂₆N₄O₃S m/z 451 (M+H)⁺.

### Example 132 (BVT.59370)

### 5-[2-(3,4-Dihydroquinolin-1(2H)-yl)-2-oxoethyl]-2-{[3,5-bis(trifluoromethyl)phenyl]-amino}-1,3-thiazol-4(5H)-one

Prepared according to method D

9 mg, 9% yield.

MS (ESI+) m/z 502 (M+H)⁺.

### Example 133 (BVT.59371)

### N-Benzyl-2-(2-{[3,5-bis(trifluoromethyl)phenyl]amino}-4-oxo-4,5-dihydro-1,3-thiazol-5-yl)acetamide

Prepared according to method D

20.5 mg, 25% yield.

¹H NMR (400 MHz, CHLOROFORM-D) δ ppm 2.82 (dd, J=16.4 Hz, J=9.9 Hz, 1H) 3.16 (dd, J=16.4 Hz, J=3.3 Hz, 1H) 4.41 (t, J=6.2 Hz, 2H) 4.50 (dd, J=9.9 Hz, J=3.5 Hz, 1H) 5.91 (m, 1H) 7.20-7.33 (m, 5H) 7.48 (s, 2H) 7.65 (s, 1H). MS (ESI+) m/z 476 (M+H)⁺.

MS (ESI+) for C₂₀H₁₅F₆N₃O₂S m/z 476 (M+H)⁺.

### Example 134 (BVT.59372)

### 2-(2-{[3,5-Bis(trifluoromethyl)phenyl]amino}-4-oxo-4,5-dihydro-1,3-thiazol-5-yl)-N-(2-phenylethyl)acetamide

Prepared according to method D

24.6 mg, 29% yield.

¹H NMR (400 MHz, CHLOROFORM-D) δ ppm 2.67-2.80 (m, 3H) 3.07 (m, 1H) 3.50 (m, 2H) 4.45 (m, 1H) 5.63 (m, 1H) 7.12-7.28 (m, 5H) 7.47 (s, 2H) 7.63 (s, 1H). MS (ESI+) for C₂₁H₁₇F₆N₃O₂S m/z 490 (M+H)⁺.

### Example 135 (BVT.59373)

### 2-{2-[(2-Fluorophenyl)amino]-4-oxo-4,5-dihydro-1,3-thiazol-5-yl}-N-methyl-N-phenylacetamide

Prepared according to method D

10.9 mg, 8% yield.

¹H NMR (400 MHz, CHLOROFORM-D) δ ppm 2.51 (dd, J=17.2 Hz, J=11.7 Hz, 1H) 3.06 (m, 1H) 3.22 (s, 3H) 4.38 (m, 1H) 7.06-7.15 (m, 6H) 7.33-7.44 (m, 3H).

MS (ESI+) for C₁₈H₁₆FN₃O₂S m/z 358 (M+H)⁺.

### Example 136 (BVT.59374)

### 5-[2-(3,4-Dihydroquinolin-1(2H)-yl)-2-oxoethyl]-2-[(2-fluorophenyl)amino]-1,3-thiazol-4(5H)-one

Prepared according to method D

5 mg, 5%).

MS (ESI+) for C₂₀H₁₈FN₃O₂S m/z 384 (M+H)⁺.

### Example 137 (BVT.59375)

### N-(2-Chloro-6-fluorobenzyl)-2-{2-[(2-fluorophenyl)amino]-4-oxo-4,5-dihydro-1,3-thiazol-5-yl}acetamide

Prepared according to method D

15.6 mg, 15% yield.

¹H NMR (400 MHz, DMSO-D6) δ ppm 2.63 (dd, J=16.5 Hz, J=10.4 Hz, 1H) 2.96 (dd, J=16.5 Hz, J=3.5 Hz, 1H) 4.34 (d, J=4.7 Hz, 2H) 4.45 (dd, J=10.3 Hz, J=3.5 Hz, 1H) 6.99 (m, 1H) 7.12-7.25 (m, 4H) 7.30-7.40 (m, 2H) 8.38 (t, J=4.9 Hz, 1H). MS (ESI+) for C₁₈H₁₄ClF₂N₃O₂S m/z 410 (M+H)⁺.

### Example 138 (BVT.59578)

### 2-{2-[(2-Fluorophenyl)amino]-4-oxo-4,5-dihydro-1,3-thiazol-5-yl}-N-(4-methylcyclohexyl)acetamide

Prepared according to method D

2.7 mg, 2% yield, as off-white powder.

MS (ESI+) for C₁₈H₂₂FN₃O₂S m/z 364 (M+H)⁺.

### Example 139 (BVT.59581)

### 5-[2-(3,4-dihydroquinolin-1(2H)-yl)-2-oxoethyl]-2-[(2,6-dimethylphenyl)amino]-1,3-thiazol-4(5H)-one

Prepared according to method D

17 mg, 18% yield.

¹H NMR (400 MHz, METHANOL-D4) δ ppm 1.98 (m, 2H) 2.21 (s, 6H) 2.72 (m, 2H) 3.05 (s, 1H) 3.72 (m, 3H) 4.44 (d, J=9.03 Hz, 1H) 7.16 (m, 7H). MS (ESI+) for C₂₂H₂₃N₃O₂S m/z 394 (M+H)⁺.

### Example 140 (BVT.59582)

### 2-{2-[(2,6-dimethylphenyl)amino]-4-oxo-4,5-dihydro-1,3-thiazol-5-yl}-N-methyl-N-phenylacetamide

Prepared according to method D

5.2 mg, 6% yield.

¹H NMR (400 MHz, METHANOL-D4) δ ppm 2.21 (s, 6H) 2.55 (dd, J=17.21, 9.89 Hz, 1H) 2.98 (dd, J=17.09, 3.17 Hz, 1H) 3.20 (s, 3H) 4.35 (dd, J=9.77, 3.42 Hz, 1H) 7.13 (s, 3H) 7.27 (d, J=7.57 Hz, 1H) 7.32 (d, J=7.57 Hz, 1H) 7.41 (d, J=7.08 Hz, 1H) 7.47 (t, J=7.45 Hz, 1H). MS (ESI+) for C₂₀H₂₁N₃O₂S m/z 368 (M+H)⁺.

### Example 141 (BVT.59583)

### N-(2-chloro-6-fluorobenzyl)-2-{2-[(2,6-dimethylphenyl)amino]-4-oxo-4,5-dihydro-1,3-thiazol-5-yl}acetamide

Prepared according to method D

8.7 mg, 9% yield

¹H NMR (400 MHz, METHANOL-D4) δ ppm 2.22 (s, 6H) 2.69 (dd, J=16.11, 9.52 Hz, 1H) 3.06 (dd, J=16.11, 3.91 Hz, 1H) 4.42 (dd, J=9.52, 3.91 Hz, 1H) 4.48 (s, 2H) 7.10 (m, 4H) 7.24 (d, J=7.81 Hz, 1H) 7.30 (m, 1H). MS (ESI+) for C₂₀H₁₉ClFN₃O₂S m/z 420 (M+H)⁺.

### Example 142 (BVT.61669)

### 5-[2-(3,4-Dihydroquinolin-1(2H)-yl)-2-oxoethyl]-2-[(2-methylphenyl)amino]-1,3-thiazol-4(5H)-one

Prepared according to method D

12.2 mg, 13% yield.

¹H NMR (400 MHz, CHLOROFORM-D) δ ppm 1.93 (m, 2H) 2.22 (s, 3H) 2.68 (m, 2H) 2.89 (dd, J=17.0 Hz, J=11.2 Hz, 1H) 3.52 (m, 1H) 3.69 (m, 1H) 3.76 (m, 1H) 4.42 (dd, J=11.2 Hz, J=2.9 Hz, 1H) 7.05-7.21 (m, 8H). MS (ESI+) for C₂₁H₂₁N₃O₂S m/z 380 (M+H)⁺.

### Example 143 (BVT.61670)

### 5-(2-Azepan-1-yl-2-oxoethyl)-2-[(2-methylphenyl)amino]-1,3-thiazol-4(5H)-one

Prepared according to method D

21.9 mg, 24% yield.

¹H NMR (400 MHz, CHLOROFORM-D) δ ppm 1.52 (m, 4H) 1.67 (m, 4H) 2.24 (s, 3H) 2.68 (dd, J=17.0 Hz, J=11.7 Hz, 1H) 3.28-3.44 (m, 4H) 3.51-3.57 (m, 1H) 4.37 (dd, J=11.7 Hz, J=3.0 Hz, 1H) 7.08-7.20 (m, 4H). MS (ESI+) for C₁₈H₂₃N₃O₂S m/z 346 (M+H)⁺.

### Example 144 (BVT.61671)

### N-(2-Chloro-6-fluorobenzyl)-2-{2-[(2-methylphenyl)amino]-4-oxo-4,5-dihydro-1,3-thiazol-5-yl}acetamide

Prepared according to method D

47.7 mg, 48% yield.

¹H NMR (400 MHz, CHLOROFORM-D) δ ppm 2.28 (s, 3H) 2.74 (dd, J=16.6 Hz, J=11.4 Hz, 1H) 3.24 (dd, J=16.6 Hz, J=3.4 Hz, 1H) 4.36 (dd, J=11.4 Hz, J=3.4 Hz, 1H) 4.50 (dd, J=14.3 Hz, J=5.4 Hz, 1H) 4.62 (dd, J=14.3 Hz, J=5.8 Hz, 1H) 6.42 (t, J=5.5 Hz, 1H) 6.97 (m, 1H) 7.15-7.29 (m, 6H). MS (ESI+) for C₁₉H₁₇ClFN₃O₂S m/z 406 (M+H)⁺.

### Example 145 (BVT.61672)

### 5-(2-Azepan-1-yl-2-oxoethyl)-2-[(2-isopropylphenyl)amino]-1,3-thiazol-4(5H)-one

Prepared according to method D

15.0 mg, 17% yield.

¹H NMR (400 MHz, CHLOROFORM-D) δ ppm 1.09 (d, J=6.8 Hz, 3H) 1.13 (d, J=6.8 Hz, 3H) 1.47-1.57 (m, 4H) 1.61-1.72 (m, 4H) 2.70 (dd, J=17.0 Hz, J=11.7 Hz, 1H) 3.05 (sept, J=6.9 Hz, 1H) 3.29-3.44 (m, 4H) 3.50-3.56 (m, 1H) 4.39 (dd, J=11.6 Hz, J=2.9 Hz, 1H) 7.01 (dd, J=7.7 Hz, J=1.4 Hz, 1H) 7.12 (dt, J=7.6 Hz, J=1.6 Hz, 1H) 7.18 (dt, J=7.6 Hz, J=1.5 Hz, 1H) 7.26 (m, 1H). MS (ESI+) for C₂₀H₂₇N₃O₂S m/z 374 (M+H)⁺.

### Example 146 (BVT.61681)

### N-benzyl-2-{2-[(cyclohexylmethyl)amino]-4-oxo-4,5-dihydro-1,3-thiazol-5-yl}acetamide

Prepared according to method D

100 mg, 24% yield.

¹H NMR (400 MHz, DMSO-D6) δ ppm 0.8-1.8 (m, 11H) 3.07 (m, 4H) 4.29 (m, 3H) 7.15-7.45 (m, 5H) 8.52 (m, 1H) 9.15 (s, 1H). MS (ESI+) for C₁₉H₂₅N₃O₂S m/z 360 (M+H)⁺.

### Example 147 (BVT.61682C)

### 2-(cyclohexylamino)-5-[2-(3,4-dihydroquinolin-1(2H)-yl)-2-oxoethyl]-1,3-thiazol-4(5H)-one

Prepared according to method D

65 mg, 45% yield.

¹H NMR (400 MHz, DMSO-D6) δ ppm 1.07-1.99 (m, 12H) 2.71 (t, J=6.65 Hz, 2H) 2.96 (dd, J=16.85, 10.62 Hz, 1H) 3.41 (dd, J=16.91, 3.36 Hz, 1H) 3.65 (m, 1H) 3.75 (m, 1H) 3.83 (s, 1H) 4.38 (dd, J=10.62, 3.30 Hz, 1H) 7.15 (m, 3H) 7.48 (m, 1H) 9.72 (s, 1H).

MS (ESI+) for C₂₀H₂₅N₃O₂S m/z 372 (M+H)⁺.

### Example 148 (BVT.61683C)

### 2-[2-(cyclohexylamino)-4-oxo-4,5-dihydro-1,3-thiazol-5-yl]-N-methyl-N-phenylacetamide

Prepared according to method D

70 mg, 52% yield.

¹H NMR (400 MHz, DMSO-D6) δ ppm 1.04-1.94 (m, 10H) 2.50 (m, 1H) 2.99 (m, 1H) 3.20 (s, 3H) 3.80 (s, 1H) 4.33 (m, 1H) 7.28-7.42 (m, 3H) 7.42-7.53 (m, 2H) 9.80 (s, 1H). MS (ESI+) for C₁₈H₂₃N₃O₂S m/z 346 (M+H)⁺.

### Example 149 (BVT.61684T)

### 2-[2-(mesitylamino)-4-oxo-4,5-dihydro-1,3-thiazol-5-yl]-N-methyl-N-phenylacetamide

Prepared according to method D

57 mg, 44% yield.

¹H NMR (400 MHz, DMSO-D6) δ ppm 2.06 (s, 6H) 2.23 (s, 3H) 2.42 (m, 1H) 2.88 (m, 1H) 3.11 (s, 3H) 4.29 (m, 1H) 6.90 (s, 2H) 7.30-7.50 (m, 5H). MS (ESI+) for C₂₁H₂₃N₃O₂S m/z 382 (M+H)⁺.

### Example 150 (BVT.61705)

### N-methyl-2-{4-oxo-2-[(6-phenoxypyridin-3-yl)amino]-4,5-dihydro-1,3-thiazol-5-yl}-N-phenylacetamide

Prepared according to method D

6.8 mg, 10% yield.

¹H NMR (400 MHz, CHLOROFORM-D) δ ppm 2.53 (dd, J=17.33, 11.23 Hz, 1H) 3.05 (dd, J=17.33, 2.93 Hz, 1H) 3.24 (s, 3H) 3.89 (s, 1H) 4.37 (dd, J=11.11, 3.05 Hz, 1H) 6.88 (d, J=8.55 Hz, 1H) 7.14 (t, J=8.30 Hz, 4H) 7.20 (t, J=7.45 Hz, 1H) 7.39 (m, 5H) 7.48 (dd, J=8.55, 2.69 Hz, 1H) 7.99 (d, J=2.44 Hz, 1H). MS (ES+) for C₂₃H₂₀N₄O₃S m/z 433 (M+H)⁺.

### Example 151 (BVT.61707)

### N-(2-chloro-6-fluorobenzyl)-2-{4-oxo-2-[(6-phenoxypyridin-3-yl)amino]-4,5-dihydro-1,3-thiazol-5-yl}acetamide

Prepared according to method D

7.3 mg, 8% yield

¹H NMR (400 MHz, CHLOROFORM-D) δ ppm 2.68 (dd, J=1 5.99, 10.86 Hz, 1H) 3.15 (d, J=14.40 Hz, 1H) 4.40 (d, J=7.81 Hz, 1H) 4.59 (m, 2H) 5.95 (s, 1H) 6.88 (d, J=8.55 Hz, 1H) 6.97 (t, J=8.55 Hz, 1H) 7.11 (d, J=8.06 Hz, 2H) 7.18 (m, 3H) 7.38 (t, J=7.81 Hz, 2H) 7.46 (d, J=7.81 Hz, 1H) 7.97 (s, 1H). MS (ES+) for C₂₃H₁₈ClFN₄O₃S m/z 485 (M+H)⁺.

### Example 152 (BVT.61792)

### 2-[(2-cyclohex-1-en-1-ylethyl)amino]-5-[2-(3,4-dihydroquinolin-1(2H)-yl)-2-oxoethyl]-1,3-thiazol-4(5H)-one

Prepared according to method D

10.2 mg, 19% yield.

¹H NMR (400 MHz, CHLOROFORM-D) δ ppm 1.53 (m, 2H) 1.61 (m, 2H) 1.99 (m, 6H) 2.34 (t, J=7.08 Hz, 2H) 2.77 (m, 2H) 2.98 (dd, J=17.21, 11.84 Hz, 1H) 3.45 (t, J=7.20 Hz, 2H) 3.58 (d, J=15.87 Hz, 1H) 3.67 (m, 2H) 3.98 (s, 1H) 4.43 (m, 1H) 5.50 (s, 1H) 7.04 (s, 1H) 7.19 (s, 3H). MS (ES+) for C₂₂H₂₇N₃O₂S m/z 398 (M+H)⁺.

### Example 153 (BVT.61793)

### N-(4-fluorophenyl)-2-{4-oxo-2-[(1,1,3,3-tetramethylbutyl)amino]-4,5-dihydro-1,3-thiazol-5-yl}acetamide

Prepared according to method D

8.6 mg, 10% yield.

¹H NMR (400 MHz, CHLOROFORM-D) δ ppm 0.97 (s, 6H) 1.01 (s, 3H) 1.51 (d, J=1.95 Hz, 6H) 1.86 (m, 2H) 2.79 (dd, J=16.24, 10.13 Hz, 1H) 3.46 (dd, J=16.36, 4.15 Hz, 1H) 4.47 (dd, J=10.13, 4.27 Hz, 1H) 6.03 (s, 1H) 6.95 (t, J=8.55 Hz, 2H) 7.49 (m, 1H) 7.55 (dd, J=8.79, 4.88 Hz, 1H) 8.52 (s, 1H). MS (ES+) for C₁₉H₂₆FN₃O₂S m/z 380 (M+H)⁺.

### Example 154 (BVT.61803)

### 5-(2-azepan-1-yl-2-oxoethyl)-2-(cyclohexylamino)-1,3-thiazol-4(5H)-one

Prepared according to method D

60 mg, yield 56%.

¹H NMR (400 MHz, DMSO-D6) δ ppm 1.0-2.0 (m, 19H) 2.73 (m, 1H) 3.33 (m, 5H) 4.24 (m, 1H) 9.25 (d, J=7.32 Hz, 1H). MS (ESI+) for C₁₇H₂₇N₃O₂S m/z 338 (M+H)⁺.

### Example 155 (BVT.61807)

### 5-[2-(3,4-dihydroquinolin-1(2H)-yl)-2-oxoethyl]-2-[(1,1,3,3-tetramethylbutyl)amino]-1,3-thiazol-4(5H)-one

Prepared according to method D

15.3 mg, 16% yield.

¹H NMR (400 MHz, CHLOROFORM-D) δ ppm 1.00 (s, 9H) 1.57 (s, 3H) 1.58 (s, 3H) 1.82 (m, 2H) 2.02 (m, 2H) 2.77 (m, 2H) 2.98 (dd, J=17.21, 11.84 Hz, 1H) 3.68 (m, 3H) 4.03 (s, 1H) 4.41 (m, 1H) 7.11 (m, J=54.69 Hz, 4H). MS (ES+) for C₂₂H₃₁N₃O₂S m/z 402 (M+H)⁺.

### Example 156 (BVT.61854)

### 5-(2-azepan-1-yl-2-oxoethyl)-2-[(1,1,3,3-tetramethylbutyl)amino]-1,3-thiazol-4(5H)-one

Prepared according to method D

7.0 mg, 8%) yield.

¹H NMR (400 MHz, CHLOROFORM-D) δ ppm 0.99 (s, 6H) 1.00 (s, 3H) 1.52 (s, 6H) 1.55 (m, 4H) 1.73 (m, 4H) 1.87 (m, 2H) 2.64 (m, J=12.02, 12.02, 4.76 Hz, 1H) 3.41 (m, 3H) 3.51 (dd, J=17.09, 2.93 Hz, 1H) 3.61 (m, 1H) 4.40 (dt, J=12.15, 3.57 Hz, 1H) 5.63 (s, 1H); MS (ES+) for C₁₉H₃₃N₃O₂S m/z 368 (M+H)⁺.

### Example 157 (BVT.61984)

### 2-(cyclohexylamino)-5-[2-(3,4-dihydroisoquinolin-2(1H)-yl)-2-oxoethyl]-1,3-thiazol-4(5H)-one

Prepared according to method D

60 mg, 41% yield.

¹H NMR (400 MHz, DMSO-D6) δ ppm 1.23 (m, 5H) 1.58 (m, 1H) 1.72 (m, 2H) 1.90 (m, 2H) 2.82 (dd, J=17.03, 11.29 Hz, 3H) 3.34 (dd, J=16.97, 3.17 Hz, 1H) 3.66 (m, 2H) 3.82 (m, 1H) 4.29 (dd, J=11.29, 3.11 Hz, 1H) 4.62 (s, 2H) 7.18 (m, 4H) 8.97 (d, J=6.71 Hz, 1H). MS (ESI+) for C₂₀H₂₅N₃O₂S m/z 372 (M+H)⁺.

### Example 158 (BVT.61985)

### N-benzyl-2-[2-(cyclohexylamino)-4-oxo-4,5-dihydro-1,3-thiazol-5-yl]-N-methylacetamide

Prepared according to method D

52 mg, 37% yield.

¹H NMR (400 MHz, DMSO-D6) δ ppm 1.18 (m, 1H) 1.30 (m, 4H) 1.58 (m, 1H) 1.72 (m, 2H) 1.90 (m, 2H) 2.79 (dd, J=16.54, 11.90 Hz, 1H) 2.86 (s, 1H) 2.92 (s, 2H) 3.29 (t, J=16.17 Hz, 1H) 3.82 (m, 1H) 4.29 (dd, J=11.29, 2.99 Hz, 1H) 4.55 (m, 2H) 7.28 (m, 5H) 8.95 (d, J=5.98 Hz, 1H). MS (ESI+) for C₁₉H₂₅N₃O₂S m/z 360 (M+H)⁺.

### Example 159 (BVT.61990)

### 2-(cyclohexylamino)-5-[2-(6,7-dimethoxy-3,4-dihydroisoquinolin-2(1H)-yl)-2-oxoethyl]-1,3-thiazol-4(5H)-one

Prepared according to method D

13 mg, 8% yield.

¹H NMR (400 MHz, CHLOROFORM-D) δ ppm 1.2-2.1 (m, 10H) 2.85 (m, 3H) 3.33 (m, 1H) 3.65 (m, 2H) 3.7-3.9 (m, 7H) 4.45 (m, 1H) 4.52 (d, J=8.55 Hz, 1H) 4.67 (s, 1H) 6.62 (m, 2H) 8.75 (s, 1H). MS (ESI+) for C₂₂H₂₉N₃O₄S m/z 432 (M+H)⁺.

### Example 160 (BVT.61991)

### 5-[2-(4-benzylpiperidin-1-yl)-2-oxoethyl]-2-(cyclohexylamino)-1,3-thiazol-4(5H)-one

Prepared according to method D

43 mg, 27% yield.

¹H NMR (400 MHz, DMSO-D6) δ ppm 1.25 (m, 8H) 1.59 (m, 3H) 1.78 (m, 6H) 2.53 (m, 1H) 2.67 (m, 1H) 2.95 (m, 1H) 3.21 (m, 1H) 3.82 (m, 2H) 4.23 (dd, J=11.23, 2.81 Hz, 1H) 4.25 (m, 1H) 7.18 (m, 3H) 7.28 (m, 2H) 8.93 (m, 1H). MS (ESI+) for C₂₃H₃₁N₃O₂S m/z 414 (M+H)⁺.

### Example 161 (BVT.61993)

### 5-(2-Azepan-1-yl-2-oxoethyl)-2-(tricyclo[3.3.1.0˜3,7˜]non-3-ylamino)-1,3-thiazol-4(5H)-one

Prepared according to method D

35 mg, 27% yield.

¹H NMR (400 MHz, DMSO-D6) δ ppm 1.43-1.66 (m, 12H) 1.90-2.11 (m, 6H) 2.23-2.27 (m, 2H) 2.46 (t, J=6.7 Hz, 1H) 2.67 (dd, J=17.0 Hz, J=11.7 Hz, 1H) 3.19 (dd, J=17.1 Hz, J=3.1 Hz, 1H) 3.33-3.52 (m, 4H) 4.17 (dd, J=11.7 Hz, J=3.1 Hz, 1H) 9.23 (s, 1H). MS (ESI+) for C₂₀H₂₉N₃O₂S m/z 376 (M+H)⁺.

### Example 162 (BVT.59293)

### 2-[2-(cyclopentylamino)-4-oxo-4,5-dihydro-1,3-thiazol-5-yl]-N-(2,6-difluorophenyl)acetamide

Prepared according to method D

30 mg in 20% yield.

¹H NMR (270 MHz, CHLOROFORM-D) δ ppm 1.57-1.94 (m, 6H) 2.02-2.18 (m, 2H) 3.17-3.35 (m, 1H) 3.58-3.70 (m, 1H) 3.78-3.88 (m, 1H) 4.44-4.53 (m, 1H) 6.95 (t, J=8.16 Hz, 1H) 8.52 (s, 1H). MS m/z (M+H) 354.

MS (ESI+) for C₁₆H₁₇F₂N₃O₂S m/z 376 (M+H)⁺.

### Example 163 (BVT.14204)

### 2-{2-[(4-chlorobenzyl)amino]-4-oxo-4,5-dihydro-1,3-thiazol-5-yl}-N-1-naphthylacetamide

Prepared according to method M

260 mg, yield 50%, as a white solid.

¹H NMR (400 MHz, DMSO-D6) δ ppm 2.89 (dd, J=16.36, 10.99 Hz, 1H) 3.46 (dd, J=16.36, 3.66 Hz, 1H) 4.50 (dd, J=11.11, 3.54 Hz, 1H) 4.45/4.65 (s/m, 2H, taut.) 7.35 (m, 2H) 7.43 (m, 2H) 7.49 (m, 1H) 7.55 (m, 2H) 7.68 (d, J=7.32 Hz, 1H) 7.77 (d, J=8.30 Hz, 1H) 7.94 (m, 1H) 8.09 (m, 1H) 9.70 (br s, NH) 10.11 (s, NH). ¹³C NMR (100 MHz, DMSO-D6) δ ppm-46.84, 51.28, 121.45, 122.58, 125.22, 125.37, 125.69, 125.89, 127.48, 127.95, 128.33 (2) 129.11, 129.32 (2) 133.05, 133.52, 136.44, 169.19, 180.11, 188.04. MS (frag, EI⁺) for C₂₂H₁₈ClN₃O₂S m/z 423 (M+H)⁺.

### Example 164 (BVT.14212)

### N-1-naphthyl-2-[2-(1-naphthylamino)-4-oxo-4,5-dihydro-1,3-thiazol-5-yl]acetamide

Prepared according to method M

240 mg, yield 56%, as a white solid

¹H NMR (400 MHz, DMSO-D6) δ ppm 3.09 (dd, J=16.60, 9.52 Hz, 1H) 3.42 (dd, J=16.60, 3.66 Hz, 1H) 4.60 (dd, J=9.40, 3.78 Hz, 1H) 7.06 (d, J=7.08 Hz, 1H) 7.49 (m, 6H) 7.61 (d, J=7.32 Hz, 1H) 7.69 (d, J=8.06 Hz, 1H) 7.75 (d, J=8.06 Hz, 1H) 7.93 (m, 3H) 8.04 (d, J=8.06 Hz, 1H) 10.08/10.17 (s/s, NH, taut.) 11.24/12.03 (s/s, NH). ¹³C NMR (100 MHz, DMSO-D6) δ ppm 38.44, 45.49, 115.72, 121.41, 122.52, 122.93, 124.11, 125.22, 125.33, 125.55, 125.64, 125.74, 125.86, 126.21, 127.00, 127.40, 127.72, 127.92, 132.90, 133.48, 133.73, 168.40. MS (frag, EI⁺) for C₂₅H₁₉N₃O₂S m/z 425 (M+H)⁺.

### Example 165 (BVT.59416)

### 2-(2-anilino-4-oxo-4,5-dihydro-1,3-thiazol-5-yl)-N-methyl-N-phenylacetamide

Prepared according to method D

42.1 mg, yield 31%.

¹H NMR (400 MHz, DMSO-D6) δ ppm 2.89 (m, 2H) 3.13 (m, 3H) 4.32 (d, J=10.01 Hz, 1H) 6.98 (d, J=7.08 Hz, 1H) 7.14 (t, J=7.32 Hz, 1H) 7.36 (m, 5H) 7.46 (d, J=7.08 Hz, 2H) 7.65 (d, J=6.84 Hz, 1H). MS (frag, EI⁺) for C₁₈H₁₇N₃O₂S m/z 340 (M+H)⁺.

### Example 166 (BVT.59417)

### 2-(2-anilino-4-oxo-4,5-dihydro-1,3-thiazol-5-yl)-N-(2-chlorophenyl)acetamide

Prepared according to method D

7.5 mg, yield 5%.

¹H NMR (400 MHz, DMSO-D6) δ ppm 2.88 (m, 2H) 4.48 (d, J=8.79 Hz, 1H) 6.99 (m, 1H) 7.17 (m, 2H) 7.36 (m, 3H) 7.48 (m, 1H) 7.66 (m, 2H) 9.76 (d, J=16.60 Hz, 1H). MS (ESI+) for C₁₇H₁₄ClN₃O₂S m/z 360 (M+H)⁺.

### Example 167 (BVT.59418)

### 5-[2-(3,4-dihydroquinolin-1(2H)-yl)-2-oxoethyl]-2-[(2-morpholin-4-ylethyl)amino]-1,3-thiazol-4(5H)-one

Prepared according to method D

7.7 mg, yield 6%, as a yellow oil.

¹H NMR (400 MHz, CHLOROFORM-D) δ ppm 1.99 (m, 2H) 2.12 (m, 1H) 2.75 (m, 2H) 2.96 (m, 2H) 3.39 (m, 2H) 3.46 (m, 1H) 3.74 (m, 4H) 3.98 (m, 6H) 4.46 (dd, J=10.25, 3.17 Hz, 1H) 7.14 (m, 4H). MS (frag, EI⁺) for C₂₀H₂₆N₄O₃S m/z 403 (M+H)⁺.

### Example 168 (BVT.59110T)

### 2-[2-(sec-butylamino)-4-oxo-4,5-dihydro-1,3-thiazol-5-yl]-N-methyl-N-phenylacetamide trifluoroacetate

Prepared according to method D

209 mg, yield 60%.

¹H NMR (400 MHz, CHLOROFORM-D) δ ppm 0.92 (m, 3H) 1.32 (m, 3H) 1.68 (m, 2H) 2.58 (m, 1H) 3.08 (m, 1H) 3.24 (m, 3H) 3.41 (m, 1H) 4.36 (dd, J=14.65, 3.42 Hz, 1H) 7.15 (m, 2H) 7.38 (m, 3H). MS (ESI+) for C₁₆H₂₁N₃O₂SC₂HF₃O₂ m/z 320 (M+H)⁺.

### Example 169 (BVT.59132T)

### 2-(sec-butylamino)-5-[2-(3,4-dihydroquinolin-1(2H)-yl)-2-oxoethyl]-1,3-thiazol-4(5H)-one trifluoroacetate

Prepared according to method D

104.7 mg, yield 52%.

¹H NMR (400 MHz, CHLOROFORM-D) δ ppm 0.97 (m, 3H) 1.36 (m, 3H) 1.73 (m, 2H) 1.97 (m, 2H) 2.77 (m, 2H) 3.01 (m, 1H) 3.43 (m, 1H) 3.63 (m, 2H) 4.00 (m, 1H) 4.43 (m, 1H) 7.11 (m, 4H). MS (ESI+) for C₁₈H₂₃N₃O₂SC₂HF₃O₂ m/z 346 (M+H)⁺.

### Example 170 (BVT.59133T)

### 2-[2-(sec-butylamino)-4-oxo-4,5-dihydro-1,3-thiazol-5-yl]-N-(2-chlorophenyl)acetamide trifluoroacetate

Prepared according to method D

62.3 mg, yield 30%.

¹H NMR (400 MHz, CHLOROFORM-D) δ ppm 0.96 (m, 3H) 1.37 (m, 3H) 1.73 (m, 2H) 3.13 (m, 1H) 3.44 (m, 1H) 3.59 (dd, J=20.26, 3.17 Hz, 1H) 4.52 (m, 1H) 4.91 (m, 1H) 7.09 (t, J=7.32 Hz, 1H) 7.25 (t, J=8.79, 6.84 Hz, 1H) 7.37 (d, J=8.06 Hz, 1H) 8.05 (d, J=8.06 Hz, 1H) 8.27 (m, 1H). MS (ESI+) for C₁₅H₁₈ClN₃O₂SC₂HF₃O₂ m/z 340 (M+H)⁺.

### Example 171 (BVT.59265T)

### 2-{2-[(cyclopropylmethyl)amino]-4-oxo-4,5-dihydro-1,3-thiazol-5-yl}-N-methyl-N-phenylacetamide trifluoroacetate

Prepared according to method D

15 mg, yield 10%.

¹H NMR (400 MHz, CHLOROFORM-D) δ ppm 0.52 (m, 4H) 1.16 (m, 1H) 2.66 (m, 1H) 3.08 (m, 1H) 3.26 (m, 3H) 3.29 (m, 1H) 3.80 (m, 1H) 4.41 (m, 1H) 7.18 (m, 2H) 7.43 (m, 3H). MS (ESI+) for C₁₆H₁₉N₃O₂SC₂HF₃O₂ m/z 318 (M+H)⁺.

### Example 172 (BVT.59352)

### N-methyl-2-[2-(1-naphthylamino)-4-oxo-4,5-dihydro-1,3-thiazol-5-yl]-N-phenylacetamide

Prepared according to method D

14 mg, yield 12%.

¹H NMR (400 MHz, METHANOL-D4) δ ppm 2.52 (m, 1H) 2.89 (m, 1H) 3.10 (m, 3H) 4.29 (dd, J=9.77, 3.66 Hz, 1H) 7.14 (m, 3H) 7.38 (m, 6H) 7.67 (m, 1H) 7.84 (m, 2H). MS (ESI+) for C₂₂H₁₉N₃O₂S m/z 390 (M+H)⁺.

### Example 173 (BVT.59386)

### 2-{2-[(4-methylbenzyl)amino]-4-oxo-4,5-dihydro-1,3-thiazol-5-yl}-N-phenylacetamide

Prepared according to method D

246 mg, yield 47%.

¹H NMR (400 MHz, DMSO-D6) δ ppm 2.27 (m, 3H) 2.69 (m, 1H) 3.26 (m, 1H) 4.40 (m, 1H) 7.03 (m, 1H) 7.17 (m, 4H) 7.29 (m, 2H) 7.55 (m, 2H). MS (ESI+) for C₁₉H₁₉N₃O₂S m/z 390 (M+H)⁺.

### Example 174 (BVT.59385)

### 2-{2-[(4-chlorobenzyl)amino]-4-oxo-4,5-dihydro-1,3-thiazol-5-yl}-N-phenylacetamide

Prepared according to method D

163 mg, yield 29%.

¹H NMR (400 MHz, DMSO-D6) δ ppm 2.70 (m, 1H) 3.26 (m, 1H) 4.42 (m, 1H) 7.03 (m, 1H) 7.31 (m, 4H) 7.42 (m, 2H) 7.54 (m, 2H). MS (ESI+) for C₁₈H₁₆ClN₃O₂S m/z 374 (M+H)⁺.

### Example 175 (BVT.59415)

### 2-Anilino-5-[2-(3,4-dihydroquinolin-1(2H)-yl)-2-oxoethyl]-1,3-thiazol-4(5H)-one

Prepared according to method D

37.1 mg, yield 26%.

¹H NMR (400 MHz, CHLOROFORM-D) δ ppm 1.95 (m, 2H) 2.69 (m, 2H) 2.92 (m, 1H) 3.55 (m, 1H) 3.70 (m, 1H) 3.81 (m, 1H) 4.44 (d, J=9.03 Hz, 1H) 7.14 (m, 4H) 7.23 (d, J=7.32 Hz, 1H) 7.29 (d, J=7.57 Hz, 2H) 7.37 (m, Hz, 2H). MS (ESI+) for C₂₀H₁₉N₃O₂S m/z 366 (M+H)⁺.

### Example 176 (BVT.63192)

### 5-[2-(3,4-Dihydroisoquinolin-2(1H)-yl)-2-oxoethyl]-2-(tricyclo[3.3.1.0˜3,7˜]non-3-ylamino)-1,3-thiazol-4(5H)-one

Prepared according to method D

30 mg, yield 32% as a white solid.

¹H NMR (400 MHz, DMSO-d₆) δ ppm 1.46-1.57 (m, 4H) 1.90-2.10 (m, 6H) 2.23-2.26 (m, 2H) 2.45-2.49 (m, 1H) 2.74-2.86 (m, 3H) 3.29-3.34 (m, 1H) 3.56-3.74 (m, 2H) 4.17-4.27 (m, 1H) 4.54-4.66 (m, 2H) 7.17 (m, 4H) 9.27 (s, 1H). MS (ESI+) for C₂₃H₂₇N₃O₂S m/z 410 (M+H)⁺.

### Example 177 (BVT.63199)

### 2-(cycloheptylamino)-5-[2-(3,4-dihydroisoquinolin-2(1H)-yl)-2-oxoethyl]-1,3-thiazol-4(5H)-one

Prepared according to method D

0.007 g, yield 5% as an off-white powder.

1H NMR (400 MHz, DMSO-D6) δ ppm 1.50 (m, 12H) 1.88 (m, J=6.10 Hz, 2H) 2.87 (m, 2H) 3.33 (m, 1H) 3.63 (m, J=5.92 Hz, 1H) 3.97 (m, 1H) 4.28 (m, 1H) 4.60 (m, 2H) 7.17 (m, J=3.91 Hz, 4H) 9.29 (s, 1H). MS (ESI) for C₂₁H₂₇N₃O₂S m/z 386 (M+H)⁺.

### Example 178 (BVT.63210)

### 5-(2-azepan-1-yl-2-oxoethyl)-2-(cycloheptylamino)-1,3-thiazol-4(5H)-one

Prepared according to method D

0.007 g, yield 5% as a yellow solid.

1H NMR (400 MHz, DMSO-D6) δ ppm 1.53 (m, 18H) 1.89 (m, 2H) 2.73 (none, J=17.09, 11.35 Hz, 1H) 3.21 (dd, J=17.21, 3.17 Hz, 1H) 3.40 (m, 4H) 3.97 (m, 1H) 4.23 (dd, J=11.60, 3.17 Hz, 1H) 9.19 (d, J=6.96 Hz, 1H). MS (ESI) for C₁₈H₂₉N₃O₂S m/z 352 (M+H)⁺.

### Example 179 (BVT.63220)

### 5-(2-azepan-1-yl-2-oxoethyl)-2-[(cyclohexylmethyl)amino]-1,3-thiazol-4(5H)-one

Prepared according to method D.

8.8 mg, yield 13%.

1H NMR (400 MHz, CHLOROFORM-D) δ ppm 1.37 (m, 19H) 2.77 (m, 1H) 3.21 (d, J=6.59 Hz, 2H) 3.47 (m, 5H) 4.42 (dd, J=12.21, 3.42 Hz, 1H). MS (ES) for C₁₈H₂₉N₃O₂S m/z 352 (M+H)⁺.

### Example 180 (BVT063223)

### 2-[2-(cycloheptylamino)-4-oxo-4,5-dihydro-1,3-thiazol-5-yl]-N-methyl-N-phenylacetamide

Prepared according to method D.

0.04 g, yield 20% as an off-white solid.

1H NMR (400 MHz, DMSO-D6) δ ppm 1.51 (m, 10H) 1.85 (m, 2H) 2.38 (dd, J=17.46, 10.74 Hz, 1H) 2.90 (m, J=16.42, 1.89 Hz, 1H) 3.17 (s, 3H) 3.96 (m, 1H) 4.25 (dd, J=10.99, 2.56 Hz, 1H) 7.38 (m, 1H) 7.36 (d, J=6.96 Hz, 2H) 7.47 (m, 2H) 9.55 (d, J=6.96 Hz, 1H). MS (ES) for C₁₉H₂₅N₃O₂S m/z 360 (M+H)⁺.

### Example 181 (BVT.63320)

### 5-[2-(4-Methylpiperidin-1-yl)-2-oxoethyl]-2-(tricyclo[3.3.1.0˜3,7˜]non-3-ylamino)-1,3-thiazol-4(5H)-one

Prepared according to method D.

44 mg, yield 41% as white solid.

¹H NMR (400 MHz, DMSO-d₆) δ ppm 1.82-1.09 (m, 5H) 1.46-1.65 (m, 7H) 1.90-2.11 (m, 6H) 2.23-2.27 (m, 2H) 2.46 (t, J=6.2 Hz, 1H) 2.53-2.58 (m, 1H) 2.63-2.72 (m, 1H) 2.88-2.98 (m, 1H) 3.18 (ddd, J=17.0 Hz, J=5.6 Hz, J=3.2 Hz, 1H) 3.70-3.76 (m, 1H) 4.11-4.22 (m, 1H) 4.25-4.33 (m, 1H) 9.23 (d, J=2.8 Hz, 1H). MS (ESI+) for C₂₀H₂₉N₃O₂S m/z 376 (M+H)⁺.

### Example 182 (BVT.63321)

### 5-[2-(1,3-Dihydro-2H-isoindol-2-yl)-2-oxoethyl]-2-(tricyclo[3.3.1.0˜3,7˜]non-3-ylamino)-1,3-thiazol-4(5H)-one

Prepared according to method D.

65 mg, yield 57%.

¹H NMR (400 MHz, DMSO-d₆) δ ppm 1.46-1.58 (m, 4H) 1.91-2.11 (m, 6H) 2.48 (m, 1H) 2.75 (dd, J=17.1 Hz, J=11.7Hz, 1H) 3.30 (dd, J=17.1Hz, J=3.1 Hz, 1H) 4.23 (dd, J=11.7 Hz, J=3.1 Hz, 1H) 4.63 (s, 2H) 4.82 (s, 2H) 7.26-7.36 (m, 4H) 9.27 (s, 1H). MS (ESI+) C₂₂H₂₅N₃O₂S m/z 396 (M+H)⁺.

### Example 183 (BVT.63322)

### 2-[(cyclohexylmethyl)amino]-5-(2-oxo-2-pyrrolidin-1-ylethyl)-1,3-thiazol-4(5H)-one

Prepared according to method D

13.3 mg, yield 14%.

1H NMR (400 MHz, CHLOROFORM-D) δ ppm 1.12 (m, 5H) 1.69 (m, 5H) 1.97 (m, 5H) 3.22 (m, 2H) 3.43 (m, 6H) 4.43 (dd, J=11.96, 3.42 Hz, 1H) MS (ES) for C₁₆H₂₅N₃O₂S m/z 324 (M+H)⁺.

### Example 184 (BVT.63323)

### 2-[(cyclohexylmethyl)amino]-5-[2-(3,4-dihydroisoquinolin-2(1H)-yl)-2-oxoethyl]-1,3-thiazol-4(5H)-one

Prepared according to method D

13.3 mg, yield 19%.

1H NMR (400 MHz, CHLOROFORM-D) δ ppm 1.12 (m, 6H) 1.72 (m, 5H) 2.90 (m, 2H) 3.23 (m, 2H) 3.75 (m, 4H) 4.52 (m, 2H) 4.73 (m, 1H) 7.17 (m, 4H) MS (ES) for C₂₁H₂₇N₃O₂S m/z 386 (M+H)⁺.

### Example 185 (BVT.63329)

### 2-[2-(Dicyclohexylamino)-4-oxo-4,5-dihydro-1,3-thiazol-5-yl]-N-benzylacetamide

Prepared according method M.

30.8 mg, yield 29%.

1H NMR (400 MHz, DMSO-D6) δ ppm 1.16 (m, 3H) 1.32 (m, 5H) 1.47 (d, J=10.25 Hz, 2H) 1.58 (t, J=9.22 Hz, 2H) 1.70 (m, 6H) 1.80 (d, J=12.45 Hz, 2H) 2.44 (dd, J=16.24, 11.72 Hz, 1H) 3.10 (dd, J=16.17, 3.48 Hz, 1H) 3.50 (m, 2H) 4.23 (dd, J=11.60, 3.42 Hz, 1H) 4.28 (d, J=5.86 Hz, 2H) 7.24 (m, 1H) 7.25 (m, 2H) 7.32 (m, 2H) 8.54 (t, J=5.86 Hz, 1H). 13C NMR (101 MHz, DMSO-D6) δ ppm 24.4, 24.8, 25.1, 25.4, 29.1, 29.3, 29.7, 38.9, 42.2, 49.8, 62.9, 126.7, 127.1, 128.1, 139.0, 169.5, 178.3, 187.5. MS (ES) for C₂₄H₃₃N₃O₂S m/z 428 (M+H)⁺.

### Example 186 (BVT.63331)

### 2-(2-Anilino-4-oxo-4,5-dihydro-1,3-thiazol-5-yl)-N-phenylacetamide

Prepared according method M.

36.0 mg, yield 12%.

1H NMR (400 MHz, DMSO-D6) δ ppm 3.23 (m, 2H) 4.48 (d, J=8.55 Hz, 1H) 7.03 (m, 2H) 7.15 (m, 1H) 7.32 (m, 4H) 7.53 (dd, J=17.82, 7.57 Hz, 2H) 7.70 (d, J=6.84 Hz, 1H) 10.11 (d, J=12.21 Hz, 1H). MS (ES) for C₁₇H₁₅N₃O₂S m/z 326 (M+H)⁺.

### Example 187 (BVT063335)

### 2-azepan-1-yl-5-(2-azepan-1-yl-2-oxoethyl)-1,3-thiazol-4(5H)-one

Prepared according method D.

11 mg, yield 34%.

¹H NMR (400 MHz, CHLOROFORM-D) δ ppm 1.58 (m, 8H) 1.71 (d, J=2.9 Hz, 4H) 1.82 (dd, J=9.0, 4.9 Hz, 4H) 2.65 (dd, J=17.1, 12.2 Hz, 1H) 3.48 (m, 7H) 3.86 (m, 2H) 4.45 (dd, J=12.1, 3.1 Hz, 1H). MS (ES+) for C₁₇H₂₇N₃O₂S m/z 338 (M+H)⁺.

### Example 188 (BVT063336)

### 2-azepan-1-yl-5-[2-(3,4-dihydroquinolin-1(2H)-yl)-2-oxoethyl]-1,3-thiazol-4(5H)-one

Prepared according method D.

60.4 mg, yield 42%.

¹H NMR (400 MHz, CHLOROFORM-D) δ ppm 1.60 (s, 4H) 1.82 (dd, J=13.6, 5.0 Hz, 4H) 1.98 (m, 2H) 2.72 (s, 2H) 2.88 (dd, J=17.0, 11.8 Hz, 1H) 3.55 (m, 4H) 3.71 (m, 1H) 3.85 (m, 2H) 4.49 (d, J=10.0 Hz, 1H) 7.16 (m, 4H). MS (ES+) for C₂₀H₂₅N₃O₂S m/z 372 (M+H)⁺.

### Example 189 (BVT063337)

### 2-azepan-1-yl-5-[2-(3,4-dihydroisoquinolin-2(1H)-yl)-2-oxoethyl]-1,3-thiazol-4(5H)-one

Prepared according method D.

61.2 mg, yield 42%.

¹H NMR (400 MHz, CDCl₃) δ ppm 1.59 (s, 4H) 1.81 (s, 4H) 2.73 (m, 1H) 2.88 (m, 2H) 3.53 (t, J=5.7 Hz, 2H) 3.63 (m, 2H) 3.75 (m, 1H) 3.88 (m, 2H) 4.46 (m, 1H) 4.58 (d, J=4.9 Hz, 1H) 4.72 (s, 1H) 7.07 (m, 1H) 7.14 (m, 1H) 7.19 (m, 2H). MS (ES+) for C₂₀H₂₅N₃O₂S m/z 372 (M+H)⁺.

### Example 190 (BVT063339)

### 5-[2-(3,4-dihydroisoquinolin-2(1H)-yl)-2-oxoethyl]-2-piperidin-1-yl-1,3-thiazol-4(5H)-one

Prepared according method D.

44.9 mg, yield 32%.

¹H NMR (400 MHz, CDCl₃) δ ppm 1.70 (s, 6H) 2.74 (m, 1H) 2.88 (m, 2H) 3.47 (s, 2H) 3.62 (m, 2H) 3.74 (m, 1H) 3.88 (m, 2H) 4.49 (m, 1H) 4.58 (d, J=6.4 Hz, 1H) 4.72 (s, 1H) 7.14 (m, 4H). MS (ES+) for C₁₉H₂₃N₃O₂S m/z 358 (M+H)⁺.

### Example 191 (BVT063341)

### 2-(cycloheptylamino)-5-[2-(2,3-dihydro-1H-indol-1-yl)-2-oxoethyl]-1,3-thiazol-4(5H)-one

Prepared according method D.

0.01 g, yield 8.7% as a coloured solid.

1H NMR (400 MHz, DMSO-D6) δ ppm 1.35-1.70 (m, 10H) 1.80-1.97 (m, 2H) 2.88 (dd, J=17.58, 11.47 Hz, 1H) 3.13 (t, J=8.42 Hz, 2H) 3.38 (dd, J=17.58, 3.05 Hz, 1H) 3.94-4.04 (m, 1H) 4.04-4.13 (m, 2H) 4.31 (dd, J=11.54, 2.99 Hz, 1H) 7.00 (td, J=7.45, 0.85 Hz, 1H) 7.15 (t, J=7.69 Hz, 1H) 7.24 (dd, J=7.45, 0.49 Hz, 1H) 8.03 (d, J=8.18 Hz, 1H) 9.14 (d, J=7.69 Hz, 1H). MS (ES) for C₂₀H₂₅N₃O₂S m/z 372 (M+H)⁺.

### Example 192 (BVT063342)

### 2-(cycloheptylamino)-5-(2-oxo-2-pyrrolidin-1-ylethyl)-1,3-thiazol-4(5H)-one

Prepared according method D.

0.03 g, yield 25% as an off-white solid.

1H NMR (400 MHz, DMSO-D6) δ ppm 1.34-1.70 (m, 10H) 1.71-1.81 (m, 2H) 1.80-1.93 (m, 4H) 2.62 (dd, J=17.09, 11.72 Hz, 1H) 3.16 (dd, J=17.21, 3.17 Hz, 1H) 3.23-3.32 (m, 2H) 3.33-3.40 (m, 2H) 3.92-4.02 (m, 1H) 4.20 (dd, J=1.72, 3.17 Hz, 1H) 9.16 (d, J=7.57 Hz, 1H). MS (ES) for C₁₆H₂₅N₃O₂S m/z 324 (M+H)⁺.

### Example 193 (BVT063343)

### 2-(cycloheptylamino)-5-[2-(4-methylpiperidin-1-yl)-2-oxoethyl]-1,3-thiazol-4(5H)-one

Prepared according method D.

0.86 g, yield 66% as an off-white solid.

1H NMR (400 MHz, DMSO-D6) δ ppm 0.89 (d, J=6.10, 1.83 Hz, 3H) 0.93-1.12 (m, 2H) 1.34-1.70 (m, 13H) 1.79-1.96 (m, 2H) 2.51-2.62 (m, 1H) 2.67-2.78 (m, 1H) 2.87-3.01 (m, 1H) 3.17-3.27 (m, 1H) 3.73 (d, J=13.31 Hz, 1H) 3.91-4.03 (m, 1H) 4.20 (ddd, J=11.44, 7.54, 2.87 Hz, 1H) 4.25-4.35 (m, 1H) 9.18 (d, J=6.96 Hz, 1H). MS (ES) for C₁₈H₂₉N₃O₂S m/z 352 (M+H)⁺.

### Example 194 (BVT.66775)

### 2-[2-(Dicyclohexylamino)-4-oxo-4,5-dihydro-1,3-thiazol-5-yl]-N-phenylacetamide

Prepared according method M.

23.1 mg, yield 23%.

1H NMR (400 MHz, DMSO-D6) δ ppm 1.12 (m, H) 1.30 (m, 5H) 1.46 (m, 2H) 1.57 (m, 2H) 1.72 (m, 9H) 2.63 (dd, J=16.48, 11.60 Hz, 2H) 3.27 (m, 1H) 3.47 (m, 1H) 4.29 (dd, J=11.47, 3.17 Hz, 1H) 7.04 (t, J=7.32 Hz, 1H) 7.29 (t, J=7.93 Hz, 2H) 7.55 (d, J=7.81 Hz, 2H) 10.10 (s, 1H). MS (ES) for C₂₃H₃₁N₃O₂S m/z 414 (M+H)⁺.

### Example 195 (BVT.63344)

### N-cyclohexyl-2-{2-[(cyclohexylmethyl)amino]-4-oxo-4,5-dihydro-1,3-thiazol-5-yl}acetamide

Prepared according method D.

9.4 mg, yield 14%.

1H NMR (400 MHz, CHLOROFORM-D) δ ppm 1.16 (m, 10H) 1.69 (m, 10H) 1.89 (m, 1H) 2.79 (m, 1H) 3.26 (m, 2H) 3.72 (m, 2H) 4.42 (dd, J=11.47, 3.42 Hz, 1H) 6.03 (m, 1H) 7.04 (m, 1H) MS (ES) for C₁₈H₂₉N₃O₂S m/z 352 (M+H)⁺.

### Example 196 (BVT.66802)

### N-Cyclohexyl-N-ethyl-2-[4-oxo-2-(tricyclo[3.3.1.0˜3,7˜]non-3-ylamino)-4,5-dihydro-1,3-thiazol-5-yl]acetamide

Prepared according method D.

24 mg, yield 22% as a white solid.

¹H NMR (400 MHz, DMSO-d₆) δ ppm 0.98-1.14 (m, 4H) 1.17-1.75 (m, 14H) 1.90-2.11 (m, 6H) 2.23-2.28 (m, 2H) 2.46 (t, J=6.6 Hz, 1H) 2.63-2.76 (m, 1H) 3.16-3.29 (m, 3H) 4.13-4.25 (m, 1H) 9.21 (s, 1H). MS (ESI+) for C₂₂H₃₃N₃O₂S m/z 404 (M+H)⁺.

### Example 197 (BVT.66803)

### N-(Cyclopropylmethyl)-N-propylacetamide-2-[4-oxo-2-(tricyclo[3.3.1.0˜3,7˜]non-3-ylamino)-4,5-dihydro-1,3-thiazol-5-yl]acetamide

Prepared according method D.

46 mg, yield 43% as a white solid.

¹H NMR (400 MHz, DMSO-d₆) δ ppm 0.17-0.24 (m, 2H) 0.38-0.43 (m, 1H) 0.46-0.50 (m, 1H) 0.79 and 0.84 (t, J=7.4 Hz and t, J=7.4 Hz, 3H) 0.88-0.98 (m, 1H) 1.44-1.57 (m, 6H) 1.90-2.11 (m, 6H), 2.23-2.27 (m, 2H), 2.46 (t, J=6.7 Hz, 1H), 2.71 (dd, J=17.0 Hz, J=11.7 Hz, 1H) 3.07-3.30 (m, 5H) 4.14-4.25 (m, 1H) 9.21 (s, 1H). MS (ESI+) for C₂₁H₃₁N₃O₂S m/z 390 (M+H)⁺.

### Example 198 (BVT.66804)

### 5-(2-Azocan-1-yl-2-oxoethyl)-2-(tricyclo[3.3.1.0˜3,7˜]non-3-ylamino)-1,3-thiazol-4(5H)-one

Prepared according method D.

60 mg, yield 57% as a white solid.

¹H NMR (400 MHz, DMSO-d₆) δ ppm 1.39-1.66 (m, 14H) 1.90-2.11 (m, 6H) 2.23-2.28 (m, 2H) 2.46 (t, J=6.7 Hz, 1H) 2.68 (dd, J=17.1 Hz, J=11.7 Hz, 1H) 3.20 (dd, J=17.1 Hz, J=3.1 Hz, 1H) 3.34-3.37 (m, 4H) 4.18 (dd, J=11.7Hz, J=3.1Hz, 1H) 9.22 (s, 1H). MS (ESI+) for C₂₁H₃₁N₃O₂S m/z 390 (M+H)⁺.

### Example 199 (BVT.66805)

### 5-[2-(1-Oxa-4-azaspiro[4.5]dec-4-yl)-2-oxoethyl]-2-(tricyclo[3.3.1.0˜3,7˜]non-3-ylamino)-1,3-thiazol-4(5H)-one

Prepared according method D.

13 mg, yield 12% as an off-white solid.

¹H NMR (400 MHz, CDCl₃) δ ppm 1.19-1.30 (m, 1H) 1.47-1.67 (m, 11H) 2.00-2.21 (m, 6H) 2.27-2.40 (m, 4H) 2.76 (t, J=6.7 Hz, 1H) 2.85 (dd, J=17.3 Hz, J=12.1 Hz, 1H) 3.41 (dd, J=17.3 Hz, J=3.3 Hz, 1H) 3.50-3.62 (m, 2H) 3.96-4.06 (m, 2H) 4.39 (dd, J=12.1 Hz, J=3.2 Hz, 1H). MS (ESI+) for C₂₂H₃₁N₃O₃S m/z 418 (M+H)⁺.

### Example 200 (BVT066950)

### 2-{[(1R)-1-cyclohexylethyl]amino}-5-[2-(3,4-dihydroquinolin-1(2H)-yl)-2-oxoethyl]-1,3-thiazol-4(5H)-one

Prepared according method D.

65.0 mg yield 46%.

¹H NMR (400 MHz, DMSO-d₆) δ ppm 0.89-1.05 (m, 2H) 1.10 (dd, J=6.8, 4.3 Hz, 3H) 1.12-1.29 (m, 2H) 1.34-1.49 (m, 1H) 1.58-1.77 (m, 6H) 1.90 (m, 2H) 2.71 (t, J=6.7 Hz, 2H) 2.78-2.88 (m, 1H) 3.07-3.18 (m, 0.3H) 3.34-3.43 (m, 1H) 3.61-3.79 (m, 2H) 3.84-3.93 (m, 0.7H) 4.27 (dt, J=11.1, 3.4 Hz, 1H) 7.07-7.13 (m, 1H) 7.13-7.22 (m, 2H) 7.45-7.52 (m, 1H). MS (ES+) for C₂₂H₂₉N₃O₂S m/z 400 (M+H)⁺.

### Example 201 (BVT066951)

### 2-{[(1R)-1-cyclohexylethyl]amino}-5-[2-(3,4-dihydroisoquinolin-2(1H)-yl)-2-oxoethyl]-1,3-thiazol-4(5H)-one

Prepared according method D.

79.8 mg yield 57%.

¹H NMR (400 MHz, DMSO-d₆) δ ppm 0.92-1.05 (m, 2H) 1.11 (dd, J=6.7, 3.8 Hz, 3H) 1.13-1.29 (m, 2H) 1.35-1.51 (m, 1H) 1.58-1.75 (m, 6H) 2.70-2.95 (m, 3H) 3.11-3.20 (m, 0.3H) 3.30-3.40 (m, 1H) 3.61-3.73 (m, 2H) 3.92 (ddd, J=13.5, 6.7, 5.7 Hz, 0.7H) 4.26 (dt, J=11.4, 3.2 Hz, 1H) 4.62 (s, 2H) 7.15-7.21 (m, 4H). MS (ES+) for C₂₂H₂₉N₃O₂S m/z 400 (M+H)⁺.

### Example 202 (BVT066952)

### 5-(2-azepan-1-yl-2-oxoethyl)-2-{[(1R)-1-cyclohexylethyl]amino}-1,3-thiazol-4(5H)-one

Prepared according method D.

73.8 mg, yield 58%.

¹H NMR (400 MHz, DMSO-d₆) δ ppm 0.91-1.05 (m, 2H) 1.11 (dd, J=6.7, 2.5 Hz, 3H) 1.13-1.28 (m, 2H) 1.36-1.46 (m, 1H) 1.47-1.76 (m, 14H) 2.58-2.70 (m, 1H) 3.13-3.19 (m, 0.3H) 3.24 (d, J=16.9 Hz, 1H) 3.36-3.50 (m, 4H) 3.88-3.97 (m, 0.7H) 4.23 (dt, J=11.4, 2.8 Hz, 1H). MS (ES+) for C₁₉H₃₁N₃O₂S m/z 366 (M+H)⁺.

### Example 203 (BVT066953)

### 2-{[(1S)-1-cyclohexylethyl]amino}-5-[2-(3,4-dihydroquinolin-1(2H)-yl)-2-oxoethyl]-1,3-thiazol-4(5H)-one

Prepared according method D.

64.1 mg, yield 46%.

¹H NMR (400 MHz, DMSO-d₆) δ ppm 0.82-1.01 (m, 5H) 1.02-1.31 (m, 3H) 1.54-1.72 (m, 6H) 1.80-1.92 (m, 2H) 2.54-2.64 (m, 1H) 2.64-2.74 (m, 2H) 2.74-2.82 (m, 0.5H) 3.19-3.39 (m, 1H) 3.54-3.71 (m, 2H) 3.71-3.81 (m, 0.5H) 3.96 (ddd, J=10.8, 2.9, 2.6 Hz, 0.5H) 4.12 (dd, J=11.3, 3.0 Hz, 0.5H) 7.04-7.22 (m, 3H) 7.40 (s, 1H). MS (ES+) for C₂₂H₂₉N₃O₂S m/z 400 (M+H)⁺.

### Example 204 (BVT066954)

### 2-{[(1S)-1-cyclohexylethyl]amino}-5-[2-(3,4-dihydroisoquinolin-2(1H)-yl)-2-oxoethyl]-1,3-thiazol-4(5H)-one

Prepared according method D.

38.8 mg, yield 28%.

¹H NMR (400 MHz, DMSO-d₆) δ ppm 0.89-1.01 (m, 2H) 1.08 (dd, J=6.5, 4.9 Hz, 3H) 1.11-1.24 (m, 2H) 1.32-1.44 (m, 1H) 1.57-1.75 (m, 6H) 2.74-2.89 (m, 3H) 3.09-3.17 (m, 0.3H) 3.32 (dd, J=17.2, 2.6 Hz, 1H) 3.57-3.77 (m, 2H) 3.83-3.92 (m, 1H) 4.19-4.28 (m, 1H) 4.57-4.66 (m, 2H) 7.14-7.22 (m, 4H). MS (ES+) for C₂₂H₂₉N₃O₂S m/z 400 (M+H)⁺.

### Example 205 (BVT066956)

### 5-(2-azepan-1-yl-2-oxoethyl)-2-{[(1S)-1-cyclohexylethyl]amino}-1,3-thiazol-4(5H)-one

Prepared according method D.

70.7 mg, yield 55%.

¹H NMR (400 MHz, DMSO-d₆) δ ppm 0.85-1.01 (m, 2H) 1.08 (dd, J=6.7, 3.7 Hz, 3H) 1.10-1.26 (m, 2H) 1.32-1.42 (m, 1H) 1.43-1.53 (m, 4H) 1.54-1.74 (m, 1O H) 2.60-2.73 (m, 1H) 3.05-3.13 (m, 0.3H) 3.16-3.23 (m, 1H) 3.31-3.50 (m, 4H) 3.80-3.94 (m, 1H) 4.11-4.25 (m, 1H). MS (ES+) for C₁₉H₃₁N₃O₂S m/z 366 (M+H)⁺.

### Example 206 (BVT.67010)

### 2-[(cyclohexylmethyl)amino]-5-[2-(4-methylpiperidin-1-yl)-2-oxoethyl]-1,3-thiazol-4(5H)-one

Prepared according method D.

23 mg, yield 35%.

1H NMR (400 MHz, CHLOROFORM-D) δ ppm 0.99 (m, 3H) 1.20 (m, 6H) 1.70 (m, 10H) 2.66 (m, J=10.2, 2.62 Hz, 1H) 2.79 (m, 1H) 3.06 (m, 1H) 3.24 (m, 2H) 3.54 (m, 1H) 3.70 (m, J=13.5, 1.65 Hz, 1H) 4.44 (m, 2H) 9.41 (m, 1H) MS (ES) for C₁₈H₂₉N₃O₂S m/z 352 (M+H)⁺.

### Example 207 (BVT.67011)

### N-cyclohexyl-2-{2-[(cyclohexylmethyl)amino]-4-oxo-4,5-dihydro-1,3-thiazol-5-yl}-N-ethylacetamide

Prepared according method D.

14 mg, yield 20%.

1H NMR (400 MHz, CHLOROFORM-D) δ ppm 1.01 (m, 2H) 1.20 (m, 10H) 1.78 (m, 12H) 2.81 (m, 1H) 3.24 (m, 4H) 3.56 (m, 1H) 4.21 (m, 1H) 4.46 (m, 1H) MS (ES) for C₂₀H₃₃N₃O₂S m/z 380 (M+H)⁺.

### Example 208 (BVT.67012)

### 2-[(cyclohexylmethyl)amino]-5-[2-(1-oxa-4-azaspiro[4.5]dec-4-yl)-2-oxoethyl]-1,3-thiazol-4(5H)-one

Prepared according method D.

8.8 mg, yield 12%.

1H NMR (400 MHz, DMSO-D6) δ ppm 0.92 (m, 3H) 1.17 (m, 5H) 1.46 (m, 12H) 2.27 (m, 1H) 2.49 (m, 1H) 2.66 (m, 1H) 3.03 (m, 1H) 3.12 (m, 1H) 3.23 (m, 1H) 3.38 (m, 1H) 3.57 (m, 1H) 3.89 (m, 1H) 4.28 (m, 1H) 9.26 (m, 1H) MS (ES) for C₂₀H₃₁N₃O₃S m/z 394 (M+H)⁺

### Example 209 (BVT.67015)

### 5-(2-azocan-1-yl-2-oxoethyl)-2-[(cyclohexylmethyl)amino]-1,3-thiazol-4(5H)-one

Prepared according method D.

43.5 mg, yield 64%.

1H NMR (400 MHz, CHLOROFORM-D) δ ppm 0.99 (m, 2H) 1.21 (m, 3H) 1.66 (m, 16H) 2.81 (dd, J=17.0, 12.1 Hz, 1H) 2.91 (dd, J=17.0, 10.4 Hz, 1H) 3.23 (m, 2H) 3.54 (dd, J=16.97, 3.4 Hz, 1H) 4.44 (dd, J=12.1, 3.42 Hz, 1H) 4.54 (dd, J=10.5, 3.05 Hz, 1H). MS (ES) for C₁₉H₃₁N₃O₂S m/z 366 (M+H)⁺.

### Example 210 (BVT.67016)

### 2-[(cyclohexylmethyl)amino]-5-[2-(1,3-dihydro-2H-isoindol-2-yl)-2-oxoethyl]- ,3-thiazol-4(5H)-one

Prepared according method D.

25.25 mg, yield 37%.

1H NMR (400 MHz, CHLOROFORM-D) δ ppm 1.02 (m, 2H) 1.23 (m, 3H) 1.73 (m, 6H) 2.89 (dd, J=17.2, 12.0 Hz, 1H) 3.25 (d, J=6.6 Hz, 2H) 3.58 (dd, J=17.3, 3.2 Hz, 1H) 4.52 (dd, J=12.0, 3.3 Hz, 1H) 4.82 (m, 4H) 7.31 (m, 4H) 13.30 (m, 1H). MS (ES) for C₂₀H₂₅N₃O₂S m/z 372 (M+H)⁺.

### Example 211 (BVT067017)

### N-(3-chloro-2-methylbenzyl)-2-{2-[(cyclohexylmethyl)amino]-4-oxo-4,5-dihydro-1,3-thiazol-5-yl}acetamide

Prepared according method D.

23.6 mg, yield 31%.

1H NMR (400 MHz, CHLOROFORM-D) δ ppm 1.01 (m, 2H) 1.25 (s, 4H) 1.75 (m, J=10.50 Hz, 5H) 2.34 (s, 3H) 2.94 (dd, J=11.72 Hz, 1H) 3.24 (d, J=6.59 Hz, 2H) 3.33 (dd, J=3.42, 1.71 Hz, 1H) 4.45 (m, J=3.66 Hz, 3H) 6.74 (t, J=6.10, 5.13 Hz, 1H) 7.10 (m, 1H) 7.30 (dd, J=6.84, 2.44 Hz, 1H). MS (ES) for C₂₀H₂₆ClN₃O₂S m/z 408 (M+H)⁺.

### Example 212 (BVT067019)

### N-(cyclohexylmethyl)-2-{2-[(cyclohexylmethyl)amino]-4-oxo-4,5-dihydro-1,3-thiazol-5-yl}acetamide

Prepared according method D.

1.83 mg, yield 3%.

1H NMR (400 MHz, CHLOROFORM-D) δ ppm 0.95 (m, 4H) 1.21 (m, 6H) 1.72 (m, 12H) 2.85 (m, 1H) 3.10 (m, 2H) 3.23 (m, J=6.59 Hz, 2H) 3.29 (dd, J=13.43, 3.42 Hz, 1H) 4.38 (dd, J=8.55, 3.42 Hz, 1H). MS (ES) for C₁₉H₃₁N₃O₂S m/z 366 (M+H)⁺.

### Example 213 (BVT067020)

### 2-[(cyclohexylmethyl)amino]-5-[2-(octahydroisoquinolin-2(1H)-yl)-2-oxoethyl]-1,3-thiazol-4(5H)-one

Prepared according method D.

27.6 mg, yield 38%.

1H NMR (400 MHz, CHLOROFORM-D) δ ppm 0.99 (m, 4H) 1.28 (m, 8H) 1.68 (m, 10H) 1.89 (m, 1H) 2.58 (m, 1H) 2.75 (m, 1H) 3.08 (m, 1H) 3.21 (m, 2H) 3.50 (m, 2H) 3.72 (m, 1H) 4.41 (m, 1H) MS (ES) for C₂₁H₃₃N₃O₂S m/z 392 (M+H)⁺.

### Example 214 (BVT067021)

### N-[(1R,2R,4S)-bicyclo[2.2.1]hept-2-yl]-2-{2-[(cyclohexylmethyl)amino]-4-oxo-4,5-dihydro-1,3-thiazol-5-yl-3 acetamide

Prepared according method D.

23.2 mg, yield 35%.

1H NMR (400 MHz, CHLOROFORM-D) δ ppm 1.14 (m, 10H) 1.48 (m, 2H) 1.74 (m, 7H) 2.20 (m, 1H) 2.29 (m, 1H) 2.75 (m, 1H) 3.23 (d, J=5.62 Hz, 2H) 3.27 (m, 1H) 3.67 (m, 1H) 4.40 (m, 1H) MS for C₁₉H₂₉N₃O₂S m/z 364 (M+H)⁺.

### Example 215 (BVT067035T)

### 4-{[2-(cycloheptylamino)-4-oxo-4,5-dihydro-1,3-thiazol-5-yl]acetyl}-1,4-diazepan-1-ium trifluoroacetate

Prepared according method D.

0.06 g, yield 47% as clear crystals.

MS (ES) for C₁₇H₂₉N₄O₂S m/z 353 (M+H)⁺.

### Example 216 (BVT067036)

### 2-[2-(cycloheptylamino)-4-oxo-4,5-dihydro-1,3-thiazol-5-yl]-N-(cyclopropylmethyl)-N-propylacetamide

Prepared according method D.

0.88 g, yield 60% as an off-white solid.

1H NMR (400 MHz, DMSO-D6) δ ppm 0.15-0.29 (m, 2H) 0.36-0.45 (m, 1H) 0.44-0.54 (m, 1H) 0.83 (t, J=7.45 Hz, 3H) 0.88-1.02 (m, 1H) 1.31-1.73 (m, 12H) 1.79-1.99 (m, 2H) 2.78 (dd, J=17.09, 11.47 Hz, 1H) 3.04-3.34 (m, 5H) 3.91-4.04 (m, 1H) 4.21-4.30 (m, 1H) 9.29 (d, J=7.45 Hz, 1H). MS (ES) for C₁₉H₃₁N₃O₂S m/z 366 (M+H)⁺.

### Example 217 (BVT067037)

### 2-(cycloheptylamino)-5-[2-(1,3-dihydro-2H-isoindol-2-yl)-2-oxoethyl]-1,3-thiazol-4(5H)-one

Prepared according method D.

0.70 g, yield 51% as an off-white solid.

1H NMR (400 MHz, DMSO-D6) δ ppm 1.27-1.70 (m, 10H) 1.74-2.01 (m, 2H) 3.18-3.40 (m, 2H) 3.97 (s, 1H) 4.18-4.39 (m, 1H) 4.63 (s, 2H) 4.82 (s, 2H) 7.22-7.40 (m, 4H) 9.10-9.22 (m, 1H). MS (ES) for C₂₀H₂₅N₃O₂S m/z 372 (M+H)⁺.

### Example 218 (BVT067038)

### 5-(2-azocan-1-yl-2-oxoethyl)-2-(cycloheptylamino)-1,3-thiazol-4(5H)-one

Prepared according method D.

0.98 g, yield 100% as an off-white solid.

1H NMR (400 MHz, DMSO-D6) δ ppm 1.28-2.00 (m, 22H) 3.10-3.26 (m, 2H) 3.27-3.42 (m, 4H) 3.84-4.02 (m, 1H) 4.13-4.30 (m, 1H) 9.06-9.28 (m, 1H). MS (ES) for C₁₉H₃₁N₃O₂S m/z 366 (M+H)⁺.

### Example 219 (BVT067055)

### 2-[2-(cycloheptylamino)-4-oxo-4,5-dihydro-1,3-thiazol-5-yl]-N-cyclohexyl-N-ethylacetamide

Prepared according method D.

0.64 g, yield 45% as an off-white solid.

MS (ES) for C₂₀H₃₃N₃O₂S m/z 380 (M+H)⁺.

### Example 220 (BVT.67371)

### 2-{2-[(cyclohexylmethyl)amino]-4-oxo-4,5-dihydro-1,3-thiazol-5-yl}-N-methyl-N-phenylacetamide

Prepared according method D.

32 mg, yield 48%.

1H NMR (400 MHz, CHLOROFORM-D) δ ppm 0.98 (m, 2H) 1.21 (m, 3H) 1.72 (m, 6H) 2.57 (m, 1H) 3.14 (dd, J=17.58, 3.42 Hz, 1H) 3.22 (dd, J=6.59, 1.71 Hz, 2H) 3.29 (m, 3H) 4.36 (dd, J=11.72, 3.17 Hz, 1H) 7.17 (m, 2H) 7.43 (m, 3H). MS (ES) for C₁₉H₂₅N₃O₂S m/z 360 (M+H)⁺.

### Example 221 (BVT.67372)

### 2-{2-[(cyclohexylmethyl)amino]-4-oxo-4,5-dihydro-1,3-thiazol-5-yl}-N-(4-methoxyphenyl)-N-methylacetamide

Prepared according method D.

20 mg, yield 28%.

1H NMR (400 MHz, CHLOROFORM-D) δ ppm 0.99 (m, 2H) 1.22 (m, 3H) 1.73 (m, 6H) 2.53 (m, 1H) 3.15 (dd, J=17.58, 3.42 Hz, 1H) 3.21 (dd, J=6.59, 0.98 Hz, 2H) 3.26 (m, 3H) 3.83 (m, 3H) 4.34 (dd, J=11.96, 3.42 Hz, 1H) 6.93 (m, 2H) 7.08 (m, 2H). MS (ES) for C₂₀H₂₇N₃O₃S m/z 390 (M+H)⁺.

### Example 222 (BVT.67373)

### 2-{2-[(cyclohexylmethyl)amino]-4-oxo-4,5-dihydro-1,3-thiazol-5-yl}-N-ethyl-N-phenylacetamide

Prepared according method D.

38 mg, yield 55%.

1H NMR (400 MHz, CHLOROFORM-D) δ ppm 0.99 (m, 2H) 1.13 (t, J=7.08 Hz, 3H) 1.25 (m, 3H) 1.72 (m, 6H) 2.50 (m, 1H) 3.09 (dd, J=17.58, 3.42 Hz, 1H) 3.21 (dd, J=6.35, 1.22 Hz, 2H) 3.76 (m, 2H) 4.35 (dd, J=11.96, 3.42 Hz, 1H) 7.14 (m, 2H) 7.44 (m, 3H). MS (ES) for C₂₀H₂₇N₃O₂S m/z 374 (M+H)⁺.

### Example 223 (BVT.67374)

### N-butyl-2-{2-[(cyclohexylmethyl)amino]-4-oxo-4,5-dihydro-1,3-thiazol-5-yl}-N-phenylacetamide

Prepared according method D.

38 mg, yield 52%.

1H NMR (400 MHz, CHLOROFORM-D) δ ppm 0.89 (t, J=7.32 Hz, 3H) 0.97 (m, 2H) 1.19 (m, 3H) 1.30 (m, 2H) 1.49 (m, 2H) 1.71 (m, 6H) 2.46 (m, 1H) 3.09 (dd, J=17.58, 3.17 Hz, 1H) 3.19 (d, J=6.35 Hz, 2H) 3.69 (m, 2H) 4.33 (dd, J=11.96, 3.42 Hz, 1H) 7.13 (m, 2H) 7.42 (m, 3H). MS (ES) for C₂₂H₃₁N₃O₂S m/z 402 (M+H)⁺.

### Example 224 (BVT067392)

### N-butyl-2-[2-(cycloheptylamino)-4-oxo-4,5-dihydro-1,3-thiazol-5-yl]-N-phenylacetamide

Prepared according method D.

0.69 g, yield 47% as an off white solid.

1H NMR (400 MHz, DMSO-D6) δ ppm 0.83 (t, 3H) 1.16-1.68 (m, 12H) 1.71-1.98 (m, 2H) 2.70-2.96 (m, 1H) 3.51-3.77 (m, 2H) 3.81-4.01 (m, 1H) 4.08-4.30 (m, 1H) 7.39 (d, J=48.34 Hz, 5H) 8.96-9.28 (m, 1H). MS (ES) for C₂₂H₃₁N₃O₂S m/z 402 (M+H)⁺.

### Example 225 (BVT067394)

### N-benzyl-2-[2-(cycloheptylamino)-4-oxo-4,5-dihydro-1,3-thiazol-5-yl]-N-phenylacetamide

Prepared according method D.

0.60 g, yield 37% as an off-white solid.

1H NMR (400 MHz, DMSO-D6) δ ppm 1.22-1.68 (m, J=6.84 Hz, 10H) 1.66-1.99 (m, 2H) 2.81-3.07 (m, 1H) 3.82-4.05 (m, 1H) 4.17-4.36 (m, 1H) 4.73-4.99 (m, 2H) 6.84-7.51 (m, 10H) 8.89-9.31 (m, 1H). MS (ES) for C₂₅H₂₉N₃O₂S m/z 436 (M+H)⁺.

### Example 226 (BVT067453)

### 5-[2-(1,3-dihydro-2H-isoindol-2-yl)-2-oxoethyl]-2-[(2,2,3,3-tetramethylcyclopropyl)-amino]-1,3-thiazol-4(5H)-one

Prepared according method D.

2.4 mg, yield 4%.

¹H NMR (400 MHz, CHLOROFORM-D) δ ppm 1.11 (d, J=2.0 Hz, 6H), 1.18 (d, J=10.5 Hz, 6H), 2.13 (s, 1H), 2.83 (dd, J=17.1, 12.0 Hz, 1H), 3.56 (dd, J=17.2, 3.1 Hz, 1H), 4.48 (dd, J=12.0, 3.2 Hz, 1H), 4.74-4.88 (m, 4H), 7.27-7.39 (m, 4H); MS (ES+) m/z 372 (M+H⁺). MS (ES) for C₂₀H₂₅N₃O₂S m/z 372 (M+H)⁺.

### Example 227 (BVT067454)

### 5-(2-azepan-1-yl-2-oxoethyl)-2-[(2,2,3,3-tetramethylcyclopropyl)amino]-1,3-thiazol-4(5H)-one

Prepared according method D.

7.2 mg, yield 14%.

¹H NMR (400 MHz, CHLOROFORM-D) δ ppm 1.10 (s, 6H), 1.18 (d, J=9.8 Hz, 6H), 1.52-1.64 (m, 4H), 1.67-1.80 (m, 4H), 2.13 (s, 1H), 2.77 (dd, J=17.0, 12.1 Hz, 1H), 3.33-3.57 (m, 4H), 3.57-3.67 (m, 1H), 4.42 (dd, J=12.0, 3.2 Hz, 1H). MS (ES) for C₁₈H₂₉N₃O₂S m/z 352 (M+H)⁺.

Compounds of Type 4

### Example 228 (BVT.51206)

### 2-[2-(bicyclo[2.2.1]hept-5-en-2-ylamino)-4-oxo-4,5-dihydro-1,3-thiazol-5-yl]ethyl benzoate

Method F

2-(bicyclo[2.2.1]hept-5-en-2-ylamino)-5-(2-hydroxyethyl)-1,3-thiazol-4(5H)-one (76 mg, 0.3 mmol) and benzoyl chloride (35 μL, 0.3 mmol) were dissolved in DCM (3 mL) and triethylamine (0.13 mL, 0.9 mmol) was added. The reaction mixture was stirred over night at RT. The solvent was removed under reduced pressure and the product was purified using preparative HPLC (10-90% MeCN over 10 min followed by 100% MeCN for 5 min) affording the product in 19% yield (20 mg).

¹H NMR (270 MHz, CHLOROFORM-D) δ ppm 1-52-1.72 (m, 3H) 1.89-1.99 (m, 1H) 2.17-2.42 (m, 1H) 2.65-2.80 (m, 1H) 2.96 (d, J=14.85 Hz, 2H) 3.27-3.37 (m, 1H) 4.18-4.28 (m, 1H) 4.40-4.62 (m, 2H) 5.95-6.05 (m, 1H) 6.18-6.28 (m, 1H) 7.43 (t, 2H) 7.57 (m, 1H) 8.03 (m, 2H). HPLC 96% R_{T}=2.15 (System A. 10-97% MeCN over 3 min) 94% R_{T}=1.98 (System B. 10-90% MeCN over 3 min). MS (ESI+) for C₁₉H₂₀N₂O₃S m/z 357 (M+H)⁺.

### Example 229 (BVT.51207)

### 2-[2-(bicyclo[2.2.1]hept-5-en-2-ylamino)-4-oxo-4,5-dihydro-1,3-thiazol-5-yl]ethyl 2-chlorobenzoate

Prepared according to method F

30 mg 25% yield.

¹H NMR (270 MHz, CHLOROFORM-D) δ ppm 0.53-0.93 (m, 1H) 1.15-1.40 (m, 2H) 1.65-1.87 (m, 3H) 2.23-2.44 (m, 1H) 2.64-2.74 (m, 1H) 2.85-3.06 (m, 2H) 3.26-3.38 (m, 1H) 4.25-4.40 (m, 1H) 4.43-4.65 (m, 2H) 5.95-6.07 (m, 1H) 6.25-6.33 (m, 1H) 7.25-7.50 (m, 2H) 7.77-7.95 (m, 1H). MS (ESI+) for C₁₉H₁₉ClN₂O₃S m/z 391 (M+H)⁺.

### Example 230 (BVT.51601G)

### 2-[2-(bicyclo[2.2.1]hept-5-en-2-ylamino)-4-oxo-4,5-dihydro-1,3-thiazol-5-yl]ethyl 3,4-dichlorobenzoate

Prepared according to method F

25 mg, 29% yield.

¹H NMR (270 MHz, METHANOL-D4) δ ppm 1.27-1.77 (m, 4H) 2.45-2.63 (m, 2H) 2.84-2.95 (m, 2H) 3.70-3.80 (m, 1H) 4.42-4.60 (m, 3H) 5.90-6.10 (m, 1H) 6.14-6.20 (m, 1H) 7.60-7.70 (m, 1H) 7.85-7.95 (m, 1H) 8.04 (dd, J=4.82, 1.86 Hz, 1H). MS (ESI+) for C₁₉H₁₈Cl₂N₂O₃SC₂HCl₃O₂ m/z 426 (M+H)⁺.

### Example 231 (BVT.51602G)

### 2-[2-(bicyclo[2.2.1]hept-5-en-2-ylamino)-4-oxo-4,5-dihydro-1,3-thiazol-5-yl]ethyl 2,6-difluorobenzoate

Prepared according to method F

76 mg, 97% yield.

¹H NMR (270 MHz, METHANOL-D4) δ ppm 1.34-1.53 (m, 3H) 1.57-1.70 (m, 1H) 2.09-2.28 (m, 1H) 2.45-2.60 (m, 1H) 2.73-2.88 (m, 2H) 3.63-3.72 (m, 1H) 4.30-4.47 (m, 3H) 5.94-6.00 (m, 1H) 6.07-6.13 (m, 1H) 6.98 (t, J=8.29 Hz, 1H) 7.40-7.53 (m, 1H). MS (ESI+) for C₁₉H₁₈F₂N₂O₃SC₂HCl₃O₂ m/z 393 (M+H)⁺.

### Example 232 (BVT.51603G)

### 2-[2-(bicyclo[2.2.1]hept-5-en-2-ylamino)-4-oxo-4,5-dihydro-1,3-thiazol-5-yl]ethyl 2,5-bis(trifluoromethyl)benzoate

Prepared according to method F

19 mg, 19% yield.

¹H NMR (270 MHz, METHANOL-D4) δ ppm 1.20-1.68 (m, 4H) 2.16-2.34 (m, 1H) 2.43-2.60 (m, 1H) 2.75-2.90 (m, 2H) 3.68-3.76 (m, 1H) 4.28-4.32 (m, 1H) 4.40-4.53 (m, 2H) 5.92-6.00 (m, 1H) 6.07-6.20 (m, 1H) 7.92-8.00 (m, 2H) 8.11 (d, J=6.48 Hz, 1H). MS (ESI+) for C₂₁H₁₈F₆N₂O₃SC₂HCl₃O₂ m/z 493 (M+H)⁺.

### Example 233 (BVT.51605G)

### 2-[2-(bicyclo[2.2.1]hept-5-en-2-ylamino)-4-oxo-4,5-dihydro-1,3-thiazol-5-yl]ethyl 3,4-difluorobenzoate trifluoroacetate

Prepared according to method F

27 mg, 33% yield.

¹H NMR (270 MHz, METHANOL-D4) δ ppm 1.15-1.57 (m, 4H) 2.33-2.57 (m, 2H) 2.68-2.87 (m, 2H) 3.55-3.60 (m, 1H) 4.40-4.50 (m, 3H) 5.96-6.12 (m, 2H) 7.45-7.55 (m, 2H) 7.80-7.87 (m, 1H) 7.90-8.00 (m, 1H) 8.48-8.53 (m, 1H). MS (ESI+) for C₂₃H₂₂N₂O₃SC₂HCl₃O₂ m/z 407 (M+H)⁺.

### Example 234 (BVT.51606G)

### 2-[2-(bicyclo[2.2.1]hept-5-en-2-ylamino)-4-oxo-4,5-dihydro-1,3-thiazol-5-yl]ethyl 3,4-difluorobenzoate

Prepared according to method F

8 mg, 10% yield.

¹H NMR (270 MHz, CHLOROFORM-D) δ ppm 1.57-1.65 (m, 2H) 1.67-1.82 (m, 2H) 2.33-2.52 (m, 1H) 2.62-2.75 (m, 1H) 2.90-3.04) 3.28-3.36 (m, 1H) 4.25-4.35 (m, 1H) 4.44-4.60 (m, 2H) 5.99-6.07 (m, 1H) 6.24-6.32 (m, 1H) 7.16-7.28 (m, 1H) 7.75-7.93 (m, 2H). MS (ESI+) for C₁₉H₁₈F₂N₂O₃SC₂HCl₃O₂ m/z 393 (M+H)⁺.

### Example 235 (BVT.51608G)

### 2-[2-(bicyclo[2.2.1]hept-5-en-2-ylamino)-4-oxo-4,5-dihydro-1,3-thiazol-5-yl]ethyl 2,5-difluorobenzoate

Prepared according to method F

9 mg, 11% yield.

¹H NMR (270 MHz, METHANOL-D4) δ ppm 1.15-1.67 (m, 4H) 2.19-2.38 (m, 1H) 2.43-2.55 (m, 1H) 2.77-2.88 (m, 2H) 3.68 (dd, J=7.67, 2.97 Hz, 1H) 4.34-4.47 (m, 3H) 5.90-6.00 (m, 1H) 6.07-6.13 (m, 1H) 7.08-7.21 (m, 1H) 7.23-7.34 (m, 1H) 7.52-7.62 (m, 1H). MS (ESI+) for C₁₉H₁₈F₂N₂O₃SC₂HCl₃O₂ m/z 393 (M+H)⁺.

### Example 236 (BVT.51607G)

### 2-[2-(bicyclo[2.2.1]hept-5-en-2-ylamino)-4-oxo-4,5-dihydro-1,3-thiazol-5-yl]ethyl 4-methylbenzoate

Prepared according to method F

20 mg, 27% yield.

¹H NMR (270 MHz, CHLOROFORM-D) δ ppm 1.54-1.77 (m, 4H) 2.30-2.47 (m, 1H) 2.40 (s, 3H) 2.63-2.77 (m, 1H) 2.90-3.04 (m, 2H) 3.25-3.33 (m, 1H) 4.27-4.35 (m, 1H) 4.42-4.60 (m, 2H) 5.97-6.04 (m, 1H) 6.23-6.28 (m, 1H) 7.25 (d, J=7.56 Hz 2H) 7.89 (d, J=8.10 Hz 2H). MS (ESI+) for C₂₀H₂₂N₂O₃SC₂HCl₃O₂ m/z 371 (M+H)⁺.

### Example 237 (BVT.51609G)

### 2-[2-(bicyclo[2.2.1]hept-5-en-2-ylamino)-4-oxo-4,5-dihydro-1,3-thiazol-5-yl]ethyl 4-chloro-3-nitrobenzoate

Prepared according to method F

18 mg, 21% yield.

¹H NMR (270 MHz, CHLOROFORM-D) δ ppm 1.61 (br s, 2H) 1.70-1.78 (m, 2H) 2.40-2.57 (m, 1H) 2.62-2.68 (m, 1H) 2.97 (br s, 1H) 3.02 (br s, 1H) 3.28-3.38 (m, 1H) 4.27-4.35 (m, 1H) 4.47-4.68 (m, 2H) 6.02-6.09 (m, 1H) 6.27-6.32 (m, 1H) 7.67 (d, J=8.41 Hz 1H) 8.12 (dd, J=8.41, 1.98 Hz, 1H) 8.46-8.52 (m, 1H). MS (ESI+) for C₁₉H₁₈ClN₃O₅SC₂HCl₃O₂ m/z 436 (M+H)⁺.

### Example 23 8 (BVT.51611G)

### 2-[2-(bicyclo[2.2.1]hept-5-en-2-ylamino)-4-oxo-4,5-dihydro-1,3-thiazol-5-yl]ethyl 3-methylbenzoate

Prepared according to method F

9 mg, 12% yield.

¹H NMR (270 MHz, CHLOROFORM-D) δ ppm 1.57-1.67 (m, 2H) 1.68-1.78 (m, 2H) 2.25-2.47 (m, 1H) 2.39 (s, 3H) 2.68-2.80 (m, 1H) 2.92-3.04 (m, 2H) 3.26-3.335 (m, 1H) 4.27-4.34 (m, 1H) 4.42-4.62 (m, 2H) 5.96-6.03 (m, 1H) 6.23-6.29 (m, 1H) 7.30-7.44 (m, 2H) 7.77-7.86 (m, 2H). MS (ESI+) for C₂₀H₂₂N₂O₃SC₂HCl₃O₂ m/z 371 (M+H)⁺.

### Example 239 (BVT.51682G)

### 2-[2-(bicyclo[2.2.1]hept-5-en-2-ylamino)-4-oxo-4,5-dihydro-1,3-thiazol-5-yl]ethyl 3-(trifluoromethyl)benzoate

Prepared according to method F

32 mg, 38% yield.

¹H NMR (270 MHz, CHLOROFORM-D) δ ppm 1.52-1.72 (m, 3H) 1.94-2.08 (m, 1H) 2.18-2.42 (m, 1H) 2.65-2.84 (m, 1H) 2.95 (d, J=13.11 Hz, 2H) 3.27-3.37 (m, 1H) 4.14-4.23 (m, 1H) 4.42-4.65 (m, 2H) 5.94-6.03 (m, 1H) 6.17-6.25 (m, 1H) 7.57 (t, J=7.79 Hz, 1H) 7.81 (d, J=7.67 Hz, 1H) 8.22 (d, J=7.67 Hz, 1H) 8.30 (s, 1H). MS (ESI+) for C₂₀H₁₉F₃N₂O₃SC₂HCl₃O₂ m/z 425 (M+H)⁺.

### Example 240 (BVT.51683G)

### 2-[2-(bicyclo[2.2.1]hept-5-en-2-ylamino)-4-oxo-4,5-dihydro-1,3-thiazol-5-yl]ethyl 2,3,4-trifluorobenzoate

Prepared according to method F

21 mg, 26% yield.

¹H NMR (270 MHz, CHLOROFORM-D) δ ppm 1.52-1.68 (m, 3H) 1.93-2.08 (m, 1H) 2.16-2.36 (m, 1H) 2.60-2.77 (m, 1H) 2.89-3.02 (m, 2H) 3.27-3.36 (m, 1H) 4.16-428 (m, 1H) 4.42-4.60 (m, 2H) 5.97-6.06 (m, 1H) 6.18-6.28 (m, 1H) 6.96-7.08 (m, 1H) 7.67-7.80 (m, 1H). MS m/z: (M+H) 411.

### Example 241 (BVT.51684G)

### 2-[2-(bicyclo[2.2.1]hept-5-en-2-ylamino)-4-oxo-4,5-dihydro-1,3-thiazol-5-yl]ethyl 2-bromo-5-methoxybenzoate

Prepared according to method F

10 mg, 11% yield.

¹H NMR (270 MHz, CHLOROFORM-D) δ ppm 1.55-1.72 (m, 3H) 1.85-1.97 (m, 1H) 2.18-2.37 (m, 1H) 2.65-2.80 (m, 1H) 2.92-3.03 (m, 2H) 3.27-3.36 (m, 1H) 3.81 (s, 3H) 4.22-4.34 (m, 1H) 4.44-4.56 (m, 2H) 5.98-6.04 (m, 1H) 6.18-6.27 (m, 1H) 6.89 (dd, J=8.78, 3.09 Hz, 1H) 7.28-7.33 (m, 1H) 7.48-7.54 (m, 1H). MS (ESI+) for C₂₀H₂₁BrN₂O₄SC₂HCl₃O₂ m/z 467 (M+H)⁺.

### Example 242 (BVT.51685G)

### 2-[2-(bicyclo[2.2.1]hept-5-en-2-ylamino)-4-oxo-4,5-dihydro-1,3-thiazol-5-yl]ethyl 2-chloro-6-fluorobenzoate

Prepared according to method F

10 mg, 12% yield.

¹H NMR (270 MHz, CHLOROFORM-D) δ ppm 1.52-1.72 (m, 3H) 1.85-1.98 (m, 1H) 2.08-2.27 (m, 1H) 2.65-2.82 (m, 1H) 2.90-3.02′(m, 2H) 3.28-3.37 (m, 1H) 4.17-4.28 (m, 1H) 4.46-4.63 (m, 2H) 5.96-6.07 (m, 1H) 6.18-6.25 (m, 1H) 7.05 (t, J=8.66 Hz, 1H) 7.17-7.27 (m, 1H) 7.29-7.39 (m, 1H). MS (ESI+) for C₁₉H₁₈ClFN₂O₃SC₂HCl₃O₂ m/z 409 (M+H)⁺.

### Example 243 (BVT.51686G)

### 2-[2-(bicyclo[2.2.1]hept-5-en-2-ylamino)-4-oxo-4,5-dihydro-1,3-thiazol-5-yl]ethyl 2-fluoro-5-(trifluoromethyl)benzoate

Prepared according to method F

6 mg, 7% yield.

¹H NMR (270 MHz, CHLOROFORM-D) δ ppm 1.55-1.75 (m, 3H) 1.85-2.04 (m, 1H) 2.14-2.37 (m, 1H) 2.70-2.84 (m, 1H) 2.97 (d, J=11.38 Hz, 2H) 3.28-3.38 (m, 1H) 4.17-4.29 (m, 1H) 4.45-4.63 (m, 2H) 5.97-6.08 (m, 1H) 6.17-6.28 (m, 1H) 7.20-7.35 (m, 1H) 7.74-7.86 (m, 1H) 8.20-8.30 (m, 1H). MS (ESI+) for C₂₀H₁₈F₄N₂O₃SC₂HCl₃O₂ m/z 443 (M+H)⁺.

### Example 244 (BVT.51687G)

### 2-[2-(bicyclo[2.2.1]hept-5-en-2-ylamino)-4-oxo-4,5-dihydro-1,3-thiazol-5-yl]ethyl 2-fluoro-4-(trifluoromethyl)benzoate

Prepared according to method F

22 mg, 25% yield.

¹H NMR (270 MHz, CHLOROFORM-D) δ ppm 1.52-1.68 (m, 3H) 1.92-2.06 (m, 1H) 2.17-2.44 (m, 1H) 2.58-2.77 (m, 1H) 2.95 (d, J=14.10 Hz, 2H) 3.27-3.36 (m, 1H) 4.15-4.27 (m, 1H) 4.44-4.65 (m, 2H) 5.96-6.08 (m, 1H) 6.17-6.26 (m, 1H) 7.35-7.52 (m, 2H) 8.06 (t, J=7.30 Hz, 1H). MS (ESI+) for C₂₀H₁₈F₄N₂O₃SC₂HCl₃O₂ m/z 443 (M+H)⁺.

### Example 245 (BVT.56824G)

### 2-[2-(bicyclo[2.2.1]hept-5-en-2-ylamino)-4-oxo-4,5-dihydro-1,3-thiazol-5-yl]ethyl 3-methoxybenzoate

Prepared according to method F

23 mg, 20% yield.

¹H NMR (270 MHz, METHANOL-D4) δ ppm 1.17-1.72 (m, 4H) 2.31-2.50 (m, 3H) 2.68-2.77 (m, 1H) 3.58-3.68 (m, 1H) 3.75 (d, J=4.45 Hz, 3H) 4.27-4.45 (m, 3H) 5.82-5.98 (m, 1H) 6.02-6.18 (m, 1H) 6.98-7.08 (m, 1H) 7.23-7.28 (m, 1H) 7.42-7.52 (m, 2H). MS (ESI+) for C₂₀H₂₂N₂O₄SC₂HCl₃O₂ m/z 387 (M+H)⁺.

### Example 246 (BVT.56825G)

### 2-[2-(bicyclo[2.2.1]hept-5-en-2-ylamino)-4-oxo-4,5-dihydro-1,3-thiazol-5-yl]ethyl 2,6-dimethoxybenzoate

Prepared according to method F

12 mg, 10% yield.

¹H NMR (270 MHz, METHANOL-D4) δ ppm 1.28-1.48 (m, 3H) 1.50-1.62 (m, 1H) 1.85-2.05 (m, 1H) 2.43-2.54 (m, 1H) 2.67-2.84 (m, 2H) 3.10-3.16 (m, 1H) 3.64 (s, 6H) 4.17-4.37 (m, 3H) 5.87-5.96 (m, 1H) 6.03-6.13 (m, 1H) 6.51 (d, J=8.41 Hz, 2H) 6.03-6.13 (m, 1H) 7.18 (t, J=8.24 Hz, 1H). MS (ESI+) for C₂₁H₂₄N₂O₅SC₂HCl₃O₂ m/z 417 (M+H)⁺.

### Example 247 (BVT.56826G)

### 2-[2-(bicyclo[2.2.1]hept-5-en-2-ylamino)-4-oxo-4,5-dihydro-1,3-thiazol-5-yl]ethyl 2,4-dimethoxybenzoate

Prepared according to method F

15 mg, 12% yield.

¹H NMR (270 MHz, METHANOL-D4) δ ppm 1.27-1.64 (m, 4H) 2.13-2.34 (m, 1H) 2.42-2.55 (m, 1H) 2.72-2.86 (m, 1H) 3.54-3.62 (m, 1H) 3.73 (s, 6H) 4.22-4.43 (m, 3H) 5.84-5.98 (m, 1H) 6.05-6.18 (m, 1H) 6.40-6.53 (m, 2H) 7.65-7.76 (m, 1H). MS (ESI+) for C₂₁H₂₄N₂O₅SC₂HCl₃O₂ m/z 417 (M+H)⁺.

### Example 248 (BVT.56827G)

### 2-[2-(bicyclo[2.2.1]hept-5-en-2-ylamino)-4-oxo-4,5-dihydro-1,3-thiazol-5-yl]ethyl 4-butoxybenzoate

Prepared according to method F

6 mg, 4% yield.

¹H NMR (270 MHz, CHLOROFORM-D) δ ppm 0.93 (t, J=7.42 Hz, 3H) 1.36-1.59 (m, 5H) 1.60-1.79 (m, 3H) 2.08-2.28 (m, 1H) 2.58-2.74 (m, 1H) 2.75-2.94 (m, 2H) 3.22-3.30 (m, 1H) 3.96 (t, J=6.43 Hz, 2H) 4.16-4.28 (m, 1H) 4.32-4.49 (m, 2H) 5.93-6.02 (m, 1H) 6.07-6.23 (m, 1H) 6.85 (d, J=8.16 Hz, 2H) 7.93 (d, J=8.16 Hz, 2H). MS (ESI+) for C₂₃H₂₈N₂O₄SC₂HCl₃O₂ m/z 430 (M+H)⁺.

### Example 249 (BVT.56857G)

### 2-[2-(bicyclo[2.2.1]hept-5-en-2-ylamino)-4-oxo-4,5-dihydro-1,3-thiazol-5-yl]ethyl 3,5-bis(trifluoromethyl)benzoate

Prepared according to method F

30 mg, 20% yield.

¹H NMR (270 MHz, CHLOROFORM-D) δ ppm 1.47-1.78 (m, 3H) 1.84-2.02 (m, 1H) 2.20-2.44 (m, 1H) 2.63-2.81 (m, 1H) 2.83-2.98 (m, 2H) 3.24-3.36 (m, 1H) 4.13-4.23 (m, 1H) 4.40-4.68 (m, 2H) 5.92-6.04 (m, 1H) 6.13-6.25 (m, 1H) 8.06 (br.s, 1H) 8.48 (br.s, 2H). MS (ESI+) for C₂₁H₁₈F₆N₂O₃SC₂HCl₃O₂ m/z 493 (M+H)⁺.

### Example 250 (BVT.56858G)

### 2-[2-(bicyclo[2.2.1]hept-5-en-2-ylamino)-4-oxo-4,5-dihydro-1,3-thiazol-5-yl]ethyl 4-tert-butylbenzoate

Prepared according to method F

7 mg, 5% yield.

¹H NMR (270 MHz, CHLOROFORM-D) δ ppm 1.32 (s, 9H) 1.55-1.72 (m, 3H) 1.86-1.94 (m, 1H) 2.18-2.36 (m, 1H) 2.67-2.78 (m, 1H) 2.88-3.00 (m, 2H) 3.27-3.36 (m, 1H) 4.17-4.28 (m, 1H) 4.40-4.62 (m, 2H) 5.92-6.08 (m, 1H) 6.18-6.26 (m, 1H) 7.44 (d, J=8.41 Hz, 2H) 7.95 (d, J=8.40 Hz, 2H). MS (ESI+) for C₂₃H₂₈N₂O₃SC₂HCl₃O₂ m/z 414 (M+H)⁺.

### Example 251 (BVT.56890G)

### 2-[2-(bicyclo[2.2.1]hept-5-en-2-ylamino)-4-oxo-4,5-dihydro-1,3-thiazol-5-yl]ethyl 2,4-dichlorobenzoate

Prepared according to method F

5 mg, 4% yield.

¹H NMR (270 MHz, CHLOROFORM-D) δ ppm 1.21-1.45 (m, 1H) 1.52-1.74 (m, 2H) 1.82-1.93 (m, 1H) 2.17-2.37 (m, 1H) 2.64-2.76 (m, 1H) 2.88-3.02 (m, 2H) 3.30-3.36 (m, 1H) 4.20-4.30 (m, 1H) 4.42-4.57 (m, 2H) 5.97-6.06 (m, 1H) 6.22-6.27 (m, 1H) 7.25-7.35 (m, 1H) 7.47 (d, J=1.98 Hz, 1H) 7.82 (dd, J=2.16, 8.37 Hz, 1H). MS (ESI+) for C₁₉H₁₈Cl₂N₂O₃SC₂HCl₃O₂ m/z 425 (M+H)⁺.

### Example 252 (BVT.59294G)

### 2-[2-(bicyclo[2.2.1]hept-5-en-2-ylamino)-4-oxo-4,5-dihydro-1,3-thiazol-5-yl]ethyl 2,4,6-trichlorobenzoate

Prepared according to method F

5 mg, 3% yield.

¹H NMR (270 MHz, CHLOROFORM-D) δ ppm 1.17-1.37 (m, 1H) 1.65 (s, 2H) 1.73-1.78 (m, 2H) 2.23-2.36 (m, 1H) 2.69-2.84 (m, 1H) 2.95-3.06 (m, 2H) 3.28-3.34 (m, 1H) 4.31-4.38 (m, 1H) 4.48-4.67 (m, 2H) 6.04 (dd, J=5.57, 3.09 Hz, 1H) 6.28 (dd, J=5.57, 2.85 Hz, 1H) 7.33-7.43 (m, 2H). MS (ESI+) for C₁₉H₁₇C₁₃N₂O₃SC₂HCl₃O₂ m/z 461 (M+H)⁺.

Compounds of Type 4B

### Example 253 (BVT051436G)

### 2-[2-(bicyclo[2.2.1]hept-5-en-2-ylamino)-4-oxo-4,5-dihydro-1,3-thiazol-5-yl]ethyl (2-chlorophenyl)carbamate

Method F

2-(bicyclo[2.2.1]hept-5-en-2-ylamino)-5-(2-hydroxyethyl)-1,3-thiazol-4(5H)-one (76 mg, 0.3 mmol) and 2-chlorophenyl isocyanate (78 }L, 0.6 mmol) were dissolved in anhydrous DCM and the reaction mixture was stirred at RT over night. The solvent was removed under reduced pressure and the crude was purified using preparative HPLC (20-80% MeCN over 10 min followed by 100% MeCN for 5 min) affording the product in 43% yield (54 mg).

¹H NMR (270 MHz, CHLOROFORM-D) δ ppm 1.60-1.84 (m, 3H) 2.00-2.13 (m, 1H) 2.20-2.40 (m, 1H) 2.65-2.79 (m, 1H) 3.00-3.11 (m, 2H) 3.42 (d, J=5.69 Hz, 1H) 4.26-4.36 (m, 1H) 4.48 (q, J=5.40 Hz, 1H) 4.37-4.55 (m, 1H) 6.07-6.16 (m, 1H) 6.30-6.34 (m, 1H) 7.09 (t, J=7.79 Hz, 1H) 7.34 (t, J=7.97 Hz, 1H) 7.43 (d, J=8.16 Hz, 1H) 8.18 (d, J=7.92 Hz, 1H). HPLC 100% R_{T}=2.18 (System A. 10-97% MeCN over 3 min) 99% R_{T}=1.43 (System B. 10-90% MeCN over 3 min). MS (ESI+) for C₁₉H₂₀ClN₃O₃S2C₂HCl₃O₂ m/z 406 (M+H)⁺.

### Example 254 (BVT051437G)

### 2-[2-(bicyclo[2.2.1]hept-5-en-2-ylamino)-4-oxo-4,5-dihydro-1,3-thiazol-5-yl]ethyl (4-chloro-3-nitrophenyl)carbamate

Prepared according to method F

23 mg, 16% yield.

¹H NMR (270 MHz, CHLOROFORM-D) δ ppm 1.54-1.75 (m, 3H) 1.87-2.00 (m, 1H) 2.17-2.37 (m, 1H) 2.58-2.70 (m, 1H) 2.99 (br s, 2H) 3.34 (d, J=7.67 Hz, 1H) 4.17-4.24 (m, 1H) 4.33-4.50 (m, 2H) 6.02-6.09 (m, 1H) 6.21-6.27 (m, 1H) 7.55-7.62 (m, 1H) 8.20 (d, J=2.47 Hz, 1H) 8.52 (dd, J=9.15, 2.72 Hz, 1H) 9.82 (d, J=3.96 Hz, 1H). MS (ESI+) for C₁₉H₁₉ClN₄O₅SC₂HCl₃O₂ m/z 451 (M+H)⁺.

### Example 255 (BVT051438G)

### 2-[2-(bicyclo[2.2.1]hept-5-en-2-ylamino)-4-oxo-4,5-dihydro-1,3-thiazol-5-yl]ethyl (4-bromo-2,6-difluorophenyl)carbamate

Prepared according to method F

55 mg, 40% yield.

¹H NMR (270 MHz, CHLOROFORM-D) δ ppm 1.53-1.74 (m, 3H) 1.83-1.97 (m, 1H) 2.05-2.23 (m, 1H) 2.54-2.67 (m, 1H) 2.92-3.02 (m, 2H) 3.27-3.37 (m, 1H) 4.12-4.22 (m, 1H) 4.25-4.45 (m, 2H) 6.02-6.08 (m, 1H) 6.21-6.28 (m, 1H) 7.07-7.16 (m, 2H). MS (ESI+) for C₁₉H₁₈BrF₂N₃O₃SC₂HCl₃O₂ m/z 388 (M+H)⁺.

### Example 256 (BVT051516G)

### 2-[2-(bicyclo[2.2.1]hept-5-en-2-ylamino)-4-oxo-4,5-dihydro-1,3-thiazol-5-yl]ethyl (3-phenoxyphenyl)carbamate

Prepared according to method F

15 mg, 11% yield.

¹H NMR (270 MHz, CHLOROFORM-D) δ ppm 1.58-1.84 (m, 4H) 2.28-2.47 (m, 1H) 2.50-2.63 (m, 1H) 3.00-3.08 (m, 2H) 3.28-3.37 (m, 1H) 4.27-4.47 (m, 3H) 5.98-6.07 (m, 1H) 6.25-6.32 (m, 1H) 6.70 (d, J=7.92 Hz, 1H) 6.94-7.37 (m, 8H). MS (ESI+) for C₂₅H₂₅N₃O₄SC₂HCl₃O₂ m/z 464 (M+H)⁺.

### Example 257 (BVT067466)

### N-{2-[2-(cycloheptylamino)-4-oxo-4,5-dihydro-1,3-thiazol-5-yl]ethyl}-N′-(2-fluorophenyl)urea

Prepared according to method F

37 mg, yield 59%.

¹H NMR (270 MHz, METHANOL-D4) δ ppm 1.38-1.78 (m, 10H) 1.91-2.18 (m, 3H) 2.31-2.44 (m, 1H) 3.36-3.47 (m, 2H) 3.94-4.06 (m, 1H) 4.37 (dd, J=9.40, 4.21 Hz, 1H) 6.90-7.12 (m, 3H) 7.90-8.02 (m, 1H). MS (ESI+) for C₁₉H₂₅FN₄O₂S m/z 393 (M+H)⁺.

### Example 258 (BVT067467)

### N-{2-[2-(cycloheptylamino)-4-oxo-4,5-dihydro-1,3-thiazol-5-yl]ethyl}-N′-(4-fluorophenyl)urea

Prepared according to method F

15 mg, yield 24%.

¹H NMR (270 MHz, METHANOL-D4) δ ppm 1.44-1.78 (m, 10H) 1.91-2.07 (m, 3H) 2.38-2.43 (m, 1H) 3.37 (t, J=6.68 Hz, 1H) 3.98-4.11 (m, 1H) 4.28 (dd, J=9.40, 3.96 Hz, 1H) 6.93-7.02 (m, 2H) 7.29-7.39 (m, 2H). MS (ESI+) for C₁₉H₂₅FN₄O₂S m/z 393 (M+H)⁺.

### Example 259 (BVT067468)

### N-{2-[2-(cycloheptylamino)-4-oxo-4,5-dihydro-1,3-thiazol-5-yl]ethyl}-N1-(2,6-difluorophenyl)urea

Prepared according to method F

21 mg, yield 32%.

¹H NMR (270 MHz, METHANOL-D4) δ ppm 1.43-1.78 (m, 10H) 1.83-2.06 (m, 3H) 2.28-2.44 (m, 1H) 3.25-3.46 (m, 2H) 4.02-4.12 (m, 1H) 4.26 (dd, J=9.77, 4.08 Hz, 1H) 6.92-7.04 (m, 2H) 7.14-7.31 (m, 1H). MS (ESI+) for C₁₉H₂₄F₂N₄O₂S m/z 411 (M+H)⁺.

### Example 260 (BVT067469)

### N-{2-[2-(cycloheptylamino)-4-oxo-4,5-dihydro-1,3-thiazol-5-yl]ethyl}-N′-(2,4-difluorophenyl)urea

Prepared according to method F

28 mg, yield 43%.

¹H NMR (270 MHz, METHANOL-D4) δ ppm 1.42-1.76 (m, 10H) 1.88-2.04 (m, 3H) 2.28-2.43 (m, 1H) 3.27-3.43 (m, 2H) 3.98-4.10 (m, 1H) 4.27 (dd, J=9.40, 3.96 Hz, 1H) 6.80-6.99 (m, 2H) 7.77-7.92 (m, 1H). MS (ESI+) for C₁₉H₂₄F₂N₄O₂S m/z 411 (M+H)⁺.

### Example 261 (BVT067470)

### N-(2-chlorophenyl)-N′-{2-[2-(cycloheptylamino)-4-oxo-4,5-dihydro-1,3-thiazol-5-yl]ethyl}urea

Prepared according to method F

26 mg, yield 40%.

¹H NMR (270 MHz, METHANOL-D4) δ ppm 1.43-1.62 (m, 10H) 1.94-2.12 (m, 3H) 2.32-2.47 (m, 1H) 3.36-3.47 (m, 2H) 3.96-4.06 (m, 1H) 4.36 (dd, J=9.28, 3.84 Hz, 1H) 6.96-7.03 (m, 1H) 7.20-7.28 (m, 1H) 7.35-7.40 (m, 1.36 Hz, 1H) 7.93-8.02 (m, 1H). MS (ESI+) for C₁₉H₂₅ClN₄O₂S m/z 409 (M+H)⁺.

### Example 262 (BVT067471)

### N-[2-chloro-5-(trifluoromethyl)phenyl]-N′-{2-[2-(cycloheptylamino)-4-oxo-4,5-dihydro-1,3-thiazol-5-yl]ethyl}urea

Prepared according to method F

26 mg, yield 34%.

¹H NMR (270 MHz, METHANOL-D4) δ ppm 1.27-1.79 (m, 10H) 1.80-2.13 (m, 3H) 2.15-2.57 (m, 1H) 3.34-3.62 (m, 2H) 3.83-4.15 (m, 1H) 4.11-4.36 (m, 1H) 6.99-7.30 (m, 1H) 7.34-7.58 (m, 1H) 8.39-8.64 (m, 1H). MS (ESI+) for C₂₀H₂₄ClF₃N₄O₂S m/z 478 (M+H)⁺.

### Example 263 (BVT067472)

### N-{2-[2-(cycloheptylamino)-4-oxo-4,5-dihydro-1,3-thiazol-5-yl]ethyl}-N′-[4-fluoro-2-(trifluoromethyl)phenyl]urea

Prepared according to method F

37 mg, yield 50%.

¹H NMR (270 MHz, METHANOL-D4) δ ppm 1.37-1.78 (m, 10H) 1.85-2.08 (m, 3H) 2.27-2.47 (m, 1H) 3.28-3.66 (m, 2H) 3.97-4.10 (m, 1H) 4.30 (dd, J=9.8, 3.8 Hz, 1H) 7.25-7.45 (m, 2H) 7.65-7.80 (m, 1H). MS (ESI+) for C₂₀H₂₄F₄N₄O₂S m/z 461 (M+H)⁺.

### Example 264 (BVT067473)

### N-{2-[2-(cycloheptylamino)-4-oxo-4,5-dihydro-1,3-thiazol-5-yl]ethyl}-N′-(2-methoxyphenyl)urea

Prepared according to method F

36 mg, yield 56%.

¹H NMR (270 MHz, METHANOL-D4) δ ppm 1.38-1.78 (m, 10H) 1.92-2.12 (m, 3H) 2.27-2.42 (m, 1H) 3.30-3.47 (m, 2H) 3.84 (s, 3H) 3.95-4.09 (m, 1H) 4.32 (dd, J=9.5, 3.8 Hz, 1H) 6.77-7.01 (m, 3H) 7.94 (d, J=7.4 Hz, 1H). MS (ESI+) for C₂₀H₂₈N₄O₃S m/z 405 (M+H)⁺.

### Example 265 (BVT067474)

### N-(5-chloro-2-methoxyphenyl)-N′-{2-[2-(cycloheptylamino)-4-oxo-4,5-dihydro-1,3-thiazol-5-yl]ethyl}urea

Prepared according to method F

32 mg, yield 46%.

¹H NMR (270 MHz, METHANOL-D4) δ ppm 1.41-1.76 (m, 10H) 1.84-2.08 (m, 3H) 2.28-2.42 (m, 1H) 3.33-3.50 (m, 2H) 3.86 (s, 3H) 3.92-4.11 (m, 1H) 4.32 (dd, J=9.2, 4.0 Hz, 1H) 6.89 (t, J=1.5 Hz, 2H) 8.06-8.13 (m, 1H). MS (ESI+) for C₂₀H₂₇ClN₄O₃S m/z 440 (M+H)⁺.

### Example 266 (BVT067475)

### N-{2-[2-(cycloheptylamino)-4-oxo-4,5-dihydro-1,3-thiazol-5-yl]ethyl}-N-(2,4-dimethoxyphenyl)urea

Prepared according to method F

23 mg, yield 33%.

¹H NMR (270 MHz, METHANOL-D4) δ ppm 1.38-1.81 (m, 10H) 1.86-2.08 (m, 3H) 2.22-2.46 (m, 1H) 3.27-3.47 (m, 2H) 3.74 (s, 3H) 3.82 (s, 3H) 3.92-4.12 (m, 1H) 4.30 (dd, J=9.5, 3.8 Hz, 1H) 6.43 (dd, J=8.8, 2.6 Hz, 1H) 6.53 (d, J=2.7 Hz, 1H) 7.58-7.74 (m, 1H). MS (ESI+) for C₂₁H₃₀N₄O₄S m/z 435 (M+H)⁺.

### Example 267 (BVT067476)

### N-{2-[2-(cycloheptylamino)-4-oxo-4,5-dihydro-1,3-thiazol-5-yl]ethyl}-N′-(2,6-dichloropyridin-4-yl)urea

Prepared according to method F

26 mg, yield 37%.

¹H NMR (270 MHz, DMSO-D6) δ ppm 1.25-1.96 (m, 13H) 2.11-2.32 (m, 1H) 3.08-3.31 (m, 2H) 3.85-4.04 (m, 1H) 4.14 (dd, J=9.8, 3.8 Hz, 1H) 6.73-6.89 (m, 1H) 7.44-7.59 (m, 1H) 9.25 (d, J=7.4 Hz, 1H) 9.48-9.64 (m, 1H). MS (ESI+) for C₁₈H₂₃Cl₂N₅O₂S m/z 445 (M+H)⁺.

### Example 268 (BVT067478)

### N-{2-[2-(cycloheptylamino)-4-oxo-4,5-dihydro-1,3-thiazol-5-yl]ethyl}-N′-cyclohexylurea

Prepared according to method F

22 mg, yield 36%.

¹H NMR (270 MHz, METHANOL-D4) δ ppm 1.02-2.12 (m, 23H) 2.22-2.41 (m, 1H) 3.16-3.65 (m, 3H) 3.96-4.13 (m, 1H) 4.26 (dd, J=9.9, 4.0 Hz, 1H). MS (ESI+) for C₁₉H₃₂N₄O₂S m/z 381 (M+H)⁺.

### Example 269 (BVT067480)

### N-{2-[2-(cycloheptylamino)-4-oxo-4,5-dihydro-1,3-thiazol-5-yl]ethyl}-N′-cyclopentylurea

Prepared according to method F

32 mg, yield 55%.

¹H NMR (270 MHz, METHANOL-D4) δ ppm 1.26-2.10 (m, 21H) 2.22-2.39 (m, 1H) 3.15-3.42 (m, 2H) 3.85-3.99 (m, 1H) 3.99-4.12 (m, 1H) 4.20-4.30 (m, 1H). MS (ESI+) for C₁₈H₃₀N₄O₂S m/z 367 (M+H)⁺.

Compounds of Type 5

### Example 270 (BVT.51309G)

### 2-(bicyclo[2.2.1]hept-5-en-2-ylamino)-5-{2-[(2-chlorobenzyl)oxy]ethyl}-1,3-thiazol-4(5H)-one

Method G

2-(bicyclo[2.2.1]hept-5-en-2-ylamino)-5-(2-hydroxyethyl)-1,3-thiazol-4(5H)-one (50 mg, 0.20 mmol) and triphenylphosphine (64 mg, 0.24 mmol) were dissolved in THF (5 mL). The reaction mixture was stirred at RT for 10 min. and 2-chloro-benzyl alcohol (34 mg, 0.24 mmol) and DEAD (37 μL, 0.24 mmol) was added. The reaction mixture was stirred at RT over night. The solvent was removed under reduced pressure and the crude was dissolved in DCM (15 mL) and was washed with brine (1×5 mL). The organic layer was dried (MgSO₄) and the solvent was removed under reduced pressure. Purification using preparative HPLC (10-90% MeCN over 10 min followed by 100% MeCN for 5 min) afforded the product in 68% yield (50 mg).

¹H NMR (270 MHz, CHLOROFORM-D) δ ppm 1.20-1.29 (m, 1H) 1.34-1.40 (m, 1H) 1.49-1.63 (m, 2H) 2.05-2.18 (m, 1H) 2.33-2.47 (m, 1H) 2.58 (br s, 1H) 2.81 (br s, 1H) 3.13-3.22 (m, 1H) 3.76-3.96 (m, 2H) 4.25-4.38 (m, 1H) 4.94-5.09 (m, 2H) 5.99 (dd, J=5.81, 3.09 Hz, 1H) 6.16 (dd, J=5.69, 2.97 Hz, 1H) 7.07-7.7.19 (m, 1H) 7.30-7.38 (m, 1H) 7.40-7.48 (m, 1H) 7.65-7.75 (m, 1H). HPLC 94% R_{T}=2.71 (System A. 10-97% MeCN over 3 min) 95% R_{T}=1.71 (System B. 10-90% MeCN over 3 min). MS (ESI+) for C₁₉H₂₁ClN₂O₂SC₂HCl₃O₂ m/z 378 (M+H)⁺.

### Example 271 (BVT.51314G)

### 2-(bicyclo[2.2.1]hept-5-en-2-ylamino)-5-{2-[(2-methylbenzyl)oxy]ethyl}-1,3-thiazol-4(5H)-one trifluoroacetate

Prepared according to method G

26 mg, 18% yield.

¹H NMR (270 MHz, CHLOROFORM-D) δ ppm 1.30-1.38 (m, 1H) 1.47-1.62 (m, 2H) 1.77 (d, J=8.16 Hz, 1H) 1.95-2.11 (m, 1H) 2.27-2.42 (m, 2H) 2.66 (br s, 1H) 2.87 (br s, 1H) 3.14-3.22 (m, 1H) 3.68-3.92 (m, 2H) 4.18 (m, 1H) 4.78-4.95 (m, 2H) 6.00 (dd, J=5.69, 3.22 Hz, 1H) 6.18 (dd, J=5.69, 2.97 Hz, 1H) 7.17-7.30 (m, 3H) 7.37-7.44 (m, 2H). MS (ESI+) for C₁₉H₂₂N₂O₂SC₂HCl₃O₂ m/z 343 (M+H)⁺.

### Example 272 (BVT.51315G)

### 2-(bicyclo[2.2.1]hept-5-en-2-ylamino)-5-{2-[(2-methoxybenzyl)oxy]ethyl}-1,3-thiazol-4(5H)-one

Prepared according to method G

6 mg, 4% yield.

¹H NMR (270 MHz, CHLOROFORM-D) δ ppm 1.40-1.72 (m, 4H) 2.13-2.28 (m, 1H) 2.35-2.50 (m, 1H) 2.76-2.83 (m, 1H) 2.90 (br.s, 1H) 3.32 (dd, J=7.42, 2.72 Hz, 1H) 3.86 (s, 3H) 3.78-3.95 (m, 2H) 4.37 (dd, J=7.67, 5.20 Hz, 1H) 4.94-5.13 (m, 2H) 5.97-6.05 (m, 1H) 6.18-6.26 (m, 1H) 6.91 (dd, J=8.10, 5.40 Hz, 2H) 7.25 (dd, J=8.10, 5.40Hz, 2H). MS (ESI+) for C₂₀H₂₄N₂O₃SC₂HCl₃O₂ m/z 373 (M+H)⁺.

### Example 273 (BVT.51316G)

### 2-(bicyclo[2.2.1]hept-5-en-2-ylamino)-5-(2-{[3-(dimethylamino)benzyl]oxy}ethyl)-1,3-thiazol-4(5H)-one

Prepared according to method G

30 mg, 20% yield.

¹H NMR (270 MHz, CHLOROFORM-D) δ ppm 1.39-1.53 (m, 2H) 1.58-1.69 (m, 2H) 2.13-2.24 (m, 1H) 2.33-2.48 (m, 1H) 2.77 (br s, 1H) 2.88 (br s, 1H) 3.17 (s, 6H) 3.28 (d, J=6.19 Hz, 1H) 3.70-3.95 (m, 2H) 4.34 (td, J=5.51, 2.35 Hz, 1H) 4.92-5.05 (m, 2H) 5.98-6.04 (m, 1H) 6.16-6.23 (m, 1H) 7.32-7.54 (m, 4H). MS (ESI+) for C₂₁H₂₇N₃O₂SC₂HCl₃O₂ m/z 386 (M+H)⁺.

### Example 274 (BVT.51005)

### 2-(Bicyclo[2.2.1]hept-5-en-2-ylamino)-5-[2-(2-chlorophenoxy)ethyl]-1,3-thiazol-4(5H)-one

Prepared according to method G

13 mg, 15% yield, as a white solid.

MS (ESI+) for C₁₈H₁₉ClN₂O₂S m/z 363 (M+H)⁺.

### Example 275 (BVT.51070)

### 2-(bicyclo[2.2.1]hept-5-en-2-ylamino)-5-(2-hydroxyethyl)-1,3-thiazol-4(5H)-one

Method E

3-bromodihydrofuran-2(3H)-one (1.0 g, 6.1 mmol) and N-Bicyclo[2.2.1]hept-5-en-2-ylthiourea (1.02 g, 6 mmol) were mixed in acetone (60 mL) and heated to reflux for 1 h. The reaction mixture was poured on water and the pH was set to 7, using NaHCO₃-solution. Extracted the aqueous phase with DCM, dried the organic phase (MgSO₄) evaporated the solvent, got 1.52 g of the product, yield 99%.

¹H NMR (270 MHz, CHLOROFORM-D) δ ppm 1.24-1.19 (m, 1H) 1.68-1.37 (m, 2H) 2.06-1.80 (m, 2H) 2.44-2.37 (m, 1H) 2.96-2.86 (m, 2H) 3.86-3.65 (m, 2H) 4.26-4.20 (m, 1H) 6.07-6.04 (m, 1H) 6.22-6.15 (m, 1H) 3.36-3.32 (m, 1H). MS (ESI+) for C₁₂H₁₆N₂O₂S m/z 253 (M+H)⁺.

### Example 276 (BVT.51120)

### 2-(Bicyclo[2.2.1]hept-5-en-2-ylamino)-5-[2-(4-phenoxyphenoxy)ethyl]-1,3-thiazol-4(5H)-one

Prepared according to method G

White solid (3 mg)

MS (ESI+) for C₂₄H₂₄N₂O₃S m/z 421 (M+H)⁺.

### Example 277 (BVT.51121)

Methyl 4-{2-[2-(bicyclo[2.2.1]hept-5-en-2-ylamino)-4-oxo-4,5-dihydro-1,3-thiazol-5-yl]ethoxy}-3-chlorobenzoate

Prepared according to method G

As a white solid (23 mg).

MS (ESI+) for C₂₀H₂₁ClN₂O₄S m/z 421 (M+H)⁺.

### Example 278 (BVT.51136)

### 2-(Bicyclo[2.2.1]hept-5-en-2-ylamino)-5-[2-(4-chloro-3-methylphenoxy)ethyl]-1,3-thiazol-4(5H)-one

Prepared according to method G

As a white solid (2 mg).

MS (ESI+) for C₁₉H₂₁ClN₂O₂S m/z 377 (M+H)⁺.

Compounds of Type 5B

### Example 279 (BVT.49923)

### 2-Chlorophenyl [2-(bicyclo[2.2.1]hept-5-en-2-ylamino)-4-oxo-4,5-dihydro-1,3-thiazol-5-yl]acetate

Method L

[2-(Bicyclo[2.2.1]hept-5-en-2-ylamino)-4-oxo-4,5-dihydro-1,3-thiazol-5-yl]acetic acid (100 mg, 0.375 mmol) HOBt (50 mg, 0.375 mmol) and EDCI (72 mg, 0.375 mmol) were suspended in DCM (5 mL). Triethylamine (104 μL, 0.75 mmol, 2 eq) was added and the resulting suspension was stirred for 30 min at ambient temperature. Then phenol (1.1 mmol, 3 eq.) was added, and stirring continued for 3 h. The reaction mixture was eluted over a column containing hydromatrix (5×1 cm) treated with 2M HCl and thoroughly washed with DCM. Evaporation in vacuo afforded the crude product.

This gave 130 mg (46%) of the title compound as a white solid: Mp 211° C. ¹H NMR (400 MHz, CHLOROFORM-D) δ ppm 1.58-1.60 (m, 1H) 1.68-1.72 (m, 2H) 1.97-2.05 (m, 1H) 2.97-3.01 (m, 2H) 3.03-3.10 (m, 1H) 3.34-3.38 (m, 1H) 3.65-3.72 (m, 1H) 4.44-4.48 (m, 1H) 6.04-6.07 (m, 1H) 6.23 (dd, J1=5.52, J2=2.51 Hz, 1H) 7.13-7.31 (m, 3H) 7.43-7.46 (m, 1H). MS (ESI+) for C₁₈H₁₇ClN₂O₃S m/z 377 (M+H)⁺.

### Example 280 (BVT.50180)

### Phenyl [2-(bicyclo[2.2.1]hept-5-en-2-ylamino)-4-oxo-4,5-dihydro-1,3-thiazol-5-yl]acetate

Prepared according to method L

48 mg, 37% yield, as a white solid.

¹H NMR (400 MHz, CHLOROFORM-D) δ ppm 1.58-1.60 (m, 1H) 1.68-1.72 (m, 2H) 1.97-2.05 (m, 1H) 2.97-3.01 (m, 2H) 3.03-3.10 (m, 1H) 3.34-3.38 (m, 1H) 3.65-3.72 (m, 1H) 4.44-4.48 (m, 1H) 6.04-6.07 (m, 1H) 6.23 (dd, J1=5.52, J2=2.51 Hz, 1H) 7.13-7.31 (m, 3H) 7.43-7.46 (m, 1H). MS (ESI+) for C₁₈H₁₈N₂O₃S m/z 343 (M+H)⁺.

### Example 281 (BVT.50205)

### 2-Methoxyphenyl [2-(bicyclo[2.2.1]hept-5-en-2-ylamino)-4-oxo-4,5-dihydro-1,3-thiazol-5-yl]acetate

Prepared according to method L

57 mg, 41% yield, as a white solid.

Mp 175-176° C. ¹H NMR (400 MHz, CHLOROFORM-D) δ ppm 1.58-1.60 (m, 1H) 1.68-1.72 (m, 2H) 1.97-2.05 (m, 1H) 2.97-3.01 (m, 2H) 3.03-3.10 (m, 1H) 3.34-3.38 (m, 1H) 3.65-3.72 (m, 1H) 4.44-4.48 (m, 1H) 6.04-6.07 (m, 1H) 6.23 (dd, J1=5.52, J2=2.51 Hz, 1H) 7.13-7.31 (m, 3H) 7.43-7.46 (m, 1H). MS (ESI+) for C₁₉H₂₀N₂O₄S m/z 373 (M+H)⁺.

### Example 282 (BVT.50213)

### 3-Morpholin-4-ylphenyl [2-(bicyclo[2.2.1]hept-5-en-2-ylamino)-4-oxo-4,5-dihydro-1,3-thiazol-5-yl]acetate

Prepared according to method L

35 mg, 22% yield.

Mp 203-204° C. ¹H NMR (400 MHz, CHLOROFORM-D) δ ppm 1.58-1.60 (m, 1H) 1.68-1.72 (m, 2H) 1.97-2.05 (m, 1H) 2.97-3.01 (m, 2H) 3.03-3.10 (m, 1H) 3.34-3.38 (m, 1H) 3.65-3.72 (m, 1H) 4.44-4.48 (m, 1H) 6.04-6.07 (m, 1H) 6.23 (dd, J1=5.52, J2=2.51 Hz, 1H) 7.13-7.31 (m, 3H) 7.43-7.46 (m, 1H). MS (ESI+) for C₂₂H₂₅N₃O₄S m/z 428 (M+H)⁺.

Compounds of Type 6

### Example 283 (BVT.51151)

### 2-(Bicyclo[2.2.1]hept-5-en-2-ylamino)-5-[2-(4-chlorophenyl)-2-oxoethyl]-1,3-thiazol-4(5H)-one

Method J

N-Bicyclo[2.2.1]hept-5-en-2-ylthiourea (93 mg, 0.55 mmol) and 3-(4-chlorobenzoyl)acrylic acid (116 mg, 0.55 mmol) in water (5 mL) was refluxed for 18 h. The precipitate was collected on a filter after cooling and recrystallized from ethanol, yielding 96 mg (48%) of white crystals: Mp 244-245° C. ¹H NMR (400 MHz, DMSO-d6) δ ppm 1.41-1.61 (m, 4H) 2.81-2.86 (m, 2H) 3.47-3.57 (m, 1H) 3.75-3.78 (m, 1H) 3.91-4.00 (m, 1H) 4.34-4.42 (m, 1H) 6.04-6.11 (m, 1H) 6.21 (dd, J1=5.65, J2=2.98 Hz, 1H) 7.59-7.62 (m, 2H) 7.97-8.01 (m, 2H) 9.29 (d, J=6.78 Hz, NH). MS (ESI+) for C₁₈H₁₇ClN₂O₂S m/z 361 (M+H)⁺.

### Comparative Example 1 (BVT.51047)

### N-Bicyclo[2.2.1]hept-5-en-2-ylthiourea

Method A

5-Isothiocyanatobicyclo[2.2.1]hept-2-ene (10.5 g, 69.43 mmol) was stirred in 2 M ammonia in ethanol (170 mL, 345 mmol) for 18 h. The reaction flask was evaporated until viscous oil was obtained. DCM was added and the obtained crystals were collected and dried. This gave 3.13 g of the title compound. Yield 30%, 95% pure. An additional 4.74 g was obtained by recrystallisation of the mother liquor. Totally 7.87 g, yield 68%.

¹H NMR (270 MHz, CHLOROFORM-D) δ ppm 6.54 (br. S, 1H, N—H) 6.22-6.20 (m, 1H) 6.04-6.03 (m, 1H) 5.95-5.80 (br.s, 2H, N—H) 3.30-3.20 (m, 1H) 2.99-2.90 (m, 2H) 1.77-1.70 (m, 1H) 1.62-1.59 (m, 1H) 1.53-1.47 (m, 1H) 1.44-1.36 (m, 1H). MS (ESI+) for C₈H₁₂N₂S m/z 169 (M+H)⁺.

Synthesis of Compounds of Type 1

### Comparative Example 2

### 4-(1,3-dioxo-1,3-dihydro-2H-isoindol-2-yl)butanoic acid

Method K

Phtalic anhydride (7.19 g, 48.5 mmol) 4-aminobutyric acid (5.00 g, 48.5 mmol) and TEA (0.68 ml) in toluene (75 ml) were placed in a round bottom flask fitted with a Dean Stark condenser and heated to reflux for 3.5 h. The reaction mixture was then left in the refrigerator over night. The crystals that formed were collected, first washed with hexane and then HCl (5%) and finally with water, and then dried in a vacuum oven. This afforded the product 6.45 g (58%) as white crystals.

¹H NMR (270 MHz, CHLOROFORM-D) δ ppm 1.94-2.09 (m, 2H) 2.41 (t, J=7.42 Hz, 2H) 3.76 (t, J=6.80 Hz, 2H) 7.67-7.76 (m, 2H) 7.80-7.88 (m, 2H). HPLC 97%, R_{T}=1.50 min (System A, 10-97% MeCN over 3 min). MS (ESI+) for C₁₂H₁₁NO₄ m/z 234 (M+H)⁺.

### Comparative Example 3

### 2-bromo-4-(1,3-dioxo-1,3-dihydro-2H-isoindol-2-yl)butanoic acid

4-(1,3-dioxo-1,3-dihydro-2H-isoindol-2-yl)butanoic acid (3.00 g, 12.9 mmol) and SOCl₂ (20 ml) were heated to reflux for 2 h. Br₂ (2.06 g, 12.9 mmol) was added slowly during 4 h while heating was continued. The reaction was heated to reflux until all starting material was consumed, approximately 48 h (followed with LC-MS). Excess SOCl₂ was removed in vacuum. Crushed ice was added to the residue and it was left over night. The white solid that had formed was filtered and dried in a vacuum oven to give 4.01 g of the crude product. This material was used in the next step without further purification.

¹H NMR (270 MHz, METHANOL-D4) δ ppm 2.19-2.38 (m, 1H) 2.39-2.58 (m, 1H) 3.83 (t, J=6.68 Hz, 2H) 4.37 (t, J=7.18 Hz, 1H) 7.73-7.91 (m, 4H). HPLC 71%, R_{T}=1.12 min (System A, 30-80% MeCN over 3 min). MS (ESI+) for C₁₂H₁₀BrNO₄ m/z 312 (M+H)⁺.

### Example 284

### 5-(2-aminoethyl)-2-(bicyclo[2.2.1]hept-5-en-2-ylamino)-1,3-thiazol-4(5H)-one

2-{2-[2-(bicyclo[2.2.1]hept-5-en-2-ylamino)-4-oxo-4,5-dihydro-1,3-thiazol-5-yl]ethyl}-1H-isoindole-1,3(2H)-dione (3.31 g, 8.68 mmol) was dissolved in a 0.2 M solution of Hydrazine in MeOH. The reaction mixture was heated to reflux for 3 h. Solvent and excess Hydrazine was removed in vacuum and co-evaporation with EtOH was repeated several times. The residue was dissolved in a small volume of MeOH and DCM was added. The white precipitate that formed was filtered and washed with DCM. The filtrate was concentrated in vacuum to give 3.0 g of the crude product. Purification was performed using column chromatography, eluent 20% MeOH in DCM and thereafter MeOH containing some TEA. This afforded 1.8 g of apricot coloured crystals which showed to be a 1:1 (according to NMR) mixture of the wanted product and 2,3-dihydrophtalazine-1,4-dione. This mixture was used in the next step without further purification.

¹H NMR (270 MHz, METHANOL-D4) δ ppm 1.20 (t, J=7.30 Hz, 2H) 1.36-1.78 (m, 4H) 1.95-2.15 (m, 1H) 2.23-2.38 (m, 1H) 2.81-3.06 (m, 5H) 3.34 (s, 1H) 3.83 (dd, J=7.05, 2.10 Hz, 1H) 6.08 (dd, J=5.44, 3.22 Hz, 1H) 6.21 (dd, J=5.69, 2.97 Hz, 1H) (from 2,3-dihydrophtalazine-1,4-dione) 7.75-7.86 (m, 2H) 8.13-8.26 (m, 2H). HPLC 37%, R_{T}=1.58 min (System A, 5-60% MeCN over 3 min). MS (ESI+) for C₁₂H₁₇N₃OS m/z 252 (M+H)⁺.

### Comparative Example 4 (BVT056635)

### 4-(1,3-dioxo-1,3-dihydro-2H-isoindol-2-yl)butanoic acid

Method K

To phtalimide (8.6 g, 58.2 mmol) and 4-aminobutyric acid (6.0 g, 58.2 mmol) was 70 mL toluene added. The reaction mixture was heated to reflux and the formed water was removed using a Dean-Stark apparatus. The reaction mixture was cooled to 10° C. after 3 h and the precipitate was filter off. The crystals were washed with pentane (40 mL) and water (20 mL) and were then dried under reduced pressure over night to afford 11.1 g, 81% yield.

¹H NMR (270 MHz, METHANOL-D4) δ ppm 1.95 (m, 2H) 2.35 (t, J=7.18 Hz, 2H) 3.72 (t, J=6.68 Hz, 2H) 7.81 (m, 4H). HPLC 96% R_{T}=1.51 (System A. 10-97% MeCN over 3 min), 98% R=1.39 (System B. 10-97% MeCN over 3 min). MS (ESI+) for C₁₂H₁₁NO₄ m/z 234 (M+H)⁺.

### Comparative Example 5 (BVT063226)

### 2-bromo-4-(1,3-dioxo-1,3-dihydro-2H-isoindol-2-yl)butanoic acid

4-(1,3-dioxo-1,3-dihydro-2H-isoindol-2-yl)butanoic acid (11.0 g, 47.2 mmol) was dissolved in thionyl chloride (50 mL) and the reaction mixture was heated to reflux for 2 h. Bromine (2.7 mL 51.9 mmol) was added over 6 h using a syring pump under reflux. The reaction mixture was refluxed overnight. The reaction mixture was then cooled to rt and the solvent was removed under reduced pressure. The crude product was used without further purifications.

¹H NMR (270 MHz, METHANOL-D4) δ ppm 2.22-2.33 (m, 1H) 2.41-2.56 (m, 1H) 3.84 (t, J=6.68 Hz, 1H) 4.37 (t, J=7.05 Hz, 1H) 7.75-7.91 (m, 4H). HPLC 95% R_{T}=1.80 (System A. 10-97% MeCN over 3 min), 96% R_{T}=1.69 (System B. 10-97% MeCN over 3 min). MS (ESI+) for C₁₂H₁₀BrNO₄ m/z 314 (M+H)⁺.

### Comparative Example 6 (BVT063218)

### 3-bromopyrrolidin-2-one

2-bromo-4-(1,3-dioxo-1,3-dihydro-2H-isoindol-2-yl)butanoic acid (47.2 mmol) was dissolved in 47% HBr (aq.) and the reaction mixture was heated to reflux for 12 h. The reaction mixture was then cooled to 10° C., the precipitate was filtered off and the solvent was removed from the filtrate under reduced pressure. The crude product was dissolved in MeOH and was shaken with resin bound tosyl acid (97.0 g, 1.46 g/mmol) over night. The resin was washed several times with 2.0M NH₃ in MeOH to release the product. The solvent was removed under reduced pressure to afford the product in 59% yield (4.55 g) from 4-(1,3-dioxo-1,3-dihydro-2H-isoindol-2-yl)butanoic acid.

¹H NMR (270 MHz, METHANOL-D4) δ ppm 2.29-2.39 (m, 2H) 2.71 (td, J=14.78, 7.55 Hz, 1H) 3.36 (ddd, J=10.39, 7.67, 2.72 Hz, 1H) 3.44-3.54 (m, 1H) 4.44 (dd, J=7.18, 2.97 Hz, 1H). HPLC 100% R_{T}=0.66 (System A. 5-60% MeCN over 3 min), 100% R_{T}=0.89 (System B. 5-60% MeCN over 3 min). MS (ESI+) for C₄H₆BrNO m/z 164 (M+H)⁺.

### Example 285 (BVT063224)

### 5-(2-aminoethyl)-2-(cyclohexylamino)-1,3-thiazol-4(5H)-one

N-cyclohexylthiourea (0.8 g, 3.3 mmol) and 3-bromopyrrolidin-2-one (0.54 g, 3.3 mmol) were dissolved in acetone and the reaction mixture was heated to reflux over night. The reaction mixture was cooled to rt and the solvent was removed under reduced pressure. Purification using preparative HPLC (x-x % MeCN in H₂O over 10 min followed by 100% MeCN for 5 min) afforded the TFA-salt of the product in 34% yield, 0.40 g.

¹H NMR (270 MHz, METHANOL-D4) δ ppm 1.15-1.50 (m, 5H) 1.60-1.73 (m, 1H) 1.71-1.88 (m, 2H) 1.92-2.03 (m, 2H) 2.12-2.26 (m, 1H) 2.27-2.44 (m, 1H) 2.93-3.08 (m, 1H) 3.10-3.22 (m, 1H) 3.81-3.94 (m, 1H) 4.37-4.48 (m, 1H). HPLC 100% R_{T}=0.95 (System A. 10-97% MeCN over 3 min), 100% R_{T}=1.17 (System B. 10-97% MeCN over 3 min). MS (ESI+) for C₁₁H₁₉N₃OS m/z 242 (M+H)⁺.

Synthesis of Compounds of Type 2

### Comparative Example 7 (BVT.51047)

### N-Bicyclo[2.2.1]hept-5-en-2-ylthiourea

Method A

5-Isothiocyanatobicyclo[2.2.1]hept-2-ene (10.5 g, 69.43 mmol) was stirred in 2 M ammonia in ethanol (170 mL, 345 mmol) for 18 h. The reaction flask was evaporated until viscous oil. DCM was added and the obtained crystals were collected and dried. This gave 3.13 g of the title compound. Yield 30%, 95% pure. An additional 4.74 g was obtained by recrystallisation of the mother liquor. Totally 7.87 g, yield 68%. ¹H NMR (270 MHz, CHLOROFORM-D) δ ppm 1.44-1.36 (m, 1H) 1.53-1.47 (m, 1H) 1.62-1.59 (m, 1H) 1.77-1.70 (m, 1H) 2.99-2.90 (m, 2H) 3.30-3.20 (m, 1H) 5.95-5.80 (br.s, 2H, N—H) 6.04-6.03 (m, 1H) 6.22-6.20 (m, 1H) 6.54 (br. S, 1H, N—H). MS (ESI+) for C₈H₁₂N₂S m/z 169 (M+H)⁺.

### Example 286 (BVT.51359)

### 2-(bicyclo[2.2.1]hept-5-en-2-ylamino)-5-(2-bromoethyl)-1,3-thiazol-4(5H)-one

Method I

2-(bicyclo[2.2.1]hept-5-en-2-ylamino)-5-(2-hydroxyethyl)-1,3-thiazol-4(5H)-one (0.5 g, 2 mmol) and triphenylphosphine dibromide (2.11 g, 5 mmol) was dissolved in DCM (200 mL) and stirred at RT for 16 h. The reaction mixture was washed with water and dried (MgSO₄) the solvent was evaporated and the obtained solid crude product was purified by flash chromatography using MeCN as eluent. The first fraction contained the product. Got 0.47 g of the desired product, yield 74%, 98% pure.

¹H NMR (270 MHz, DMSO-D6) δ ppm 1.62-1.41 (m, 4H) 2.20-2.15 (m, 1H) 2.61-2.56 (m, 1H) 2.90-2.80 (m, 2H) 3.65-3.57 (m, 2H) 3.78-3.70 (m, 1H) 4.31-4.25 (m, 1H) 6.11-6.08 (m, 1H) 6.23-6.20 (m, 1H) 9.37 (br.d, J=6.93 Hz, 1H, N—H). MS (ESI+) for C₁₂H₁₅BrN₂OS m/z 315 (M+H)⁺.

### Comparative Example 8 (BVT.59513)

### N-[(1R,2R,3R,5S)-2,6,6-Trimethylbicyclo[3.1.1]hept-3-yl]thiourea

Method B

To (1R,2R,3R,5S)-2,6,6-trimethylbicyclo[3.1.1]hept-3-ylamine (547 μL, 3.26 mmol) was added ethyl isothiocyanatidocarbonate (385 μL, 3.26 mmol) and the mixture was stirred for 5 min until which a yellow solid had been formed. After addition of 5 M NaOH (aq) (8 mL) the reaction was stirred at 70° C. for 6 h. Water (20 mL) and ethyl acetate (25 mL) were added, and the phases were separated. The water phase was extracted with ethyl acetate (5 mL) and the combined organic phases were dried (MgSO₄). After evaporation of the solvent and drying in vacuo, the product was obtained as a white solid (615 mg, 89% yield)

¹H NMR (400 MHz, CHLOROFORM-D) δ ppm 0.89 (d, J=10.0 Hz, 1H) 1.00 (s br, 3H) 1.13 (d, J=7.2 Hz, 3H) 1.20 (s, 3H) 1.47-1.80 (m, 1H) 1.82 (m, 1H) 1.87-2.00 (m, 2H) 2.39 (m, 1H) 2.57 (s br, 1H) 3.59 (s br, 0.5H) 4.65 (s br, 0.5H) 6.07, (s, 2H) 6.61 (s br, 0.5H) 6.86 (s br, 0.5H). MS (ESI+) for C₁₁H₂₀N₂S m/z 213 (M+H)⁺.

### Example 287

### 2-(bicyclo[2.2.1]hept-2-ylamino)-5-(2-hydroxyethyl)-1,3-thiazol-4(5H)-one

Method E

To N-bicyclo[2.2.1]hept-2-ylthiourea (2.003 g, 11.77 mmol) acetone (7 ml) and alpha-bromo-gamma-butyrolactone (974 ul, 11.75 mmol) was added the reaction mixture was refluxed at 70° C. for 20 h. The solvent was evaporated under reduced pressure. Saturated NaHCO₃ was added and the product was extracted with DCM (3×ml). The organic phases were combined and washed with brine, dried over MgSO₄. The solvent was evaporated under reduced pressure. The residue was dissolved in ethylacetate (30 ml) and the organic phase was washed with 1M HCl (2+30 ml) and then with brine (30 ml). The water phase was made basic pH 10 using 1 M NaOH, and was then extracted with ethylacetate (3×100 ml) washed with brine, dried over MgSO₄. The solvent was evaporated under reduced pressure to give white crystals (1.7647 g, 59%) of the title compound.

¹H NMR (0307dd031, F12091003h): (270 MHz, CHLOROFORM-D) δ ppm 1.09-1.25 (m, 5H) 1.77-1.79 (m, 2H) 1.98-2.10 (m, 2H) 2.32-2.48 (m, 4H) 3.31-3.35 (m, 1H) 3.71-3.85 (m, 3H) 4.19-4.25 (m, 1H). MS (ESI+) for C₁₂H₁₈₂O₂S m/z 255 (M+H)⁺.

Synthesis of Compounds of Type 3

### Comparative Example 9 (BVT.51047)

### N-Bicyclo[2.2.1]hept-5-en-2-ylthiourea

Method A

5-Isothiocyanatobicyclo[2.2.1]hept-2-ene (10.5 g, 69.43 mmol) was stirred in 2 M ammonia in ethanol (170 mL, 345 mmol) for 18 h. The reaction flask was evaporated until viscous oil. DCM was added and the obtained crystals were collected and dried. This gave 3.13 g of the title compound. Yield 30%, 95% pure. An additional 4.74 g was obtained by recrystallisation of the mother liquor. Totally 7.87 g, yield 68%.

¹H NMR (270 MHz, CHLOROFORM-D) δ ppm 1.44-1.36 (m, 1H) 1.53-1.47 (m, 1H) 1.62-1.59 (m, 1H) 1.77-1.70 (m, 1H) 2.99-2.90 (m, 2H) 3.30-3.20 (m, 1H) 5.95-5.80 (br.s, 2H, N—H) 6.04-6.03 (m, 1H) 6.22-6.20 (m, 1H) 6.54 (br. S, 1H, N—H). MS (ESI+) for C₈H₁₂N₂S m/z 169 (M+H)⁺.

### Example 288 (BVT.47509)

### [2-(Bicyclo[2.2.1]hept-5-en-2-ylamino)-4-oxo4,5-dihydro-1,3-thiazol-5-yl]acetic acid

Method C

N-Bicyclo[2.2.1]hept-5-en-2-ylthiourea (1.00 g, 5.94 mmol) and maleic anhydride (0.58 g, 5.94 mmol) were heated to reflux in acetone for 5 h, yielding a white emulsion. Evaporation in vacuo afforded 1.58 g of a white solid. The product was triturated with DCM, collected on a filter and air-dried giving 1.43 g (91%) of a white powder: Mp 232° C. ¹H NMR (400 MHz, DMSO-D6) δ ppm 1.41-1.59 (m, 4H) 2.58-2.67 (m, 1H) 2.79-2.86 (m, 2H) 3.00-3.09 (m, 1H) 3.71-3.77 (m, 1H) 4.27-4.34 (m, 1H) 6.08 (dd, J1=5.27, J2=3.07 Hz, 1H) 6.19-6.23 (m, 1H) 9.28 (d, J=6.78 Hz, NH) 12.38 (br s, OH). MS (EI⁺) for C₁₂H₁₄N₂O₃S m/z 267.2 (M+H)⁺.

Further Miscellaneous Examples

### Example 289 (BVT063338)

### 5-[2-(3,4-dihydroquinolin-1(2H)-yl)-2-oxoethyl]-2-piperidin-1-yl-1,3-thiazol-4(5H)-one

Prepared according to method D.

50 mg, yield 35%.

¹H NMR (400 MHz, CDCl₃) δ ppm 1.67 (s, 6H), 1.94 (m, 2H), 2.68 (s, 2H), 2.83 (dd, J=17.1, 11.7 Hz, 1H), 3.43 (s, 2H), 3.57 (d, J=14.2 Hz, 1H), 3.69 (m, 1H), 3.83 (m, 3H), 4.47 (dd, J=11.7, 2.4 Hz, 1H), 7.12 (m, 4H). MS (ES+) m/z 358 (M+H⁺).

### Example 290 (BVT067662)

### 5-(2-morpholin-4-yl-2-oxoethyl)-2-[(2,2,3,3-tetramethylcyclopropyl)amino]-1,3-thiazol-4(5H)-one

Prepared according to method D.

5 mg, yield 9%.

¹H NMR (400 MHz, CDCl₃) δ ppm 1.10 (s, 6H), 1.18 (d, J=8.1 Hz, 6H), 2.13 (s, 1H), 2.75 (dd, J=17.1, 11.7 Hz, 1H), 3.38-3.76 (m, 9H), 4.42 (dd, J=11.8, 3.1 Hz, 1H). MS (ES+) m/z 340 (M+H⁺).

### Example 291 (BVT067664)

### N-[(2-{[(1S)-1-cyclohexylethyl]amino}-4-oxo-4,5-dihydro-1,3-thiazol-5-yl)methyl]-2-methoxybenzamide

Prepared according to method T.

2 mg, yield 4%.

¹H NMR (400 MHz, CDCl₃) δ ppm 0.73-1.20 (m, 6H), 1.27 (dd, J=33.0, 6.6 Hz, 3H), 1.36-1.82 (m, 5H), 3.17-3.30 (m, 1H), 3.97 (d, J=3.4 Hz, 3H), 4.12-4.25 (m, 1H), 4.27-4.37 (m, 1H), 4.37-4.45 (m, 1H), 6.99 (dd, J=8.2, 3.1 Hz, 1H), 7.08 (dt, J=7.3, 3.2 Hz, 1H), 7.43-7.54 (m, 1H), 8.13 (dt, J=6.9, 1.6 Hz, 1H). MS (ES+) m/z 390 (M+H⁺).

### Example 292 (BVT06767)

### 2-(cyclooctylamino)-5-(2-morpholin-4-yl-2-oxoethyl)-1,3-thiazol-4(5H)-one

Prepared according to method D.

28 mg, yield 43%.

¹H NMR (400 MHz, CDCl₃) δ ppm 1.41-1.70 (m, 8H), 1.69-1.83 (m, 2H), 1.83-1.96 (m, 4H), 2.76 (dd, J=17.0, 12.1 Hz, 1H), 3.38-3.76 (m, 10H), 4.42 (dd, J=12.1, 3.0 Hz, 1H). MS (ES+) m/z 354 (M+H⁺).

### Example 293 (BVT059380C)

### 2-[2-(bicyclo[2.2.1]hept-2-ylamino)-4-oxo-4,5-dihydro-1,3-thiazol-5-yl]-N-cycloheptylacetamide hydrochloride

Prepared according to method D.

¹H NMR (270 MHz, CHLOROFORM-D) δ ppm 1.29 (m, 19H) 2.36 (d, J=9.15 Hz, 1H) 2.77 (m, 1H) 3.28 (m, 1H) 3.75 (m, 1H) 4.00 (m, 3H) 4.36 (m, 1H). MS (EI+) for C₁₉H₂₉N₃O₂S m/z 364 (M+H)⁺.

### Example 294 (BVT067663)

### N-[(2-{[(1S)-1-cyclohexylethyl]amino}-4-oxo-4,5-dihydro-1,3-thiazol-5-yl)methyl]-2-fluorobenzamide

Prepared according to method T.

7 mg, yield 15%.

¹H NMR (400 MHz, CDCl₃) δ ppm 0.81-1.24 (m, 6H), 1.30 (dd, J=13.7, 6.6 Hz, 3H), 1.46-1.82 (m, 5H), 3.20-3.31 (m, 1H), 3.99-4.28 (m, 2H), 4.41-4.48 (m, 1H), 7.14 (dd, J=11.8, 8.4 Hz, 1H), 7.24-7.31 (m, 1H), 7.51 (q, J=6.5 Hz, 1H), 8.03 (td, J=7.8, 1.7 Hz, 1H). MS (ES+) m/z 378 (M+H⁺).

### Example 295 (BVT070752)

### 2-fluoro-N-(2-{4-oxo-2-[(2,2,3,3-tetramethylcyclopropyl)amino]-4,5-dihydro-1,3-thiazol-5-yl}ethyl)benzamide

Prepared according to method K.

13 mg, yield 16%.

¹H NMR (400 MHz, CDCl₃) δ ppm 1.09 (d, J=4.9 Hz, 6H), 1.16 (s, 6H), 2.14 (s, 1H), 2.16-2.28 (m, 1H), 2.47-2.59 (m, 1H), 3.59-3.70 (m, 1H), 3.81-3.92 (m, 1H), 4.29 (dd, J=9.6, 4.3 Hz, 1H), 6.98-7.08 (m, 1H), 7.14 (dd, J=12.2, 8.3 Hz, 1H), 7.26-7.30 (m, 1H), 7.46-7.54 (m, 1H), 8.05 (dt, J=7.9, 1.8 Hz, 1H). MS (ES+) m/z 378 (M+H⁺).

### Example 296 (BVT059139)

### 2-(1-adamantylamino)-5-[2-(3,4-dihydroquinolin-1(2H)-yl)-2-oxoethyl]-1,3-thiazol-4(5H)-one

Prepared according to method D.

13 mg, yield 10%.

¹H NMR (270 MHz, CHLOROFORM-D) δ ppm 1.68 (m, 5H) 2.11 (m, 9H) 2.80 (m, 3H) 3.65 (m, 2H) 3.87 (m, J=5.69 Hz, 1H) 4.42 (d, J=12.12 Hz, 1H) 4.58 (m, 1H) 4.72 (d, J=6.19 Hz, 1H) 7.11 (m, 4H). MS (EI+) for C₂₄H₂₉N₃O₂S m/z 424 (M+H)⁺.

### Example 297 (BVT059406C)

### 2-[2-(bicyclo[2.2.1]hept-2-ylamino)-4-oxo-4,5-dihydro-1,3-thiazol-5-yl]-N-(2-chloro-6-fluorobenzyl)acetamide hydrochloride

Prepared according to method D.

¹H NMR (270 MHz, CHLOROFORM-D) δ ppm 1.17 (m, 2H) 1.33 (d, J=10.14 Hz, 1H) 1.54 (m, J=30.19 Hz, 5H) 1.85 (m, 1H) 2.40 (m, 2H) 2.91 (s, 1H) 3.40 (m, 1H) 3.54 (m, J=14.35 Hz, 1H) 4.36 (m, 1H) 4.59 (m, 2H) 6.97 (m, 1H) 7.17 (m, 2H) 7.76 (s, 1H). MS (EI+) for C₁₉H₂₁Cl₁F₁N₃O₂S m/z 410/412 (M+H)⁺.

### Example 298 (BVT059509)

### 2-(cycloheptylamino)-5-[2-(3,4-dihydroquinolin-1(2H)-yl)-2-oxoethyl]-1,3-thiazol-4(5H)-one

Prepared according to method D.

25 mg, yield 16%.

MS (EI+) for C₂₁H₂₇N₃O₂S m/z 387 (M+H)⁺.

### Example 299 (BVT067665)

### 2-(cyclooctylamino)-5-[2-(3,4-dihydroquinolin-1(2H)-yl)-2-oxoethyl]-1,3-thiazol-4(5H)-one

Prepared according to method D.

16 mg, yield 22%.

¹H NMR (400 MHz, CDCl₃) δ ppm 1.42-2.11 (m, 16H), 2.61-2.88 (m, 2H), 2.99 (dd, J=17.1, 12.0 Hz, 1H), 3.48-3.74 (m, 3H), 3.93-4.11 (m, 1H), 4.33-4.54 (m, 1H), 6.95-7.24 (m, J=60.3 Hz, 4H). MS (ES+) m/z 400 (M+H⁺).

### Example 300 (BVT067669)

### N-cyclohexyl-2-[2-(cyclooctylamino)-4-oxo-4,5-dihydro-1,3-thiazol-5-yl]-N-ethylacetamide

Prepared according to method D.

11 mg, yield 16%.

¹H NMR (400 MHz, CDCl₃) δ ppm 1.14 (t, J=7.0 Hz, 3H), 1.26-2.01 (m, 24H), 2.80 (dd, J=16.7, 12.3 Hz, 1H), 3.18-3.47 (m, 3H), 3.47-3.64 (m, 2H), 4.44 (ddd, J=11.8, 8.0, 3.2 Hz, 1H), 8.37 (s, 1H). MS (ES+) m/z 394 (M+H⁺).

### Example 301 (BVT059405C)

### 5-(2-azepan-1-yl-2-oxoethyl)-2-(bicyclo[2.2.1]hept-2-ylamino)-1,3-thiazol-4(5H)-one hydrochloride

Prepared according to method D.

¹³C NMR (67.5 MHz, CHLOROFORM-D) δ ppm 26.19, 26.91, 27.26, 27.40, 28.09, 28.74, 35.80, 36.13, 36.77, 38.91, 38.97, 42.50, 46.40, 48.06, 60.92, 168.21, 172.01, 173.93. MS (EI+) for C₁₈H₂₇N₃O₂S m/z 350 (M+H)⁺.

### Example 302 (BVT067918)

### N-cyclohexyl-N-ethyl-2-{4-oxo-2-[(2,2,3,3-tetramethylcyclopropyl)amino]-4,5-dihydro-1,3-thiazol-5-yl}acetamide

Prepared according to method D.

5 mg, yield 11%.

¹H NMR (400 MHz, CDCl₃) δ ppm 1.07 (d, J=3.2 Hz, 6H), 1.10-1.22 (m, 9H), 1.23-1.55 (m, 4H), 1.58-1.88 (m, 5H), 2.10 (s, 1H), 2.67-2.80 (m, 1H), 3.17-3.28 (m, 2H), 3.29-3.42 (m, 1H), 3.45-3.56 (m, 1H), 4.33-4.44 (m, 1H). MS (ES+) m/z 380 (M+H⁺).

### Example 303 (BVT073837)

### 2-chloro-N-{[2-(cyclooctylamino)-4-oxo-4,5-dihydro-1,3-thiazol-5-yl]methyl}-6-fluorobenzamide

Prepared according to method T.

3 mg, yield 5%.

¹H NMR (400 MHz, CDCl₃) δ ppm 1.45-1.78 (m, 11H), 1.80-2.00 (m, 3H), 3.53-3.64 (m, 1H), 3.95-4.18 (m, 2H), 4.32-4.37 (m, J=6.2, 4.5 Hz, 1H), 4.42 (dd, J=6.0, 4.5 Hz, 1H), 6.97-7.06 (m, 1H), 7.14-7.23 (m, 1H), 7.27-7.42 (m, 1H). MS (ES+) m/z 412 (M+H⁺).

### Example 304 (BVT067917)

### 5-[2-(4-methylpiperidin-1-yl)-2-oxoethyl]-2-[(2,2,3,3-tetramethylcyclopropyl)amino]-1,3-thiazol-4(5H)-one

Prepared according to method D.

7 mg, yield 7%.

¹H NMR (400 MHz, CDCl₃) δ ppm 0.96 (d, J=6.3 Hz, 3H), 1.06-1.14 (m, 7H), 1.14-1.25 (m, 7H), 1.54-1.81 (m, 3H), 2.13 (d, J=6.6 Hz, 1H), 2.59-2.83 (m, 2H), 3.05 (q, J=13.4 Hz, 1H), 3.52 (dd, J=17.1, 2.9 Hz, 1H), 3.69 (d, J=12.7 Hz, 1H), 4.35-4.54 (m, 2H).

MS (ES+) m/z 352 (M+H⁺).

### Example 305 (BVT073920)

### 2-chloro-N-{[2-(cycloheptylamino)-4-oxo-4,5-dihydro-1,3-thiazol-5-yl]methyl}-6-fluorobenzamide

Prepared according to method T.

7 mg, yield 7%.

¹H NMR (400 MHz, CDCl₃) δ ppm 1.37-1.81 (m, 10H), 1.83-2.04 (m, 2H), 3.42-3.55 (m, 1H), 3.94-4.09 (m, 2H), 4.28-4.45 (m, 1H), 7.01 (dt, J=8.4, 1.7 Hz, 1H), 7.18 (d, J=8.1 Hz, 1H), 7.26-7.34 (m, 1H). MS (ES+) m/z 398 (M+H⁺).

### Example 306 (BVT067789)

### 5-(2-anilinoethyl)-2-[(2,2,3,3-tetramethylcyclopropyl)amino]-1,3-thiazol-4(5H)-one

Prepared according to method H.

16 mg, yield 17%.

¹H NMR (400 MHz, CDCl₃) δ ppm 1.02 (d, J=26.1 Hz, 6H), 1.09 (d, J=7.8 Hz, 6H), 2.03 (s, 1H) 2.11-2.24 (m, 1H), 2.84 (dd, J=13.7, 7.1 Hz, 1H), 3.94-4.08 (m, 2H), 4.37 (dd, J=9.5, 6.1 Hz, 1H), 4.80 (s, 1H), 7.23-7.28 (m, 1H), 7.36-7.45 (m, 2H), 7.54 (d, J=7.6 Hz, 2H). MS (ES+) m/z 332 (M+H⁺).

### Example 307 (BVT070751)

### 2-chloro-6-fluoro-N-(2-{4-oxo-2-[(2,2,3,3-tetramethylcyclopropyl)amino]-4,5-dihydro-1,3-thiazol-5-yl}ethyl)benzamide

Prepared according to method K.

3 mg, yield 3%.

¹H NMR (400 MHz, CDCl₃) δ ppm 1.11 (d, J=7.3 Hz, 6H), 1.20 (s, 6H), 2.17 (s, 1H), 2.18-2.27 (m, 1H), 2.45-2.56 (m, 1H), 3.49-3.59 (m, 1H), 3.89-4.00 (m, 1H), 4.38 (dd, J=9.8, 4.9 Hz, 1H), 6.22-6.31 (m, 1H), 7.05 (t, J=8.5 Hz, 1H), 7.20-7.26 (m, 1H), 7.28-7.39 (m, 1H). MS (ES+) m/z 412 (M+H⁺).

### Example 308 (BVT067670)

### 5-(2-azepan-1-yl-2-oxoethyl)-2-(cyclooctylamino)-1,3-thiazol-4(5H)-one

Prepared according to method D.

23 mg, yield 35%.

¹H NMR (400 MHz, CDCl₃) ppm 1.41-1.67 (m, 12H), 1.67-1.81 (m, J=5.1 Hz, 6H), 1.83-1.96 (m, 4H), 2.79 (dd, J=17.0, 12.1 Hz, 1H), 3.33-3.66 (m, 6H), 4.43 (dd, J=12.0, 3.2 Hz, 1H), 10.25-11.05 (m, 1H). MS (ES+) m/z 366 (M+H⁺).

### Example 309 (BVT067954)

### 2-chloro-N-{2-[2-(cyclooctylamino)-4-oxo-4,5-dihydro-1,3-thiazol-5-yl]ethyl}-6-fluorobenzamide

Prepared according to method K.

45 mg, yield 53%.

¹H NMR (400 MHz, DMSO-d₆) δ 1.47-1.82 (m, 15H), 2.24-2.33 (m, 1H), 3.27 (m, 1H), 3.39 (m, 1H), 4.02 (m, 1H), 4.20 (m, 1H), 7.26-7.31 (m, 1H), 7.36 (m, 1H), 7.44-7.49 (m, 1H), 8.82 (m, 1H), 9.19 (d, J=7.6 Hz, 1H).

MS (ESI+) for C₂₀H₂₅ClFN₃O₂S m/z 426 (M+H)⁺.

### Example 310 (BVT067955)

### 2-chloro-N-{2-[2-(cyclooctylamino)-4-oxo-4,5-dihydro-1,3-thiazol-5-yl]ethyl}benzenesulfonamide

Prepared according to method K using a sulfonyl chloride.

48 mg, yield 54%.

¹H NMR (400 MHz, DMSO-d₆) δ 1.48-1.77 (m, 15H), 2.10-2.22 (m, 1H), 2.91 (m, 2H), 3.98 (m, 1H), 4.13 (m, 1H), 7.51-7.68 (m, 3H), 7.94-8.03 (m, 2H), 9.14 (d, J=7.2 Hz, 1H).

MS (ESI+) for C₁₉H₂₆ClN₃O₃S₂ m/z 444 (M+H)⁺.

### Example 311 (BVT056890)

### 2-[2-(bicyclo[2.2.1]hept-5-en-2-ylamino)-4-oxo-4,5-dihydro-1,3-thiazol-5-yl]ethyl 2,4-dichlorobenzoate

Prepared according to method F.

5 mg, yield 4%.

¹H NMR (270 MHz, CHLOROFORM-D) δ ppm 1.21-1.45 (m, 1H) 1.52-1.74 (m, 2H) 1.82-1.93 (m, 1H) 2.17-2.37 (m, 1H) 2.64-2.76 (m, 1H) 2.88-3.02 (m, 2H) 3.30-3.36 (m, 1H) 4.20-4.30 (m, 1H) 4.42-4.57 (m, 2H) 5.97-6.06 (m, 1H) 6.22-6.27 (m, 1H) 7.25-7.35 (m, 1H) 7.47 (d, J=1.98 Hz, 1H) 7.82 (dd, J=2.16, 8.37 Hz, 1H).

MS m/z: (M+H) 425.

### Example 312 (BVT067956)

### N-{2-[2-(cyclooctylamino)-4-oxo-4,5-dihydro-1,3-thiazol-5-yl]ethyl}-2,6-difluorobenzenesulfonamide

Prepared according to method K using a sulfonyl chloride.

54 mg, yield 60%.

¹H NMR (400 MHz, DMSO-d₆) δ 1.45-1.76 (m, 15H), 2.15-2.25 (m, 1H), 2.95-3.08 (m, 2H), 3.99 (m, 1H), 4.14 (m, 1H), 7.28 (m, 2H), 7.70 (m, 1H), 8.36 (s br., 1H), 9.16 (d, J=7.4 Hz, 1H).

MS (ESI+) for C₁₉H₂₅F₂N₃O₃S₂ m/z 446 (M+H)⁺.

### Example 313 (BVT070765)

### N-{2-[2-(cyclooctylamino)-4-oxo-4,5-dihydro-1,3-thiazol-5-yl]ethyl}-2,6-difluorobenzamide

Prepared according to method K.

26 mg, yield 69%.

1H NMR (400 MHz, CHLOROFORM-D) δ ppm 1.50-1.67 (m, 8H) 1.70-1.81 (m, 2H) 1.84-1.96 (m, 4H) 2.10-2.22 (m, 1H) 2.43-2.56 (m, 1H) 3.51-3.63 (m, 2H) 3.82-3.94 (m, 1H) 4.28-4.35 (m, 1H) 6.89-6.98 (m, 2H) 7.31-7.42 (m, 1H) MS m/z 410 (M+H)⁺

### Example 314 (BVT051005)

### 2-(bicyclo[2.2.1]hept-5-en-2-ylamino)-5-[2-(2-chlorophenoxy)ethyl]-1,3-thiazol-4(5H)-one

Prepared according to method G.

13 mg, yield 15%.

MS (EI⁺) m/z 363.

### Example 315 (BVT070764)

### 2-chloro-N-{2-[2-(cyclooctylamino)-4-oxo-4,5-dihydro-1,3-thiazol-5-yl]ethyl}benzamide

Prepared according to method K.

16 mg, yield 44%.

1H NMR (400 MHz, CHLOROFORM-D) δ ppm 1.47-1.68 (m, 8H) 1.71-1.83 (m, 2H) 1.85-1.97 (m, 4H) 2.12-2.27 (m, 1H) 2.44-2.58 (m, 1H) 3.52-3.63 (m, 2H) 3.80-3.95 (m, 1H) 4.29-4.38 (m, 1H) 6.64-6.74 (m, 1H) 7.28-7.46 (m, 2H) 7.58-7.66 (m, 1H) MS m/z 408 (M+H)⁺

### Example 316 (BVT067659)

### 5-[2-(4-benzylpiperidin-1-yl)-2-oxoethyl]-2-(cycloheptylamino)-1,3-thiazol-4(5H)-one

Prepared according to method D.

60 mg, yield 38%.

1H NMR (400 MHz, DMSO-D6) δ ppm 0.89-1.20 (m, 2H) 1.33-1.80 (m, 14H) 2.60-2.76 (m, 2H) 2.82-2.96 (m, 1H) 3.10-3.26 (m, J=1.22 Hz, 2H) 3.68-3.78 (m, 2H) 3.96 (s, 1H) 4.12-4.24 (m, 1H) 4.24-4.35 (m, 1H) 7.11-7.21 (m, J=7.08 Hz, 3H) 7.22-7.31 (m, 2H) 9.08-9.23 (m, 1H).

M/S m/z 428 (M+H)⁺

### Example 317 (BVT059331)

### N-{2-[2-(bicyclo[2.2.1]hept-5-en-2-ylamino)-4-oxo-4,5-dihydro-1,3-thiazol-5-yl]ethyl}-2,4-dichlorobenzamide

Prepared according to method K.

11 mg, yield 12%.

¹H NMR (270 MHz, METHANOL-D4) δ ppm 1.36-1.71 (m, 4H) 1.96-2.05 (m, 1H) 2.21-2.39 (m, 1H) 2.80-2.83 (m, 2H) 3.31-3.55 (m, 2H) 3.68-3.72 (m, 1H) 4.27-4.32 (m, 1H) 5.96-6.02 (m, 1H) 6.10-6.14 (m, 1H) 7.29-7.44 (m, 3H). MS (ESI+) for C₁₉H₁₉Cl₂₃O₂S m/z 424 (M+H)⁺.

### Example 318 (BVT049923)

### 2-chlorophenyl [2-(bicyclo[2.2.1]hept-5-en-2-ylamino)-4-oxo-4,5-dihydro-1,3-thiazol-5-yl]acetate

Prepared according to method L.

130 mg, yield 46%.

Mp 211° C.

¹H NMR (400 MHz, CDCl₃) δ 1.58-1.60 (m, 1H) 1.68-1.72 (m, 2H) 1.97-2.05 (m, 1H) 2.97-3.01 (m, 2H) 3.03-3.10 (m, 1H) 3.34-3.38 (m, 1H) 3.65-3.72 (m, 1H) 4.44-4.48 (m, 1H) 6.04-6.07 (m, 1H) 6.23 (dd, J1=5.52, J2=2.51 Hz, 1H) 7.13-7.31 (m, 3H) 7.43-7.46 (m, 1H).

MS (EI⁺) m/z 377.2.

### Example 319 (BVT059210)

### N-{2-[2-(bicyclo[2.2.1]hept-5-en-2-ylamino)-4-oxo-4,5-dihydro-1,3-thiazol-5-yl]ethyl}-2-chlorobenzamide

Prepared according to method K.

14 mg, yield 36%.

¹H NMR (270 MHz, CHLOROFORM-D) δ 1.61-1.85 (m, 4H) 2.10-2.28 (m, 1H) 2.43-2.60 (m, 1H) 2.99-3.08 (m, 2H) 3.38 (dd, J=6.93, 3.46 Hz, 1H) 3.49-3.65 (m, 1H) 3.86-4.01 (m, 1H) 4.35-4.46 (m, 1H) 6.06 (dd, J=5.44, 2.97 Hz, 1H) 6.29 (dd, J=5.69, 2.97 Hz, 1H) 6.75 (t, J=5.07 Hz, 1H) 7.29-7.44 (m, 4H) 7.57-7.63 (m, 1H).

MS (ESI+) for C₁₉H₂₀ClN₃O₂S m/z 390 (M+H)⁺

### Example 320 (BVT070733)

### N-{2-[2-(cyclooctylamino)-4-oxo-4,5-dihydro-1,3-thiazol-5-yl]ethyl}-5-methyl-3-phenylisoxazole-4-carboxamide

Prepared according to method K.

23 mg, yield 57%.

1H NMR (400 MHz, CHLOROFORM-D) δ ppm 1.50-1.69 (m, 8H) 1.73-1.83 (m, 2H) 1.87-2.04 (m, 5H) 2.19-2.30 (m, 1H) 2.69-2.74 (m, 3H) 3.23-3.32 (m, 1H) 3.52-3.70 (m, 2H) 4.11-4.17 (m, 1H) 7.51-7.60 (m, 5H).

MS m/z 455 (M+H)⁺.

### Example 321 (BVT067922)

### 5-[2-(4-benzylpiperidin-1-yl)-2-oxoethyl]-2-[(cyclohexylmethyl)amino]-1,3-thiazol-4(5H)-one

Prepared according to method D.

19 mg, yield 30%.

1H NMR (400 MHz, CHLOROFORM-D) δ ppm 12.32-14.24 (m, 1H), 7.27-7.34 (m, 2H), 7.19-7.26 (m, 1H), 7.10-7.17 (m, 2H), 4.48-4.60 (m, 1H), 4.43 (dd, J=11.72, 3.30 Hz, 0.5H), 4.40 (dd, J=11.72, 3.30 Hz, 0.5H), 3.65-3.78 (m, 1H), 3.54 (dd, J=16.97, 3.54 Hz, 0.5H), 3.52 (dd, J=16.97, 3.54 Hz, 0.51H), 3.22 (t, J=7.26 Hz, 2H), 2.94-3.10 (m, 1H), 2.77 (dd, J=12.02, 6.16 Hz, 0.5H), 2.73 (dd, J=12.21, 6.47 Hz, 0.5H), 2.58 (d, J=6.96 Hz, 2H), 2.52-2.67 (m, 1H), 1.62-1.88 (m, 9H), 1.08-1.39 (m, 5H), 0.89-1.10 (m, 2H) MS m/z 428 (M+H)⁺

### Example 322 (BVT051121)

### methyl 4-{2-[2-(bicyclo[2.2.1]hept-5-en-2-ylamino)-4-oxo-4,5-dihydro-1,3-thiazol-5-yl]ethoxy}-3-chlorobenzoate

Prepared according to method G.

MS (EI⁺) m/z 421.

### Example 323 (BVT050180)

### phenyl [2-(bicyclo[2.2.1]hept-5-en-2-ylamino)-4-oxo-4,5-dihydro-1,3-thiazol-5-yl]acetate

Prepared according to method L.

48 mg, yield 37%.

¹H NMR (400 MHz, CDCl₃) δ 1.58-1.60 (m, 1H) 1.68-1.72 (m, 2H) 1.97-2.05 (m, 1H) 2.97-3.01 (m, 2H) 3.03-3.10 (m, 1H) 3.34-3.38 (m, 1H) 3.65-3.72 (m, 1H) 4.44-4.48 (m, 1H) 6.04-6.07 (m, 1H) 6.23 (dd, J1=5.52, J2=2.51 Hz, 1H) 7.13-7.31 (m, 3H) 7.43-7.46 (m, 1H). MS (EI⁺) m/z 343.0.

### Example 324 (BVT059211)

### N-{2-[2-(bicyclo[2.2.1]hept-5-en-2-ylamino)-4-oxo-4,5-dihydro-1,3-thiazol-5-yl]ethyl}-2-bromo-5-methoxybenzamide

Prepared according to method K.

23.7 mg, yield 52%.

¹H NMR (270 MHz, CHLOROFORM-D) δ 1.67 (s, 2H) 1.71-1.85 (m, 2H) 2.07-2.25 (m, 1H) 2.45-2.62 (m, 1H) 3.03 (d, J=8.41 Hz, 2H) 3.38 (dd, J=7.18, 3.46 Hz, 1H) 3.43-3.60 (m, 1H) 3.79 (s, 3H) 3.84-4.00 (m, 1H) 4.42-4.52 (m, 1H) 6.06 (dd, J=5.20, 3.22 Hz, 1H) 6.28 (dd, J=5.57, 2.85 Hz, 1H) 6.61 (s, 1H) 6.84 (dd, J=8.78, 2.60 Hz, 1H) 7.03 (d, J=2.97 Hz, 1H) 7.45 (d, J=8.91 Hz, 1H).

MS (ESI+) for C₂₀H₂₂BrN₃O₃S m/z 466 (M+H)⁺

### Example 325 (BVT070734)

### N-{2-[2-(cyclooctylamino)-4-oxo-4,5-dihydro-1,3-thiazol-5-yl]ethyl}-2-phenoxyacetamide

Prepared according to method K.

31 mg, yield 83%.

1H NMR (400 MHz, CHLOROFORM-D) δ ppm 1.49-1.68 (m, 8H) 1.71-1.81 (m, 2H) 1.87-1.94 (m, 4H) 2.03-2.14 (m, 1H) 2.40-2.50 (m, 1H) 3.42-3.60 (m, 2H) 3.67-3.78 (m, 1H) 4.16-4.22 (m, 1H) 4.48-4.52 (m, 2H) 6.89-6.94 (m, 2H) 7.00-7.06 (m, 1H) 7.28-7.35 (m, 2H) MS m/z 404 (M+H)⁺

### Example 326 (BVT067355)

### 2-(cycloheptylamino)-5-[2-(1-oxa-4-azaspiro[4.5]dec-4-yl)-2-oxoethyl]-1,3-thiazol-4(5H)-one

Prepared according to method D.

40 mg, yield 25%.

M/S m/z 394 (M+H)⁺

### Example 327 (BVT059330)

### N-{2-[2-(bicyclo[2.2.1]hept-5-en-2-ylamino)-4-oxo-4,5-dihydro-1,3-thiazol-5-yl]ethyl}-2-fluoro-4-(trifluoromethyl)benzamide

Prepared according to method K.

10 mg, yield 11%.

¹H NMR(270 MHz, METHANOL-D4) δ ppm 1.34-71.70 (m, 4H) 1.95-2.10 (m, 1H) 2.32-2.42 (m, 1H) 2.79-2.80 (m, 2H) 3.46-3.55 (m, 2H) 3.70-3.74 (m, 1H) 4.23-4.30 (m, 1H) 5.95-6.00 (m, 1H) 6.10-6.13 (m, 1H) 7.47-7.52 (m, 2H) 7.76-7.82 (m, 1H). MS (ESI+) for C₂₀H₁₉F₄N₃O₂S m/z 442 (M+H)⁺.

### Example 328 (BVT051136)

### 2-(bicyclo[2.2.1]hept-5-en-2-ylamino)-5-[2-(4-chloro-3-methylphenoxy)ethyl]-1,3-thiazol-4(5H)-one

Prepared according to method G.

2 mg, yield

MS (EI⁺) m/z 377.

### Example 329 (BVT067353)

### 2-[2-(cycloheptylamino)-4-oxo-4,5-dihydro-1,3-thiazol-5-yl]-N-ethyl-N-phenylacetamide

Prepared according to method D.

70 mg, yield 51%.

1H NMR (400 MHz, DMSO-D6) d ppm 0.93 (t, 2H) 1.25-1.64 (m, 11H) 1.66-1.89 (m, 2H) 2.12-2.31 (m, 1H) 2.71-2.85 (m, 2H) 3.78-3.95 (m, 2H) 4.05-4.21 (m, 1H) 7.20-7.50 (m, J=62.26 Hz, 5H) 9.07-9.23 (m, 1H).

M/S m/z 374 (M+H)⁺

### Example 330 (BVT067656)

### N-(2-chloro-6-fluorobenzyl)-2-[2-(cycloheptylamino)-4-oxo-4,5-dihydro-1,3-thiazol-5-yl]acetamide

Prepared according to method K.

80 mg, yield 50%.

M/S m/z 412 (M+H)⁺

### Example 331 (BVT050205)

### 2-methoxyphenyl [2-(bicyclo[2.2.1]hept-5-en-2-ylamino)-4-oxo-4,5-dihydro-1,3-thiazol-5-yl]acetate

Prepared according to method L.

57 mg, yield 41%.

Mp 175-176° C. ¹H NMR (400 MHz, CDCl₃) δ 1.58-1.60 (m, 1H) 1.68-1.72 (m, 2H) 1.97-2.05 (m, 1H) 2.97-3.01 (m, 2H) 3.03-3.10 (m, 1H) 3.34-3.38 (m, 1H) 3.65-3.72 (m, 1H) 4.44-4.48 (m, 1H) 6.04-6.07 (m, 1H) 6.23 (dd, J1=5.52, J2=2.51 Hz, 1H) 7.13-7.31 (m, 3H) 7.43-7.46 (m, 1H).

MS (EI⁺) m/z 373.0.

### Example 332 (BVT067951)

### N-{2-[2-(cyclooctylamino)-4-oxo-4,5-dihydro-1,3-thiazol-5-yl]ethyl}adamantane-1-carboxamide

Prepared according to method K.

15 mg, yield 4%.

1H NMR (400 MHz, CHLOROFORM-D) δ ppm 1.44-1.85 (m, 22H) 1.85-1.95 (m, 4H) 2.01-2.15 (m, 4H) 2.33-2.44 (m, 1H) 3.34-3.44 (m, 1H) 3.53-3.71 (m, 21H) 4.17 (dd, J=10.01, 4.15 Hz, 1H).

MS m/z 432 (M+H)⁺.

### Example 333 (BVT059374)

### 5-[2-(3,4-dihydroquinolin-1(2H)-yl)-2-oxoethyl]-2-[(2-fluorophenyl)amino]-1,3-thiazol-4(5H)-one

Prepared according to method D.

5 mg, yield 5%.

MS (ESI+) m/z 384 (M+H)⁺.

### Example 334 (BVT059209)

### N-{2-[2-(bicyclo[2.2.1]hept-5-en-2-ylamino)-4-oxo-4,5-dihydro-1,3-thiazol-5-yl]ethyl}-2,5-difluorobenzamide

5-(2-aminoethyl)-2-(bicyclo[2.2.1]hept-5-en-2-ylamino)-1,3-thiazol-4(5H)-one (0.025 g, 0.101 mmol) was dissolved in a few drops of DMF and Pyridine (2ml). 2,5-difluorobenzoyl chloride (0.053 g, 0.302 mmol) was added and the reaction mixture was shaken at room temperature. Additional 2,5-difluorobenzoyl chloride (0.036 g, 0.202 mmol) was added after 1 h and the reaction mixture was shaken at room temperature over night.

10% HCl was added and extraction with DCM performed. The organic phase was concentrated under vacuum. Purification was performed using preparative LC-MS.

22 mg, yield 56%.

¹H NMR (270 MHz, METHANOL-D4) δ 1.43-1.79 (m, 4H) 2.07-2.22 (m, 1H) 2.37-2.52 (m, 1H) 2.86-3.02 (m, 2H) 3.48-3.69 (m, 2H) 3.78 (dd, J=7.79, 2.85 Hz, 1H) 4.35-4.46 (m, 1H) 6.05-6.12 (m, 1H) 6.20-6.30 (m, 1H) 7.18-7.35 (m, 2H) 7.42-7.51 (m, 1H) MS (ESI+) for C₁₉H₁₉F₂N₃O₂S m/z 392 (M+H)⁺

### Example 335 (BVT074258B)

### 5-(2-anilinoethyl)-2-{[1-(4-chlorophenyl)cyclobutyl]amino}-1,3-thiazol-4(5H)-one hydrobromide

Prepared according to method H.

97 mg, yield 69%.

¹H NMR (400 MHz, DMSO-d₆) δ 1.89-2.17 (m, 3H), 2.54-2.75 (m, 5H), 3.99 (m, 2H), 5.01 (m, 1H), 7.23 (t, J=7.3 Hz, 1H), 7.39-7.53 (m, 6H), 7.66 (d, J=8.1 Hz, 1H), 8.08 (s br., 1H), 9.77 (s br, 1H), 10.88 (s, 1H).

MS (ESI+) for C₂₁H₂₂ClN₃OS m/z 400 (M+H)⁺.

### Example 336 (BVT067360)

### N-{2-[2-(cycloheptylamino)-4-oxo-4,5-dihydro-1,3-thiazol-5-yl]ethyl}-2,5-difluorobenzamide

Prepared according to method K.

11 mg, yield 16%.

¹H NMR (270 MHz, METHANOL-D4) δ ppm 1.44-1.82 (m, 10H) 1.93-2.22 (m, 3H) 2.38-2.53 (m, 1H) 3.46-3.69 (m, 2H) 3.98-4.11 (m, 1H) 4.35 (dd, J=9.28, 4.08 Hz, 1H) 7.17-7.35 (m, 2H) 7.42-7.50 (m, 1H).

MS m/z: (M+H) 396.

### Example 337 (BVT067354)

### 2-[2-(cycloheptylamino)-4-oxo-4,5-dihydro-1,3-thiazol-5-yl]-N-(4-methoxyphenyl)-N-methylacetamide

Prepared according to method D.

70 mg, yield 47%.

1H NMR (400 MHz, DMSO-D6) d ppm 1.25-1.70 (m, 10H) 1.76-1.92 (m, 2H) 2.16-2.38 (m, 2H) 2.79-2.96 (m, 1H) 3.11 (s, 3H) 3.77 (s, 3H) 4.12-4.23 (m, 1H) 6.92-7.07 (m, 2H) 7.20-7.32 (m, 2H) 9.08-9.21 (m, 1H). M/S m/z 390 (M+H)⁺

### Example 338 (BVT051120)

### 2-(bicyclo[2.2.1]hept-5-en-2-ylamino)-5-[2-(4-phenoxyphenoxy)ethyl]-1,3-thiazol-4(5H)-one

Prepared according to method G

MS (EI⁺) m/z 421.

### Example 339 (BVT070727)

### 4-chloro-N-{2-[2-(cyclooctylamino)-4-oxo-4,5-dihydro-1,3-thiazol-5-yl]ethyl}benzamide

Prepared according to method K.

Yield 19.5 mg (52%)

1H NMR (400 MHz, CHLOROFORM-D) δ ppm 1.45-1.68 (m, 8H) 1.70-1.81 (m, 2H) 1.85-1.97 (m, 4H) 2.14-2.25 (m, 1H) 2.39-2.50 (m, 1H) 3.53-3.67 (m, 2H) 3.74-3.84 (m, 1H) 4.27-4.33 (m, 1H) 7.40 (d, J=8.30 Hz, 2H) 7.72 (d, J=8.55 Hz, 2H) MS m/z 408 (M+H)⁺

### Example 340 BVT070731

### N-{2-[2-(cyclooctylamino)-4-oxo-4,5-dihydro-1,3-thiazol-5-yl]ethyl}cyclohexanecarboxamide

Prepared according to method K.

23 mg, yield 65%.

1H NMR (400 MHz, CHLOROFORM-D) δ ppm 1.18-1.32 (m, 3H) 1.34-1.45 (m, 2H) 1.50-1.71 (m, 9H) 1.72-1.96 (m, 10H) 2.03-2.19 (m, 2H) 2.33-2.43 (m, 1H) 3.33-3.43 (m, 1H) 3.54-3.71 (m, 2H) 4.21 (dd, J=10.01, 4.15 Hz, 1H) MS m/z 380 (M+H)⁺

### Example 341 (BVT070728)

### N-{2-[2-(cyclooctylamino)-4-oxo-4,5-dihydro-1,3-thiazol-5-yl]ethyl}-4-(trifluoromethyl)benzamide

Prepared according to method K.

14 mg, yield 34%.

1H NMR (400 MHz, CHLOROFORM-D) δ ppm 1.45-1.69 (m, 8H) 1.70-1.83 (m, 2H) 1.85-1.98 (m, 4H) 2.18-2.29 (m, 1H) 2.39-2.50 (m, 1H) 3.53-3.73 (m, 2H) 3.75-3.86 (m, 1H) 4.29-4.36 (m, 1H) 7.70 (d, J=8.30 Hz, 2H) 7.90 (d, J=8.06 Hz, 2H) MS m/z 442 (M+H)⁺

### Example 342 (BVT059370)

### 2-{[3,5-bis(trifluoromethyl)phenyl]amino}-5-[2-(3,4-dihydroquinolin-1(2H)-yl)-2-oxoethyl]-1,3-thiazol-4(5H)-one

To a suspension of PS-Carbodiimide resin (1.10 mmol/g, 314 mg, 0.345 mmol) in 10% DMF in DCM (4 mL) was added (2-{[3,5-bis(trifluoromethyl)phenyl]amino}-4-oxo-4,5-dihydro-1,3-thiazol-5-yl)acetic acid (100.0 mg, 0.259 mmol) and the mixture was gently shaken for 45 min. After addition of 1,2,3,4-tetrahydroquinoline (22 μL, 0.175 mmol), the mixture was shaken overnight and then filtered with the aid of methanol. The solvent was removed, and the residue was purified by preparative reverse-phase HPLC to give the title compound as an off-white solid.

9 mg, yield 9%.

MS (ESI+) m/z 502 (M+H)⁺.

### Example 343 (BVT074103)

### 2-anilino-5-[2-(1,3-dihydro-2H-isoindol-2-yl)-2-oxoethyl]-1,3-thiazol-4(5H)-one

(2-anilino-4-oxo-4,5-dihydro-1,3-thiazol-5-yl)acetic acid (25 mg, 1 eq) was dissolved in DCM (1 mL) and 1-[3-(dimethylamino)propyl]-3-ethylcarbodiimide hydrochloride (EDC, 29 mg, 1.5 eq), 1-hydroxybenzotriazole hydrate (HOBt, 20 mg, 1.5 eq), and N-methylmorpholine (NMM, 44 μL, 4 eq) were added sequentially. After 10 min stirring at room temperature isoindoline (13 μL, 1.1 eq) was added and the reaction mixture was stirred overnight at room temperature H₂O (5 mL) and DCM (5 mL) was added, the organic layer separated on a 1-PS syringe and concentrated. Purified by flashtube DCM-MeOH (14:1).

17 mg, yield 47%.

1H NMR (400 MHz, CHLOROFORM-D) δ ppm 2.09-2.45 (m, 2H), 4.19-4.42 (m, 1H), 4.53-4.77 (m, 4H), 7.01-7.22 (m, 7H), 7.30-7.42 (m, 2H). MS [M+H]⁺ m/z=352.

### Example 344 (BVT067657)

### 2-(cycloheptylamino)-5-[2-(4-hydroxy-4-phenylpiperidin-1-yl)-2-oxoethyl]-1,3-thiazol-4(5H)-one

Prepared according to method D.

30 mg, yield 18%.

M/S m/z 430 (M+H)⁺

### Example 345 (BVT067923)

### 2-{2-[(cyclohexylmethyl)amino]-4-oxo-4,5-dihydro-1,3-thiazol-5-yl}-N,N-diethylacetamide

Prepared according to method D.

19 mg, yield 39%.

1H NMR (400 MHz, CHLOROFORM-D) δ ppm 12.44-13.74 (m, 1H), 4.45 (dd, J=12.15, 3.36 Hz, 1H), 3.53 (dd, J=16.97, 3.42 Hz, 0.8H),3.38-3.46 (m, 2H), 3.40 (dd, J=16.72, 3.66 Hz, 0.2H), 3.33 (q, J=7.20 Hz, 2H), 3.25 (d, J=6.47 Hz, 2H), 2.93 (dd, J=18.07, 10.74 Hz, 0.2H), 2.80 (dd, J=16.97, 12.09 Hz, 0.8H), 1.64-1.85 (m, 7H), 1.24 (t, J=7.20 Hz, 3H), 1.16 (t, J=7.14 Hz, 3H), 1.11-1.38 (m, 2H), 0.92-1.10 (m, 2H) MS m/z 326 (M+H)⁺

### Example 346 (BVT070730)

### N-{2-[2-(cyclooctylamino)-4-oxo-4,5-dihydro-1,3-thiazol-5-yl]ethyl}-2,2-dimethylpropanamide

Prepared according to method K.

23 mg, yield 70%.

1H NMR (400 MHz, CHLOROFORM-D) δ ppm 1.19-1.21 (m, 9H) 1.49-1.68 (m, 8H) 1.72-1.83 (m, 2H) 1.85-1.97 (m, 4H) 2.00-2.11 (m, 1H) 2.34-2.44 (m, 1H) 3.31-3.40 (m, 1H) 3.54-3.73 (m, 2H) 4.16-4.22 (m, 1H).

MS m/z 354 (M+H)⁺

### Example 347 (BVT067971)

### 2-anilino-5-(2-azepan-1-yl-2-oxoethyl)-1,3-thiazol-4(5H)-one

Prepared according to method D.

22 mg, yield 33%.

1H NMR (400 MHz, CHLOROFORM-D) δ ppm 1.50-1.61 (m, 4H), 1.64-1.78 (m, 4H), 2.78 (dd, J=17.0, 12.1 Hz, 1H), 3.30-3.61 (m, 5H), 4.44 (dd, J=11.8, 2.8 Hz, 1H), 7.30-7.38 (m, 3H), 7.39-7.47 (m, 2H).

MS [M+H]⁺ m/z=332.

### Example 348 (BVT050213)

### 3-morpholin-4-ylphenyl [2-(bicyclo[2.2.1]hept-5-en-2-ylamino)-4-oxo-4,5-dihydro-1,3-thiazol-5-yl]acetate

Prepared according to method L.

57 mg, yield 41%

Mp 175-176° C.

¹H NMR (400 MHz, CDCl₃, major tautomer given) δ 1.58-1.60 (m, 1H) 1.68-1.72 (m, 2H) 1.97-2.05 (m, 1H) 2.97-3.01 (m, 2H) 3.03-3.10 (m, 1H) 3.34-3.38 (m, 1H) 3.65-3.72 (m, 1H) 4.44-4.48 (m, 1H) 6.04-6.07 (m, 1H) 6.23 (dd, J1=5.52, J2=2.51 Hz, 1H) 7.13-7.31 (m, 3H) 7.43-7.46 (m, 1H).

MS (EI⁺) m/z 373.0.

### Example 349 (BVT059442)

### N-{2-[2-(bicyclo[2.2.1]hept-5-en-2-ylamino)-4-oxo-4,5-dihydro-1,3-thiazol-5-yl]ethyl}-4-cyanobenzamide

Prepared according to method K.

15 mg, yield 19%.

1H NMR (270 MHz, DMSO-D6) δ ppm 1.39-1.59 (m, 6H) 1.85-1.90 (m, 1H) 2.26-2.35 (m, 1H) 2.78-2.85 (m, 2H) 3.72-3.73 (m, 1H) 4.18-4.24 (m, 1H) 6.06-6.21 (m, 2H) 7.97 (m, 4H) 8.82-8.84 (m, 1H) 9.29-9.31 (m, 1H).

MS (ESI+) for m/z 381 (M+H)⁺.

### Example 350 (BVT070726)

### N-{2-[2-(cyclooctylamino)-4-oxo-4,5-dihydro-1,3-thiazol-5-yl]ethyl}-4-methoxybenzamide

Prepared according to method K.

23 mg, yield 61%.

1H NMR (400 MHz, CHLOROFORM-D) δ ppm 1.45-1.68 (m, 9H) 1.70-1.82 (m, 2H) 1.85-1.96 (m, 4H) 2.15-2.24 (m, 1H) 2.41-2.52 (m, 1H) 3.53-3.66 (m, 2H) 3.83-3.87 (m, 3H) 4.29-4.35 (m, 1H) 6.93 (d, J=8.79 Hz, 2H) 7.72 (d, J=8.79 Hz, 2H).

MS m/z 404 (M+H)⁺

### Example 351 (BVT051309)

### 2-(bicyclo[2.2.1]hept-5-en-2-ylamino)-5-{2-[(2-chlorobenzyl)oxy]ethyl}-1,3-thiazol-4(5H)-one

Prepared according to method G.

50 mg, yield 68%.

¹H NMR (270 MHz, CHLOROFORM-D) d ppm 1.20-1.29 (m, 1H) 1.34-1.40 (m, 1H) 1.49-1.63 (m, 2H) 2.05-2.18 (m, 1H) 2.33-2.47 (m, 1H) 2.58 (br s, 1H) 2.81 (br s, 1H) 3.13-3.22 (m, 1H) 3.76-3.96 (m, 2H) 4.25-4.38 (m, 1H) 4.94-5.09 (m, 2H) 5.99 (dd, J=5.81, 3.09 Hz, 1H) 6.16 (dd, J=5.69, 2.97 Hz, 1H) 7.07-7.7.19 (m, 1H) 7.30-7.38 (m, 1H) 7.40-7.48 (m, 1H) 7.65-7.75 (m, 1H).

MS m/z: (M+H) 378.

### Example 352 (BVT059578)

### 2-{2-[(2-fluorophenyl)amino]-4-oxo-4,5-dihydro-1,3-thiazol-5-yl}-N-(4-methylcyclohexyl)acetamide

Prepared according to method D.

3 mg, yield 2%.

MS (ESI+) m/z 364 (M+H)⁺.

### Example 353 (BVT067464)

### 2-[(cyclohexylmethyl)amino]-5-(2-morpholin-4-yl-2-oxoethyl)-1,3-thiazol-4(5H)-one

Prepared according to method D.

29 mg, yield 58%.

1H NMR (400 MHz, CHLOROFORM-D) δ ppm 0.98 (m, 2H) 1.23 (m, 3H) 1.72 (m, 6H) 2.81 (m, J=9.00, 6.00 Hz, 1H) 3.23 (d, J=6.35 Hz, 2H) 3.44 (m, 2H) 3.51 (m, 1H) 3.61 (m, 2H) 3.71 (m, 4H) 4.44 (dd, J=11.72, 3.17 Hz, 1H).

MS m/z 340 (M+H)⁺

### Example 354 (BVT067463)

### 2-(cycloheptylamino)-5-isobutyl-1,3-thiazol-4(5H)-one

(2S)-2-amino-4-methylpentanoic acid (1.0 g, 7.6 mmol) was added to a solution of KBr (2.72 g, 22.9 mmol) in H₂SO₄ (1.25 M, 8.6 ml) and the reaction mixture was cooled in an ice-bath containing NaCl. NaNO₂ (0.53 g, 7.6 mmol) was added portion wise over 30 min and the mixture was stirred for 3 h while cooling was continued. The mixture was then stirred at room temperature for 1.5 h. The reaction mixture was then extracted with EtOAc and the organic phase was dried (Na₂SO₄) and concentrated in vacuum. This afforded 0.098 g of crude product, which was used in the next step without further purification. 2-bromo-4-methylpentanoic acid (0.030 g, 0.15 mmol) from above and N-cycloheptylthiourea (0.026 g, 0.15 mmol) in acetone (3 ml) were heated with stirring to 70° C. for 24 h. The reaction mixture was then concentrated in vacuum. Purification was performed using preparative LC (System A, 40-70% MeCN over 5 min).

32 mg, yield 77%.

¹H NMR (270 MHz, METHANOL-D4) δ 0.91-1.04 (m, 6H) 1.43-1.86 (m, 12H) 1.92-2.12 (m, 3H) 3.95-4.11 (m, 1H) 4.29-4.48 (m, 1H)

MS (ESI+) for C₁₄H₂₄N₂OS m/z 269 (M+H)⁺

### Example 355 (BVT070723)

### (5R)-2-(cycloheptylamino)-5-(cyclohexylmethyl)-1,3-thiazol-4(5H)-one

Prepared using the same procedure as Example 354.

13 mg, yield 29%.

¹H NMR (270 MHz, CHLOROFORM-D) δ ppm 0.85-1.90 (m, 22H) 1.93-2.10 (m, 2H) 2.14-2.30 (m, 1H) 3.35-3.57 (m, 1H) 4.23 (dd, J=11.32, 3.77 Hz, 1H)

MS (ESI+) for C₁₇H₂₈N₂OS m/z 309 (M+H)⁺

### Example 356 (BVT070735)

### (5S)-2-(cycloheptylamino)-5-(cyclohexylmethyl)-1,3-thiazol-4(5H)-one

Prepared using the same procedure as Example 354.

18 mg, yield 41%.

¹H NMR (270 MHz, CHLOROFORM-D) δ ppm 0.87-1.86 (m, 22H) 1.89-2.11 (m, 2H) 2.11-2.30 (m, 1H) 3.34-3.60 (m, 1H) 4.23 (dd, J=11.32, 3.77 Hz, 1H) 8.81 (br.s, 1H) MS (ESI+) for C₁₇H₂₈N₂OS m/z 309 (M+H)⁺

### Example 357 (BVT070725)

### 2-(cyclooctylamino)-5-(4-hydroxybenzyl)-1,3-thiazol-4(5H)-one

Prepared using the same procedure as Example 354.

10 mg, yield 15%.

¹H NMR (270 MHz, CHLOROFORM-D) δ ppm 1.41-1.95 (m, 14H) 3.07 (dd, J=14.47, 9.65 Hz, 1H) 3.43-3.59 (m, 2H) 4.46 (dd, J=9.65, 3.96 Hz, 1H) 6.81 (d, J=8.41 Hz, 2H) 7.08 (d, J=8.41 Hz, 2H)

MS (ESI+) for C₁₈H₂₄N₂O₂S m/z 333 (M+H)⁺

### Example 358 (BVT067796)

### 2-(cycloheptylamino)-5-(1H-indol-3-ylmethyl)-1,3-thiazol-4(5H)-one

Prepared using the same procedure as Example 354 from (2S)-2-amino-3-(1H-indol-3-yl)propanoic acid

8 mg, yield 19%.

¹H NMR (270 MHz, CHLOROFORM-D) δ 1.27-1.80 (m, 11H) 1.84-1.99 (m, 1H) 3.23-3.40 (m, 2H) 3.76 (dd, J=15.09, 3.46 Hz, 1H) 4.65 (d, J=9.15, 3.96 Hz, 1H) 7.10-7.28 (m, 3H) 7.40 (d, J=7.92 Hz, 1H) 7.59 (d, J=7.92 Hz, 1H) 8.26 (s, 1H)

MS (ESI+) for C₁₉H₂₃N₃OS m/z 342 (M+H)⁺

### Example 359 (BVT067868)

### 2-(cycloheptylamino)-5-(4-hydroxybenzyl)-1,3-thiazol-4(5H)-one

Prepared using the same procedure as Example 354 from (2S)-2-amino-3-(4-hydroxyphenyl)propanoic acid.

10 mg, yield 21%.

¹H NMR (270 MHz, METHANOL-D4) δ ppm 1.37-2.07 (m, 12H) 2.91-3.11 (m, 1H) 3.32-3.43 (m, 1H) 3.86-4.02 (m, 1H) 4.48-4.66 (m, 1H) 6.60-6.76 (m, 2H) 6.99-7.11 (m, 2H)

MS (ESI+) for C₁₇H₂₂N₂O₂S m/z 319 (M+H)⁺

### Example 360 (BVT063177)

### 2-(bicyclo[2.2.1]hept-2-ylamino)-5-(4-hydroxybenzyl)-1,3-thiazol-4(5H)-one

Prepared using the same procedure as Example 354 from 2-amino-3-(4-hydroxyphenyl)propanoic acid.

233 mg, yield 27%.

¹H NMR (270 MHz, DMSO-D6) δ 1.11 (d, J=9.65 Hz, 3H) 1.24-1.54 (m, 4H) 1.56-1.76 (m, 1H) 2.04-2.27 (m, 2H) 2.61-2.84 (m, 1H) 3.26 (dd, J=14.10, 3.96 Hz, 1H) 3.70 (s, 1H) 4.42-4.52 (obscured by HDO peak) (m, 1H) 6.57-6.72 (m, 2H) 6.92-7.08 (m, 2H) 9.07 (d, J=6.19 Hz, 1H)

¹H NMR (270 MHz, METHANOL-D4) 1.07-1.62 (m, 7H) 1.67-1.88 (m, 1H) 2.07-2.36 (m, 2H) 2.92-3.11 (m, 1H) 3.32-3.44 (partly obscured by MeOD peak) (m, 1H) 3.64-3.76 (m, 1H) 4.51-4.68 (m, 1H) 6.62-6.76 (m, 2H) 6.99-7.12 (m, 2H).

MS (ESI+) for C₁₇H₂₀N₂O₂S m/z 317 (M+H)⁺

### Example 361 (BVT067403)

### 2-(cycloheptylamino)-5-(3,4-dihydroxybenzyl)-1,3-thiazol-4(5H)-one

Prepared using the same procedure as Example 354 from (2S)-2-amino-3-(3,4-dihydroxyphenyl)propanoic acid.

2 mg, yield 4%).

¹H NMR (500 MHz CHLOROFORM-D) δ 1.43-1.57 (m, 6H) 1.56-1.73 (m, 5H) 1.84-2.01 (m, 2H) 2.87 (dd, J=14.13, 9.42 Hz, 1H) (1H hidden in MeOD peak) 3.97-4.06 (m, 1H) 4.44-4.51 (m, 1H) 6.52-6.57 (m, 1H) 6.64-6.68 (m, 2H)

MS (ESI+) for C₁₇H₂₂N₂O₃S m/z 335 (M+H)⁺

### Example 362 (BVT073749)

### 2-(cycloheptylamino)-5-(pyridin-3-ylmethyl)-1,3-thiazol-4(5H)-one

Prepared using the same procedure as Example 354 from (2S)-2-amino-3-pyridin-3-ylpropanoic acid.

5 mg, yield 52%.

1H NMR (270 MHz, CHLOROFORM-D) δ ppm 1.39-1.84 (m, 9H) 1.88-2.06 (m, 2H) 3.39-3.54 (m, 1H) 3.64 (s, 2H) 3.97 (s, 1H) 4.75 (s, 1H) 7.84-7.95 (m, 1H) 8.34 (d, J=7.67 Hz, 1H) 8.75 (d, J=5.07 Hz, 1H) 9.10 (s, 1H)

MS (ESI+) for C₁₆H₂₁N₃OS m/z 304 (M+H)⁺

### Example 363 (BVT105288B)

### 2-(cyclooctylamino)-5-propyl-1,3-thiazol-4(5H)-one hydrobromide

The thiourea (0.81 mmol) and the alpha-bromo ester (0.81 mmol) was dissolved in acetone and heated to 60° C. for 40-72 hours. The reactions was cooled and the products collected by filtration.

¹H NMR (400 MHz, DMSO-D6) δ ppm 0.82-0.93 (m, 3H) 1.22-1.32 (m, 1H) 1.35-1.72 (m, 14H) 1.73-1.85 (m, 2H) 1.95 (s, 1H) 3.99 (s, 1H) 4.23-4.33 (m, 1H) 9.74 (s, 1H).

### Example 364 (BVT105308B)

### 5-butyl-2-(cyclooctylamino)-1,3-thiazol-4(5H)-one hydrobromide

Prepared using the same procedure as for Example 363.

1H NMR (400 MHz, DMSO-D6) δ ppm 0.82-0.89 (m, 3H) 1.25-1.33 (m, 2H) 1.33-1.84 (m, 17H) 1.93-2.04 (m, 1H) 4.01 (s, 1H) 4.22-4.32 (m, 1H) 9.70 (s, 1H).

### Example 365 (BVT062674D)

### 2-(bicyclo[2.2.1]hept-2-ylamino)-5-ethyl-1,3-thiazol-4(5H)-one hydrobromide

Prepared using the same procedure as for Example 363.

105 mg, yield 38%.

1H NMR (400 MHz, DMSO-D6) δ ppm 0.90 (m, 3H) 1.03-1.23 (m, 3H) 1.35-1.56 (m, 4H) 1.65-1.84 (m, 2H) 1.98 (m, 1H) 2.24 (m, 2H) 3.75 (m, 1H) 4.22-4.40 (m, 1H) 9.84 (s, 1H). MS (ESI+) for C₁₂H₁₈N₂OS m/z 239 (M+H)⁺

### Example 366 (BVT062676B)

### 2-(cyclohexylamino)-5-ethyl-1,3-thiazol-4(5H)-one hydrobromide

Prepared using the same procedure as for Example 363.

201 mg, yield 75%.

1H NMR (400 MHz, DMSO-D6) δ ppm 0.91 (m, 3H) 1.05-2.06 (m, 12H) 3.76 (m, 1H) 4.36 (m, 1H) 10.11 (s, 1H). MS (ESI+) for C₁₁H₁₈N₂OS m/z 227 (M+H)⁺

### Example 367 (BVT061842)

### 5-ethyl-2-[(2-methylphenyl)amino]-1,3-thiazol-4(5H)-one

Prepared using the same procedure as for Example 363.

13 mg, yield 18%.

¹H NMR (270 MHz, METHANOL-D4) δ ppm 1.07-1.18 (m, J=7.36, 7.36 Hz, 3H) 1.98-2.36 (m, 2H) 2.11-2.13 (m, 3H) 4.52-4.75 (m, 1H) 7.12 (dd, J=20.54, 7.67 Hz, 1H) 7.22-7.46 (m, 3H).

MS (ESI+) for C₁₂H₁₄N₂OS m/z 235 (M+H)⁺.

### Example 368 (BVT067912)

### (5S)-2-(cycloheptylamino)-5-methyl-1,3-thiazol-4(5H)-one

Prepared using the same procedure as for Example 363.

56 mg, yield 80%.

[α]D=+0.4°, c=2.0 at 20.0° C. and in MeOH.

¹H NMR (400 MHz, CHLOROFORM-D) δ ppm 1.35-1.70 (m, 12H) 1.45 (d, J=7.3 Hz, 3H) 1.72-2.00 (m, 1H) 3.90-4.03 (m, 1H).

MS (ESI+) for C₁₁H₁₈N₂OS m/z 227 (M+H)⁺.

### Example 369 (BVT073763)

### 5-ethyl-2-[(2-isopropylphenyl)amino]-1,3-thiazol-4(5H)-one

Prepared using the same procedure as for Example 363.

26 mg, yield 19%.

1H NMR (270 MHz, DMSO-D6) δ ppm 0.88 (t, J=7.30 Hz, 3H) 1.14 (d, J=6.93 Hz, 6H) 1.63-2.06 (m, 2H) 2.93-3.11 (m, 1H) 4.32 (dd, J=7.36, 4.27 Hz, 1H) 6.75-6.92 (m, 1H) 7.06-7.21 (m, 2H) 7.24-7.42 (m, 1H).

MS (ESI+) for C₁₄H₁₈N₂OS m/z 263 (M+H)⁺.

### Example 370 (BVT093650)

### 2-(cyclooctylamino)-5-methyl-1,3-thiazol-4(5H)-one

Prepared using the same procedure as for Example 363.

20 mg, yield

¹H NMR (400 MHz, DMSO-D6) δ ppm 1.41-1.52 (m, 11H) 1.57-1.65 (m, 4H) 1.71-1.80 (m, 2H) 4.00 (ddd, J=8.55, 4.64, 4.39 Hz, 1H) 4.12 (q, J=7.16 Hz, 1H) 9.09 (brs, 0.72H). MS(ESI) for C₁₂H₂₀N₂OS m/z 241 (M+H).

### Example 371 (BVT073636)

### 2-(cyclooctylamino)-5-ethyl-1,3-thiazol-4(5H)-one

Prepared using the same procedure as for Example 363.

620 mg, yield 6%.

1H NMR (270 MHz, METHANOL-D4) δ ppm 0.89-1.03 (m, 3H) 1.43-1.99 (m, 15H) 1.98-2.16 (m, 1H) 4.21-4.32 (m, 1H) 4.55-4.66 (m, 1H).

MS (ESI+) for C₁₃H₂₂N₂OS m/z 255 (M+H)⁺.

### Example 372 (BVT073633)

### 2-(cycloheptylamino)-5-ethyl-1,3-thiazol-4(5H)-one

Prepared using the same procedure as for Example 363.

1 mg, yield 6%.

1H NMR (270 MHz, METHANOL-D4) δ ppm 0.90-1.06 (m, 3H) 1.40-2.17 (m, 14H) 4.26 (dd, J=7.86, 4.02 Hz, 1H) 4.52-4.68 (m, 1H).

MS (ESI+) for C₁₂H₂₀N₂OS m/z 241 (M+H)⁺.

### Example 373 (BVT056664)

### 2-{2-[2-(bicyclo[2.2.1]hept-5-en-2-ylamino)-4-oxo-4,5-dihydro-1,3-thiazol-5-yl]ethyl}-1H-isoindole-1,3(2H)-dione

2-bromo-4-(1,3-dioxo-1,3-dihydro-2H-isoindol-2-yl)butanoic acid (0.204 g, 0.654 mmol) and N-bicyclo[2.2.1]hept-5-en-2-ylthiourea (0.112 g, 0.666 mmol) were dissolved in acetone (15 ml) and heated to reflux for 8 h. The reaction mixture was allowed to cool to room temperature. NaHCO₃ (sat. solution) was added and extraction with DCM was performed. The organic phase was concentrated in vacuum to give the crude product (0.269 g) of which 10 mg was purified using preparative LC-MS (System C, 20-80% MeCN).

7 mg, yield

¹H NMR (270 MHz, CHLOROFORM-D) 1.65-1.84 (m, 4H) 2.16-2.37 (m, 1H) 2.57-2.73 (m, 1H) 2.99-3.11 (m, 2H) 3.37 (t, J=4.58 Hz, 1H) 3.73-3.88 (m, 1H) 3.96-4.12 (m, 1H) 4.20 (dd, J=10.27, 3.59 Hz, 1H) 6.03-6.10 (m, 1H) 6.29 (dd, J=5.69, 2.97 Hz, 1H) 7.73-7.81 (m, 2H) 7.81-7.91 (m, 2H)

MS (ESI+) for C₂₀H₁₉N₃O₃S m/z 382 (M+H)⁺

### Example 374 (BVT059579)

### 5-{[5-(2-chlorophenyl)-1,3,4-oxadiazol-2-yl]methyl}-2-[(2-fluorophenyl)amino]-1,3-thiazol-4(5H)-one

Prepared by modification of the method described by Kataky et al. Heterocyclic Chem. 1986, 23, 793.

To a mixture of {2-[(2-fluorophenyl)amino]-4-oxo-4,5-dihydro-1,3-thiazol-5-yl}acetic acid (100.0 mg, 0.373 mmol) and 2-chlorobenzohydrazide (64 mg, 0.375 mmol) in a small tube with screw-cap was added POCl₃ (0.5 mL) and the tube heated at 100° C. for 1.5 h. The product mixture was poured onto ice/water (ca 5 mL) and made basic by addition of saturated NaHCO₃ (aq). After filtration, the solid was dissolved in a minimum amount of MeOH and purified by reverse-phase preparative HPLC to give the title compound as an off-white solid 22 mg, yield 15%.

¹H NMR (400 MHz, CDCl₃) δ 3.44 (dd, J=17.0 Hz, J=10.2 Hz, 1H), 3.90 (dd, J=17.0 Hz, J=3.6 Hz, 1H), 4.74 (dd, J=10.2 Hz, J=3.6 Hz, 1H), 7.05-7.20 (m, 4H), 7.38 (t, J=7.6 Hz, 1H), 7.46 (dt, J=7.7 Hz, J=1.7 Hz, 1H), 7.52 (dd, J=7.9 Hz, J=1.2 Hz, 1H), 7.92 (dd, J=7.8 Hz, J=1.6 Hz, 1H).

MS (ESI+) m/z 403 (M+H)⁺.

### Example 375 (BVT061992)

### 5-{[5-(2-chlorophenyl)-1,3,4-oxadiazol-2-yl]methyl}-2-(tricyclo[3.3.1.0˜3,7˜]non-3-ylamino)-1,3-thiazol-4(5H)-one

Preparation according to the procedure described for Example 374 starting from [4-oxo-2-(tricyclo[3.3.1.0˜3,7˜]non-3-ylamino)-4,5-dihydro-1,3-thiazol-5-yl]acetic acid.

22 mg, yield 15%.

¹H NMR (400 MHz, DMSO-d₆) δ 1.45-1.56 (m, 4H), 1.88-2.08 (m, 6H), 2.21-2.25 (m, 2H), 2.42 (t, J=6.7 Hz, 1H), 3.46 (dd, J=16.4 Hz, J=8.2 Hz, 1H), 3.71 (dd, J=16.4 Hz, J=4.5 Hz, 1H), 4.62 (dd, J=8.2 Hz, J=4.2 Hz, 1H), 7.54 (tt, J=7.6 Hz, J=1.3 Hz, 1H), 7.63 (m, 1H), 7.69 (dd, J=8.1 Hz, J=1.3 Hz, 1H), 7.85 (dd, J=7.7 Hz, J=1.7 Hz, 1H), 9.40 (s, 1H).

MS (ESI+) m/z 429 (M+H)⁺.

### Example 376 (BVT061948)

### 5-{[5-(2-chlorophenyl)-1,3,4-oxadiazol-2-yl]methyl}-2-{[(1S,2S,3S,5R)-2,6,6-trimethylbicyclo[3.1.1]hept-3-yl]amino}-1,3-thiazol-4(5H)-one

Preparation according to the procedure described for Example 374 starting from (4-oxo-2-{[(1S,2S,3S,5R)-2,6,6-trimethylbicyclo[3.1.1]hept-3-yl]amino}-4,5-dihydro-1,3-thiazol-5-yl)acetic acid.

30 mg, yield 21%.

¹H NMR (400 MHz, DMSO-d₆) δ 0.89-1.08 (m, 7H), 1.18 (s, 3H) 1.49-1.63 (m, 1H), 1.71-2.09 (m, 3H), 2.29-2.47 (m, 2H), 3.45-3.55 (m, 1H), 3.70-3.78 (m, 1H), 4.32 (m, 1H), 4.68-4.73 (m, 1H), 7.52-7.57 (m, 1H), 7.61-7.65 (m, 1H), 7.68-7.71 (m, 1H), 7.88-7.92 (m, 1H), 9.35 (m, 1H).

MS (ESI+) m/z 445 (M+H)⁺.

### Example 377 (BVT061931)

### 2-(bicyclo[2.2.1]hept-2-ylamino)-5-{[5-(2-chlorophenyl)-1,3,4-oxadiazol-2-yl]methyl}-1,3-thiazol-4(5H)-one

Preparation according to the procedure described for Example 374 starting from [2-(bicyclo[2.2.1]hept-2-ylamino)-4-oxo-4,5-dihydro-1,3-thiazol-5-yl]acetic acid.

44 mg, yield 29%.

¹H NMR (400 MHz, DMSO-d₆) δ 1.05-1.51 (m, 7H), 1.62-1.73 (m, 1H), 2.10-2.22 (m, 2H), 3.43-3.52 (m, 1H), 3.69-3.77 (m, 2H), 4.64-4.70 (m, 1H), 7.55 (tt, J=7.6 Hz, J=1.4 Hz, 1H), 7.64 (m, 1H), 7.70 (m, 1H), 7.89 (dd, J=7.7 Hz, J=1.7 Hz, 1H), 9.14 (m, 1H).

MS (ESI+) m/z 403 (M+H)⁺.

### Example 378 (BVT061947)

### 5-{[5-(2-chlorophenyl)-1,3,4-oxadiazol-2-yl]methyl}-2-{[(1R,2R,3R,5S)-2,6,6-trimethylbicyclo[3.1.1]hept-3-yl]amino}-1,3-thiazol-4(5H)-one

Preparation according to the procedure described for Example 374 starting from (4-oxo-2-{[(1R,2R,3R,5S)-2,6,6-trimethylbicyclo[3.1.I]hept-3-yl]amino}-4,5-dihydro-1,3-thiazol-5-yl)acetic acid.

25 mg, yield 17%.

¹H NMR (400 MHz, DMSO-d₆) δ 0.89-1.08 (m, 7H), 1.19 (s, 3H) 1.49-1.63 (m, 1H), 1.71-2.03 (m, 3H), 2.28-2.47 (m, 2H), 3.45-3.55 (m, 1H), 3.70-3.78 (m, 1H), 4.32 (m, 1H), 4.68-4.73 (m, 1H), 7.52-7.57 (m, 1H), 7.61-7.65 (m, 1H), 7.68-7.71 (m, 1H), 7.88-7.92 (m, 1H), 9.35 (m, 1H).

MS (ESI+) m/z 445 (M+H)⁺.

### Example 379 (BVT063208)

### 5-(1H-benzimidazol-2-ylmethyl)-2-(cyclohexylamino)-1,3-thiazol-4(5H)-one

((1-cyclohexylamino)-4-oxo-4,5-dihydro-1,3-thiazol-5-yl)acetic acid (30 mg, 1 eq) was dissolved in a mixture of DCM/DMF (2 mL/2 mL) and 0-phenylendiamine (15 mg, 1.1 eq), and 1-[3-(dimethylamino)propyl]-3-ethylcarbodiimide hydrochloride (EDC, 30 mg, 1.3 eq) were then added sequentially. The reaction mixture was stirred 40° C. for 2 h. Separated between DCM and H₂O, the organic layer concentrated to give a crude orange brown oil. This material was taken up in HOAc (2 mL) and heated by microwave at 150° C. for 1200 s. The reaction mixture was evaporated and then purified by HP-LCMS to give the title compound

29 mg, yield 46%.

1H NMR (400 MHz, METHANOL-D4) δ ppm 1.28 (m, 5H) 1.77 (m, 5H) 3.83 (m, 3H) 4.81 (m, 1H) 7.59 (m, 2H) 7.76 (m, 2H). MS [M+H]⁺ m/z=329.

### Example 380 (BVT063207)

### 2-anilino-5-(1,3-benzoxazol-2-ylmethyl)-1,3-thiazol-4(5H)-one

The title compound was prepared according to the method described for Example 379.

4 mg, yield 11%.

¹H NMR (400 MHz, METHANOL-D4) δ ppm 3.39 (m, 1H) 3.87 (m, 1H) 4.80 (m, 1H) 6.90 (m, 1H) 7.15 (m, 2H) 7.32 (m, 4H) 7.50 (m, 1H) 7.62 (m, 1H); MS [M+H]⁺ m/z=324.

### Example 381 (BVT067972)

### 5-(1,3-benzoxazol-2-ylmethyl)-2-(cycloheptylamino)-1,3-thiazol-4(5H)-one

The title compound was prepared according to the method described for Example 379.

5.2 mg, yield 8%.

1H NMR (400 MHz, METHANOL-D4) δ ppm 1.45-1.75 (m, 10H), 1.91-2.06 (m, 2H), 3.37-3.50 (m, 1H), 3.77-3.89 (m, 1H), 4.04-4.15 (m, 1H), 4.50-4.75 (m, 1H), 7.31-7.40 (m, 2H), 7.53-7.59 (m, J=5.9, 2.2 Hz, 1H), 7.60-7.68 (m, 1H); MS [M+H]⁺ m/z=344.

### Example 382 (BVT066787)

### 2-anilino-5-(1,3-benzothiazol-2-ylmethyl)-1,3-thiazol-4(5H)-one

The title compound was prepared according to the method described for Example 379.

6 mg, yield 9%.

1H NMR (400 MHz, METHANOL-D4) δ ppm 3.54 (m, 1H) 4.04 (m, 1H) 4.79 (m, 1H) 7.16 (m, 2H) 7.39 (m, 4H) 7.63 (d, J=7.81 Hz, 1H) 7.89 (m, 2H); MS [M+H]⁺ m/z=340.

### Example 383 (BVT063209)

### 5-(1H-benzimidazol-2-ylmethyl)-2-(bicyclo[2.2.1]hept-2-ylamino)-1,3-thiazol-4(5H)-one

The title compound was prepared according to the method described for Example 379.

34 mg, yield 54%.

1H NMR (400 MHz, METHANOL-D4) δ ppm 1.36 (m, 8H) 1.76 (m, 1H) 2.29 (m, 2H) 3.79 (m, 3H) 7.59 (dd, J=6.35, 3.17 Hz, 2H) 7.76 (dd, J=5.98, 3.05 Hz, 2H); MS [M+H]⁺ m/z=341.

### Example 384 (BVT066789)

### 5-(1H-benzimidazol-2-ylmethyl)-2-(cycloheptylamino)-1,3-thiazol-4(5H)-one

The title compound was prepared according to the method described for Example 379.

15 mg, yield (24% yield).

1H NMR (400 MHz, METHANOL-D4) δ ppm 1.56 (m, 10H) 1.97 (m, 2H) 3.80 (m, 2H) 4.04 (m, 1H) 4.46 (m, 1H) 7.58 (dd, J=6.10, 3.17 Hz, 2H) 7.76 (dd, J=6.10, 3.17 Hz, 2H); MS [M+H]⁺ m/z=343.

### Example 385 (BVT067953)

### N-{2-[2-(cyclooctylamino)-4-oxo-4,5-dihydro-1,3-thiazol-5-yl]ethyl}-2-fluorobenzamide

Prepared according to method K after initial methylation of the starting amine.

43 mg, yield 37%.

¹H NMR (400 MHz, DMSO-d₆) δ 1.39-1.90 (m, 15H), 2.55 (m, 1H, obscured by solvent signal), 2.82 (s, 3H), 3.45 (m, 2H), 4.14 (m, 1H), 4.32 (m, 1H), 7.07-7.23 (m, 2H), 7.39-7.23 (m, 1H), 10.09 (d, J=6.6 Hz, 1H).

MS (ESI+) for C₂₁H₂₇F₂N₃O₂S m/z 424 (M+H)⁺.

### Example 386 (BVT073642)

### N-{2-[2-(cyclooctylamino)-4-oxo-4,5-dihydro-1,3-thiazol-5-yl]ethyl}-2,6-difluoro-N-methylbenzamide

Prepared according to method K after initial methylation of the starting amine.

43 mg, yield 37%.

¹H NMR (400 MHz, DMSO-d₆) δ 1.39-1.90 (m, 15H), 2.55 (m, 1H, obscured by solvent signal), 2.82 (s, 3H), 3.45 (m, 2H), 4.14 (m, 1H), 4.32 (m, 1H), 7.07-7.23 (m, 2H), 7.39-7.23 (m, 1H), 10.09 (d, J=6.6 Hz, 1H).

MS (ESI+) for C₂₁H₂₇F₂N₃O₂S m/z 424 (M+H)⁺.

### Example 387 (BVT073641)

### 2-chloro-N-{2-[2-(cyclooctylamino)-4-oxo-4,5-dihydro-1,3-thiazol-5-yl]ethyl}-N-methylbenzamide

Prepared according to method K after initial methylation of the starting amine.

54 mg, yield 47%.

¹H NMR (400 MHz, DMSO-d₆) δ 1.42-1.91 (m, 15H), 2.53 (m, 1H, obscured by solvent signal), 2.82 (s, 3H), 3.43 (m, 2H), 4.18-4.35 (m, 2H), 7.33-7.51 (m, 3H), 7.73 (d, J=7.3 Hz, 1H), 9.91 (d, J=6.0 Hz, 1H).

MS (ESI+) for C₂₁H₂₉ClN₃O₂S m/z 422 (M+H)⁺.

### Example 388 (BVT073643)

### 2,4-dichloro-N-{2-[2-(cyclooctylamino)-4-oxo-4,5-dihydro-1,3-thiazol-5-yl]ethyl}-N-methylbenzamide

Prepared according to method K after initial methylation of the starting amine.

71 mg, yield 57%.

¹H NMR (400 MHz, DMSO-d₆) δ 1.40-1.91 (m, 15H), 2.53 (m, 1H, obscured by solvent signal), 2.82 (s, 3H), 3.44 (m, 2H), 4.18-4.33 (m, 2H), 7.45 (d, J=8.3 Hz, 1H), 7.78 (s, 1H), 7.78 (d, J=8.2 Hz, 1H), 10.00 (d, J=6.1 Hz, 1H).

MS (ESI+) for C₂₁H₂₇Cl₂N₃O₂S m/z 456 (M+H)⁺.

### Preparation of a Pharmaceutical Composition

### Example 389

### Preparation of Tablets

| Ingredients | mg/tablet |
|---|---|
| 1. Active compound of formula (I) | 10.0 |
| 2. Cellulose, microcrystalline | 57.0 |
| 3. Calcium hydrogen phosphate | 15.0 |
| 4. Sodium starch glycolate | 5.0 |
| 5. Silicon dioxide, colloidal | 0.25 |
| 6. Magnesium stearate | 0.75 |

The active ingredient 1 is mixed with ingredients 2, 3, 4 and 5 for about 10 minutes. The magnesium stearate is then added, and the resultant mixture is mixed for about 5 minutes and compressed into tablet form with or without film-coating.

1. A compound of the general formula (I) 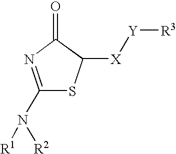
wherein
R¹ and R² are each independently selected from hydrogen; C₁₋₈-alkyl; C₃₋₁₀-cycloalkyl optionally independently substituted by one or more of C₁₋₈-alkyl; C₂₋₈-alkenyl; C₃₋₁₀-cycloalkyl-C₁₋₈-alkyl; C₃₋₁₀-cycloalkenyl; C₃₋₁₀-cycloalkenyl-C₁₋₈-alkyl; C₁₋₈-acyl; heterocyclyl optionally independently substituted by one or more of C₁₋₈-alkyl; heterocyclyl-C₁₋₈-alkyl; aryl optionally independently substituted by one or more of halogen, C₁₋₈-alkyl, halo-C₁₋₈-alkyl, C₁₋₈-alkoxy, and heterocyclyl; indanyl; aryl-C₁₋₈-alkyl optionally independently substituted by one or more of halogen and C₁₋₈-alkyl; aryl-C₃₋₁₀-cycloalkyl optionally independently substituted by one or more of halogen; heteroaryl optionally independently substituted by one or more of aryloxy; heterocyclyl-C₁₋₈-alkyl; or form together with the nitrogen atom bonded thereto heterocyclyl;
X is CH₂;
Y is CH₂, CO or a single bond;
R³ is hydrogen; C₁₋₈-alkyl; C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl substituted by one or more of halogen or hydroxy;
or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally independently substituted by one or more of halogen, C₁₋₈-alkyl and aryl; heteroaryl-C₁₋₈-alkylcarbonyl; C₃₋₁₀-cycloalkylcarbonyl; heterocyclylcarbonyl; heteroaryl; COOR⁶, wherein R⁶ is selected from C₁₋₈-alkyl and aryl; CONR⁷R⁸, wherein R⁷ and R⁸ are each independently selected from hydrogen and C₁₋₈-alkyl; or wherein R³ is OCONR⁹R¹⁰, wherein R⁹ and R¹⁰ are each independently selected from aryl optionally independently substituted by one or more of halogen, nitro, and aryloxy; or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro;
or pharmaceutically acceptable salts, solvates, hydrates, geometrical isomers, tautomers, optical isomers, N-oxides and prodrug forms thereof;
with the provisos that:
R¹ and R² are each independently selected from hydrogen; 2-butyl; isobutyl; tert-butyl; 2-methylbutyl; 1,1,3,3-tetramethylbutyl; cyclopropyl; cyclopentyl; cycloheptyl; cyclooctyl; C₃₋₁₀-bicycloalkyl; C₃₋₁₀-tricycloalkyl; cyclopropylmethyl; cyclohexylmethyl; 2,2,3,3-tetramethylcyclopropyl; (1R,2R,3R,5S)-2,6,6-trimethylbicyclo[3.1.1]hept-3-yl; (1S,2S,3S,5R)-2,6,6-trimethylbicyclo[3.1.1]hept-3-yl; C₃₋₁₀-cycloalkyl-C₁₋₈-alkyl; C₃₋₁₀-cycloalkenyl; C₃₋₁₀-cycloalkenyl-C₁₋₈-alkyl; heterocyclyl substituted by one or more of C₁₋₈-alkyl; heterocyclyl-C₁₋₈-alkyl; 1-naphthyl; phenyl substituted by one or more of halogen, C₁₋₈-alkyl, halo-C₁₋₈-alkyl, C₁₋₈-alkoxy, and heterocyclyl; indanyl; 4-chlorobenzyl; 4-methylbenzyl; (1R)-1-phenylethyl; (1S)-1-phenylethyl; 2-phenylethyl; (2R)-2-phenylpropyl; (2S)-2-phenylpropyl; aryl-C₃₋₁₀-cycloalkyl optionally independently substituted by one or more of halogen; heteroaryl substituted by one or more of aryloxy; heterocyclyl-C₁₋₈-alkyl; or form together with the nitrogen atom bonded thereto azepan-1-yl;
when either R¹ or R² is 1-naphthyl; phenyl substituted by one or more of halogen, C₁₋₈-alkyl, halo-C₁₋₈-alkyl, C₁₋₈-alkoxy, and heterocyclyl, then R⁴ and R⁵ are each independently selected from C₃₋₁₀-cycloalkyl optionally substituted by one or more of C₁₋₈-alkyl; cyclopropylmethyl; C₃₋₁₀-cycloalkylcarbonyl; 2-phenylethyl; 2-chloro-6-fluorobenzyl; 3-chloro-2-methylbenzyl; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally independently substituted by one or more of halogen, C₁₋₈-alkyl and aryl; heteroaryl-C₁₋₈-alkylcarbonyl; C₃₋₁₀-cycloalkylcarbonyl; heterocyclylcarbonyl; heteroaryl; or either of R⁴ and R⁵ is hydrogen and the other of R⁴ and R⁵ is selected from C₃₋₁₀-cycloalkyl optionally substituted by one or more of C₁₋₈-alkyl; cyclopropylmethyl; C₃₋₁₀-cycloalkylcarbonyl; 2-phenylethyl; 2-chloro-6-fluorobenzyl; 3-chloro-2-methylbenzyl; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally substituted by one or more of halogen, C₁₋₈-alkyl and aryl; heteroaryl-C₁₋₈-alkylcarbonyl; C₃₋₁₀-cycloalkylcarbonyl;
when both R¹ and R² are hydrogen, then R³ is C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl substituted by one or more of halogen or hydroxy;
or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally independently substituted by one or more of halogen, C₁₋₈-alkyl and aryl; heteroaryl-C₁₋₈-alkylcarbonyl; C₃₋₁₀-cycloalkylcarbonyl; heterocyclylcarbonyl; heteroaryl; COOR⁶, wherein R⁶ is selected from C₁₋₈-alkyl and aryl; CONR⁷R⁸, wherein R⁷ and R⁸ are each independently selected from hydrogen and C₁₋₈-alkyl; or wherein R³ is OCONR⁹R¹⁰, wherein R⁹ and R¹⁰ are each independently selected from aryl optionally independently substituted by one or more of halogen, nitro, and aryloxy;
or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro.

2. The compound according to claim 1, wherein:
R¹ and R² are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkenyl; C₃₋₁₀-cycloalkenyl-C₁₋₈-alkyl; heterocyclyl optionally independently substituted by one or more of C₁₋₈-alkyl; heterocyclyl-C₁₋₈-alkyl; aryl optionally independently substituted by one or more of halogen, C₁₋₈-alkyl, halo-C₁₋₈-alkyl, C₁₋₈-alkoxy, and heterocyclyl; indanyl; aryl-C₁₋₈-alkyl optionally independently substituted by one or more of halogen and C₁₋₈-alkyl; aryl-C₃₋₁₀-cycloalkyl optionally independently substituted by one or more of halogen; heteroaryl optionally independently substituted by one or more of aryloxy; heterocyclyl-C₁₋₈-alkyl; or form together with the nitrogen atom bonded thereto heterocyclyl;
X is CH₂;
Y is CH₂, CO or a single bond;
R³ is hydrogen; C₁₋₈-alkyl; C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl optionally independently substituted by one or more of halogen or hydroxy;
or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally substituted by one or more of halogen, C₁₋₈-alkyl and aryl; heteroaryl-C₁₋₈-alkylcarbonyl; C₃₋₁₀-cycloalkylcarbonyl; heteroaryl; or wherein R³ is OCONR⁹R¹⁰, wherein R⁹ and R¹⁰ are each independently selected from aryl substituted by one or more of halogen, nitro, and aryloxy;
or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro.

3. The compound according to any claim 1, wherein
R¹ and R² are selected from hydrogen; 2-butyl; isobutyl; tert-butyl; 2-methylbutyl; 1,1,3,3-tetramethylbutyl; cyclopropyl; cyclopentyl; cyclohexyl; cycloheptyl; bicyclo[2.2.1]hept-2-yl; cyclooctyl; 1-adamantyl; tricyclo[3.3.1.0˜3,7˜]non-3-yl; cyclopropylmethyl; cyclohexylmethyl; 2,2,3,3-tetramethylcyclopropyl; (1R,2R,3R,5S)-2,6,6-trimethylbicyclo[3.1.1]hept-3-yl; (1S,2S,3S,5R)-2,6,6-trimethylbicyclo[3.1.1]hept-3-yl; bicyclo[2.2.1]hept-5-en-2-yl; (1R)-1-cyclohexylethyl; (1S)-1-cyclohexylethyl; 2-(1-cyclohexenyl)ethyl; 4-(2,2,6,6-tetramethyl)piperidyl; 2-(4-morpholinyl)ethyl; 1-naphthyl; 2-fluorophenyl; 3-chloro-2-methylphenyl; mesityl; 3,5-di(trifluoromethyl)phenyl; 2,6-dimethylphenyl, 4-(4-morpholinyl)phenyl; 2-methylphenyl; 2-isopropylphenyl; 2-methoxyphenyl; 2-indanyl; 4-chlorobenzyl; 4-methylbenzyl; (1R)-1-phenylethyl; (1S)-1-phenylethyl; 2-phenylethyl; (2R)-2-phenylpropyl; (2S)-2-phenylpropyl; 1-(4-chlorophenyl)cyclobutyl; 6-phenoxy-3-pyridyl; 2-(4-morpholinyl)ethyl; or R¹ and R² form together with the nitrogen atom bonded thereto azepan-1-yl;
R³ is hydrogen; methyl; ethyl; isopropyl; cyclohexyl; bromo; 1-hexahydroazepinyl; 4-morpholinyl; N-phthalimidyl; piperidin-1-yl; 4-methylpiperidin-1-yl; 1-(1,2,3,4-tetrahydroquinolinyl); 2-(1,2,3,4-tetrahydroisoquinolinyl); 8-methyl-1-(1,2,3,4-tetrahydroquinolinyl); 1-[7-(trifluoromethyl)-1,2,3,4-tetrahydroquinolinyl; 3,4-dihydroisoquinolin-2(1H)-yl; 6,7-dimethoxy-3,4-dihydroisoquinolin-2(1H)-yl; 4-benzylpiperidin-1-yl; azepan-1-yl; azocan-1-yl; 1-oxa-4-azaspiro[4.5]dec-4-yl; 2-decahydroisoquinolinyl; 1,4-diazepan-1-ium; 1,3-dihydro-2H-isoindol-2-yl; 2,3-dihydro-1H-indol-1-yl; pyrrolidin-1-yl; 3-pyridinyl; 3-indolyl; 1,3-benzoxazol-2-yl; 1,3-benzothiazol-2-yl; 1H-benzimidazol-2-yl; 4-hydroxy-4-phenylpiperidin-1-yl; 5-(2-chlorophenyl)-1,3,4-oxadiazol-2-yl; 4-chlorophenyl; 4-hydroxyphenyl; 3,4-dihydroxyphenyl;
or wherein R³ is NR⁴R⁵, wherein R⁴ and R⁵ are each independently selected from hydrogen; methyl; ethyl; n-propyl; isopropyl; n-butyl; cyclohexyl; cycloheptyl; (1R,2R,4S)-bicyclo[2.2.1]hept-2-yl; 4-methylcyclohexyl; cyclopropylmethyl; cyclohexylmethyl; cyclohexylcarbonyl; 1-adamantylcarbonyl; phenyl; 1-naphthyl; 4-bromophenyl; 2-chlorophenyl; 3-chlorophenyl; 4-chlorophenyl; 4-fluorophenyl; 2,6-difluorophenyl; 3-chloro-2-methylphenyl; 2-methylphenyl; 3-methylphenyl; 4-methylphenyl; 4-methoxyphenyl; 4-(trifluoromethoxy)phenyl; 2-benzoylphenyl; 3-carboxyphenyl; benzyl; 2-phenylethyl; 2-chloro-6-fluorobenzyl; 3-chloro-2-methylbenzyl; 2,2-dimethylpropionamido; phenoxyacetyl; 2-chlorobenzoyl; 2-fluorobenzoyl; 4-chlorobenzoyl; 2,5-difluorobenzoyl; 2,6-difluorobenzoyl; 2-chloro-6-fluorobenzoyl; 2,4-dichlorobenzoyl; 2,4,6-trichlorobenzoyl; 2-methoxybenzoyl; 4-methoxybenzoyl; 2-bromo-5-methoxybenzoyl; 2,4-dimethoxybenzoyl; 2,6-dimethoxybenzoyl; 4-(trifluoromethyl)benzoyl; 2-fluoro-4-(trifluoromethyl)benzoyl; 2,5-di(trifluoromethyl)benzoyl; 2-fluoro-5-(trifluoromethyl)benzoyl; 4-cyanobenzoyl; 2-chloro-6-fluorophenylacetyl; 2-chlorophenylsulfonyl; 2,6-difluorophenylsulfonyl; 2-chloro-3-pyridylcarbonyl; 2-furylcarbonyl; 2-thienylcarbonyl; 5-isoxazolylcarbonyl; 5-methyl-3-phenylisoxazol-4-ylcarbonyl; 2-thienylmethylcarbonyl; cyclopropylcarbonyl; cyclohexylcarbonyl; isopentanoyl; indazol-6-yl;
OCONR⁹R¹⁰, wherein R⁹ and R¹⁰ are each independently selected from 2-chlorophenyl; 4-bromo-2,6-difluorophenyl; 4-chloro-3-nitrophenyl; 3-phenoxyphenyl;
NHCONR¹¹R¹², wherein R¹¹ and R¹² are each independently selected from hydrogen, cyclopentyl, cyclohexyl, 2-chlorophenyl, 2-fluorophenyl, 4-fluorophenyl, 2,4-difluorophenyl, 2,6-difluorophenyl, 2-chloro-5-(trifluoromethyl)phenyl, 4-fluoro-2-(trifluoromethyl)phenyl, 2-methoxyphenyl, 2,4-dimethoxyphenyl, 5-chloro-2-methoxyphenyl, 2,6-dichloropyridin-4-yl;
OR¹³, wherein R¹³ is selected from hydrogen; phenyl; 2-chlorophenyl; 4-chloro-3-methylphenyl; 2-methoxyphenyl; 4-carbomethoxy-2-chlorophenyl; 3-(4-morpholinyl)phenyl; 4-phenoxyphenyl; 2-chlorobenzyl; 2-methylbenzyl; 2-methoxybenzyl; 3-(dimethylamino)benzyl; benzoyl; 2-chlorobenzoyl; 2,4-dichlorobenzoyl; 3,4-dichlorobenzoyl; 2,5-difluorobenzoyl; 2,6-difluorobenzoyl; 3,4-difluorobenzoyl; 2-chloro-6-fluorobenzoyl; 2,4,6-trichlorobenzoyl; 2,3,4-trifluorobenzoyl; 3-methylbenzoyl; 4-methylbenzoyl; 4-tert-butylbenzoyl; 3-methoxybenzoyl; 4-n-butoxybenzoyl; 2,4-dimethoxybenzoyl; 2,6-dimethoxybenzoyl; 2-bromo-5-methoxybenzoyl; 3-(trifluoromethyl)benzoyl; 2,5-di(trifluoromethyl)benzoyl; 3,5-di(trifluoromethyl)benzoyl; 2-fluoro-4-(trifluoromethyl)benzoyl; 2-fluoro-5-(trifluoromethyl)benzoyl; and 4-chloro-3-nitrobenzoyl.

4. A compound according to claim 1, which is selected from:
N-{2-[2-(bicyclo[2.2.1]hept-5-en-2-ylamino)-4-oxo-4,5-dihydro-1,3-thiazol-5-yl]ethyl}-2-chloro-6-fluorobenzamide,
N-{2-[2-(bicyclo[2.2.1]hept-5-en-2-ylamino)-4-oxo-4,5-dihydro-1,3-thiazol-5-yl]ethyl}-2,6-dimethoxybenzamide,
N-{2-[2-(bicyclo[2.2.1]hept-5-en-2-ylamino)-4-oxo-4,5-dihydro-1,3-thiazol-5-yl]ethyl}-2,4-dimethoxybenzamide,
N-{2-[2-(bicyclo[2.2.1]hept-5-en-2-ylamino)-4-oxo-4,5-dihydro-1,3-thiazol-5-yl]ethyl}-2,6-difluorobenzamide,
2-{2-[2-(bicyclo[2.2.1]hept-5-en-2-ylamino)-4-oxo-4,5-dihydro-1,3-thiazol-5-yl]ethyl}-1H-isoindole-1,3(2H)-dione,
N-{2-[2-(bicyclo[2.2.1]hept-5-en-2-ylamino)-4-oxo-4,5-dihydro-1,3-thiazol-5-yl]ethyl}-2,5-difluorobenzamide,
N-{2-[2-(bicyclo[2.2.1]hept-5-en-2-ylamino)-4-oxo-4,5-dihydro-1,3-thiazol-5-yl]ethyl}-2-chlorobenzamide,
N-{2-[2-(bicyclo[2.2.1]hept-5-en-2-ylamino)-4-oxo-4,5-dihydro-1,3-thiazol-5-yl]ethyl}-2-bromo-5-methoxybenzamide,
N-{2-[2-(bicyclo[2.2.1]hept-5-en-2-ylamino)-4-oxo-4,5-dihydro-1,3-thiazol-5-yl]ethyl}-2-fluoro-4-(trifluoromethyl)benzamide,
N-{2-[2-(bicyclo[2.2.1]hept-5-en-2-ylamino)-4-oxo-4,5-dihydro-1,3-thiazol-5-yl]ethyl}-2,4-dichlorobenzamide,
2-chloro-N-{2-[4-oxo-2-(tricyclo[3.3.1.0˜3,7˜]non-3-ylamino)-4,5-dihydro-1,3-thiazol-5-yl]ethyl}benzamide,
2,6-difluoro-N-{2-[4-oxo-2-(tricyclo[3.3.1.0˜3,7˜]non-3-ylamino)-4,5-dihydro-1,3-thiazol-5-yl]ethyl}benzamide,
2-chloro-6-fluoro-N-{2-[4-oxo-2-(tricyclo[3.3.1.0˜3,7˜]non-3-ylamino)-4,5-dihydro-1,3-thiazol-5-yl]ethyl}benzamide,
2,4-dichloro-N-{2-[4-oxo-2-(tricyclo[3.3.1.0˜3,7˜]non-3-ylamino)-4,5-dihydro-1,3-thiazol-5-yl]ethyl}benzamide,
2-chloro-N-(2-{2-[(cyclohexylmethyl)amino]-4-oxo-4,5-dihydro-1,3-thiazol-5-yl}ethyl)benzamide,
2-bromo-N-(2-{2-[(cyclohexylmethyl)amino]-4-oxo-4,5-dihydro-1,3-thiazol-5-yl}ethyl)-5-methoxybenzamide,
2,4-dichloro-N-(2-{2-[(cyclohexylmethyl)amino]-4-oxo-4,5-dihydro-1,3-thiazol-5-yl}ethyl)benzamide,
2-chloro-N-{2-[2-(cycloheptylamino)-4-oxo-4,5-dihydro-1,3-thiazol-5-yl]ethyl}benzamide,
N-{2-[2-(cycloheptylamino)-4-oxo-4,5-dihydro-1,3-thiazol-5-yl]ethyl}-2,6-difluorobenzamide,
N-{2-[2-(cycloheptylamino)-4-oxo-4,5-dihydro-1,3-thiazol-5-yl]ethyl}-2,6-dimethoxybenzamide,
2-bromo-N-{2-[2-(cycloheptylamino)-4-oxo-4,5-dihydro-1,3-thiazol-5-yl]ethyl}-5-methoxybenzamide,
2-chloro-N-{2-[2-(cycloheptylamino)-4-oxo-4,5-dihydro-1,3-thiazol-5-yl]ethyl}-6-fluorobenzamide,
2,4-dichloro-N-{2-[2-(cycloheptylamino)-4-oxo-4,5-dihydro-1,3-thiazol-5-yl]ethyl}benzamide,
N-{2-[2-(cycloheptylamino)-4-oxo-4,5-dihydro-1,3-thiazol-5-yl]ethyl}-2,5-difluorobenzamide,
N-{2-[2-(cycloheptylamino)-4-oxo-4,5-dihydro-1,3-thiazol-5-yl]ethyl}-2,5-bis(trifluoromethyl)benzamide,
N-{2-[2-(cycloheptylamino)-4-oxo-4,5-dihydro-1,3-thiazol-5-yl]ethyl}-2-fluoro-5-(trifluoromethyl)benzamide,
2-chloro-N-{2-[2-(cycloheptylamino)-4-oxo-4,5-dihydro-1,3-thiazol-5-yl]ethyl}nicotinamide,
N-{2-[2-(cycloheptylamino)-4-oxo-4,5-dihydro-1,3-thiazol-5-yl]ethyl}-2-furamide,
N-{2-[2-(cycloheptylamino)-4-oxo-4,5-dihydro-1,3-thiazol-5-yl]ethyl}thiophene-2-carboxamide,
N-{2-[2-(cycloheptylamino)-4-oxo-4,5-dihydro-1,3-thiazol-5-yl]ethyl}-2-(2-thienyl)acetamide,
N-{2-[2-(cycloheptylamino)-4-oxo-4,5-dihydro-1,3-thiazol-5-yl]ethyl}cyclopropanecarboxamide,
N-{2-[2-(cycloheptylamino)-4-oxo-4,5-dihydro-1,3-thiazol-5-yl]ethyl}-3-methylbutanamide,
N-{2-[2-(bicyclo[2.2.1]hept-5-en-2-ylamino)-4-oxo-4,5-dihydro-1,3-thiazol-5-yl]ethyl}cyclohexanecarboxamide,
N-{2-[2-(bicyclo[2.2.1]hept-5-en-2-ylamino)-4-oxo-4,5-dihydro-1,3-thiazol-5-yl]ethyl}isoxazole-5-carboxamide,
N-{2-[2-(bicyclo[2.2.1]hept-5-en-2-ylamino)-4-oxo-4,5-dihydro-1,3-thiazol-5-yl]ethyl}-2,4,6-trichlorobenzamide,
N-[(2-azepan-1-yl-4-oxo-4,5-dihydro-1,3-thiazol-5-yl)methyl]-2-fluorobenzamide,
2-(bicyclo[2.2.1]hept-5-en-2-ylamino)-5-{2-[methyl(phenyl)amino]ethyl}-1,3-thiazol-4(5H)-one,
2-(bicyclo[2.2.1]hept-5-en-2-ylamino)-5-[2-(2,3-dihydro-1H-indol-1-yl)ethyl]-1,3-thiazol-4(5H)-one,
2-(bicyclo[2.2.1]hept-5-en-2-ylamino)-5-[2-(3,4-dihydroquinolin-1(2H)-yl)ethyl]-1,3-thiazol-4(5H)-one,
2-(bicyclo[2.2.1]hept-5-en-2-ylamino)-5-[2-(1,3-dihydro-2H-isoindol-2-yl)ethyl]-1,3-thiazol-4(5H)-one,
2-(bicyclo[2.2.1]hept-5-en-2-ylamino)-5-(2-piperidin-1-ylethyl)-1,3-thiazol-4(5H)-one,
5-(2-Anilinoethyl)-2-{[(1R,2R,3R,5S)-2,6,6-trimethylbicyclo[3.1.1]hept-3-yl]amino}-1,3-thiazol-4(5H)-one hydrobromide,
5-(2-Anilinoethyl)-2-{[(1S,2S,3S,5R)-2,6,6-trimethylbicyclo[3.1.1]hept-3-yl]amino}-1,3-thiazol-4(5H)-one hydrobromide,
5-(2-Anilinoethyl)-2-(tricyclo[3.3.1.0˜3,7˜]non-3-ylamino)-1,3-thiazol-4(5H)-one,
5-(2-anilinoethyl)-2-(bicyclo[2.2.1]hept-2-ylamino)-1,3-thiazol-4(5H)-one,
5-(2-anilinoethyl)-2-[(2-cyclohex-1-en-1-ylethyl)amino]-1,3-thiazol-4(5H)-one,
5-(2-anilinoethyl)-2-[(1,1,3,3-tetramethylbutyl)amino]-1,3-thiazol-4(5H)-one hydrobromide,
5-(2-anilinoethyl)-2-[(cyclohexylmethyl)amino]-1,3-thiazol-4(5H)-one,
5-(2-anilinoethyl)-2-[(2,2,6,6-tetramethylpiperidin-4-yl)amino]-1,3-thiazol-4(5H)-one,
5-(2-anilinoethyl)-2-{[(1R)-1-phenylethyl]amino}-1,3-thiazol-4(5H)-one hydrochloride,
5-(2-anilinoethyl)-2-{[(1S)-1-phenylethyl]amino}-1,3-thiazol-4(5H)-one hydrochloride,
5-(2-anilinoethyl)-2-{[(2R)-2-phenylpropyl]amino}-1,3-thiazol-4(5H)-one hydrochloride,
5-(2-anilinoethyl)-2-(cycloheptylamino)-1,3-thiazol-4(5H)-one,
5-(2-anilinoethyl)-2-[(4-methylbenzyl)amino]-1,3-thiazol-4(5H)-one,
5-(2-anilinoethyl)-2-(cyclooctylamino)-1,3-thiazol-4(5H)-one hydrobromide,
5-(2-anilinoethyl)-2-{[(1R)-1-cyclohexylethyl]amino}-1,3-thiazol-4(5H)-one,
5-(2-anilinoethyl)-2-{[(1S)-1-cyclohexylethyl]amino}-1,3-thiazol-4(5H)-one,
5-(2-anilinoethyl)-2-azepan-1-yl-1,3-thiazol-4(5H)-one,
5-(2-anilinoethyl)-2-{[(2S)-2-phenylpropyl]amino}-1,3-thiazol-4(5H)-one,
2-[(cyclohexylmethyl)amino]-5-{2-[(4-fluorophenyl)amino]ethyl}-1,3-thiazol-4(5H)-one trifluoroacetate,
2-(bicyclo[2.2.1]hept-5-en-2-ylamino)-5-[2-(3,4-dihydroquinolin-1(2H)-yl)-2-oxoethyl]-1,3-thiazol-4(5H)-one,
2-[2-(Bicyclo[2.2.1]hept-5-en-2-ylamino)-4-oxo-4,5-dihydro-1,3-thiazol-5-yl]-N-(3-chloro-2-methylphenyl)acetamide,
N-Benzyl-2-[2-(bicyclo[2.2.1]hept-5-en-2-ylamino)-4-oxo-4,5-dihydro-1,3-thiazol-5-yl]acetamide,
N-benzyl-2-[2-(bicyclo[2.2.1]hept-5-en-2-ylamino)-4-oxo-4,5-dihydro-1,3-thiazol-5-yl]-N-phenylacetamide,
2-[2-(bicyclo[2.2.1]hept-5-en-2-ylamino)-4-oxo-4,5-dihydro-1,3-thiazol-5-yl]-N-(4-methoxyphenyl)-N-methylacetamide,
2-[2-(bicyclo[2.2.1]hept-5-en-2-ylamino)-4-oxo-4,5-dihydro-1,3-thiazol-5-yl]-N-methyl-N-phenylacetamide,
2-(bicyclo[2.2.1]hept-5-en-2-ylamino)-5-[2-(1,3-dihydro-2H-isoindol-2-yl)-2-oxoethyl]-1,3-thiazol-4(5H)-one,
2-(bicyclo[2.2.1]hept-5-en-2-ylamino)-5-[2-(2,3-dihydro-1H-indol-1-yl)-2-oxoethyl]-1,3-thiazol-4(5H)-one,
2-[2-(bicyclo[2.2.1]hept-5-en-2-ylamino)-4-oxo-4,5-dihydro-1,3-thiazol-5-yl]-N-ethyl-N-(3-methylphenyl)acetamide,
2-(bicyclo[2.2.1]hept-5-en-2-ylamino)-5-[2-(3,4-dihydroisoquinolin-2(1H)-yl)-2-oxoethyl]-1,3-thiazol-4(5H)-one,
2-[2-(bicyclo[2.2.1]hept-5-en-2-ylamino)-4-oxo-4,5-dihydro-1,3-thiazol-5-yl]-N-methyl-N-[4-(trifluoromethoxy)phenyl]acetamide,
2-[2-(bicyclo[2.2.1]hept-5-en-2-ylamino)-4-oxo-4,5-dihydro-1,3-thiazol-5-yl]-N-ethyl-N-[4-(trifluoromethoxy)phenyl]acetamide,
2-(bicyclo[2.2.1]hept-5-en-2-ylamino)-5-{2-oxo-2-[7-(trifluoromethyl)-3,4-dihydroquinolin-1(2H)-yl]ethyl}-1,3-thiazol-4(5H)-one,
2-[2-(bicyclo[2.2.1]hept-5-en-2-ylamino)-4-oxo-4,5-dihydro-1,3-thiazol-5-yl]-N-methyl-N-(2-methylphenyl)acetamide,
2-[2-(bicyclo[2.2.1]hept-5-en-2-ylamino)-4-oxo-4,5-dihydro-1,3-thiazol-5-yl]-N-ethyl-N-phenylacetamide,
2-[2-(bicyclo[2.2.1]hept-5-en-2-ylamino)-4-oxo-4,5-dihydro-1,3-thiazol-5-yl]-N-methyl-N-(4-methylphenyl)acetamide,
2-[2-(bicyclo[2.2.1]hept-5-en-2-ylamino)-4-oxo-4,5-dihydro-1,3-thiazol-5-yl]-N-(4-bromophenyl)-N-methylacetamide,
2-[2-(bicyclo[2.2.1]hept-5-en-2-ylamino)-4-oxo-4,5-dihydro-1,3-thiazol-5-yl]-N-(4-chlorophenyl)-N-methylacetamide,
2-[2-(bicyclo[2.2.1]hept-5-en-2-ylamino)-4-oxo-4,5-dihydro-1,3-thiazol-5-yl]-N-(4-fluorophenyl)-N-methylacetamide,
2-[2-(bicyclo[2.2.1]hept-5-en-2-ylamino)-4-oxo-4,5-dihydro-1,3-thiazol-5-yl]-N-(3-chlorophenyl)-N-methylacetamide,
2-[2-(bicyclo[2.2.1]hept-5-en-2-ylamino)-4-oxo-4,5-dihydro-1,3-thiazol-5-yl]-N-ethyl-N-(2-methylphenyl)acetamide,
2-(bicyclo[2.2.1]hept-5-en-2-ylamino)-5-[2-(8-methyl-3,4-dihydroquinolin-1(2H)-yl)-2-oxoethyl]-1,3-thiazol-4(5H)-one,
2-(bicyclo[2.2.1]hept-5-en-2-ylamino)-5-(2-oxo-2-piperidin-1-ylethyl)-1,3-thiazol-4(5H)-one,
2-[2-(bicyclo[2.2.1]hept-5-en-2-ylamino)-4-oxo-4,5-dihydro-1,3-thiazol-5-yl]-N-isopropyl-N-phenylacetamide,
2-[2-(bicyclo[2.2.1]hept-5-en-2-ylamino)-4-oxo-4,5-dihydro-1,3-thiazol-5-yl]-N-(2,6-difluorophenyl)acetamide,
2-[2-(Bicyclo[2.2.1]hept-5-en-2-ylamino)-4-oxo-4,5-dihydro-1,3-thiazol-5-yl]-N-phenylacetamide,
2-[2-(Bicyclo[2.2.1]hept-5-en-2-ylamino)-4-oxo-4,5-dihydro-1,3-thiazol-5-yl]-N-1H-indazol-6-ylacetamide,
2-[2-(Bicyclo[2.2.1]hept-5-en-2-ylamino)-4-oxo-4,5-dihydro-1,3-thiazol-5-yl]-N-(4-fluorophenyl)acetamide,
N-(2-benzoylphenyl)-2-[2-(Bicyclo[2.2.1]hept-5-en-2-ylamino)-4-oxo-4,5-dihydro-1,3-thiazol-5-yl]acetamide,
3-({[2-(Bicyclo[2.2.1]hept-5-en-2-ylamino)-4-oxo-4,5-dihydro-1,3-thiazol-5-yl]acetyl}amino)benzoic acid,
2-[2-(Bicyclo[2.2.1]hept-5-en-2-ylamino)-4-oxo-4,5-dihydro-1,3-thiazol-5-yl]-N-ethylacetamide,
2-[2-(Bicyclo[2.2.1]hept-5-en-2-ylamino)-4-oxo-4,5-dihydro-1,3-thiazol-5-yl]-N-methylacetamide,
2-(Bicyclo[2.2.1]hept-5-en-2-ylamino)-5-(2-morpholin-4-yl-2-oxoethyl)-1,3-thiazol-4(5H)-one,
2-[2-(Bicyclo[2.2.1]hept-5-en-2-ylamino)-4-oxo-4,5-dihydro-1,3-thiazol-5-yl]-N-(2-chlorophenyl)-N-methylacetamide,
2-(Bicyclo[2.2.1]hept-2-ylamino)-5-[2-(3,4-dihydroquinolin-1(2H)-yl)-2-oxoethyl]-1,3-thiazol-4(5H)-one,
2-[(3-chloro-2-methylphenyl)amino]-5-[2-(3,4-dihydroquinolin-1(2H)-yl)-2-oxoethyl]-1,3-thiazol-4(5H)-one,
2-[2-(1-Adamantylamino)-4-oxo-4,5-dihydro-1,3-thiazol-5-yl]-N-methyl-N-phenylacetamide,
2-[2-(1-adamantylamino)-4-oxo-4,5-dihydro-1,3-thiazol-5-yl]-N-(2,6-difluorophenyl)acetamide,
2-[2-(tert-butylamino)-4-oxo-4,5-dihydro-1,3-thiazol-5-yl]-N-methyl-N-phenylacetamide,
2-[2-(cyclopropylamino)-4-oxo-4,5-dihydro-1,3-thiazol-5-yl]-N-methyl-N-phenylacetamide,
2-(cyclopentylamino)-5-[2-(3,4-dihydroisoquinolin-2(1H)-yl)-2-oxoethyl]-1,3-thiazol-4(5H)-one,
2-[2-(cyclopentylamino)-4-oxo-4,5-dihydro-1,3-thiazol-5-yl]-N-methyl-N-phenylacetamide,
5-[2-(3,4-dihydroisoquinolin-2(1H)-yl)-2-oxoethyl]-2-(isobutylamino)-1,3-thiazol-4(5H)-one,
2-[2-(isobutylamino)-4-oxo-4,5-dihydro-1,3-thiazol-5-yl]-N-methyl-N-phenylacetamide,
2-(1-adamantylamino)-5-[2-(3,4-dihydroquinolin-1(2H)-yl)-2-oxoethyl]-1,3-thiazol-4(5H)-one,
2-(cyclopropylamino)-5-[2-(3,4-dihydroisoquinolin-2(1H)-yl)-2-oxoethyl]-1,3-thiazol-4(5H)-one,
5-[2-(3,4-dihydroquinolin-1(2H)-yl)-2-oxoethyl]-2-(mesitylamino)-1,3-thiazol-4(5H)-one,
N-(2-chlorophenyl)-2-{2-[(2-methylbutyl)amino]-4-oxo-4,5-dihydro-1,3-thiazol-5-yl}acetamide,
N-methyl-2-{2-[(2-methylbutyl)amino]-4-oxo-4,5-dihydro-1,3-thiazol-5-yl}-N-phenylacetamide,
5-[2-(3,4-dihydroquinolin-1(2H)-yl)-2-oxoethyl]-2-[(2-methylbutyl)amino]-1,3-thiazol-4(5H)-one,
N-methyl-2-{4-oxo-2-[(2-phenylethyl)amino]-4,5-dihydro-1,3-thiazol-5-yl}-N-phenylacetamide,
5-[2-(3,4-dihydroquinolin-1(2H)-yl)-2-oxoethyl]-2-[(2-phenylethyl)amino]-1,3-thiazol-4(5H)-one,
2-(2-{[3,5-Bis(trifluoromethyl)phenyl]amino}-4-oxo-4,5-dihydro-1,3-thiazol-5-yl)-N-(2-chloro-6-fluorobenzyl)acetamide trifluoroacetate,
5-[2-(3,4-dihydroquinolin-1(2H)-yl)-2-oxoethyl]-2-[(4-morpholin-4-ylphenyl)amino]-1,3-thiazol-4(5H)-one,
5-[2-(3,4-Dihydroquinolin-1(2H)-yl)-2-oxoethyl]-2-{[3,5-bis(trifluoromethyl)phenyl]amino}-1,3-thiazol-4(5H)-one,
2-(2-{[3,5-Bis(trifluoromethyl)phenyl]amino}-4-oxo-4,5-dihydro-1,3-thiazol-5-yl)-N-(2-phenylethyl)acetamide,
1-[2-(3,4-Dihydroquinolin-1(2H)-yl)-2-oxoethyl]-2-[(2-fluorophenyl)amino]-1,3-thiazol-4(5H)-one,
N-(2-Chloro-6-fluorobenzyl)-2-{2-[(2-fluorophenyl)amino]-4-oxo-4,5-dihydro-1,3-thiazol-5-yl}acetamide,
2-{2-[(2-Fluorophenyl)amino]-4-oxo-4,5-dihydro-1,3-thiazol-5-yl}-N-(4-methylcyclohexyl)acetamide,
5-[2-(3,4-dihydroquinolin-1(2H)-yl)-2-oxoethyl]-2-[(2,6-dimethylphenyl)amino]-1,3-thiazol-4(5H)-one,
N-(2-chloro-6-fluorobenzyl)-2-{2-[(2,6-dimethylphenyl)amino]-4-oxo-4,5-dihydro-1,3-thiazol-5-yl}acetamide,
2-[2-(3,4-Dihydroquinolin-1(2H)-yl)-2-oxoethyl]-2-[(2-methylphenyl)amino]-1,3-thiazol-4(5H)-one,
5-(2-Azepan-1-yl-2-oxoethyl)-2-[(2-methylphenyl)amino]-1,3-thiazol-4(5H)-one,
N-(2-Chloro-6-fluorobenzyl)-2-{2-[(2-methylphenyl)amino]-4-oxo-4,5-dihydro-1,3-thiazol-5-yl}acetamide,
3-(2-Azepan-1-yl-2-oxoethyl)-2-[(2-isopropylphenyl)amino]-1,3-thiazol-4(5H)-one,
N-benzyl-2-{2-[(cyclohexylmethyl)amino]-4-oxo-4,5-dihydro-1,3-thiazol-5-yl}acetamide,
N-methyl-2-{4-oxo-2-[(6-phenoxypyridin-3-yl)amino]4,5-dihydro-1,3-thiazol-5-yl}-N-phenylacetamide,
N-(2-chloro-6-fluorobenzyl)-2-{4-oxo-2-[(6-phenoxypyridin-3-yl)amino]-4,5-dihydro-1,3-thiazol-5-yl}acetamide,
2-[(2-cyclohex-1-en-1-ylethyl)amino]-5-[2-(3,4-dihydroquinolin-1(2H)-yl)-2-oxoethyl]-1,3-thiazol-4(5H)-one,
N-(4-fluorophenyl)-2-{4-oxo-2-[(1,1,3,3-tetramethylbutyl)amino]-4,5-dihydro-1,3-thiazol-5-yl}acetamide,
5-[2-(3,4-dihydroquinolin-1(2H)-yl)-2-oxoethyl]-2-[(1,1,3,3-tetramethylbutyl)amino]-1,3-thiazol-4(5H)-one,
5-(2-azepan-1-yl-2-oxoethyl)-2-[(1,1,3,3-tetramethylbutyl)amino]-1,3-thiazol-4(5H)-one,
5-(2-Azepan-1-yl-2-oxoethyl)-2-(tricyclo[3.3.1.0˜3,7˜]non-3-ylamino)-1,3-thiazol-4(5H)-one,
2-[2-(cyclopentylamino)-4-oxo-4,5-dihydro-1,3-thiazol-5-yl]-N-(2,6-difluorophenyl)acetamide,
2-{2-[(4-chlorobenzyl)amino]-4-oxo-4,5-dihydro-1,3-thiazol-5-yl}-N-1-naphthylacetamide,
5-[2-(3,4-dihydroquinolin-1(2H)-yl)-2-oxoethyl]-2-[(2-morpholin-4-ylethyl)amino]-1,3-thiazol-4(5H)-one,
2-[2-(sec-butylamino)-4-oxo-4,5-dihydro-1,3-thiazol-5-yl]-N-methyl-N-phenylacetamide trifluoroacetate,
2-(sec-butylamino)-5-[2-(3,4-dihydroquinolin-1(2H)-yl)-2-oxoethyl]-1,3-thiazol-4(5H)-one trifluoroacetate,
2-[2-(sec-butylamino)-4-oxo-4,5-dihydro-1,3-thiazol-5-yl]-N-(2-chlorophenyl)acetamide trifluoroacetate,
2-{2-[(cyclopropylmethyl)amino]-4-oxo-4,5-dihydro-1,3-thiazol-5-yl}-N-methyl-N-phenylacetamide trifluoroacetate,
2-{2-[(4-methylbenzyl)amino]-4-oxo-4,5-dihydro-1,3-thiazol-5-yl}-N-phenylacetamide,
2-{2-[(4-chlorobenzyl)amino]-4-oxo-4,5-dihydro-1,3-thiazol-5-yl}-N-phenylacetamide,
5-[2-(3,4-Dihydroisoquinolin-2(1H)-yl)-2-oxoethyl]-2-(tricyclo[3.3.1.0˜3,7˜]non-3-ylamino)-1,3-thiazol-4(5H)-one,
2-(cycloheptylamino)-5-[2-(3,4-dihydroisoquinolin-2(1H)-yl)-2-oxoethyl]-1,3-thiazol-4(5H)-one,
5-(2-azepan-1-yl-2-oxoethyl)-2-(cycloheptylamino)-1,3-thiazol-4(5H)-one,
5-(2-azepan-1-yl-2-oxoethyl)-2-[(cyclohexylmethyl)amino]-1,3-thiazol-4(5H)-one,
2-[2-(cycloheptylamino)-4-oxo-4,5-dihydro-1,3-thiazol-5-yl]-N-methyl-N-phenylacetamide,
5-[2-(4-Methylpiperidin-1-yl)-2-oxoethyl]-2-(tricyclo[3.3.1.0˜3,7˜]non-3-ylamino)-1,3-thiazol-4(5H)-one,
5-[2-(1,3-Dihydro-2H-isoindol-2-yl)-2-oxoethyl]-2-(tricyclo[3.3.1.0˜3,7˜]non-3-ylamino)-1,3-thiazol-4(5H)-one,
2-[(cyclohexylmethyl)amino]-5-(2-oxo-2-pyrrolidin-1-ylethyl)-1,3-thiazol-4(5H)-one,
2-[(cyclohexylmethyl)amino]-5-[2-(3,4-dihydroisoquinolin-2(1H)-yl)-2-oxoethyl]-1,3-thiazol-4(5H)-one,
2-azepan-1-yl-5-(2-azepan-1-yl-2-oxoethyl)-1,3-thiazol-4(5H)-one,
2-azepan-1-yl-5-[2-(3,4-dihydroquinolin-1(2H)-yl)-2-oxoethyl]-1,3-thiazol-4(5H)-one,
2-azepan-1-yl-5-[²-(3,4-dihydroisoquinolin-2(1H)-yl)-2-oxoethyl]-1,3-thiazol-4(5H)-one,
2-(cycloheptylamino)-5-[2-(2,3-dihydro-1H-indol-1-yl)-2-oxoethyl]-1,3-thiazol-4(5H)-one,
2-(cycloheptylamino)-5-(2-oxo-2-pyrrolidin-1-ylethyl)-1,3-thiazol-4(5H)-one,
2-(cycloheptylamino)-5-[2-(4-methylpiperidin-1-yl)-2-oxoethyl]-1,3-thiazol-4(5H)-one,
N-cyclohexyl-2-{2-[(cyclohexylmethyl)amino]-4-oxo-4,5-dihydro-1,3-thiazol-5-yl}acetamide,
N-Cyclohexyl-N-ethyl-2-[4-oxo-2-(tricyclo[3.3.1.0˜3,7˜]non-3-ylamino)-4,5-dihydro-1,3-thiazol-5-yl]acetamide,
N-(Cyclopropylmethyl)-N-propylacetamide-2-[4-oxo-2-(tricyclo[3.3.1.0˜3,7˜]non-3-ylamino)-4,5-dihydro-1,3-thiazol-5-yl]acetamide,
5-(2-Azocan-1-yl-2-oxoethyl)-2-(tricyclo[3.3.1.0˜3,7˜]non-3-ylamino)-1,3-thiazol-4(5H)-one,
5-[2-(1-Oxa-4-azaspiro[4.5]dec-4-yl)-2-oxoethyl]-2-(tricyclo[3.3.1.0˜3,7˜]non-3-ylamino)-1,3-thiazol-4(5H)-one,
5-{[(1R)-1-cyclohexylethyl]amino}-5-[2-(3,4-dihydroquinolin-1(2H)-yl)-2-oxoethyl]-1,3-thiazol-4(5H)-one,
2-{[(1R)-1-cyclohexylethyl]amino}-5-[2-(3,4-dihydroisoquinolin-2(1H)-yl)-2-oxoethyl]-1,3-thiazol-4(5H)-one,
5-(2-azepan-1-yl-2-oxoethyl)-2-{[(1R)-1-cyclohexylethyl]amino}-1,3-thiazol-4(5H)-one,
2-{[(1S)-1-cyclohexylethyl]amino}-5-[2-(3,4-dihydroquinolin-1(2H)-yl)-2-oxoethyl]-1,3-thiazol-4(5H)-one,
2-{[(1S)-1-cyclohexylethyl]amino}-5-[2-(3,4-dihydroisoquinolin-2(1H)-yl)-2-oxoethyl]-1,3-thiazol-4(5H)-one,
1-(2-azepan-1-yl-2-oxoethyl)-2-{[(1S)-1-cyclohexylethyl]amino}-1,3-thiazol-4(5H)-one,
2-[(cyclohexylmethyl)amino]-5-[2-(4-methylpiperidin-1-yl)-2-oxoethyl]-1,3-thiazol-4(5H)-one,
N-cyclohexyl-2-{2-[(cyclohexylmethyl)amino]-4-oxo-4,5-dihydro-1,3-thiazol-5-yl}-N-ethylacetamide,
2-[(cyclohexylmethyl)amino]-5-[2-(1-oxa-4-azaspiro[4.5]dec-4-yl)-2-oxoethyl]-1,3-thiazol-4(5H)-one,
5-(2-azocan-1-yl-2-oxoethyl)-2-[(cyclohexylmethyl)amino]-1,3-thiazol-4(5H)-one,
2-[(cyclohexylmethyl)amino]-5-[2-(1,3-dihydro-2H-isoindol-2-yl)-2-oxoethyl]-1,3-thiazol-4(5H)-one,
N-(3-chloro-2-methylbenzyl)-2-{2-[(cyclohexylmethyl)amino]-4-oxo-4,5-dihydro-1,3-thiazol-5-yl}acetamide,
N-(cyclohexylmethyl)-2-{2-[(cyclohexylmethyl)amino]-4-oxo-4,5-dihydro-1,3-thiazol-5-yl}acetamide,
3-[(cyclohexylmethyl)amino]-5-[2-(octahydroisoquinolin-2(1H)-yl)-2-oxoethyl]-1,3-thiazol-4(5H)-one,
N-[(1R,2R,4S)-bicyclo[2.2.1]hept-2-yl]-2-{2-[(cyclohexylmethyl)amino]-4-oxo-4,5-dihydro-1,3-thiazol-5-yl}acetamide,
4-{[2-(cycloheptylamino)-4-oxo-4,5-dihydro-1,3-thiazol-5-yl]acetyl}-1,4-diazepan-1-ium trifluoroacetate,
2-[2-(cycloheptylamino)-4-oxo-4,5-dihydro-1,3-thiazol-5-yl]-N-(cyclopropylmethyl)-N-propylacetamide,
2-(cycloheptylamino)-5-[2-(1,3-dihydro-2H-isoindol-2-yl)-2-oxoethyl]-1,3-thiazol-4(5H)-one,
5-(2-azocan-1-yl-2-oxoethyl)-2-(cycloheptylamino)-1,3-thiazol-4(5H)-one,
2-[2-(cycloheptylamino)-4-oxo-4,5-dihydro-1,3-thiazol-5-yl]-N-cyclohexyl-N-ethylacetamide,
2-{2-[(cyclohexylmethyl)amino]-4-oxo-4,5-dihydro-1,3-thiazol-5-yl}-N-methyl-N-phenylacetamide,
2-{2-[(cyclohexylmethyl)amino]-4-oxo-4,5-dihydro-1,3-thiazol-5-yl}-N-(4-methoxyphenyl)-N-methylacetamide,
2-{2-[(cyclohexylmethyl)amino]-4-oxo-4,5-dihydro-1,3-thiazol-5-yl}-N-ethyl-N-phenylacetamide,
N-butyl-2-{2-[(cyclohexylmethyl)amino]-4-oxo-4,5-dihydro-1,3-thiazol-5-yl}-N-phenylacetamide,
N-butyl-2-[2-(cycloheptylamino)-4-oxo-4,5-dihydro-1,3-thiazol-5-yl]-N-phenylacetamide,
N-benzyl-2-[2-(cycloheptylamino)-4-oxo-4,5-dihydro-1,3-thiazol-5-yl]-N-phenylacetamide,
5-[2-(1,3-dihydro-2H-isoindol-2-yl)-2-oxoethyl]-2-[(2,2,3,3-tetramethylcyclopropyl)amino]-1,3-thiazol-4(5H)-one,
5-(2-azepan-1-yl-2-oxoethyl)-2-[(2,2,3,3-tetramethylcyclopropyl)amino]-1,3-thiazol-4(5H)-one,
2-[2-(bicyclo[2.2.1]hept-5-en-2-ylamino)-4-oxo-4,5-dihydro-1,3-thiazol-5-yl]ethyl benzoate,
2-[2-(bicyclo[2.2.1]hept-5-en-2-ylamino)-4-oxo-4,5-dihydro-1,3-thiazol-5-yl]ethyl 2-chlorobenzoate,
2-[2-(bicyclo[2.2.1]hept-5-en-2-ylamino)-4-oxo-4,5-dihydro-1,3-thiazol-5-yl]ethyl 3,4-dichlorobenzoate,
2-[2-(bicyclo[2.2.1]hept-5-en-2-ylamino)-4-oxo-4,5-dihydro-1,3-thiazol-5-yl]ethyl 2,6-difluorobenzoate,
2-[2-(bicyclo[2.2.1]hept-5-en-2-ylamino)-4-oxo-4,5-dihydro-1,3-thiazol-5-yl]ethyl 2,5-bis(trifluoromethyl)benzoate,
2-[2-(bicyclo[2.2.1]hept-5-en-2-ylamino)-4-oxo-4,5-dihydro-1,3-thiazol-5-yl]ethyl 3,4-difluorobenzoate trifluoroacetate,
2-[2-(bicyclo[2.2.1]hept-5-en-2-ylamino)-4-oxo-4,5-dihydro-1,3-thiazol-5-yl]ethyl 3,4-difluorobenzoate,
2-[2-(bicyclo[2.2.1]hept-5-en-2-ylamino)-4-oxo-4,5-dihydro-1,3-thiazol-5-yl]ethyl 2,5-difluorobenzoate,
2-[2-(bicyclo[2.2.1]hept-5-en-2-ylamino)-4-oxo-4,5-dihydro-1,3-thiazol-5-yl]ethyl 4-methylbenzoate,
2-[2-(bicyclo[2.2.1]hept-5-en-2-ylamino)-4-oxo-4,5-dihydro-1,3-thiazol-5-yl]ethyl 4-chloro-3-nitrobenzoate,
2-[2-(bicyclo[2.2.1]hept-5-en-2-ylamino)-4-oxo-4,5-dihydro-1,3-thiazol-5-yl]ethyl 3-methylbenzoate,
2-[2-(bicyclo[2.2.1]hept-5-en-2-ylamino)-4-oxo-4,5-dihydro-1,3-thiazol-5-yl]ethyl 3-(trifluoromethyl)benzoate,
2-[2-(bicyclo[2.2.1]hept-5-en-2-ylamino)-4-oxo-4,5-dihydro-1,3-thiazol-5-yl]ethyl 2,3,4-trifluorobenzoate,
2-[2-(bicyclo[2.2.1]hept-5-en-2-ylamino)-4-oxo-4,5-dihydro-1,3-thiazol-5-yl]ethyl 2-bromo-5-methoxybenzoate,
2-[2-(bicyclo[2.2.1]hept-5-en-2-ylamino)-4-oxo-4,5-dihydro-1,3-thiazol-5-yl]ethyl 2-chloro-6-fluorobenzoate,
2-[2-(bicyclo[2.2.1]hept-5-en-2-ylamino)-4-oxo-4,5-dihydro-1,3-thiazol-5-yl]ethyl 2-fluoro-5-(trifluoromethyl)benzoate,
2-[2-(bicyclo[2.2.1]hept-5-en-2-ylamino)-4-oxo-4,5-dihydro-1,3-thiazol-5-yl]ethyl 2-fluoro-4-(trifluoromethyl)benzoate,
2-[2-(bicyclo[2.2.1]hept-5-en-2-ylamino)-4-oxo-4,5-dihydro-1,3-thiazol-5-yl]ethyl 3-methoxybenzoate,
2-[2-(bicyclo[2.2.1]hept-5-en-2-ylamino)-4-oxo-4,5-dihydro-1,3-thiazol-5-yl]ethyl 2,6-dimethoxybenzoate,
2-[2-(bicyclo[2.2.1]hept-5-en-2-ylamino)-4-oxo-4,5-dihydro-1,3-thiazol-5-yl]ethyl 2,4-dimethoxybenzoate,
2-[2-(bicyclo[2.2.1]hept-5-en-2-ylamino)-4-oxo-4,5-dihydro-1,3-thiazol-5-yl]ethyl 4-butoxybenzoate,
2-[2-(bicyclo[2.2.1]hept-5-en-2-ylamino)-4-oxo-4,5-dihydro-1,3-thiazol-5-yl]ethyl 3,5-bis(trifluoromethyl)benzoate,
2-[2-(bicyclo[2.2.1]hept-5-en-2-ylamino)-4-oxo-4,5-dihydro-1,3-thiazol-5-yl]ethyl 4-tert-butylbenzoate,
2-[2-(bicyclo[2.2.1]hept-5-en-2-ylamino)-4-oxo-4,5-dihydro-1,3-thiazol-5-yl]ethyl 2,4-dichlorobenzoate,
2-[2-(bicyclo[2.2.1]hept-5-en-2-ylamino)-4-oxo-4,5-dihydro-1,3-thiazol-5-yl]ethyl 2,4,6-trichlorobenzoate,
2-[2-(bicyclo[2.2.1]hept-5-en-2-ylamino)-4-oxo-4,5-dihydro-1,3-thiazol-5-yl]ethyl (2-chlorophenyl)carbamate,
2-[2-(bicyclo[2.2.1]hept-5-en-2-ylamino)-4-oxo-4,5-dihydro-1,3-thiazol-5-yl]ethyl (4-chloro-3-nitrophenyl)carbamate,
2-[2-(bicyclo[2.2.1]hept-5-en-2-ylamino)-4-oxo-4,5-dihydro-1,3-thiazol-5-yl]ethyl (4-bromo-2,6-difluorophenyl)carbamate,
2-[2-(bicyclo[2.2.1]hept-5-en-2-ylamino)-4-oxo-4,5-dihydro-1,3-thiazol-5-yl]ethyl (3-phenoxyphenyl)carbamate,
N-{2-[2-(cycloheptylamino)-4-oxo-4,5-dihydro-1,3-thiazol-5-yl]ethyl}-N′-(2-fluorophenyl)urea,
N-{2-[2-(cycloheptylamino)-4-oxo-4,5-dihydro-1,3-thiazol-5-yl]ethyl}-N′-(4-fluorophenyl)urea,
N-{2-[2-(cycloheptylamino)-4-oxo-4,5-dihydro-1,3-thiazol-5-yl]ethyl}-N′-(2,6-difluorophenyl)urea,
N-{2-[2-(cycloheptylamino)-4-oxo-4,5-dihydro-1,3-thiazol-5-yl]ethyl}-N′-(2,4-difluorophenyl)urea,
N-(2-chlorophenyl)-N′-{2-[2-(cycloheptylamino)-4-oxo-4,5-dihydro-1,3-thiazol-5-yl]ethyl}urea,
N-[2-chloro-5-(trifluoromethyl)phenyl]-N′-{2-[2-(cycloheptylamino)-4-oxo-4,5-dihydro-1,3-thiazol-5-yl]ethyl}urea,
N-{2-[2-(cycloheptylamino)-4-oxo-4,5-dihydro-1,3-thiazol-5-yl]ethyl}-N′-[4-fluoro-2-(trifluoromethyl)phenyl]urea,
N-{2-[2-(cycloheptylamino)-4-oxo-4,5-dihydro-1,3-thiazol-5-yl]ethyl}-N′-(2-methoxyphenyl)urea,
N-(5-chloro-2-methoxyphenyl)-N′-{2-[2-(cycloheptylamino)-4-oxo-4,5-dihydro-1,3-thiazol-5-yl]ethyl}urea,
N-{2-[2-(cycloheptylamino)-4-oxo-4,5-dihydro-1,3-thiazol-5-yl]ethyl}-N′-(2,4-dimethoxyphenyl)urea,
N-{2-[2-(cycloheptylamino)-4-oxo-4,5-dihydro-1,3-thiazol-5-yl]ethyl}-N′-(2,6-dichloropyridin-4-yl)urea,
N-{2-[2-(cycloheptylamino)-4-oxo-4,5-dihydro-1,3-thiazol-5-yl]ethyl}-N′-cyclohexylurea,
N-{2-[2-(cycloheptylamino)-4-oxo-4,5-dihydro-1,3-thiazol-5-yl]ethyl}-N′-cyclopentylurea,
2-(bicyclo[2.2.1]hept-5-en-2-ylamino)-5-{2-[(2-chlorobenzyl)oxy]ethyl}-1,3-thiazol-4(5H)-one,
2-(bicyclo[2.2.1]hept-5-en-2-ylamino)-5-{2-[(2-methylbenzyl)oxy]ethyl}-1,3-thiazol-4(5H)-one trifluoroacetate,
2-(bicyclo[2.2.1]hept-5-en-2-ylamino)-5-{2-[(2-methoxybenzyl)oxy]ethyl}-1,3-thiazol-4(5H)-one,
2-(bicyclo[2.2.1]hept-5-en-2-ylamino)-5-(2-{[3-(dimethylamino)benzyl]oxy}ethyl)-1,3-thiazol-4(5H)-one,
2-(Bicyclo[2.2.1]hept-5-en-2-ylamino)-5-[2-(2-chlorophenoxy)ethyl]-1,3-thiazol-4(5H)-one,
2-(bicyclo[2.2.1]hept-5-en-2-ylamino)-5-(2-hydroxyethyl)-1,3-thiazol-4(5H)-one,
2-(Bicyclo[2.2.1]hept-5-en-2-ylamino)-5-[2-(4-phenoxyphenoxy)ethyl]-1,3-thiazol-4(5H)-one,
Methyl 4-{2-[2-(bicyclo[2.2.1]hept-5-en-2-ylamino)-4-oxo-4,5-dihydro-1,3-thiazol-5-yl]ethoxy}-3-chlorobenzoate,
2-(Bicyclo[2.2.1]hept-5-en-2-ylamino)-5-[2-(4-chloro-3-methylphenoxy)ethyl]-1,3-thiazol-4(5H)-one,
2-Chlorophenyl [2-(bicyclo[2.2.1]hept-5-en-2-ylamino)-4-oxo-4,5-dihydro-1,3-thiazol-5-yl]acetate,
Phenyl [2-(bicyclo[2.2.1]hept-5-en-2-ylamino)-4-oxo-4,5-dihydro-1,3-thiazol-5-yl]acetate,
2-Methoxyphenyl [2-(bicyclo[2.2.1]hept-5-en-2-ylamino)-4-oxo-4,5-dihydro-1,3-thiazol-5-yl]acetate,
3-Morpholin-4-ylphenyl [2-(bicyco[2.2.1]hept-5-en-2-ylamino)-4-oxo-4,5-dihydro-1,3-thiazol-5-yl]acetate,
2-(Bicyclo[2.2.1]hept-5-en-2-ylamino)-5-[2-(4-chlorophenyl)-2-oxoethyl]-1,3-thiazol-4(5H)-one,
5-(2-aminoethyl)-2-(bicyclo[2.2.1]hept-5-en-2-ylamino)-1,3-thiazol-4(5H)-one,
2-(bicyclo[2.2.1]hept-5-en-2-ylamino)-5-(2-bromoethyl)-1,3-thiazol-4(5H)-one,
2-(bicyclo[2.2.1]hept-2-ylamino)-5-(2-hydroxyethyl)-1,3-thiazol-4(5H)-one,
[2-(Bicyclo[2.2.1]hept-5-en-2-ylamino)-4-oxo-4,5-dihydro-1,3-thiazol-5-yl]acetic acid,
5-(2-morpholin-4-yl-2-oxoethyl)-2-[(2,2,3,3-tetramethylcyclopropyl)amino]-1,3-thiazol-4(5H)-one,
N-[(2-{[(1S)-1-cyclohexylethyl]amino}-4-oxo-4,5-dihydro-1,3-thiazol-5-yl)methyl]-2-methoxybenzamide,
2-(cyclooctylamino)-5-(2-morpholin-4-yl-2-oxoethyl)-1,3-thiazol-4(5H)-one,
2-[2-(bicyclo[2.2.1]hept-2-ylamino)-4-oxo-4,5-dihydro-1,3-thiazol-5-yl]-N-cycloheptylacetamide hydrochloride,
N-[(2-{[(1S)-1-cyclohexylethyl]amino}-4-oxo-4,5-dihydro-1,3-thiazol-5-yl)methyl]-2-fluorobenzamide,
2-fluoro-N-(2-{4-oxo-2-[(2,2,3,3-tetramethylcyclopropyl)amino]-4,5-dihydro-1,3-thiazol-5-yl}ethyl)benzamide,
2-(1-adamantylamino)-5-[2-(3,4-dihydroquinolin-1(2H)-yl)-2-oxoethyl]-1,3-thiazol-4(5H)-one,
2-[2-(bicyclo[2.2.1]hept-2-ylamino)-4-oxo-4,5-dihydro-1,3-thiazol-5-yl]-N-(2-chloro-6-fluorobenzyl)acetamide hydrochloride,
2-(cycloheptylamino)-5-[2-(3,4-dihydroquinolin-1(2H)-yl)-2-oxoethyl]-1,3-thiazol-4(5H)-one,
2-(cyclooctylamino)-5-[2-(3,4-dihydroquinolin-1(2H)-yl)-2-oxoethyl]-1,3-thiazol-4(5H)-one,
N-cyclohexyl-2-[2-(cyclooctylamino)-4-oxo-4,5-dihydro-1,3-thiazol-5-yl]-N-ethylacetamide,
5-(2-azepan-1-yl-2-oxoethyl)-2-(bicyclo[2.2.1]hept-2-ylamino)-1,3-thiazol-4(5H)-one hydrochloride,
N-cyclohexyl-N-ethyl-2-{4-oxo-2-[(2,2,3,3-tetramethylcyclopropyl)amino]-4,5-dihydro-1,3-thiazol-5-yl}acetamide,
5-chloro-N-{[2-(cyclooctylamino)-4-oxo-4,5-dihydro-1,3-thiazol-5-yl]methyl}-6-fluorobenzamide,
5-[2-(4-methylpiperidin-1-yl)-2-oxoethyl]-2-[(2,2,3,3-tetramethylcyclopropyl)amino]-1,3-thiazol-4(5H)-one,
2-chloro-N-{[2-(cycloheptylamino)-4-oxo-4,5-dihydro-1,3-thiazol-5-yl]methyl}-6-fluorobenzamide,
5-(2-anilinoethyl)-2-[(2,2,3,3-tetramethylcyclopropyl)amino]-1,3-thiazol-4(5H)-one,
2-chloro-6-fluoro-N-(2-{4-oxo-2-[(2,2,3,3-tetramethylcyclopropyl)amino]-4,5-dihydro-1,3-thiazol-5-yl}ethyl)benzamide,
5-(2-azepan-1-yl-2-oxoethyl)-2-(cyclooctylamino)-1,3-thiazol-4(5H)-one,
2-chloro-N-{2-[2-(cyclooctylamino)-4-oxo-4,5-dihydro-1,3-thiazol-5-yl]ethyl}-6-fluorobenzamide,
2-chloro-N-{2-[2-(cyclooctylamino)-4-oxo-4,5-dihydro-1,3-thiazol-5-yl]ethyl}benzenesulfonamide,
2-[2-(bicyclo[2.2.1]hept-5-en-2-ylamino)-4-oxo-4,5-dihydro-1,3-thiazol-5-yl]ethyl 2,4-dichlorobenzoate,
N-{2-[2-(cyclooctylamino)-4-oxo-4,5-dihydro-1,3-thiazol-5-yl]ethyl}-2,6-difluorobenzenesulfonamide,
N-{2-[2-(cyclooctylamino)-4-oxo-4,5-dihydro-1,3-thiazol-5-yl]ethyl}-2,6-difluorobenzamide,
2-(bicyclo[2.2.1]hept-5-en-2-ylamino)-5-[2-(2-chlorophenoxy)ethyl]-1,3-thiazol-4(5H)-one,
2-chloro-N-{2-[2-(cyclooctylamino)-4-oxo-4,5-dihydro-1,3-thiazol-5-yl]ethyl}benzamide,
3-[2-(4-benzylpiperidin-1-yl)-2-oxoethyl]-2-(cycloheptylamino)-1,3-thiazol-4(5H)-one,
N-{2-[2-(bicyclo[2.2.1]hept-5-en-2-ylamino)-4-oxo-4,5-dihydro-1,3-thiazol-5-yl]ethyl}-2,4-dichlorobenzamide,
2-chlorophenyl [2-(bicyclo[2.2.1]hept-5-en-2-ylamino)-4-oxo-4,5-dihydro-1,3-thiazol-5-yl]acetate,
N-{2-[2-(bicyclo[2.2.1]hept-5-en-2-ylamino)-4-oxo-4,5-dihydro-1,3-thiazol-5-yl]ethyl}-2-chlorobenzamide,
N-{2-[2-(cyclooctylamino)-4-oxo-4,5-dihydro-1,3-thiazol-5-yl]ethyl}-5-methyl-3-phenylisoxazole-4-carboxamide,
5-[2-(4-benzylpiperidin-1-yl)-2-oxoethyl]-2-[(cyclohexylmethyl)amino]-1,3-thiazol-4(5H)-one,
methyl 4-{2-[2-(bicyclo[2.2.1]hept-5-en-2-ylamino)-4-oxo-4,5-dihydro-1,3-thiazol-5-yl]ethoxy}-3-chlorobenzoate,
phenyl [2-(bicyclo[2.2.1]hept-5-en-2-ylamino)-4-oxo-4,5-dihydro-1,3-thiazol-5-yl]acetate,
N-{2-[2-(bicyclo[2.2.1]hept-5-en-2-ylamino)-4-oxo-4,5-dihydro-1,3-thiazol-5-yl]ethyl}-2-bromo-5-methoxybenzamide,
N-{2-[2-(cyclooctylamino)-4-oxo-4,5-dihydro-1,3-thiazol-5-yl]ethyl}-2-phenoxyacetamide,
2-(cycloheptylamino)-5-[2-(1-oxa-4-azaspiro[4.5]dec-4-yl)-2-oxoethyl]-1,3-thiazol-4(5H)-one,
N-{2-[2-(bicyclo[2.2.1]hept-5-en-2-ylamino)-4-oxo-4,5-dihydro-1,3-thiazol-5-yl]ethyl}-2-fluoro-4-(trifluoromethyl)benzamide,
2-(bicyclo[2.2.1]hept-5-en-2-ylamino)-5-[2-(4-chloro-3-methylphenoxy)ethyl]-1,3-thiazol-4(5H)-one,
2-[2-(cycloheptylamino)-4-oxo-4,5-dihydro-1,3-thiazol-5-yl]-N-ethyl-N-phenylacetamide,
N-(2-chloro-6-fluorobenzyl)-2-[2-(cycloheptylamino)-4-oxo-4,5-dihydro-1,3-thiazol-5-yl]acetamide,
2-methoxyphenyl [2-(bicyclo[2.2.1]hept-5-en-2-ylamino)-4-oxo-4,5-dihydro-1,3-thiazol-5-yl]acetate,
N-{2-[2-(cyclooctylamino)-4-oxo-4,5-dihydro-1,3-thiazol-5-yl]ethyl}adamantane-1-carboxamide,
5-[2-(3,4-dihydroquinolin-1(2H)-yl)-2-oxoethyl]-2-[(2-fluorophenyl)amino]-1,3-thiazol-4(5H)-one,
N-{2-[2-(bicyclo[2.2.1]hept-5-en-2-ylamino)-4-oxo-4,5-dihydro-1,3-thiazol-5-yl]ethyl}-2,5-difluorobenzamide,
5-(2-anilinoethyl)-2-{[1-(4-chlorophenyl)cyclobutyl]amino}-1,3-thiazol-4(5H)-one hydrobromide,
N-{2-[2-(cycloheptylamino)-4-oxo-4,5-dihydro-1,3-thiazol-5-yl]ethyl}-2,5-difluorobenzamide,
2-[2-(cycloheptylamino)-4-oxo-4,5-dihydro-1,3-thiazol-5-yl]-N-(4-methoxyphenyl)-N-methylacetamide,
2-(bicyclo[2.2.1]hept-5-en-2-ylamino)-5-[2-(4-phenoxyphenoxy)ethyl]-1,3-thiazol-4(5H)-one,
4-chloro-N-{2-[2-(cyclooctylamino)-4-oxo-4,5-dihydro-1,3-thiazol-5-yl]ethyl}benzamide,
N-{2-[2-(cyclooctylamino)-4-oxo-4,5-dihydro-1,3-thiazol-5-yl]ethyl}cyclohexanecarboxamide,
N-{2-[2-(cyclooctylamino)-4-oxo-4,5-dihydro-1,3-thiazol-5-yl]ethyl}-4-(trifluoromethyl)benzamide,
2-{[3,5-bis(trifluoromethyl)phenyl]amino}-5-[2-(3,4-dihydroquinolin-1(2H)-yl)-2-oxoethyl]-1,3-thiazol-4(5H)-one
2-(cycloheptylamino)-5-[2-(4-hydroxy-4-phenylpiperidin-1-yl)-2-oxoethyl]-1,3-thiazol-4(5H)-one,
2-{2-[(cyclohexylmethyl)amino]-4-oxo-4,5-dihydro-1,3-thiazol-5-yl}-N,N-diethylacetamide,
N-{2-[2-(cyclooctylamino)-4-oxo-4,5-dihydro-1,3-thiazol-5-yl]ethyl}-2,2-dimethylpropanamide,
2-morpholin-4-ylphenyl [2-(bicyclo[2.2.1]hept-5-en-2-ylamino)-4-oxo-4,5-dihydro-1,3-thiazol-5-yl]acetate,
N-{2-[2-(bicyclo[2.2.1]hept-5-en-2-ylamino)-4-oxo-4,5-dihydro-1,3-thiazol-5-yl]ethyl}-4-cyanobenzamide,
N-{2-[2-(cyclooctylamino)-4-oxo-4,5-dihydro-1,3-thiazol-5-yl]ethyl}-4-methoxybenzamide,
2-(bicyclo[2.2.1]hept-5-en-2-ylamino)-5-{2-[(2-chlorobenzyl)oxy]ethyl}-1,3-thiazol-4(5H)-one,
2-{2-[(2-fluorophenyl)amino]-4-oxo-4,5-dihydro-1,3-thiazol-5-yl}-N-(4-methylcyclohexyl)acetamide,
2-[(cyclohexylmethyl)amino]-5-(2-morpholin-4-yl-2-oxoethyl)-1,3-thiazol-4(5H)-one,
2-(cycloheptylamino)-5-isobutyl-1,3-thiazol-4(5H)-one,
(5R)-2-(cycloheptylamino)-5-(cyclohexylmethyl)-1,3-thiazol-4(5H)-one,
(5S)-2-(cycloheptylamino)-5-(cyclohexylmethyl)-1,3-thiazol-4(5H)-one,
2-(cyclooctylamino)-5-(4-hydroxybenzyl)-1,3-thiazol-4(5H)-one,
2-(cycloheptylamino)-5-(1H-indol-3-ylmethyl)-1,3-thiazol-4(5H)-one,
2-(cycloheptylamino)-5-(4-hydroxybenzyl)-1,3-thiazol-4(5H)-one,
2-(bicyclo[2.2.1]hept-2-ylamino)-5-(4-hydroxybenzyl)-1,3-thiazol-4(5H)-one,
2-(cycloheptylamino)-5-(3,4-dihydroxybenzyl)-1,3-thiazol-4(5H)-one,
2-(cycloheptylamino)-5-(pyridin-3-ylmethyl)-1,3-thiazol-4(5H)-one,
2-(cyclooctylamino)-5-propyl-1,3-thiazol-4(5H)-one hydrobromide,
5-butyl-2-(cyclooctylamino)-1,3-thiazol-4(5H)-one hydrobromide,
2-(bicyclo[2.2.1]hept-2-ylamino)-5-ethyl-1,3-thiazol-4(5H)-one hydrobromide,
(5S)-2-(cycloheptylamino)-5-methyl-1,3-thiazol-4(5H)-one,
2-(cyclooctylamino)-5-methyl-1,3-thiazol-4(5H)-one,
2-(cyclooctylamino)-5-ethyl-1,3-thiazol-4(5H)-one,
2-(cycloheptylamino)-5-ethyl-1,3-thiazol-4(5H)-one,
2-{2-[2-(bicyclo[2.2.1]hept-5-en-2-ylamino)-4-oxo-4,5-dihydro-1,3-thiazol-5-yl]ethyl}-1H-isoindole-1,3(2H)-dione,
5-{[5-(2-chlorophenyl)-1,3,4-oxadiazol-2-yl]methyl}-2-[(2-fluorophenyl)amino]-1,3-thiazol-4(5H)-one,
5-{[5-(2-chlorophenyl)-1,3,4-oxadiazol-2-yl]methyl}-2-(tricyclo[3.3.1.0˜3,7˜]non-3-ylamino)-1,3-thiazol-4(5H)-one,
5-{[5-(2-chlorophenyl)-1,3,4-oxadiazol-2-yl]methyl}-2-{[(1S,2S,3S,5R)-2,6,6-trimethylbicyclo[3.1.1]hept-3-yl]amino}-1,3-thiazol-4(5H)-one,
2-(bicyclo[2.2.1]hept-2-ylamino)-5-{[5-(2-chlorophenyl)-1,3,4-oxadiazol-2-yl]methyl}-1,3-thiazol-4(5H)-one,
5-{[5-(2-chlorophenyl)-1,3,4-oxadiazol-2-yl]methyl}-2-{[(1R,2R,3R,5S)-2,6,6-trimethylbicyclo[3.1.1]hept-3-yl]amino}-1,3-thiazol-4(5H)-one,
5-(1,3-benzoxazol-2-ylmethyl)-2-(cycloheptylamino)-1,3-thiazol-4(5H)-one,
5-(1H-benzimidazol-2-ylmethyl)-2-(bicyclo[2.2.1]hept-2-ylamino)-1,3-thiazol-4(5H)-one,
5-(1H-benzimidazol-2-ylmethyl)-2-(cycloheptylamino)-1,3-thiazol-4(5H)-one,
N-{2-[2-(cyclooctylamino)-4-oxo-4,5-dihydro-1,3-thiazol-5-yl]ethyl}-2-fluorobenzamide,
N-{2-[2-(cyclooctylamino)-4-oxo-4,5-dihydro-1,3-thiazol-5-yl]ethyl}-2,6-difluoro-N-methylbenzamide,
2-chloro-N-{2-[2-(cyclooctylamino)-4-oxo-4,5-dihydro-1,3-thiazol-5-yl]ethyl}-N-methylbenzamide, and
2,4-dichloro-N-{2-[2-(cyclooctylamino)-4-oxo-4,5-dihydro-1,3-thiazol-5-yl]ethyl}-N-methylbenzamide.

5. A process for the preparation of a compound according to any one of claims 1 to 4, comprising at least one of the following steps:
a) reaction of an isothiocyanate with ammonia to give a thiourea,
b) reaction of an amine with ethoxycarbonylisothiocyanate to give a thiourea,
c) reaction of a thiourea with maleic anhydride to give a thiazolone carboxylic acid,
d) reaction of a thiazolone carboxylic acid with 2-chloro-1-methylpyridinium iodide in the presence of an amine to give a thiazolone amide,
e) reaction of a thiourea with 2-bromo-1-butyrolactone to give a thiazolone alcohol,
f) reaction of a thiazolone alcohol with an acid chloride or an isocyanate in the presence of a base to give a thiazolone ester,
g) reaction of a thiazolone alcohol with triphenylphosphine and then with a benzyl alcohol in the presence of dietyl azodicarboxylate to give a thiazolone ether,
h) reaction of a thiourea with an N-substituted 3-bromo-1-phenylpyrrolidin-2-one to give a thiazolone amine,
i) reaction of a thiazolone alcohol with triphenylphosphine dibromide to give a thiazolone bromide,
j) reaction of a thiazolone bromide with an N-substituted aniline to give a thiazolone amine,
k) reaction of a thiourea with 3-(4-chlorobenzoyl)acrylic acid to give a thiazolone,
l) reaction of a thiourea with 3-bromopyrrolidin-2-one to give a thiazolone amine,
m) reaction of a thiazolone amine with a benzoyl chloride or a sulfonyl chloride to give a thiazolone amide or a thiazolone sulfonamide, respectively,
n) hydrolysis of a thiazolone amide with hydrazine to give a thiazolone amine,
o) esterification of a thiazolone carboxylic acid with a phenol in the presence of a base and a coupling agent to give a thiazolone phenol ester,
p) reaction of a thiourea with a 1H-pyrrole-2,5-dione to give a thiazolone amide,
q) reaction of a thiazolone carboxylic acid with diphenylphosphoryl azide and then with a benzoyl chloride to give a thiazolone amide,
r) amidification of a thiazolone carboxylic acid with an amine in the presence of a base and a coupling agent to give a thiazolone amide,
s) reaction of an N—C₃₋₁₀-cycloalkylthiourea with a carboxylic acid to give a thiazolone,
t) reaction of an N—C₃₋₁₀-cycloalkylthiourea with a bromo substituted carboxylic ester to give a thiazolone,
u) reaction of a thiazolone carboxylic acid with 2-chlorobenzohydrazide in the presence of POCl₃ to give a thiazolone containing a triazole group,
v) reaction of a thiazolone carboxylic acid with an aromatic amine to give a thiazoline containing a benzimidazole, benzoxazole or a benzothiazole group, and
w) alkylation of a thiazolone amine.

6. A compound of the general formula (I) 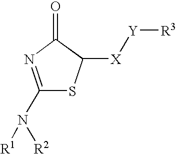
wherein
R¹ and R² are each independently selected from hydrogen; C₁₋₈-alkyl; C₃₋₁₀-cycloalkyl optionally independently substituted by one or more of C₁₋₈-alkyl; C₂₋₈-alkenyl; C₃₋₁₀-cycloalkyl-C₁₋₈-alkyl; C₃₋₁₀-cycloalkenyl; C₃₋₁₀-cycloalkenyl-C₁₋₈-alkyl; C₁₋₈-acyl; heterocyclyl optionally independently substituted by one or more of C₁₋₈-alkyl; heterocyclyl-C₁₋₈-alkyl; aryl optionally independently substituted by one or more of halogen, C₁₋₈-alkyl, halo-C₁₋₈-alkyl, C₁₋₈-alkoxy, and heterocyclyl; indanyl; aryl-C₁₋₈-alkyl optionally independently substituted by one or more of halogen and C₁₋₈-alkyl; aryl-C₃₋₁₀-cycloalkyl optionally independently substituted by one or more of halogen; heteroaryl optionally independently substituted by one or more of aryloxy; heterocyclyl-C₁₋₈-alkyl; or form together with the nitrogen atom bonded thereto heterocyclyl;
X is CH₂;
Y is CH₂, CO or a single bond;
R³ is hydrogen; C₁₋₈-alkyl; C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl substituted by one or more of halogen or hydroxy;
or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally independently substituted by one or more of halogen, C₁₋₈-alkyl and aryl; heteroaryl-C₁₋₈-alkylcarbonyl; C₃₋₁₀-cycloalkylcarbonyl; heterocyclylcarbonyl; heteroaryl; COOR⁶, wherein R⁶ is selected from C_{1.}₈-alkyl and aryl; CONR⁷R⁸, wherein R⁷ and R⁸ are each independently selected from hydrogen and C₁₋₈-alkyl; or wherein R³ is OCONR⁹R¹⁰, wherein R⁹ and R¹⁰ are each independently selected from aryl optionally independently substituted by one or more of halogen, nitro, and aryloxy; or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro;
or pharmaceutically acceptable salts, solvates, hydrates, geometrical isomers, tautomers, optical isomers, N-oxides and prodrug forms thereof;
for use in therapy.

7. The compound according to claim 6, wherein
R¹ and R² are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkenyl; C₃₋₁₀-cycloalkenyl-C₁₋₈-alkyl; heterocyclyl optionally independently substituted by one or more of C₁₋₈-alkyl; heterocyclyl-C₁₋₈-alkyl; aryl optionally independently substituted by one or more of halogen, C₁₋₈-alkyl, halo-C₁₋₈-alkyl, C₁₋₈-alkoxy, and heterocyclyl; indanyl; aryl-C₁₋₈-alkyl optionally independently substituted by one or more of halogen and C₁₋₈-alkyl; aryl-C₃₋₁₀-cycloalkyl optionally independently substituted by one or more of halogen; heteroaryl optionally independently substituted by one or more of aryloxy; heterocyclyl-C₁₋₈-alkyl; or form together with the nitrogen atom bonded thereto heterocyclyl;
X is CH₂;
Y is CH₂, CO or a single bond;
R³ is hydrogen; C₁₋₈-alkyl; C₃₋₁₀-cycloalkyl; halogen; heterocyclyl optionally independently substituted by one or more of C₁₋₈-alkyl, halo-C₁₋₈-alkyl, C₁₋₈-alkoxy, hydroxy, aryl optionally independently substituted by one or more of halogen, and aryl-C₁₋₈-alkyl; aryl optionally independently substituted by one or more of halogen or hydroxy;
or wherein R³ is NR⁴R⁵, wherein R⁴ and R⁵ are each independently selected from hydrogen; C₁₋₈-alkyl; C₃₋₁₀-cycloalkyl optionally independently substituted by one or more of C₁₋₈-alkyl; C₃₋₁₀-cycloalkyl-C₁₋₈-alkyl; C₃₋₁₀-cycloalkylcarbonyl; aryl optionally independently substituted by one or more of halogen, C₁₋₈-alkyl, C₁₋₈-alkoxy, halo-C₁₋₈-alkoxy, arylcarbonyl, and carboxy; aryl-C₁₋₈-alkyl optionally independently substituted by one or more of C₁₋₈-alkyl and halogen; C₁₋₈-acyl optionally independently substituted by one or more of aryloxy; aryl-C₁₋₈-acyl optionally independently substituted by one or more of halogen, C₁₋₈-alkoxy, cyano, and halo-C₁₋₈-alkyl; arylsulfonyl optionally independently substituted by one or more of halogen; heteroarylcarbonyl optionally substituted by one or more of halogen, C₁₋₈-alkyl and aryl; heteroaryl-C₁₋₈-alkylcarbonyl; C₃₋₁₀-cycloalkylcarbonyl; heteroaryl; or wherein R³ is OCONR⁹R¹⁰, wherein R⁹ and R¹⁰ are each independently selected from aryl substituted by one or more of halogen, nitro, and aryloxy;
or wherein R³ is NHCONR¹¹R¹², wherein R¹¹ and R¹² are each independently selected from hydrogen; C₃₋₁₀-cycloalkyl; aryl optionally independently substituted by one or more of halogen, halo-C₁₋₈-alkyl, and C₁₋₈-alkoxy; heteroaryl optionally independently substituted by one or more of halogen; or wherein R³ is OR¹³, wherein R¹³ is selected from hydrogen; aryl optionally independently substituted by one or more of halogen, C₁₋₈-alkyl, C₁₋₈-alkoxy, C₁₋₈-alkoxycarbonyl, heterocyclyl, and aryloxy; aryl-C₁₋₈-alkyl optionally independently substituted by one or more of halogen, C₁₋₈-alkoxy, mono-, or di-C₁₋₈-alkylamino; arylcarbonyl optionally independently substituted by one or more of halogen, C₁₋₈-alkyl, halo-C₁₋₈-alkyl, C₁₋₈-alkoxy, and nitro.

8. The compound according to claim 6, wherein
R¹ and R² are selected from hydrogen; 2-butyl; isobutyl; tert-butyl; 2-methylbutyl; 1,1,3,3-tetramethylbutyl; cyclopropyl; cyclopentyl; cyclohexyl; cycloheptyl; bicyclo[2.2.1]hept-2-yl; cyclooctyl; 1-adamantyl; tricyclo[3.3.1.0˜3,7˜]non-3-yl; cyclopropylmethyl; cyclohexylmethyl; 2,2,3,3-tetramethylcyclopropyl; (1R,2R,3R,5S)-2,6,6-trimethylbicyclo[3.1.1]hept-3-yl; (1S,2S,3S,5R)-2,6,6-trimethylbicyclo[3.1.1]hept-3-yl; bicyclo[2.2.1]hept-5-en-2-yl; (1R)-1-cyclohexylethyl; (1S)-1-cyclohexylethyl; 2-(1-cyclohexenyl)ethyl; 4-(2,2,6,6-tetramethyl)piperidyl; 2-(4-morpholinyl)ethyl; 1-naphthyl; 2-fluorophenyl; 3-chloro-2-methylphenyl; mesityl; 3,5-di(trifluoromethyl)phenyl; 2,6-dimethylphenyl, 4-(4-morpholinyl)phenyl; 2-methylphenyl; 2-isopropylphenyl; 2-methoxyphenyl; 2-indanyl; 4-chlorobenzyl; 4-methylbenzyl; (1R)-1-phenylethyl; (1S)-1-phenylethyl; 2-phenylethyl; (2R)-2-phenylpropyl; (2S)-2-phenylpropyl; 1-(4-chlorophenyl)cyclobutyl; 6-phenoxy-3-pyridyl; 2-(4-morpholinyl)ethyl; or R¹ and R² form together with the nitrogen atom bonded thereto azepan-1-yl;
R³ is hydrogen; methyl; ethyl; isopropyl; cyclohexyl; bromo; 1-hexahydroazepinyl; 4-morpholinyl; N-phthalimidyl; piperidin-1-yl; 4-methylpiperidin-1-yl; 1-(1,2,3,4-tetrahydroquinolinyl); 2-(1,2,3,4-tetrahydroisoquinolinyl); 8-methyl-1-(1,2,3,4-tetrahydroquinolinyl); 1-[7-(trifluoromethyl)-1,2,3,4-tetrahydroquinolinyl; 3,4-dihydroisoquinolin-2(1H)-yl; 6,7-dimethoxy-3,4-dihydroisoquinolin-2(1H)-yl; 4-benzylpiperidin-1-yl; azepan-1-yl; azocan-1-yl; 1-oxa-4-azaspiro[4.5]dec-4-yl; 2-decahydroisoquinolinyl; 1,4-diazepan-1-ium; 1,3-dihydro-2H-isoindol-2-yl; 2,3-dihydro-1H-indol-1-yl; pyrrolidin-1-yl; 3-pyridinyl; 3-indolyl; 1,3-benzoxazol-2-yl; 1,3-benzothiazol-2-yl; 1H-benzimidazol-2-yl; 4-hydroxy-4-phenylpiperidin-1-yl; 5-(2-chlorophenyl)-1,3,4-oxadiazol-2-yl; 4-chlorophenyl; 4-hydroxyphenyl; 3,4-dihydroxyphenyl;
or wherein R³ is NR⁴R⁵, wherein R⁴ and R⁵ are each independently selected from hydrogen; methyl; ethyl; n-propyl; isopropyl; n-butyl; cyclohexyl; cycloheptyl; (1R,2R,4S)-bicyclo[2.2.1]hept-2-yl; 4-methylcyclohexyl; cyclopropylmethyl; cyclohexylmethyl; cyclohexylcarbonyl; 1-adamantylcarbonyl; phenyl; 1-naphthyl; 4-bromophenyl; 2-chlorophenyl; 3-chlorophenyl; 4-chlorophenyl; 4-fluorophenyl; 2,6-difluorophenyl; 3-chloro-2-methylphenyl; 2-methylphenyl; 3-methylphenyl; 4-methylphenyl; 4-methoxyphenyl; 4-(trifluoromethoxy)phenyl; 2-benzoylphenyl; 3-carboxyphenyl; benzyl; 2-phenylethyl; 2-chloro-6-fluorobenzyl; 3-chloro-2-methylbenzyl; 2,2-dimethylpropionamido; phenoxyacetyl; 2-chlorobenzoyl; 2-fluorobenzoyl; 4-chlorobenzoyl; 2,5-difluorobenzoyl; 2,6-difluorobenzoyl; 2-chloro-6-fluorobenzoyl; 2,4-dichlorobenzoyl; 2,4,6-trichlorobenzoyl; 2-methoxybenzoyl; 4-methoxybenzoyl; 2-bromo-5-methoxybenzoyl; 2,4-dimethoxybenzoyl; 2,6-dimethoxybenzoyl; 4-(trifluoromethyl)benzoyl; 2-fluoro-4-(trifluoromethyl)benzoyl; 2,5-di(trifluoromethyl)benzoyl; 2-fluoro-5-(trifluoromethyl)benzoyl; 4-cyanobenzoyl; 2-chloro-6-fluorophenylacetyl; 2-chlorophenylsulfonyl; 2,6-difluorophenylsulfonyl; 2-chloro-3-pyridylcarbonyl; 2-furylcarbonyl; 2-thienylcarbonyl; 5-isoxazolylcarbonyl; 5-methyl-3-phenylisoxazol-4-ylcarbonyl; 2-thienylmethylcarbonyl; cyclopropylcarbonyl; cyclohexylcarbonyl; isopentanoyl; indazol-6-yl;
OCONR⁹R¹⁰, wherein R⁹ and R¹⁰ are each independently selected from 2-chlorophenyl; 4-bromo-2,6-difluorophenyl; 4-chloro-3-nitrophenyl; 3-phenoxyphenyl;
NHCONR¹¹R¹², wherein R¹¹ and R¹² are each independently selected from hydrogen, cyclopentyl, cyclohexyl, 2-chlorophenyl, 2-fluorophenyl, 4-fluorophenyl, 2,4-difluorophenyl, 2,6-difluorophenyl, 2-chloro-5-(trifluoromethyl)phenyl, 4-fluoro-2-(trifluoromethyl)phenyl, 2-methoxyphenyl, 2,4-dimethoxyphenyl, 5-chloro-2-methoxyphenyl, 2,6-dichloropyridin-4-yl;
OR¹³, wherein R¹³ is selected from hydrogen; phenyl; 2-chlorophenyl; 4-chloro-3-methylphenyl; 2-methoxyphenyl; 4-carbomethoxy-2-chlorophenyl; 3-(4-morpholinyl)phenyl; 4-phenoxyphenyl; 2-chlorobenzyl; 2-methylbenzyl; 2-methoxybenzyl; 3-(dimethylamino)benzyl; benzoyl; 2-chlorobenzoyl; 2,4-dichlorobenzoyl; 3,4-dichlorobenzoyl; 2,5-difluorobenzoyl; 2,6-difluorobenzoyl; 3,4-difluorobenzoyl; 2-chloro-6-fluorobenzoyl; 2,4,6-trichlorobenzoyl; 2,3,4-trifluorobenzoyl; 3-methylbenzoyl; 4-methylbenzoyl; 4-tert-butylbenzoyl; 3-methoxybenzoyl; 4-n-butoxybenzoyl; 2,4-dimethoxybenzoyl; 2,6-dimethoxybenzoyl; 2-bromo-5-methoxybenzoyl; 3-(trifluoromethyl)benzoyl; 2,5-di(trifluoromethyl)benzoyl; 3,5-di(trifluoromethyl)benzoyl; 2-fluoro-4-(trifluoromethyl)benzoyl; 2-fluoro-5-(trifluoromethyl)benzoyl; and 4-chloro-3-nitrobenzoyl.

9. The compound according to claim 6, which is selected from:
N-{2-[2-(bicyclo[2.2.1]hept-5-en-2-ylamino)-4-oxo-4,5-dihydro-1,3-thiazol-5-yl]ethyl}-2-chloro-6-fluorobenzamide,
N-{2-[2-(bicyclo[2.2.1]hept-5-en-2-ylamino)-4-oxo-4,5-dihydro-1,3-thiazol-5-yl]ethyl}-2,6-dimethoxybenzamide,
N-{2-[2-(bicyclo[2.2.1]hept-5-en-2-ylamino)-4-oxo-4,5-dihydro-1,3-thiazol-5-yl]ethyl}-2,4-dimethoxybenzamide,
N-{2-[2-(bicyclo[2.2.1]hept-5-en-2-ylamino)-4-oxo-4,5-dihydro-1,3-thiazol-5-yl]ethyl}-2,6-difluorobenzamide,
2-{2-[2-(bicyclo[2.2.1]hept-5-en-2-ylamino)-4-oxo-4,5-dihydro-1,3-thiazol-5-yl]ethyl}-1H-isoindole-1,3(2H)-dione,
N-{2-[2-(bicyclo[2.2.1]hept-5-en-2-ylamino)-4-oxo-4,5-dihydro-1,3-thiazol-5-yl]ethyl}-2,5-difluorobenzamide,
N-{2-[2-(bicyclo[2.2.1]hept-5-en-2-ylamino)-4-oxo-4,5-dihydro-1,3-thiazol-5-yl]ethyl}-2-chlorobenzamide,
N-{2-[2-(bicyclo[2.2.1]hept-5-en-2-ylamino)-4-oxo-4,5-dihydro-1,3-thiazol-5-yl]ethyl}-2-bromo-5-methoxybenzamide,
N-{2-[2-(bicyclo[2.2.1]hept-5-en-2-ylamino)-4-oxo-4,5-dihydro-1,3-thiazol-5-yl]ethyl}-2-fluoro-4-(trifluoromethyl)benzamide,
N-{2-[2-(bicyclo[2.2.1]hept-5-en-2-ylamino)-4-oxo-4,5-dihydro-1,3-thiazol-5-yl]ethyl}-2,4-dichlorobenzamide,
2-chloro-N-{2-[2-(cyclohexylamino)-4-oxo-4,5-dihydro-1,3-thiazol-5-yl]ethyl}benzamide,
N-{2-[2-(cyclohexylamino)-4-oxo-4,5-dihydro-1,3-thiazol-5-yl]ethyl}-2,6-difluorobenzamide,
N-{2-[2-(cyclohexylamino)-4-oxo-4,5-dihydro-1,3-thiazol-5-yl]ethyl}-2,6-dimethoxybenzamide,
2-bromo-N-{2-[2-(cyclohexylamino)-4-oxo-4,5-dihydro-1,3-thiazol-5-yl]ethyl}-5-methoxybenzamide,
2-chloro-N-{2-[2-(cyclohexylamino)-4-oxo-4,5-dihydro-1,3-thiazol-5-yl]ethyl}-6-fluorobenzamide,
2,4-dichloro-N-{2-[2-(cyclohexylamino)-4-oxo-4,5-dihydro-1,3-thiazol-5-yl]ethyl}benzamide,
2-chloro-N-{2-[4-oxo-2-(tricyclo[3.3.1.0˜3,7˜]non-3-ylamino)-4,5-dihydro-1,3-thiazol-5-yl]ethyl}benzamide,
2,6-difluoro-N-{2-[4-oxo-2-(tricyclo[3.3.1.0˜3,7˜]non-3-ylamino)-4,5-dihydro-1,3-thiazol-5-yl]ethyl}benzamide,
2-chloro-6-fluoro-N-{2-[4-oxo-2-(tricyclo[3.3.1.0˜3,7˜]non-3-ylamino)-4,5-dihydro-1,3-thiazol-5-yl]ethyl}benzamide,
2,4-dichloro-N-{2-[4-oxo-2-(tricyclo[3.3.1.0˜3,7˜]non-3-ylamino)-4,5-dihydro-1,3-thiazol-5-yl]ethyl}benzamide,
2-chloro-N-(2-{2-[(cyclohexylmethyl)amino]-4-oxo-4,5-dihydro-1,3-thiazol-5-yl}ethyl)benzamide,
2-bromo-N-(2-{2-[(cyclohexylmethyl)amino]-4-oxo-4,5-dihydro-1,3-thiazol-5-yl}ethyl)-5-methoxybenzamide,
2,4-dichloro-N-(2-{2-[(cyclohexylmethyl)amino]4-oxo-4,5-dihydro-1,3-thiazol-5-yl}ethyl)benzamide,
2-chloro-N-{2-[2-(cycloheptylamino)-4-oxo-4,5-dihydro-1,3-thiazol-5-yl]ethyl}benzamide,
N-{2-[2-(cycloheptylamino)-4-oxo-4,5-dihydro-1,3-thiazol-5-yl]ethyl}-2,6-difluorobenzamide,
N-{2-[2-(cycloheptylamino)-4-oxo-4,5-dihydro-1,3-thiazol-5-yl]ethyl}-2,6-dimethoxybenzamide,
2-bromo-N-{2-[2-(cycloheptylamino)-4-oxo-4,5-dihydro-1,3-thiazol-5-yl]ethyl}-5-methoxybenzamide,
2-chloro-N-{2-[2-(cycloheptylamino)-4-oxo-4,5-dihydro-1,3-thiazol-5-yl]ethyl}-6-fluorobenzamide,
2,4-dichloro-N-{2-[2-(cycloheptylamino)-4-oxo-4,5-dihydro-1,3-thiazol-5-yl]ethyl}benzamide,
N-{2-[2-(cycloheptylamino)-4-oxo-4,5-dihydro-1,3-thiazol-5-yl]ethyl}-2,5-difluorobenzamide,
N-{2-[2-(cycloheptylamino)-4-oxo-4,5-dihydro-1,3-thiazol-5-yl]ethyl}-2,5-bis(trifluoromethyl)benzamide,
N-{2-[2-(cycloheptylamino)-4-oxo-4,5-dihydro-1,3-thiazol-5-yl]ethyl}-2-fluoro-5-(trifluoromethyl)benzamide,
2-chloro-N-{2-[2-(cycloheptylamino)-4-oxo-4,5-dihydro-1,3-thiazol-5-yl]ethyl}nicotinamide,
N-{2-[2-(cycloheptylamino)-4-oxo-4,5-dihydro-1,3-thiazol-5-yl]ethyl}-2-furamide,
N-{2-[2-(cycloheptylamino)-4-oxo-4,5-dihydro-1,3-thiazol-5-yl]ethyl}thiophene-2-carboxamide,
N-{2-[2-(cycloheptylamino)-4-oxo-4,5-dihydro-1,3-thiazol-5-yl]ethyl}-2-(2-thienyl)acetamide,
N-{2-[2-(cycloheptylamino)-4-oxo-4,5-dihydro-1,3-thiazol-5-yl]ethyl}cyclopropanecarboxamide,
N-{2-[2-(cycloheptylamino)-4-oxo-4,5-dihydro-1,3-thiazol-5-yl]ethyl}-3-methylbutanamide,
N-{2-[2-(bicyclo[2.2.1]hept-5-en-2-ylamino)-4-oxo-4,5-dihydro-1,3-thiazol-5-yl]ethyl}cyclohexanecarboxamide,
N-{2-[2-(bicyclo[2.2.1]hept-5-en-2-ylamino)-4-oxo-4,5-dihydro-1,3-thiazol-5-yl]ethyl}isoxazole-5-carboxamide,
N-{2-[2-(bicyclo[2.2.1]hept-5-en-2-ylamino)-4-oxo-4,5-dihydro-1,3-thiazol-5-yl]ethyl}-2,4,6-trichlorobenzamide,
N-[(2-azepan-1-yl-4-oxo-4,5-dihydro-1,3-thiazol-5-yl)methyl]-2-fluorobenzamide,
2-(bicyclo[2.2.1]hept-5-en-2-ylamino)-5-{2-[methyl(phenyl)amino]ethyl}-1,3-thiazol-4(5H)-one,
2-(bicyclo[2.2.1]hept-5-en-2-ylamino)-5-[2-(2,3-dihydro-1H-indol-1-yl)ethyl]-1,3-thiazol-4(5H)-one,
2-(bicyclo[2.2.1]hept-5-en-2-ylamino)-5-[2-(3,4-dihydroquinolin-1(2H)-yl)ethyl]-1,3-thiazol-4(5H)-one,
2-(bicyclo[2.2.1]hept-5-en-2-ylamino)-5-[2-(1,3-dihydro-2H-isoindol-2-yl)ethyl]-1,3-thiazol-4(5H)-one,
2-(bicyclo[2.2.1]hept-5-en-2-ylamino)-5-(2-piperidin-1-ylethyl)-1,3-thiazol-4(5H)-one,
5-(2-Anilinoethyl)-2-{[(1R,2R,3R,5S)-2,6,6-trimethylbicyclo[3.1.1]hept-3-yl]amino}-1,3-thiazol-4(5H)-one hydrobromide,
5-(2-Anilinoethyl)-2-{[(1S,2S,3S,5R)-2,6,6-trimethylbicyclo[3.1.1]hept-3-yl]amino}-1,3-thiazol-4(5H)-one hydrobromide,
5-(2-Anilinoethyl)-2-(tricyclo[3.3.1.0˜3,7˜]non-3-ylamino)-1,3-thiazol-4(5H)-one,
5-(2-anilinoethyl)-2-(bicyclo[2.2.1]hept-2-ylamino)-1,3-thiazol-4(5H)-one,
5-(2-anilinoethyl)-2-[(2-cyclohex-1-en-i -ylethyl)amino]-1,3-thiazol-4(5H)-one,
5-(2-anilinoethyl)-2-[(1,1,3,3-tetramethylbutyl)amino]-1,3-thiazol-4(5H)-one hydrobromide,
5-(2-anilinoethyl)-2-[(cyclohexylmethyl)amino]-1,3-thiazol-4(5H)-one,
5-(2-anilinoethyl)-2-[(2,2,6,6-tetramethylpiperidin-4-yl)amino]-1,3-thiazol-4(5H)-one,
5-(2-anilinoethyl)-2-(cyclohexylamino)-1,3-thiazol-4(5H)-one hydrobromide,
5-(2-anilinoethyl)-2-{[(1R)-1-phenylethyl]amino}-1,3-thiazol-4(5H)-one hydrochloride,
5-(2-anilinoethyl)-2-{[(1S)-1-phenylethyl]amino}-1,3-thiazol-4(5H)-one hydrochloride,
5-(2-anilinoethyl)-2-{[(2R)-2-phenylpropyl]amino}-1,3-thiazol-4(5H)-one hydrochloride,
5-(2-anilinoethyl)-2-(cycloheptylamino)-1,3-thiazol-4(5H)-one,
5-(2-anilinoethyl)-2-[(4-methylbenzyl)amino]-1,3-thiazol-4(5H)-one,
5-(2-anilinoethyl)-2-(cyclooctylamino)-1,3-thiazol-4(5H)-one hydrobromide,
5-(2-anilinoethyl)-2-{[(1R)-1-cyclohexylethyl]amino}-1,3-thiazol-4(5H)-one,
5-(2-anilinoethyl)-2-{[(1S)-1-cyclohexylethyl]amino}-1,3-thiazol-4(5H)-one,
5-(2-anilinoethyl)-2-azepan-1-yl-1,3-thiazol-4(5H)-one,
5-(2-anilinoethyl)-2-{[(2S)-2-phenylpropyl]amino}-1,3-thiazol-4(5H)-one,
2-[(cyclohexylmethyl)amino]-5-{2-[(4-fluorophenyl)amino]ethyl}-1,3-thiazol-4(5H)-one trifluoroacetate,
2-(bicyclo[2.2.1]hept-5-en-2-ylamino)-5-[2-(3,4-dihydroquinolin-1(2H)-yl)-2-oxoethyl]-1,3-thiazol-4(5H)-one,
2-[2-(Bicyclo[2.2.1]hept-5-en-2-ylamino)-4-oxo-4,5-dihydro-1,3-thiazol-5-yl]-N-(3-chloro-2-methylphenyl)acetamide,
N-Benzyl-2-[2-(bicyclo[2.2.1]hept-5-en-2-ylamino)-4-oxo-4,5-dihydro-1,3-thiazol-5-yl]acetamide,
N-benzyl-2-[2-(bicyclo[2.2.1]hept-5-en-2-ylamino)-4-oxo-4,5-dihydro-1,3-thiazol-5-yl]-N-phenylacetamide,
2-[2-(bicyclo[2.2.1]hept-5-en-2-ylamino)-4-oxo-4,5-dihydro-1,3-thiazol-5-yl]-N-(4-methoxyphenyl)-N-methylacetamide,
2-[2-(bicyclo[2.2.1]hept-5-en-2-ylamino)-4-oxo-4,5-dihydro-1,3-thiazol-5-yl]-N-methyl-N-phenylacetamide,
2-(bicyclo[2.2.1]hept-5-en-2-ylamino)-5-[2-(1,3-dihydro-2H-isoindol-2-yl)-2-oxoethyl]-1,3-thiazol-4(5H)-one,
2-(bicyclo[2.2.1]hept-5-en-2-ylamino)-5-[2-(2,3-dihydro-1H-indol-1-yl)-2-oxoethyl]-1,3-thiazol-4(5H)-one,
2-[2-(bicyclo[2.2.1]hept-5-en-2-ylamino)-4-oxo-4,5-dihydro-1,3-thiazol-5-yl]-N-ethyl-N-(3-methylphenyl)acetamide,
2-(bicyclo[2.2.1]hept-5-en-2-ylamino)-5-[2-(3,4-dihydroisoquinolin-2(1H)-yl)-2-oxoethyl]-1,3-thiazol-4(5H)-one,
2-[2-(bicyclo[2.2.1]hept-5-en-2-ylamino)-4-oxo-4,5-dihydro-1,3-thiazol-5-yl]-N-methyl-N-[4-(trifluoromethoxy)phenyl]acetamide,
2-[2-(bicyclo[2.2.1]hept-5-en-2-ylamino)-4-oxo-4,5-dihydro-1,3-thiazol-5-yl]-N-ethyl-N-[4-(trifluoromethoxy)phenyl]acetamide,
2-(bicyclo[2.2.1]hept-5-en-2-ylamino)-5-{2-oxo-2-[7-(trifluoromethyl)-3,4-dihydroquinolin-1(2H)-yl]ethyl}-1,3-thiazol-4(5H)-one,
2-[2-(bicyclo[2.2.1]hept-5-en-2-ylamino)-4-oxo-4,5-dihydro-1,3-thiazol-5-yl]-N-methyl-N-(2-methylphenyl)acetamide,
2-[2-(bicyclo[2.2.1]hept-5-en-2-ylamino)-4-oxo-4,5-dihydro-1,3-thiazol-5-yl]-N-ethyl-N-phenylacetamide,
2-[2-(bicyclo[2.2.1]hept-5-en-2-ylamino)-4-oxo-4,5-dihydro-1,3-thiazol-5-yl]-N-methyl-N-(4-methylphenyl)acetamide,
2-[2-(bicyclo[2.2.1]hept-5-en-2-ylamino)-4-oxo-4,5-dihydro-1,3-thiazol-5-yl]-N-(4-bromophenyl)-N-methylacetamide,
2-[2-(bicyclo[2.2.1]hept-5-en-2-ylamino)-4-oxo-4,5-dihydro-1,3-thiazol-5-yl]-N-(4-chlorophenyl)-N-methylacetamide,
2-[2-(bicyclo[2.2.1]hept-5-en-2-ylamino)-4-oxo-4,5-dihydro-1,3-thiazol-5-yl]-N-(4-fluorophenyl)-N-methylacetamide,
2-[2-(bicyclo[2.2.1]hept-5-en-2-ylamino)-4-oxo-4,5-dihydro-1,3-thiazol-5-yl]-N-(3-chlorophenyl)-N-methylacetamide,
2-[2-(bicyclo[2.2.1]hept-5-en-2-ylamino)-4-oxo-4,5-dihydro-1,3-thiazol-5-yl]-N-ethyl-N-(2-methylphenyl)acetamide,
2-(bicyclo[2.2.1]hept-5-en-2-ylamino)-5-[2-(8-methyl-3,4-dihydroquinolin-1(2H)-yl)-2-oxoethyl]-1,3-thiazol-4(5H)-one,
2-(bicyclo[2.2.1]hept-5-en-2-ylamino)-5-(2-oxo-2-piperidin-1-ylethyl)-1,3-thiazol-4(5H)-one,
2-[2-(bicyclo[2.2.1]hept-5-en-2-ylamino)-4-oxo-4,5-dihydro-1,3-thiazol-5-yl]-N-isopropyl-N-phenylacetamide,
2-[2-(bicyclo[2.2.1]hept-5-en-2-ylamino)-4-oxo-4,5-dihydro- ,3-thiazol-5-yl]-N-(2,6-difluorophenyl)acetamide,
N-(2-Chlorophenyl)-2-[2-(cyclohexylamino)-4-oxo-4,5-dihydro-1,3-thiazol-5-yl]acetamide,
2-[2-(Bicyclo[2.2.1]hept-5-en-2-ylamino)-4-oxo-4,5-dihydro-1,3-thiazol-5-yl]-N-phenylacetamide,
2-[2-(Bicyclo[2.2.1]hept-5-en-2-ylamino)-4-oxo-4,5-dihydro-1,3-thiazol-5-yl]-N-1H-indazol-6-ylacetamide,
2-[2-(Bicyclo[2.2.1]hept-5-en-2-ylamino)-4-oxo-4,5-dihydro-1,3-thiazol-5-yl]-N-(4-fluorophenyl)acetamide,
N-(2-benzoylphenyl)-2-[2-(Bicyclo[2.2.1]hept-5-en-2-ylamino)-4-oxo-4,5-dihydro-1,3-thiazol-5-yl]acetamide,
3-({[2-(Bicyclo[2.2.1]hept-5-en-2-ylamino)-4-oxo-4,5-dihydro-1,3-thiazol-5-yl]acetyl}amino)benzoic acid,
2-[2-(Bicyclo[2.2.1]hept-5-en-2-ylamino)-4-oxo-4,5-dihydro-1,3-thiazol-5-yl]-N-ethylacetamide,
2-[2-(Bicyclo[2.2.1]hept-5-en-2-ylamino)-4-oxo-4,5-dihydro-1,3-thiazol-5-yl]-N-methylacetamide,
2-(Bicyclo[2.2.1]hept-5-en-2-ylamino)-5-(2-morpholin-4-yl-2-oxoethyl)-1,3-thiazol-4(5H)-one,
2-[2-(Bicyclo[2.2.1]hept-5-en-2-ylamino)-4-oxo-4,5-dihydro-1,3-thiazol-5-yl]-N-(2-chlorophenyl)-N-methylacetamide,
2-(Bicyclo[2.2.1]hept-2-ylamino)-5-[2-(3,4-dihydroquinolin-1(2H)-yl)-2-oxoethyl]-1,3-thiazol-4(5H)-one,
2-[(3-chloro-2-methylphenyl)amino]-5-[2-(3,4-dihydroquinolin-1(2H)-yl)-2-oxoethyl]-1,3-thiazol-4(5H)-one,
2-[2-(1-Adamantylamino)-4-oxo-4,5-dihydro-1,3-thiazol-5-yl]-N-methyl-N-phenylacetamide,
2-[2-(1-adamantylamino)-4-oxo-4,5-dihydro-1,3-thiazol-5-yl]-N-(2,6-difluorophenyl)acetamide,
2-[2-(tert-butylamino)-4-oxo-4,5-dihydro-1,3-thiazol-5-yl]-N-methyl-N-phenylacetamide,
2-[2-(cyclopropylamino)-4-oxo-4,5-dihydro-1,3-thiazol-5-yl]-N-methyl-N-phenylacetamide,
2-(cyclopentylamino)-5-[2-(3,4-dihydroisoquinolin-2(1H)-yl)-2-oxoethyl]-1,3-thiazol-4(5H)-one,
2-[2-(cyclopentylamino)-4-oxo-4,5-dihydro-1,3-thiazol-5-yl]-N-methyl-N-phenylacetamide,
5-[2-(3,4-dihydroisoquinolin-2(1H)-yl)-2-oxoethyl]-2-(isobutylamino)-1,3-thiazol-4(5H)-one,
2-[2-(isobutylamino)-4-oxo-4,5-dihydro-1,3-thiazol-5-yl]-N-methyl-N-phenylacetamide,
2-(1-adamantylamino)-5-[2-(3,4-dihydroquinolin-1(2H)-yl)-2-oxoethyl]-1,3-thiazol-4(5H)-one,
2-(cyclopropylamino)-5-[2-(3,4-dihydroisoquinolin-2(1H)-yl)-2-oxoethyl]-1,3-thiazol-4(5H)-one,
5-[2-(3,4-dihydroquinolin-1(2H)-yl)-2-oxoethyl]-2-(mesitylamino)-1,3-thiazol-4(5H)-one,
N-(2-chlorophenyl)-2-{2-[(2-methylbutyl)amino]-4-oxo-4,5-dihydro-1,3-thiazol-5-yl}acetamide,
N-methyl-2-{2-[(2-methylbutyl)amino]-4-oxo-4,5-dihydro-1,3-thiazol-5-yl}-N-phenylacetamide,
5-[2-(3,4-dihydroquinolin-1(2H)-yl)-2-oxoethyl]-2-[(2-methylbutyl)amino]-1,3-thiazol-4(5H)-one,
N-methyl-2-{4-oxo-2-[(2-phenylethyl)amino]-4,5-dihydro-1,3-thiazol-5-yl}-N-phenylacetamide,
5-[2-(3,4-dibydroquinolin-1(2H)-yl)-2-oxoethyl]-2-[(2-phenylethyl)amino]-1,3-thiazol-4(5H)-one,
2-(2-{[3,5-Bis(trifluoromethyl)phenyl]amino}-4-oxo-4,5-dihydro-1,3-thiazol-5-yl)-N-(2-chloro-6-fluorobenzyl)acetamide trifluoroacetate,
5-[2-(3,4-dihydroquinolin-1(2H)-yl)-2-oxoethyl]-2-[(4-morpholin-4-ylphenyl)amino]-1,3-thiazol-4(5H)-one,
5-[2-(3,4-Dihydroquinolin-1(2H)-yl)-2-oxoethyl]-2-{[3,5-bis(trifluoromethyl)phenyl]amino}-1,3-thiazol-4(5H)-one,
2-(2-{[3,5-Bis(trifluoromethyl)phenyl]amino}-4-oxo-4,5-dihydro-1,3-thiazol-5-yl)-N-(2-phenylethyl)acetamide,
5-[2-(3,4-Dihydroquinolin-1(2H)-yl)-2-oxoethyl]-2-[(2-fluorophenyl)amino]-1,3-thiazol-4(5H)-one,
N-(2-Chloro-6-fluorobenzyl)-2-{2-[(2-fluorophenyl)amino]-4-oxo-4,5-dihydro-1,3-thiazol-5-yl}acetamide,
2-{2-[(2-Fluorophenyl)amino]-4-oxo-4,5-dihydro-1,3-thiazol-5-yl}-N-(4-methylcyclohexyl)acetamide,
5-[2-(3,4-dihydroquinolin-1(2H)-yl)-2-oxoethyl]-2-[(2,6-dimethylphenyl)amino]-1,3-thiazol-4(5H)-one,
N-(2-chloro-6-fluorobenzyl)-2-{2-[(2,6-dimethylphenyl)amino]-4-oxo-4,5-dihydro-1,3-thiazol-5-yl}acetamide,
5-[2-(3,4-Dihydroquinolin-1(2H)-yl)-2-oxoethyl]-2-[(2-methylphenyl)amino]-1,3-thiazol-4(5H)-one,
5-(2-Azepan-1-yl-2-oxoethyl)-2-[(2-methylphenyl)amino]-1,3-thiazol-4(5H)-one,
N-(2-Chloro-6-fluorobenzyl)-2-{2-[(2-methylphenyl)amino]-4-oxo-4,5-dihydro-1,3-thiazol-5-yl}acetamide,
5-(2-Azepan-1-yl-2-oxoethyl)-2-[(2-isopropylphenyl)amino]-1,3-thiazol-4(5H)-one,
N-benzyl-2-{2-[(cyclohexylmethyl)amino]-4-oxo-4,5-dihydro-1,3-thiazol-5-yl}acetamide,
2-(cyclohexylamino)-5-[2-(3,4-dihydroquinolin-1(2H)-yl)-2-oxoethyl]-1,3-thiazol-4(5H)-one,
2-[2-(cyclohexylamino)-4-oxo-4,5-dihydro-1,3-thiazol-5-yl]-N-methyl-N-phenylacetamide,
N-methyl-2-{4-oxo-2-[(6-phenoxypyridin-3-yl)amino]-4,5-dihydro-1,3-thiazol-5-yl}-N-phenylacetamide,
N-(2-chloro-6-fluorobenzyl)-2-{4-oxo-2-[(6-phenoxypyridin-3-yl)amino]-4,5-dihydro-1,3-thiazol-5-yl}acetamide,
2-[(2-cyclohex-1-en-1-ylethyl)amino]-5-[2-(3,4-dihydroquinolin-1(2H)-yl)-2-oxoethyl]-1,3-thiazol-4(5H)-one,
N-(4-fluorophenyl)-2-{4-oxo-2-[(1,1,3,3-tetramethylbutyl)amino]-4,5-dihydro-1,3-thiazol-5-yl}acetamide,
5-(2-azepan-1-yl-2-oxoethyl)-2-(cyclohexylamino)-1,3-thiazol-4(5H)-one,
5-[2-(3,4-dihydroquinolin-1(2H)-yl)-2-oxoethyl]-2-[(1,1,3,3-tetramethylbutyl)amino]-1,3-thiazol-4(5H)-one,
5-(2-azepan-1-yl-2-oxoethyl)-2-[(1,1,3,3-tetramethylbutyl)amino]-1,3-thiazol-4(5H)-one,
2-(cyclohexylamino)-5-[2-(3,4-dihydroisoquinolin-2(1H)-yl)-2-oxoethyl]-1,3-thiazol-4(5H)-one,
N-benzyl-2-[2-(cyclohexylamino)-4-oxo-4,5-dihydro-1,3-thiazol-5-yl]-N-methylacetamide,
2-(cyclohexylamino)-5-[2-(6,7-dimethoxy-3,4-dihydroisoquinolin-2(1H)-yl)-2-oxoethyl]-1,3-thiazol-4(5H)-one,
5-[2-(4-benzylpiperidin-1-yl)-2-oxoethyl]-2-(cyclohexylamino)-1,3-thiazol-4(5H)-one,
5-(2-Azepan-1-yl-2-oxoethyl)-2-(tricyclo[3.3.1.0˜3,7˜]non-3-ylamino)-1,3-thiazol-4(5H)-one,
2-[2-(cyclopentylamino)-4-oxo-4,5-dihydro-1,3-thiazol-5-yl]-N-(2,6-difluorophenyl)acetamide,
2-{2-[(4-chlorobenzyl)amino]-4-oxo-4,5-dihydro-1,3-thiazol-5-yl}-N-1-naphthylacetamide,
5-[2-(3,4-dihydroquinolin-1(2H)-yl)-2-oxoethyl]-2-[(2-morpholin-4-ylethyl)amino]-1,3-thiazol-4(5H)-one,
2-[2-(sec-butylamino)-4-oxo-4,5-dihydro-1,3-thiazol-5-yl]-N-methyl-N-phenylacetamide trifluoroacetate,
2-(sec-butylamino)-5-[2-(3,4-dihydroquinolin-1(2H)-yl)-2-oxoethyl]-1,3-thiazol-4(5H)-one trifluoroacetate,
2-[2-(sec-butylamino)-4-oxo-4,5-dihydro-1,3-thiazol-5-yl]-N-(2-chlorophenyl)acetamide trifluoroacetate,
2-{2-[(cyclopropylmethyl)amino]-4-oxo-4,5-dihydro-1,3-thiazol-5-yl}-N-methyl-N-phenylacetamide trifluoroacetate,
2-{2-[(4-methylbenzyl)amino]-4-oxo-4,5-dihydro-1,3-thiazol-5-yl}-N-phenylacetamide,
2-{2-[(4-chlorobenzyl)amino]-4-oxo-4,5-dihydro-1,3-thiazol-5-yl}-N-phenylacetamide,
5-[2-(3,4-Dihydroisoquinolin-2(1H)-yl)-2-oxoethyl]-2-(tricyclo[3.3.1.0˜3,7˜]non-3-ylamino)-1,3-thiazol-4(5H)-one,
2-(cycloheptylamino)-5-[2-(3,4-dihydroisoquinolin-2(1H)-yl)-2-oxoethyl]-1,3-thiazol-4(5H)-one,
5-(2-azepan-1-yl-2-oxoethyl)-2-(cycloheptylamino)-1,3-thiazol-4(5H)-one,
5-(2-azepan-1-yl-2-oxoethyl)-2-[(cyclohexylmethyl)amino]-1,3-thiazol-4(5H)-one,
2-[2-(cycloheptylamino)-4-oxo-4,5-dihydro-1,3-thiazol-5-yl]-N-methyl-N-phenylacetamide,
5-[2-(4-Methylpiperidin-1-yl)-2-oxoethyl]-2-(tricyclo[3.3.1.0˜3,7˜]non-3-ylamino)-1,3-thiazol-4(5H)-one,
5-[2-(1,3-Dihydro-2H-isoindol-2-yl)-2-oxoethyl]-2-(tricyclo[3.3.1.0˜3,7˜]non-3-ylamino)-1,3-thiazol-4(5H)-one,
2-[(cyclohexylmethyl)amino]-5-(2-oxo-2-pyrrolidin-1-ylethyl)-1,3-thiazol-4(5H)-one,
2-[(cyclohexylmethyl)amino]-5-[2-(3,4-dihydroisoquinolin-2(1H)-yl)-2-oxoethyl]-1,3-thiazol-4(5H)-one,
2-[2-(Dicyclohexylamino)-4-oxo-4,5-dihydro-1,3-thiazol-5-yl]-N-benzylacetamide,
2-azepan-1-yl-5-(2-azepan-1-yl-2-oxoethyl)-1,3-thiazol-4(5H)-one,
2-azepan-1-yl-5-[2-(3,4-dihydroquinolin-1(2H)-yl)-2-oxoethyl]-1,3-thiazol-4(5H)-one,
2-azepan-1-yl-5-[2-(3,4-dihydroisoquinolin-2(1H)-yl)-2-oxoethyl]-1,3-thiazol-4(5H)-one,
2-(cycloheptylamino)-5-[2-(2,3-dihydro-1H-indol-1-yl)-2-oxoethyl]-1,3-thiazol-4(5H)-one,
2-(cycloheptylamino)-5-(2-oxo-2-pyrrolidin-1-ylethyl)-1,3-thiazol-4(5H)-one,
2-(cycloheptylamino)-5-[2-(4-methylpiperidin-1-yl)-2-oxoethyl]-1,3-thiazol-4(5H)-one,
2-[2-(Dicyclohexylamino)-4-oxo-4,5-dihydro-1,3-thiazol-5-yl]-N-phenylacetamide,
N-cyclohexyl-2-{2-[(cyclohexylmethyl)amino]-4-oxo-4,5-dihydro-1,3-thiazol-5-yl}acetamide,
N-Cyclohexyl-N-ethyl-2-[4-oxo-2-(tricyclo[3.3.1.0˜3,7˜]non-3-ylamino)-4,5-dihydro-1,3-thiazol-5-yl]acetamide,
N-(Cyclopropylmethyl)-N-propylacetamide-2-[4-oxo-2-(tricyclo[3.3.1.0˜3,7˜]non-3-ylamino)-4,5-dihydro-1,3-thiazol-5-yl]acetamide,
5-(2-Azocan-1-yl-2-oxoethyl)-2-(tricyclo[3.3.1.0˜3,7˜]non-3-ylamino)-1,3-thiazol-4(5H)-one,
5-[2-(1-Oxa-4-azaspiro[4.5]dec-4-yl)-2-oxoethyl]-2-(tricyclo[3.3.1.0˜3,7˜]non-3-ylamino)-1,3-thiazol-4(5H)-one,
2-{[(1R)-1-cyclohexylethyl]amino}-5-[2-(3,4-dihydroquinolin-1(2H)-yl)-2-oxoethyl]-1,3-thiazol-4(5H)-one,
2-{[(1R)-1-cyclohexylethyl]amino}-5-[2-(3,4-dihydroisoquinolin-2(1H)-yl)-2-oxoethyl]-1,3-thiazol-4(5H)-one,
5-(2-azepan-1-yl-2-oxoethyl)-2-{[(1R)-1-cyclohexylethyl]amino}-1,3-thiazol-4(5H)-one,
2-{[(1S)-1-cyclohexylethyl]amino}-5-[2-(3,4-dihydroquinolin-1(2H)-yl)-2-oxoethyl]-1,3-thiazol-4(5H)-one,
2-{[(1S)-1-cyclohexylethyl]amino}-5-[2-(3,4-dihydroisoquinolin-2(1H)-yl)-2-oxoethyl]-1,3-thiazol-4(5H)-one,
5-(2-azepan-1-yl-2-oxoethyl)-2-{[(1S)-1-cyclohexylethyl]amino}-1,3-thiazol-4(5H)-one,
2-[(cyclohexylmethyl)amino]-5-[2-(4-methylpiperidin-1-yl)-2-oxoethyl]-1,3-thiazol-4(5H)-one,
N-cyclohexyl-2-{2-[(cyclohexylmethyl)amino]-4-oxo-4,5-dihydro-1,3-thiazol-5-yl}-N-ethylacetamide,
2-[(cyclohexylmethyl)amino]-5-[2-(1-oxa-4-azaspiro[4.5]dec-4-yl)-2-oxoethyl]-1,3-thiazol-4(5H)-one,
5-(2-azocan-1-yl-2-oxoethyl)-2-[(cyclohexylmethyl)amino]-1,3-thiazol-4(5H)-one,
2-[(cyclohexylmethyl)amino]-5-[2-(1,3-dihydro-2H-isoindol-2-yl)-2-oxoethyl]-1,3-thiazol-4(5H)-one,
N-(3-chloro-2-methylbenzyl)-2-{2-[(cyclohexylmethyl)amino]-4-oxo-4,5-dihydro-1,3-thiazol-5-yl}acetamide,
N-(cyclohexylmethyl)-2-{2-[(cyclohexylmethyl)amino]-4-oxo-4,5-dihydro-1,3-thiazol-5-yl}acetamide,
2-[(cyclohexylmethyl)amino]-5-[2-(octahydroisoquinolin-2(1H)-yl)-2-oxoethyl]-1,3-thiazol-4(5H)-one,
N-[(1R,2R,4S)-bicyclo[2.2.1]hept-2-yl]-2-{2-[(cyclohexylmethyl)amino]-4-oxo-4,5-dihydro-1,3-thiazol-5-yl}acetamide,
4-{[2-(cycloheptylamino)-4-oxo-4,5-dihydro-1,3-thiazol-5-yl]acetyl}-1,4-diazepan-1-ium trifluoroacetate,
2-[2-(cycloheptylamino)-4-oxo-4,5-dihydro-1,3-thiazol-5-yl]-N-(cyclopropylmethyl)-N-propylacetamide,
2-(cycloheptylamino)-5-[2-(1,3-dihydro-2H-isoindol-2-yl)-2-oxoethyl]-1,3-thiazol-4(5H)-one,
5-(2-azocan-1-yl-2-oxoethyl)-2-(cycloheptylamino)-1,3-thiazol-4(5H)-one,
2-[2-(cycloheptylamino)-4-oxo-4,5-dihydro-1,3-thiazol-5-yl]-N-cyclohexyl-N-ethylacetamide,
2-{2-[(cyclohexylmethyl)amino]-4-oxo-4,5-dihydro-1,3-thiazol-5-yl}-N-methyl-N-phenylacetamide,
2-{2-[(cyclohexylmethyl)amino]-4-oxo-4,5-dihydro-1,3-thiazol-5-yl}-N-(4-methoxyphenyl)-N-methylacetamide,
2-{2-[(cyclohexylmethyl)amino]-4-oxo-4,5-dihydro-1,3-thiazol-5-yl}-N-ethyl-N-phenylacetamide,
N-butyl-2-{2-[(cyclohexylmethyl)amino]-4-oxo-4,5-dihydro-1,3-thiazol-5-yl}-N-phenylacetamide,
N-butyl-2-[2-(cycloheptylamino)-4-oxo-4,5-dihydro-1,3-thiazol-5-yl]-N-phenylacetamide,
N-benzyl-2-[2-(cycloheptylamino)-4-oxo-4,5-dihydro-1,3-thiazol-5-yl]-N-phenylacetamide,
5-[2-(1,3-dihydro-2H-isoindol-2-yl)-2-oxoethyl]-2-[(2,2,3,3-tetramethylcyclopropyl)amino]-1,3-thiazol-4(5H)-one,
5-(2-azepan-1-yl-2-oxoethyl)-2-[(2,2,3,3-tetramethylcyclopropyl)amino]-1,3-thiazol-4(5H)-one,
2-[2-(bicyclo[2.2.1]hept-5-en-2-ylamino)-4-oxo-4,5-dihydro-1,3-thiazol-5-yl]ethyl benzoate,
2-[2-(bicyclo[2.2.1]hept-5-en-2-ylamino)-4-oxo-4,5-dihydro-1,3-thiazol-5-yl]ethyl 2-chlorobenzoate,
2-[2-(bicyclo[2.2.1]hept-5-en-2-ylamino)-4-oxo-4,5-dihydro-1,3-thiazol-5-yl]ethyl 3,4-dichlorobenzoate,
2-[2-(bicyclo[2.2.1]hept-5-en-2-ylamino)-4-oxo-4,5-dihydro-1,3-thiazol-5-yl]ethyl 2,6-difluorobenzoate,
2-[2-(bicyclo[2.2.1]hept-5-en-2-ylamino)-4-oxo-4,5-dihydro-1,3-thiazol-5-yl]ethyl 2,5-bis(trifluoromethyl)benzoate,
2-[2-(bicyclo[2.2.1]hept-5-en-2-ylamino)-4-oxo-4,5-dihydro-1,3-thiazol-5-yl]ethyl 3,4-difluorobenzoate trifluoroacetate,
2-[2-(bicyclo[2.2.1]hept-5-en-2-ylamino)-4-oxo-4,5-dihydro-1,3-thiazol-5-yl]ethyl 3,4-difluorobenzoate,
2-[2-(bicyclo[2.2.1]hept-5-en-2-ylamino)-4-oxo-4,5-dihydro-1,3-thiazol-5-yl]ethyl 2,5-difluorobenzoate,
2-[2-(bicyclo[2.2.1]hept-5-en-2-ylamino)-4-oxo-4,5-dihydro-1,3-thiazol-5-yl]ethyl 4-methylbenzoate,
2-[2-(bicyclo[2.2.1]hept-5-en-2-ylamino)-4-oxo-4,5-dihydro-1,3-thiazol-5-yl]ethyl 4-chloro-3-nitrobenzoate,
2-[2-(bicyclo[2.2.1]hept-5-en-2-ylamino)-4-oxo-4,5-dihydro-1,3-thiazol-5-yl]ethyl 3-methylbenzoate,
2-[2-(bicyclo[2.2.1]hept-5-en-2-ylamino)-4-oxo-4,5-dihydro-1,3-thiazol-5-yl]ethyl 3-(trifluoromethyl)benzoate,
2-[2-(bicyclo[2.2.1]hept-5-en-2-ylamino)-4-oxo-4,5-dihydro-1,3-thiazol-5-yl]ethyl 2,3,4-trifluorobenzoate,
2-[2-(bicyclo[2.2.1]hept-5-en-2-ylamino)-4-oxo-4,5-dihydro-1,3-thiazol-5-yl]ethyl 2-bromo-5-methoxybenzoate,
2-[2-(bicyclo[2.2.1]hept-5-en-2-ylamino)-4-oxo-4,5-dihydro-1,3-thiazol-5-yl]ethyl 2-chloro-6-fluorobenzoate,
2-[2-(bicyclo[2.2.1]hept-5-en-2-ylamino)-4-oxo-4,5-dihydro-1,3-thiazol-5-yl]ethyl 2-fluoro-5-(trifluoromethyl)benzoate,
2-[2-(bicyclo[2.2.1]hept-5-en-2-ylamino)-4-oxo-4,5-dihydro-1,3-thiazol-5-yl]ethyl 2-fluoro-4-(trifluoromethyl)benzoate,
2-[2-(bicyclo[2.2.1]hept-5-en-2-ylamino)-4-oxo-4,5-dihydro-1,3-thiazol-5-yl]ethyl 3-methoxybenzoate,
2-[2-(bicyclo[2.2.1]hept-5-en-2-ylamino)-4-oxo-4,5-dihydro-1,3-thiazol-5-yl]ethyl 2,6-dimethoxybenzoate,
2-[2-(bicyclo[2.2.1]hept-5-en-2-ylamino)-4-oxo-4,5-dihydro-1,3-thiazol-5-yl]ethyl 2,4-dimethoxybenzoate,
2-[2-(bicyclo[2.2.1]hept-5-en-2-ylamino)-4-oxo-4,5-dihydro-1,3-thiazol-5-yl]ethyl 4-butoxybenzoate,
2-[2-(bicyclo[2.2.1]hept-5-en-2-ylamino)-4-oxo-4,5-dihydro-1,3-thiazol-5-yl]ethyl 3,5-bis(trifluoromethyl)benzoate,
2-[2-(bicyclo[2.2.1]hept-5-en-2-ylamino)-4-oxo-4,5-dihydro-1,3-thiazol-5-yl]ethyl 4-tert-butylbenzoate,
2-[2-(bicyclo[2.2.1]hept-5-en-2-ylamino)-4-oxo-4,5-dihydro-1,3-thiazol-5-yl]ethyl 2,4-dichlorobenzoate,
2-[2-(bicyclo[2.2.1]hept-5-en-2-ylamino)-4-oxo-4,5-dihydro-1,3-thiazol-5-yl]ethyl 2,4,6-trichlorobenzoate,
2-[2-(bicyclo[2.2.1]hept-5-en-2-ylamino)-4-oxo-4,5-dihydro-1,3-thiazol-5-yl]ethyl (2-chlorophenyl)carbamate,
2-[2-(bicyclo[2.2.1]hept-5-en-2-ylamino)-4-oxo-4,5-dihydro-1,3-thiazol-5-yl]ethyl (4-chloro-3-nitrophenyl)carbamate,
2-[2-(bicyclo[2.2.1]hept-5-en-2-ylamino)-4-oxo-4,5-dihydro-1,3-thiazol-5-yl]ethyl (4-bromo-2,6-difluorophenyl)carbamate,
2-[2-(bicyclo[2.2.1]hept-5-en-2-ylamino)-4-oxo-4,5-dihydro-1,3-thiazol-5-yl]ethyl (3-phenoxyphenyl)carbamate,
N-{2-[2-(cycloheptylamino)-4-oxo-4,5-dihydro-1,3-thiazol-5-yl]ethyl}-N′-(2-fluorophenyl)urea,
N-{2-[2-(cycloheptylamino)-4-oxo-4,5-dihydro-1,3-thiazol-5-yl]ethyl}-N′-(4-fluorophenyl)urea,
N-{2-[2-(cycloheptylamino)-4-oxo-4,5-dihydro-1,3-thiazol-5-yl]ethyl}-N′-(2,6-difluorophenyl)urea,
N-{2-[2-(cycloheptylamino)-4-oxo-4,5-dihydro-1,3-thiazol-5-yl]ethyl}-N′-(2,4-difluorophenyl)urea,
N-(2-chlorophenyl)-N′-{2-[2-(cycloheptylamino)-4-oxo-4,5-dihydro-1,3-thiazol-5-yl]ethyl}urea,
N-[2-chloro-5-(trifluoromethyl)phenyl]-N′-{2-[2-(cycloheptylamino)-4-oxo-4,5-dihydro-1,3-thiazol-5-yl]ethyl}urea,
N-{2-[2-(cycloheptylamino)-4-oxo-4,5-dihydro-1,3-thiazol-5-yl]ethyl}-N′-[4-fluoro-2-(trifluoromethyl)phenyl]urea,
N-{2-[2-(cycloheptylamino)-4-oxo-4,5-dihydro-1,3-thiazol-5-yl]ethyl}-N′-(2-methoxyphenyl)urea,
N-(5-chloro-2-methoxyphenyl)-N′-{2-[2-(cycloheptylamino)-4-oxo-4,5-dihydro-1,3-thiazol-5-yl]ethyl}urea,
N-{2-[2-(cycloheptylamino)-4-oxo-4,5-dihydro-1,3-thiazol-5-yl]ethyl}-N′-(2,4-dimethoxyphenyl)urea,
N-{2-[2-(cycloheptylamino)-4-oxo-4,5-dihydro-1,3-thiazol-5-yl]ethyl}-N′-(2,6-dichloropyridin-4-yl)urea,
N-{2-[2-(cycloheptylamino)-4-oxo-4,5-dihydro-1,3-thiazol-5-yl]ethyl}-N′-cyclohexylurea,
N-{2-[2-(cycloheptylamino)-4-oxo-4,5-dihydro-1,3-thiazol-5-yl]ethyl}-N′-cyclopentylurea,
2-(bicyclo[2.2.1]hept-5-en-2-ylamino)-5-{2-[(2-chlorobenzyl)oxy]ethyl}-1,3-thiazol-4(5H)-one,
2-(bicyclo[2.2.1]hept-5-en-2-ylamino)-5-{2-[(2-methylbenzyl)oxy]ethyl}-1,3-thiazol-4(5H)-one trifluoroacetate,
2-(bicyclo[2.2.1]hept-5-en-2-ylamino)-5-{2-[(2-methoxybenzyl)oxy]ethyl}-1,3-thiazol-4(5H)-one,
2-(bicyclo[2.2.1]hept-5-en-2-ylamino)-5-(2-{[3-(dimethylamino)benzyl]oxy}ethyl)-1,3-thiazol-4(5H)-one,
5-(Bicyclo[2.2.1]hept-5-en-2-ylamino)-5-[2-(2-chlorophenoxy)ethyl]-1,3-thiazol-4(5H)-one,
2-(bicyclo[2.2.1]hept-5-en-2-ylamino)-5-(2-hydroxyethyl)-1,3-thiazol-4(5H)-one,
2-(Bicyclo[2.2.1]hept-5-en-2-ylamino)-5-[2-(4-phenoxyphenoxy)ethyl]-1,3-thiazol-4(5H)-one,
Methyl 4-{2-[2-(bicyclo[2.2.1]hept-5-en-2-ylamino)-4-oxo-4,5-dihydro-1,3-thiazol-5-yl]ethoxy}-3-chlorobenzoate,
2-(Bicyclo[2.2.1]hept-5-en-2-ylamino)-5-[2-(4-chloro-3-methylphenoxy)ethyl]-1,3-thiazol-4(5H)-one,
2-Chlorophenyl [2-(bicyclo[2.2.1]hept-5-en-2-ylamino)-4-oxo-4,5-dihydro-1,3-thiazol-5-yl]acetate,
Phenyl [2-(bicyclo[2.2.1]hept-5-en-2-ylamino)-4-oxo-4,5-dihydro-1,3-thiazol-5-yl]acetate,
2-Methoxyphenyl [2-(bicyclo[2.2.1]hept-5-en-2-ylamino)-4-oxo-4,5-dihydro-1,3-thiazol-5-yl]acetate,
2-Morpholin-4-ylphenyl [2-(bicyclo[2.2.1]hept-5-en-2-ylamino)-4-oxo-4,5-dihydro-1,3-thiazol-5-yl]acetate,
2-(Bicyclo[2.2.1]hept-5-en-2-ylamino)-5-[2-(4-chlorophenyl)-2-oxoethyl]-1,3-thiazol-4(5H)-one,
5-(2-aminoethyl)-2-(bicyclo[2.2.1]hept-5-en-2-ylamino)-1,3-thiazol-4(5H)-one,
2-(2-aminoethyl)-2-(cyclohexylamino)-1,3-thiazol-4(5H)-one,
2-(bicyclo[2.2.1]hept-5-en-2-ylamino)-5-(2-bromoethyl)-1,3-thiazol-4(5H)-one,
2-(bicyclo[2.2.1]hept-2-ylamino)-5-(2-hydroxyethyl)-1,3-thiazol-4(5H)-one,
[2-(Bicyclo[2.2.1]hept-5-en-2-ylamino)-4-oxo-4,5-dihydro-1,3-thiazol-5-yl]acetic acid,
3-(2-Anilinoethyl)-2-[(2-methylphenyl)amino]-1,3-thiazol-4(5H)-one hydrobromide,
5-(2-Anilinoethyl)-2-[(2-methoxyphenyl)amino]-1,3-thiazol-4(5H)-one hydrobromide,
5-(2-anilinoethyl)-2-(2,3-dihydro-1H-inden-2-ylamino)-1,3-thiazol-4(5H)-one,
2-Anilino-5-(2-anilinoethyl)-1,3-thiazol-4(5H)-one hydrobromide,
N-(2-Chlorophenyl)-2-(4-oxo-2-piperidin-1-yl-4,5-dihydro-1,3-thiazol-5-yl)acetamide,
2-{2-[(3-chloro-2-methylphenyl)amino]-4-oxo-4,5-dihydro-1,3-thiazol-5-yl}-N-methyl-N-phenylacetamide,
2-(2-{[3,5-Bis(trifluoromethyl)phenyl]amino}-4-oxo-4,5-dihydro-1,3-thiazol-5-yl)-N-methyl-N-phenylacetamide trifluoroacetate,
2-{2-[(2,6-dimethylphenyl)amino]-4-oxo-4,5-dihydro-1,3-thiazol-5-yl}-N-phenylacetamide,
N-methyl-2-{2-[(4-morpholin-4-ylphenyl)amino]-4-oxo-4,5-dihydro-1,3-thiazol-5-yl}-N-phenylacetamide,
N-Benzyl-2-(2-{[3,5-bis(trifluoromethyl)phenyl]amino}-4-oxo-4,5-dihydro-1,3-thiazol-5-yl)acetamide,
2-{2-[(2-Fluorophenyl)amino]-4-oxo-4,5-dihydro-1,3-thiazol-5-yl}-N-methyl-N-phenylacetamide,
2-{2-[(2,6-dimethylphenyl)amino]-4-oxo-4,5-dihydro-1,3-thiazol-5-yl}-N-methyl-N-phenylacetamide,
2-[2-(mesitylamino)-4-oxo-4,5-dihydro-1,3-thiazol-5-yl]-N-methyl-N-phenylacetamide,
N-1-naphthyl-2-[2-(1-naphthylamino)-4-oxo-4,5-dihydro-1,3-thiazol-5-yl]acetamide,
2-(2-anilino-4-oxo-4,5-dihydro-1,3-thiazol-5-yl)-N-methyl-N-phenylacetamide,
2-(2-anilino-4-oxo-4,5-dihydro-1,3-thiazol-5-yl)-N-(2-chlorophenyl)acetamide,
N-methyl-2-[2-(1-naphthylamino)-4-oxo-4,5-dihydro-1,3-thiazol-5-yl]-N-phenylacetamide,
2-Anilino-5-[2-(3,4-dihydroquinolin-1(2H)-yl)-2-oxoethyl]-1,3-thiazol-4(5H)-one,
2-(2-Anilino-4-oxo-4,5-dihydro-1,3-thiazol-5-yl)-N-phenylacetamide,
5-[2-(3,4-dihydroisoquinolin-2(1H)-yl)-2-oxoethyl]-2-piperidin-1-yl-1,3-thiazol-4(5H)-one,
5-[2-(3,4-dihydroquinolin-1(2H)-yl)-2-oxoethyl]-2-piperidin-1-yl-1,3-thiazol-4(5H)-one,
5-(2-morpholin-4-yl-2-oxoethyl)-2-[(2,2,3,3-tetramethylcyclopropyl)amino]-1,3-thiazol-4(5H)-one,
N-[(2-{[(1S)-1-cyclohexylethyl]amino}-4-oxo-4,5-dihydro-1,3-thiazol-5-yl)methyl]-2-methoxybenzamide,
2-(cyclooctylamino)-5-(2-morpholin-4-yl-2-oxoethyl)-1,3-thiazol-4(5H)-one,
2-[2-(bicyclo[2.2.1]hept-2-ylamino)-4-oxo-4,5-dihydro-1,3-thiazol-5-yl]-N-cycloheptylacetamide hydrochloride,
N-[(2-{[(1S)-1-cyclohexylethyl]amino}-4-oxo-4,5-dihydro-1,3-thiazol-5-yl)methyl]-2-fluorobenzamide,
2-fluoro-N-(2-{4-oxo-2-[(2,2,3,3-tetramethylcyclopropyl)amino]-4,5-dihydro-1,3-thiazol-5-yl}ethyl)benzamide,
2-(1-adamantylamino)-5-[2-(3,4-dihydroquinolin-1(2H)-yl)-2-oxoethyl]-1,3-thiazol-4(5H)-one,
2-[2-(bicyclo[2.2.1]hept-2-ylamino)-4-oxo-4,5-dihydro-1,3-thiazol-5-yl]-N-(2-chloro-6-fluorobenzyl)acetamide hydrochloride,
2-(cycloheptylamino)-5-[2-(3,4-dihydroquinolin-1(2H)-yl)-2-oxoethyl]-1,3-thiazol-4(5H)-one,
2-(cyclooctylamino)-5-[2-(3,4-dihydroquinolin-1(2H)-yl)-2-oxoethyl]-1,3-thiazol-4(5H)-one,
N-cyclohexyl-2-[2-(cyclooctylamino)-4-oxo-4,5-dihydro-1,3-thiazol-5-yl]-N-ethylacetamide,
5-(2-azepan-1-yl-2-oxoethyl)-2-(bicyclo[2.2.1]hept-2-ylamino)-1,3-thiazol-4(5H)-one hydrochloride,
N-cyclohexyl-N-ethyl-2-{4-oxo-2-[(2,2,3,3-tetramethylcyclopropyl)amino]-4,5-dihydro-1,3-thiazol-5-yl}acetamide,
2-chloro-N-{[2-(cyclooctylamino)-4-oxo-4,5-dihydro-1,3-thiazol-5-yl]methyl}-6-fluorobenzamide,
5-[2-(4-methylpiperidin-1-yl)-2-oxoethyl]-2-[(2,2,3,3-tetramethylcyclopropyl)amino]-1,3-thiazol-4(5H)-one,
2-chloro-N-{[2-(cycloheptylamino)-4-oxo-4,5-dihydro-1,3-thiazol-5-yl]methyl}-6-fluorobenzamide,
5-(2-anilinoethyl)-2-[(2,2,3,3-tetramethylcyclopropyl)amino]-1,3-thiazol-4(5H)-one,
2-chloro-6-fluoro-N-(2-{4-oxo-2-[(2,2,3,3-tetramethylcyclopropyl)amino]-4,5-dihydro-1,3-thiazol-5-yl}ethyl)benzamide,
5-(2-azepan-1-yl-2-oxoethyl)-2-(cyclooctylamino)-1,3-thiazol-4(5H)-one,
2-chloro-N-{2-[2-(cyclooctylamino)-4-oxo-4,5-dihydro-1,3-thiazol-5-yl]ethyl}-6-fluorobenzamide,
2-chloro-N-{2-[2-(cyclooctylamino)-4-oxo-4,5-dihydro-1,3-thiazol-5-yl]ethyl}benzenesulfonamide,
2-[2-(bicyclo[2.2.1]hept-5-en-2-ylamino)-4-oxo-4,5-dihydro-1,3-thiazol-5-yl]ethyl 2,4-dichlorobenzoate,
N-{2-[2-(cyclooctylamino)-4-oxo-4,5-dihydro-1,3-thiazol-5-yl]ethyl}-2,6-difluorobenzenesulfonamide,
N-{2-[2-(cyclooctylamino)-4-oxo-4,5-dihydro-1,3-thiazol-5-yl]ethyl}-2,6-difluorobenzamide,
2-(bicyclo[2.2.1]hept-5-en-2-ylamino)-5-[2-(2-chlorophenoxy)ethyl]-1,3-thiazol-4(5H)-one,
2-chloro-N-{2-[2-(cyclooctylamino)-4-oxo-4,5-dihydro-1,3-thiazol-5-yl]ethyl}benzamide,
5-[2-(4-benzylpiperidin-1-yl)-2-oxoethyl]-2-(cycloheptylamino)-1,3-thiazol-4(5H)-one,
N-{2-[2-(bicyclo[2.2.1]hept-5-en-2-ylamino)-4-oxo-4,5-dihydro-1,3-thiazol-5-yl]ethyl}-2,4-dichlorobenzamide,
2-chlorophenyl [2-(bicyclo[2.2.1]hept-5-en-2-ylamino)-4-oxo-4,5-dihydro-1,3-thiazol-5-yl]acetate,
N-{2-[2-(bicyclo[2.2.1]hept-5-en-2-ylamino)-4-oxo-4,5-dihydro-1,3-thiazol-5-yl]ethyl}-2-chlorobenzamide,
N-{2-[2-(cyclooctylamino)-4-oxo-4,5-dihydro-1,3-thiazol-5-yl]ethyl}-5-methyl-3-phenylisoxazole-4-carboxamide,
5-[2-(4-benzylpiperidin-1-yl)-2-oxoethyl]-2-[(cyclohexylmethyl)amino]-1,3-thiazol-4(5H)-one,
methyl 4-{2-[2-(bicyclo[2.2.1]hept-5-en-2-ylamino)-4-oxo-4,5-dihydro-1,3-thiazol-5-yl]ethoxy}-3-chlorobenzoate,
phenyl [2-(bicyclo[2.2.1]hept-5-en-2-ylamino)-4-oxo-4,5-dihydro-1,3-thiazol-5-yl]acetate,
N-{2-[2-(bicyclo[2.2.1]hept-5-en-2-ylamino)-4-oxo-4,5-dihydro-1,3-thiazol-5-yl]ethyl}-2-bromo-5-methoxybenzamide,
N-{2-[2-(cyclooctylamino)-4-oxo-4,5-dihydro-1,3-thiazol-5-yl]ethyl}-2-phenoxyacetamide,
2-(cycloheptylamino)-5-[2-(1-oxa-4-azaspiro[4.5]dec-4-yl)-2-oxoethyl]-1,3-thiazol-4(5H)-one,
N-{2-[2-(bicyclo[2.2.1]hept-5-en-2-ylamino)-4-oxo-4,5-dihydro-1,3-thiazol-5-yl]ethyl}-2-fluoro-4-(trifluoromethyl)benzamide,
2-(bicyclo[2.2.1]hept-5-en-2-ylamino)-5-[2-(4-chloro-3-methylphenoxy)ethyl]-1,3-thiazol-4(5H)-one,
2-[2-(cycloheptylamino)-4-oxo-4,5-dihydro-1,3-thiazol-5-yl]-N-ethyl-N-phenylacetamide,
N-(2-chloro-6-fluorobenzyl)-2-[2-(cycloheptylamino)-4-oxo-4,5-dihydro-1,3-thiazol-5-yl]acetamide,
2-methoxyphenyl [2-(bicyclo[2.2.1]hept-5-en-2-ylamino)-4-oxo-4,5-dihydro-1,3-thiazol-5-yl]acetate,
N-{2-[2-(cyclooctylamino)-4-oxo-4,5-dihydro-1,3-thiazol-5-yl]ethyl}adamantane-1-carboxamide,
5-[2-(3,4-dihydroquinolin-1(2H)-yl)-2-oxoethyl]-2-[(2-fluorophenyl)amino]-1,3-thiazol-4(5H)-one,
N-{2-[2-(bicyclo[2.2.1]hept-5-en-2-ylamino)-4-oxo-4,5-dihydro-1,3-thiazol-5-yl]ethyl}-2,5-difluotobenzamide,
5-(2-anilinoethyl)-2-{[1-(4-chlorophenyl)cyclobutyl]amino}-1,3-thiazol-4(5H)-one hydrobromide,
N-{2-[2-(cycloheptylamino)-4-oxo-4,5-dihydro-1,3-thiazol-5-yl]ethyl}-2,5-difluorobenzamide,
2-[2-(cycloheptylamino)-4-oxo-4,5-dihydro-1,3-thiazol-5-yl]-N-(4-methoxyphenyl)-N-methylacetamide,
2-(bicyclo[2.2.1]hept-5-en-2-ylamino)-5-[2-(4-phenoxyphenoxy)ethyl]-1,3-thiazol-4(5H)-one,
4-chloro-N-{2-[2-(cyclooctylamino)-4-oxo-4,5-dihydro-1,3-thiazol-5-yl]ethyl}benzamide,
N-{2-[2-(cyclooctylamino)-4-oxo-4,5-dihydro-1,3-thiazol-5-yl]ethyl}cyclohexanecarboxamide,
N-{2-[2-(cyclooctylamino)-4-oxo-4,5-dihydro-1,3-thiazol-5-yl]ethyl}-4-(trifluoromethyl)benzamide,
2-{[3,5-bis(trifluoromethyl)phenyl]amino}-5-[2-(3,4-dihydroquinolin-1(2H)-yl)-2-oxoethyl]-1,3-thiazol-4(5H)-one
2-anilino-5-[2-(1,3-dihydro-2H-isoindol-2-yl)-2-oxoethyl]-1,3-thiazol-4(5H)-one,
2-(cycloheptylamino)-5-[2-(4-hydroxy-4-phenylpiperidin-1-yl)-2-oxoethyl]-1,3-thiazol-4(5H)-one,
2-{2-[(cyclohexylmethyl)amino]-4-oxo-4,5-dihydro-1,3-thiazol-5-yl}-N,N-diethylacetamide,
N-{2-[2-(cyclooctylamino)-4-oxo-4,5-dihydro-1,3-thiazol-5-yl]ethyl}-2,2-dimethylpropanamide,
2-anilino-5-(2-azepan-1-yl-2-oxoethyl)-1,3-thiazol-4(5H)-one,
3-morpholin-4-ylphenyl [2-(bicyclo[2.2.1]hept-5-en-2-ylamino)-4-oxo-4,5-dihydro-1,3-thiazol-5-yl]acetate,
N-{2-[2-(bicyclo[2.2.1]hept-5-en-2-ylamino)-4-oxo-4,5-dihydro-1,3-thiazol-5-yl]ethyl}-4-cyanobenzamide,
N-{2-[2-(cyclooctylamino)-4-oxo-4,5-dihydro-1,3-thiazol-5-yl]ethyl}-4-methoxybenzamide,
2-(bicyclo[2.2.1]hept-5-en-2-ylamino)-5-{2-[(2-chlorobenzyl)oxy]ethyl}-1,3-thiazol-4(5H)-one,
2-{2-[(2-fluorophenyl)amino]-4-oxo-4,5-dihydro-1,3-thiazol-5-yl}-N-(4-methylcyclohexyl)acetamide,
2-[(cyclohexylmethyl)amino]-5-(2-morpholin-4-yl-2-oxoethyl)-1,3-thiazol-4(5H)-one,
2-(cycloheptylamino)-5-isobutyl-1,3-thiazol-4(5H)-one,
(5R)-2-(cycloheptylamino)-5-(cyclohexylmethyl)-1,3-thiazol-4(5H)-one,
(5S)-2-(cycloheptylamino)-5-(cyclohexylmethyl)-1,3-thiazol-4(5H)-one,
2-(cyclooctylamino)-5-(4-hydroxybenzyl)-1,3-thiazol-4(5H)-one,
2-(cycloheptylamino)-5-(1H-indol-3-ylmethyl)-1,3-thiazol-4(5H)-one,
2-(cycloheptylamino)-5-(4-hydroxybenzyl)-1,3-thiazol-4(5H)-one,
2-(bicyclo[2.2.1]hept-2-ylamino)-5-(4-hydroxybenzyl)-1,3-thiazol-4(5H)-one,
2-(cycloheptylamino)-5-(3,4-dihydroxybenzyl)-1,3-thiazol-4(5H)-one,
2-(cycloheptylamino)-5-(pyridin-3-ylmethyl)-1,3-thiazol-4(5H)-one,
2-(cyclooctylamino)-5-propyl-1,3-thiazol-4(5H)-one hydrobromide,
5-butyl-2-(cyclooctylamino)-1,3-thiazol-4(5H)-one hydrobromide,
2-(bicyclo[2.2.1]hept-2-ylamino)-5-ethyl-1,3-thiazol-4(5H)-one hydrobromide,
2-(cyclohexylamino)-5-ethyl-1,3-thiazol-4(5H)-one hydrobromide,
5-ethyl-2-[(2-methylphenyl)amino]-1,3-thiazol-4(5H)-one,
(5S)-2-(cycloheptylamino)-5-methyl-1,3-thiazol-4(5H)-one,
5-ethyl-2-[(2-isopropylphenyl)amino]-1,3-thiazol-4(5H)-one,
2-(cyclooctylamino)-5-methyl-1,3-thiazol-4(5H)-one,
2-(cyclooctylamino)-5-ethyl-1,3-thiazol-4(5H)-one,
2-(cycloheptylamino)-5-ethyl-1,3-thiazol-4(5H)-one,
2-{2-[2-(bicyclo[2.2.1]hept-5-en-2-ylamino)-4-oxo-4,5-dihydro-1,3-thiazol-5-yl]ethyl}-1H-isoindole-1,3(2H)-dione,
5-{[5-(2-chlorophenyl)-1,3,4-oxadiazol-2-yl]methyl}-2-[(2-fluorophenyl)amino]-1,3-thiazol-4(5H)-one,
5-{[5-(2-chlorophenyl)-1,3,4-oxadiazol-2-yl]methyl}-2-(tricyclo[3.3.1.0˜3,7˜]non-3-ylamino)-1,3-thiazol-4(5H)-one,
5-{[5-(2-chlorophenyl)-1,3,4-oxadiazol-2-yl]methyl}-2-{[(1S,2S,3S,5R)-2,6,6-trimethylbicyclo[3.1.1]hept-3-yl]amino}-1,3-thiazol-4(5H)-one,
2-(bicyclo[2.2.1]hept-2-ylamino)-5-{[5-(2-chlorophenyl)-1,3,4-oxadiazol-2-yl]methyl}-1,3-thiazol-4(5H)-one,
5-{[5-(2-chlorophenyl)-1,3,4-oxadiazol-2-yl]methyl}-2-{[(1R,2R,3R,5S)-2,6,6-trimethylbicyclo[3.1.1]hept-3-yl]amino}-1,3-thiazol-4(5H)-one,
5-(1H-benzimidazol-2-ylmethyl)-2-(cyclohexylamino)-1,3-thiazol-4(5H)-one,
2-anilino-5-(1,3-benzoxazol-2-ylmethyl)-1,3-thiazol-4(5H)-one,
5-(1,3-benzoxazol-2-ylmethyl)-2-(cycloheptylamino)-1,3-thiazol-4(5H)-one,
2-anilino-5-(1,3-benzothiazol-2-ylmethyl)-1,3-thiazol-4(5H)-one,
5-(1H-benzimidazol-2-ylmethyl)-2-(bicyclo[2.2.1]hept-2-ylamino)-1,3-thiazol-4(5H)-one,
5-(1H-benzimidazol-2-ylmethyl)-2-(cycloheptylamino)-1,3-thiazol-4(5H)-one,
N-{2-[2-(cyclooctylamino)-4-oxo-4,5-dihydro-1,3-thiazol-5-yl]ethyl}-2-fluorobenzamide,
N-{2-[2-(cyclooctylamino)-4-oxo-4,5-dihydro-1,3-thiazol-5-yl]ethyl}-2,6-difluoro-N-methylbenzamide,
2-chloro-N-{2-[2-(cyclooctylamino)-4-oxo-4,5-dihydro-1,3-thiazol-5-yl]ethyl}-N-methylbenzamide, and
2,4-dichloro-N-{2-[2-(cyclooctylamino)-4-oxo-4,5-dihydro-1,3-thiazol-5-yl]ethyl}-N-methylbenzamide.

10. The compound according to claim 6 for use in the prophylaxis or treatment of a 11-β-hydroxysteroid dehydrogenase type 1 enzyme-mediated disorder or achieving immuno-modulation.

11. The compound according to claim 10, wherein the disorder is selected from diabetes, syndrome X, obesity, glaucoma, hyperlipidemia, hyperglycemia, hyperinsulinemia, hypertension, osteoporosis, dementia, depression, virus diseases, and inflammatory diseases.

12. The compound according to claim 10 for the treatment or prophylaxis of a medical disorder involving delayed or impaired wound healing.

13. The compound according to claim 12, wherein the medical disorder involving delayed or impaired wound healing is diabetes.

14. The compound according to claim 12, wherein the medical disorder involving delayed or impaired wound healing is caused by treatment with glucocorticoids.

15. The compound according to claim 10 for the promotion of wound healing in chronic wounds, such as diabetic ulcers, venous ulcers or pressure ulcers.

16. The compound according to claim 10, wherein the immuno-modulation is selected from tuberculosis, lepra, and psoriasis.

17. A pharmaceutical formulation comprising a compound according to claim 6 as active ingredient, in combination with a pharmaceutically acceptable diluent or carrier.

18. A pharmaceutical formulation for use in the prophylaxis or treatment of a 11-β-hydroxysteroid dehydrogenase type 1 enzyme-mediated disorder or achieving immuno-modulation.

19. The pharmaceutical formulation according to claim 18, wherein the disorder is selected from diabetes, syndrome X, obesity, glaucoma, hyperlipidemia, hyperglycemia, hyperinsulinemia, hypertension, osteoporosis, dementia, depression, virus diseases, and inflammatory diseases.

20. The pharmaceutical formulation according to claim 18 for the treatment or prophylaxis of a medical disorder involving delayed or impaired wound healing.

21. The pharmaceutical formulation according to claim 20, wherein the medical disorder involving delayed or impaired wound healing is diabetes.

22. The pharmaceutical formulation according to claim 20, wherein the medical disorder involving delayed or impaired wound healing is caused by treatment with glucocorticoids.

23. The pharmaceutical formulation according to claim 18 for the promotion of wound healing in chronic wounds, such as diabetic ulcers, venous ulcers or pressure ulcers.

24. The pharmaceutical formulation according to claim 18, wherein the immuno-modulation is selected from tuberculosis, lepra, and psoriasis.

25. A method for the prophylaxis or treatment of a 11-β-hydroxysteroid dehydrogenase type 1 enzyme-mediated disorder and or achieving immuno-modulation comprising administering the compound of claim 6 to an individual.

26. The method according to claim 25, wherein the disorder is selected from diabetes, syndrome X, obesity, glaucoma, hyperlipidemia, hyperglycemia, hyperinsulinemia, hypertension, osteoporosis, dementia, depression, virus diseases, and inflammatory diseases.

27. The method according to claim 25, wherein the disorder involves delayed or impaired wound healing.

28. The method according to claim 27, wherein the disorder involving delayed or impaired wound healing is diabetes.

29. The method according to claim 27, wherein the disorder involving delayed or impaired wound healing is caused by treatment with glucocorticoids.

30. The method according to claim 25, wherein the compound is used for the promotion of wound healing in chronic wounds, such as diabetic ulcers, venous ulcers or pressure ulcers.

31. The method according to claim 25, wherein the individual suffers from a disorder selected from tuberculosis, lepra, and psoriasis.

32. A method for inhibiting a 11-β-hydroxysteroid dehydrogenase type 1 enzyme, which comprises administering to a subject in need of such treatment an effective amount of a compound according to claim 6.

33. Use of a compound according to claim 6 for the manufacture of a medicament for use in the prophylaxis or treatment of a 11-β-hydroxysteroid dehydrogenase type 1 enzyme-mediated disorder or achieving immuno-modulation.

34. The use according to claim 33, wherein the disorder is selected from diabetes, syndrome X, obesity, glaucoma, hyperlipidemia, hyperglycemia, hyperinsulinemia, hypertension, osteoporosis, dementia, depression, virus diseases, and inflammatory diseases.

35. The use according to claim 33 for the treatment or prophylaxis of a disorder involving delayed or impaired wound healing.

36. The use according to claim 35, wherein the disorder involving delayed or impaired wound healing is diabetes.

37. The use according to claim 35, wherein the disorder involving delayed or impaired wound healing is caused by treatment with glucocorticoids.

38. The use according to claim 33 for the promotion of wound healing in chronic wounds, such as diabetic ulcers, venous ulcers or pressure ulcers.

39. The use according to claim 33, wherein the immuno-modulation is selected from tuberculosis, lepra, and psoriasis.
